(19) United States
(12) Patent Application Publication
(10) Pub. No.: US 2021/0252123 A1
(43) Pub. Date: Aug. 19, 2021

(54) ARID1A, CDKN2A, KMT2B, KMT2D, TP53 and PTEN VACCINES FOR CANCER

(71) Applicant: Frame Pharmaceuticals B.V., Amsterdam (NL)

(72) Inventor: Ronald Hans Anton PLASTERK, Amsterdam (NL)

(21) Appl. No.: 17/262,999

(22) PCT Filed: Jul. 25, 2019
(86) PCT No.: PCT/NL2019/050496

§ 371 Date: Jan. 25, 2021

(30) Foreign Application Priority Data
Jul. 26, 2018 (NL) 2021400
Jan. 24, 2019 (NL) 2022447
Apr. 5, 2019 (EP) 19167615.4
Apr. 5, 2019 (EP) 19167620.4

(51) Int. Cl. A61K 39/00; C12Q 1/6886

(52) U.S. Cl. CPC A61K 39/0011; A61K 39/39; C12Q 1/6886

(57) ABSTRACT

The invention relates to the field of cancer. In particular, it relates to the field of immune system directed approaches for tumor reduction and control. Some aspects of the invention relate to vaccines, vaccinations and other means of stimulating an antigen specific immune response against a tumor in individuals. Such vaccines comprise neoantigens resulting from frameshift mutations that bring out-of-frame sequences of the ARID1A, CDKN2A, KMT2B, KMT2D, TP53 and PTEN genes in-frame. Such vaccines are also useful for ‘off the shelf’ use.

### FIELD OF THE INVENTION

The invention relates to the field of cancer. In particular, it relates to the field of immune system directed approaches for tumor reduction and control. Some aspects of the invention relate to vaccines, vaccinations and other means of stimulating an antigen specific immune response against a tumor in individuals.

Such vaccines comprise neoantigens resulting from frameshift mutations that bring out-of-frame sequences of the ARID1A, CDKN2A, KMT2B, KMT2D, TP53 and PTEN genes in-frame. Such vaccines are also useful for ‘off the shelf’ use.

### BACKGROUND OF THE INVENTION

There are a number of different existing cancer therapies, including ablation techniques (e.g., surgical procedures and radiation) and chemical techniques (e.g., pharmaceutical agents and antibodies), and various combinations of such techniques. Despite intensive research such therapies are still frequently associated with serious risk, adverse or toxic side effects, as well as varying efficacy.

There is a growing interest in cancer therapies that aim to target cancer cells with a patient's own immune system (such as cancer vaccines or checkpoint inhibitors, or T-cell based immunotherapy). Such therapies may indeed eliminate some of the known disadvantages of existing therapies, or be used in addition to the existing therapies for additional therapeutic effect. Cancer vaccines or immunogenic compositions intended to treat an existing cancer by strengthening the body's natural defenses against the cancer and based on tumor-specific neoantigens hold great promise as next-generation of personalized cancer immunotherapy. Evidence shows that such neoantigen-based vaccination can elicit T-cell responses and can cause tumor regression in patients.

Typically the immunogenic compositions/vaccines are composed of tumor antigens (antigenic peptides or nucleic acids encoding them) and may include immune stimulatory molecules like cytokines that work together to induce antigen-specific cytotoxic T-cells that target and destroy tumor cells. Vaccines containing tumor-specific and patient-specific neoantigens require the sequencing of the patients' genome and tumor genome in order to determine whether the neoantigen is tumor specific, followed by the production of personalized compositions.

Sequencing, identifying the patient's specific neoantigens and preparing such personalized compositions may require a substantial amount of time, time which may unfortunately not be available to the patient, given that for some tumors the average survival time after diagnosis is short, sometimes around a year or less.

Accordingly, there is a need for improved methods and compositions for providing subject-specific immunogenic compositions/cancer vaccines. In particular it would be desirable to have available a vaccine for use in the treatment of cancer, wherein such vaccine is suitable for treatment of a larger number of patients, and can thus be prepared in advance and provided off the shelf. There is a clear need in the art for personalized vaccines which induce an immune response to tumor specific neoantigens. One of the objects of the present disclosure is to provide personalized cancer vaccines that can be provided off the shelf. An additional object of the present disclosure is to provide cancer vaccines that can be provided prophylactically. Such vaccines are especially useful for individuals that are at risk of developing cancer.

### SUMMARY OF THE INVENTION

In a preferred embodiment, the disclosure provides a vaccine for use in the treatment of cancer, said vaccine comprising:

(i) a peptide, or a collection of tiled peptides, having the amino acid sequence selected from Sequence 29, an amino acid sequence having 90% identity to Sequence 29, or a fragment thereof comprising at least 10 consecutive amino acids of Sequence 29; and

a peptide, or a collection of tiled peptides, having the amino acid sequence selected from Sequence 30, an amino acid sequence having 90% identity to Sequence 30, or a fragment thereof comprising at least 10 consecutive amino acids of Sequence 30; preferably also comprising

a peptide, or a collection of tiled peptides, having the amino acid sequence selected from Sequences 31-33, an amino acid sequence having 90% identity to Sequences 31-33, or a fragment thereof comprising at least 10 consecutive amino acids of Sequences 31-33;

(ii) a peptide, or a collection of tiled peptides, having the amino acid sequence selected from Sequence 130, an amino acid sequence having 90% identity to Sequence 130, or a fragment thereof comprising at least 10 consecutive amino acids of Sequence 130; and

a peptide, or a collection of tiled peptides, having the amino acid sequence selected from Sequence 131, an amino acid sequence having 90% identity to Sequence, or a fragment thereof comprising at least 10 consecutive amino acids of Sequence,

(iii) a peptide, or a collection of tiled peptides, having the amino acid sequence selected from Sequence 157, an amino acid sequence having 90% identity to Sequence 157, or a fragment thereof comprising at least 10 consecutive amino acids of Sequence 157; and

a peptide, or a collection of tiled peptides, having the amino acid sequence selected from Sequence 158, an amino acid sequence having 90% identity to Sequence 158, or a fragment thereof comprising at least 10 consecutive amino acids of Sequence 158;

(iv) a peptide, or a collection of tiled peptides, having the amino acid sequence selected from Sequence 273, an amino acid sequence having 90% identity to Sequence 273, or a fragment thereof comprising at least 10 consecutive amino acids of Sequence 273; and

a peptide, or a collection of tiled peptides, having the amino acid sequence selected from Sequence 274, an amino acid sequence having 90% identity to Sequence 274, or a fragment thereof comprising at least 10 consecutive amino acids of Sequence 274;

(v) a peptide, or a collection of tiled peptides, having the amino acid sequence selected from Sequence 528, an amino acid sequence having 90% identity to Sequence 528, or a fragment thereof comprising at least 10 consecutive amino acids of Sequence 528; and

a peptide, or a collection of tiled peptides, having the amino acid sequence selected from Sequence 529, an amino acid sequence having 90% identity to Sequence 529, or a fragment thereof comprising at least 10 consecutive amino acids of Sequence 529 and/or

(vi) a peptide, or a collection of tiled peptides, comprising an amino acid sequence selected from Sequences 1-28, an amino acid sequence having 90% identity to Sequences 1-28, or a fragment thereof comprising at least 10 consecutive amino acids of Sequences 1-28 (i.e., TP53 neo-open reading frame peptides).

In a preferred embodiment, the disclosure provides a collection of frameshift-mutation peptides comprising:

(i) a peptide, or a collection of tiled peptides, having the amino acid sequence selected from Sequence 29, an amino acid sequence having 90% identity to Sequence 29, or a fragment thereof comprising at least 10 consecutive amino acids of Sequence 29; and

a peptide, or a collection of tiled peptides, having the amino acid sequence selected from Sequence 30, an amino acid sequence having 90% identity to Sequence 30, or a fragment thereof comprising at least 10 consecutive amino acids of Sequence 30; preferably also comprising

a peptide, or a collection of tiled peptides, having the amino acid sequence selected from Sequences 31-33, an amino acid sequence having 90% identity to Sequences 31-33, or a fragment thereof comprising at least 10 consecutive amino acids of Sequences 31-33;

(ii) a peptide, or a collection of tiled peptides, having the amino acid sequence selected from Sequence 130, an amino acid sequence having 90% identity to Sequence 130, or a fragment thereof comprising at least 10 consecutive amino acids of Sequence 130; and

a peptide, or a collection of tiled peptides, having the amino acid sequence selected from Sequence 131, an amino acid sequence having 90% identity to Sequence, or a fragment thereof comprising at least 10 consecutive amino acids of Sequence,

(iii) a peptide, or a collection of tiled peptides, having the amino acid sequence selected from Sequence 157, an amino acid sequence having 90% identity to Sequence 157, or a fragment thereof comprising at least 10 consecutive amino acids of Sequence 157; and

a peptide, or a collection of tiled peptides, having the amino acid sequence selected from Sequence 158, an amino acid sequence having 90% identity to Sequence 158, or a fragment thereof comprising at least 10 consecutive amino acids of Sequence 158;

(iv) a peptide, or a collection of tiled peptides, having the amino acid sequence selected from Sequence 273, an amino acid sequence having 90% identity to Sequence 273, or a fragment thereof comprising at least 10 consecutive amino acids of Sequence 273; and

a peptide, or a collection of tiled peptides, having the amino acid sequence selected from Sequence 274, an amino acid sequence having 90% identity to Sequence 274, or a fragment thereof comprising at least 10 consecutive amino acids of Sequence 274; and/or

(v) a peptide, or a collection of tiled peptides, having the amino acid sequence selected from Sequence 528, an amino acid sequence having 90% identity to Sequence 528, or a fragment thereof comprising at least 10 consecutive amino acids of Sequence 528; and

a peptide, or a collection of tiled peptides, having the amino acid sequence selected from Sequence 529, an amino acid sequence having 90% identity to Sequence 529, or a fragment thereof comprising at least 10 consecutive amino acids of Sequence 529.

In one embodiment, the disclosure provides a collection of TP53 frameshift-mutation peptides comprising: at least two peptides, wherein each peptide, or a collection of tiled peptides, comprises a different amino acid sequence selected from Sequences 1-3, an amino acid sequence having 90% identity to Sequences 1-3, or a fragment thereof comprising at least 10 consecutive amino acids of Sequences 1-3. Preferably, said collection further comprises a peptide, or a collection of tiled peptides, having the amino acid sequence selected from Sequence 4, an amino acid sequence having 90% identity to Sequence 4, or a fragment thereof comprising at least 10 consecutive amino acids of Sequence 4. Preferably, said collection further comprises one or more of Sequences 5-15. In some embodiments, the collection of TP53 frameshift-mutation peptides further comprises one or more ARID1A frameshift-mutation peptides as disclosed herein, one or more CDKN2A frameshift-mutation peptides as disclosed herein, one or more KMT2B frameshift-mutation peptides as disclosed herein, one or more KMT2D frameshift-mutation peptides as disclosed herein, and/or one or more PTEN frameshift-mutation peptides as disclosed herein.

In a preferred embodiment, the disclosure provides a peptide comprising an amino acid sequence selected from the groups:

(i) Sequences 29-129, an amino acid sequence having 90% identity to Sequences 29-129, or a fragment thereof comprising at least 10 consecutive amino acids of Sequences 29-129;

(ii) Sequences 130-156, an amino acid sequence having 90% identity to Sequences 130-156, or a fragment thereof comprising at least 10 consecutive amino acids of Sequences 130-156;

(iii) Sequences 157-272, an amino acid sequence having 90% identity to Sequences 157-272, or a fragment thereof comprising at least 10 consecutive amino acids of Sequences 157-272;

(iv) Sequences 273-527, an amino acid sequence having 90% identity to Sequences 273-527, or a fragment thereof comprising at least 10 consecutive amino acids of Sequences 273-527; and

(v) Sequences 528-558, an amino acid sequence having 90% identity to Sequences 528-558, or a fragment thereof comprising at least 10 consecutive amino acids of Sequences 528-558.

In one embodiment, the disclosure provides a peptide, or a collection of tiled peptides, comprising an amino acid sequence selected from Sequences 1-28, an amino acid sequence having 90% identity to Sequences 1-28, or a fragment thereof comprising at least 10 consecutive amino acids of Sequences 1-28 (i.e., TP53 neo-open reading frame peptides).

Preferably the peptide is a peptide, or a collection of tiled peptides, having the amino acid sequence selected from Sequence 130, an amino acid sequence having 90% identity to Sequence 130, or a fragment thereof comprising at least 10 consecutive amino acids of Sequence 130, or a collection comprising said peptide.

In some embodiments of the disclosure, the peptides are linked, preferably wherein said peptides are comprised within the same polypeptide.

In a preferred embodiment, the disclosure provides one more isolated nucleic acid molecules encoding the peptides or collection of peptides as disclosed herein. In a preferred embodiment, the disclosure provides one or more vectors comprising the nucleic acid molecules disclosed herein, preferably wherein the vector is a viral vector. In a preferred embodiment, the disclosure provides a host cell comprising the isolated nucleic acid molecules or the vectors as disclosed herein.

In a preferred embodiment, the disclosure provides a binding molecule or a collection of binding molecules that bind the peptide or collection of peptides disclosed herein, where in the binding molecule is an antibody, a T-cell receptor, or an antigen binding fragment thereof.

In a preferred embodiment, the disclosure provides a chimeric antigen receptor or collection of chimeric antigen receptors each comprising i) a T cell activation molecule; ii) a transmembrane region; and iii) an antigen recognition moiety; wherein said antigen recognition moieties bind the peptide or collection of peptides disclosed herein. In a preferred embodiment, the disclosure provides a host cell or combination of host cells that express the binding molecule or collection of binding molecules, or the chimeric antigen receptor or collection of chimeric antigen receptors as disclosed herein.

In a preferred embodiment, the disclosure provides a vaccine or collection of vaccines comprising the peptide or collection of peptides, the nucleic acid molecules, the vectors, or the host cells as disclosed herein; and a pharmaceutically acceptable excipient and/or adjuvant, preferably an immune-effective amount of adjuvant.

In a preferred embodiment, the disclosure provides the vaccines as disclosed herein for use in the treatment of cancer in an individual. In a preferred embodiment, the disclosure provides the vaccines as disclosed herein for prophylactic use in the prevention of cancer in an individual. In a preferred embodiment, the disclosure provides the vaccines as disclosed herein for use in the preparation of a medicament for treatment of cancer in an individual or for prophylactic use. In a preferred embodiment, the disclosure provides methods of treating an individual for cancer or reducing the risk of developing said cancer, the method comprising administering to the individual in need thereof a therapeutically effective amount of a vaccine as disclosed herein.

In a preferred embodiment, the individual has cancer and one or more cancer cells of the individual:
(i) expresses a peptide having the amino acid sequence selected from Sequences 29-558, an amino acid sequence having 90% identity to any one of Sequences 29-558, or a fragment thereof comprising at least 10 consecutive amino acids of amino acid sequence selected from Sequences 29-558;
(ii) or comprises a DNA or RNA sequence encoding an amino acid sequences of (i).

In one embodiment, the individual has cancer and one or more cancer cells of the individual:
(i) expresses a peptide having the amino acid sequence selected from Sequences 1-28, an amino acid sequence having 90% identity to any one of Sequences 1-28, or a fragment thereof comprising at least 10 consecutive amino acids of amino acid sequence selected from Sequences 1-28;
(ii) or comprises a DNA or RNA sequence encoding an amino acid sequences of (i).

In one embodiment, the disclosure provides the vaccines as disclosed herein for prophylactic use in the prevention of cancer in an individual. In one embodiment, the disclosure provides the vaccines as disclosed herein for use in the preparation of a medicament for prophylactic use. In one embodiment, the disclosure provides methods of treating an individual for cancer or reducing the risk of developing said cancer, the method comprising administering to the individual in need thereof a therapeutically effective amount of a vaccine as disclosed herein. In some embodiments, the individual prophylactically administered a vaccine as disclosed herein has not been diagnosed with cancer. In some embodiments, the individual at risk of developing cancer has a germline mutation in a gene that increases the chance that the individual will develop cancer, preferably the mutation is in one or more of the following genes: TP53, BRCA1, BRCA2, CHEK2, MLH1, MSH2, MSHG, PMS1, PMS2, ERCC1, CDKN2A, XPA, FANCG, BAP1, POLD1, EPCAM, MAP2K2, SH2B3, PRDM9, PTCH1, RAD51D, PRF1, PTEN, PALB2, ERCC4, DIS3L2, TRIM37, NTHL1, FANCC, BRIP1, NBN, ERCC2, FANCD2, SDHA, UROD, DROSHA, ATM, DICER1, WRN, BRCA2, APC, ATR, ABCB11, SUFU, RAD51C, POLE, RET, MPL, XPC, SMARCA4, FH, HMBS, NF1, POT1, FAH, GJB2, CBL, RECQL, FANCM, KIT, RECQL4, MUTYH, DOCK8, RB1, ERCC3, EXT1, ERCC5, SDHB, FANCA, BUB1B, KRAS, ALK, SOS1, CDC73, COL7A1, TMEM127, CYLD, BLM, TSC1, SLC25A13, ITK, FANCI, FANCF, RHBDF2, HFE, SBDS, GBA, FANCL, and FLCN.

In a preferred embodiment, the disclosure provides a method of stimulating the proliferation of human T-cells, comprising contacting said T-cells with the peptide or collection of peptides, the nucleic acid molecules, the vectors, the host cell, or the vaccine as disclosed herein.

In a preferred embodiment, the disclosure provides a storage facility for storing vaccines. Preferably the facility stores at least two different cancer vaccines as disclosed herein. Preferably the storing facility stores:

a vaccine comprising:

(i) a peptide, or a collection of tiled peptides, having the amino acid sequence selected from Sequence 29, an amino acid sequence having 90% identity to Sequence 29, or a fragment thereof comprising at least 10 consecutive amino acids of Sequence 29; and

a peptide, or a collection of tiled peptides, having the amino acid sequence selected from Sequence 30, an amino acid sequence having 90% identity to Sequence 30, or a fragment thereof comprising at least 10 consecutive amino acids of Sequence 30; preferably also comprising

a peptide, or a collection of tiled peptides, having the amino acid sequence selected from Sequences 31-33, an amino acid sequence having 90% identity to Sequences 31-33, or a fragment thereof comprising at least 10 consecutive amino acids of Sequences 31-33;

and one or more vaccines selected from:

a vaccine comprising:

(ii) a peptide, or a collection of tiled peptides, having the amino acid sequence selected from Sequence 130, an amino acid sequence having 90% identity to Sequence 130, or a fragment thereof comprising at least 10 consecutive amino acids of Sequence 130; and

a peptide, or a collection of tiled peptides, having the amino acid sequence selected from Sequence 131, an amino acid sequence having 90% identity to Sequence, or a fragment thereof comprising at least 10 consecutive amino acids of Sequence,

a vaccine comprising:

(iii) a peptide, or a collection of tiled peptides, having the amino acid sequence selected from Sequence 157, an amino acid sequence having 90% identity to Sequence 157, or a fragment thereof comprising at least 10 consecutive amino acids of Sequence 157; and

a peptide, or a collection of tiled peptides, having the amino acid sequence selected from Sequence 158, an amino acid sequence having 90% identity to Sequence 158, or a fragment thereof comprising at least 10 consecutive amino acids of Sequence 158;

a vaccine comprising:

(iv) a peptide, or a collection of tiled peptides, having the amino acid sequence selected from Sequence 273, an amino acid sequence having 90% identity to Sequence 273, or a fragment thereof comprising at least 10 consecutive amino acids of Sequence 273; and

a peptide, or a collection of tiled peptides, having the amino acid sequence selected from Sequence 274, an amino acid sequence having 90% identity to Sequence 274, or a fragment thereof comprising at least 10 consecutive amino acids of Sequence 274; and/or

a vaccine comprising:

(v) a peptide, or a collection of tiled peptides, having the amino acid sequence selected from Sequence 528, an amino acid sequence having 90% identity to Sequence 528, or a fragment thereof comprising at least 10 consecutive amino acids of Sequence 528; and

a peptide, or a collection of tiled peptides, having the amino acid sequence selected from Sequence 529, an amino acid sequence having 90% identity to Sequence 529, or a fragment thereof comprising at least 10 consecutive amino acids of Sequence 529.

In one embodiment, the disclosure provides a storage facility for storing vaccines. Preferably the facility stores at least two different TP53 frameshift-mutation cancer vaccines as disclosed herein. Preferably the storing facility stores a vaccine comprising at least two peptides, wherein each peptide, or a collection of tiled peptides, comprises a different amino acid sequence selected from Sequences 1-3, an amino acid sequence having 90% identity to Sequences 1-3, or a fragment thereof comprising at least 10 consecutive amino acids of Sequences 1-3. In some embodiments, the storage facility also stores one or more, preferably 5 or more, vaccines selected from a peptide, or a collection of tiled peptides, having the amino acid sequence selected from Sequence 4-28, an amino acid sequence having 90% identity to Sequence 4-28, or a fragment thereof comprising at least 10 consecutive amino acids of Sequence 4-28.

In a preferred embodiment, the disclosure provides a method for providing a vaccine for immunizing a patient against a cancer in said patient comprising determining the sequence of ARID1A, CDKN2A, KMT2B, KMT2D, and/or PTEN in cancer cells of said cancer and when the determined sequence comprises a frameshift mutation that produces a neoantigen of Sequence 29-558 or a fragment thereof, providing a vaccine comprising said neoantigen or a fragment thereof. Preferably, the vaccine is obtained from a storage facility as disclosed herein.

In one embodiment, the disclosure provides a method for providing a vaccine for immunizing a patient against a cancer in said patient comprising determining the sequence of TP53 in cancer cells of said cancer and when the determined sequence comprises a frameshift mutation that produces a neoantigen of Sequence 1-28 or a fragment thereof, providing a vaccine comprising said neoantigen or a fragment thereof. Preferably, the vaccine is obtained from a storage facility as disclosed herein.

In a preferred embodiment, the disclosure provides a method of immunizing an individual at risk of developing cancer comprising:
identifying whether said individual has a risk factor for developing cancer,
selecting novel open reading frame peptides associated with an identified risk factor, and
immunizing said individual with
one or more peptides comprising the amino acid sequence of said novel open reading frame peptides,
a collection of tiled peptides comprising said amino acid sequences,
peptide fragments comprising at least 10 consecutive amino acids of said sequences, and/or
one or more nucleic acids encoding said peptides, collection of tiled peptides, or peptide fragments.

Preferably, the risk factor is based on the genetic background of said individual, previous history of cancer in said individual, age of said individual, exposure of said individual to carcinogens, and/or life style risks of said individual.

### REFERENCE TO A SEQUENCE LISTING

The Sequence listing, which is a part of the present disclosure, includes a text file comprising amino acid and/or nucleic acid sequences. The subject matter of the Sequence listing is incorporated herein by reference in its entirety. The information recorded in computer readable form is identical to the written sequence listing. In the event of a discrepancy between the Sequence listing and the description, e.g., in regard to a sequence or sequence numbering, the description (e.g., Table 1) is leading.

### DETAILED DESCRIPTION OF THE DISCLOSED EMBODIMENTS

One issue that may arise when considering personalized cancer vaccines is that once a tumor from a patient has been analysed (e.g. by whole genome or exome sequencing), neoantigens need to be selected and made in a vaccine. This may be a time consuming process, while time is something the cancer patient usually lacks as the disease progresses.

Somatic mutations in cancer can result in neoantigens against which patients can be vaccinated. Unfortunately, the quest for tumor specific neoantigens has yielded no targets that are common to all tumors, yet foreign to healthy cells. Single base pair substitutions (SNVs) at best can alter 1 amino acid which can result in a neoantigen. However, with the exception of rare site-specific oncogenic driver mutations (such as RAS or BRAF) such mutations are private and thus not generalizable.

An “off-the-shelf” solution, where vaccines are available against each potential—neoantigen would be beneficial. The present disclosure is based on the surprising finding that, despite the fact that there are infinite possibilities for frame shift mutations in the human genome, a vaccine can be developed that targets the novel amino acid sequence following a frame shift mutation in a tumor with potential use in a large population of cancer patients.

Neoantigens resulting from frame shift mutations have been previously described as potential cancer vaccines. See, for example, WO95/32731, WO2016172722 (Nantomics), WO2016/187508 (Broad), WO2017/173321 (Neon Therapeutics), US2018340944 (University of Connecticut), and WO2019/012082 (Nouscom), as well as Rahma et al. (Journal of Translational Medicine 2010 8:8) which describes peptides resulting from frame shift mutations in the von Hippel-Lindau tumor suppressor gene (VHL) and Rajasagi et al. (Blood 2014 124(3):453-462) which reports the systematic identification of personal tumor specific neoantigens.

The present disclosure provides a unique set of sequences resulting from frame shift mutations and that are shared among all cancer patients. The finding of shared frame shift sequences is used to define an off-the-shelf pan cancer vaccine that can be used for both therapeutic and prophylactic use in a large number of individuals.

In the present disclosure we provide a source of common neoantigens induced by frame shift mutations, based on analysis of 10,186 TCGA tumor samples and 2774 tumor samples (see Priestley et al. 2019 at https://doi.org/10.1101/415133). We find that these frame shift mutations can produce long neoantigens. These neoantigens are typically new to the body, and can be highly immunogenic. The heterogeneity in the mutations that are found in tumors of different organs or tumors from a single organ in different individuals has always hampered the development of specific medicaments directed towards such mutations. The number of possible different tumorigenic mutations, even in a single gene as P53 was regarded prohibitive for the development of specific treatments. In the present disclosure it was found that many of the possible different frame shift mutations in a gene converge to the same small set of 3′ neo open reading frame peptides (neopeptides or NOPs). We find a fixed set of only 1,244 neopeptides in as much as 30% of all TCGA cancer patients. For some tumor classes this is higher; e.g. for colon and cervical cancer, peptides derived from only ten genes (saturated at 90 peptides) can be applied to 39% of all patients. 50% of all TCGA patients can be targeted at saturation (using all those peptides in the library found more than once). A pre-fabricated library of vaccines (peptide, RNA or DNA) based on this set can provide off the shelf, quality certified, ‘personalized’ vaccines within hours, saving months of vaccine preparation. This is important for critically ill cancer patients with short average survival expectancy after diagnosis.

The concept of utilizing the immune system to battle cancer is very attractive and studied extensively. Indeed, neoantigens can result from somatic mutations, against which patients can be vaccinated 1-11. Recent evidence suggests that frame shift mutations, that result in peptides which are completely new to the body, can be highly immunogenic 12-15. The immune response to neoantigen vaccination, including the possible predictive value of epitope selection has been studied in great detail 8, 13, 16-21 and WO2007/101227, and there is no doubt about the promise of neoantigen-directed immunotherapy. Some approaches find subject-specific neoantigens based on alternative reading frames caused by errors in translation/transcription (WO2004/111075). Others identify subject specific neoantigens based on mutational analysis of the subjects tumor that is to be treated (WO1999/058552; WO2011/143656; US20140170178; WO2016/187508; WO2017/173321). The quest for common antigens, however, has been disappointing, since virtually all mutations are private. For SNV-derived amino acid changes, one can derive algorithms that predict likely good epitopes, but still every case is different.

A change of one amino acid in an otherwise wild-type protein may or may not be immunogenic. The antigenicity depends on a number of factors including the degree of fit of the proteasome-produced peptides in the MHC and ultimately on the repertoire of the finite T-cell system of the patient. In regards to both of these points, novel peptide sequences resulting from a frame shift mutation (referred to herein as novel open reading frames or pNOPs) are a priori expected to score much higher. For example, a fifty amino acid long novel open reading frame sequence is as foreign to the body as a viral antigen. In addition, novel open reading frames can be processed by the proteasome in many ways, thus increasing the chance of producing peptides that bind MHC molecules, and increasing the number of epitopes will be seen by T-cell in the body repertoire.

It is has been established that novel proteins/peptides can arise from frameshift mutations^{32,36}. Furthermore, tumors with a high load of frameshift mutations (micro-satellite instable tumors) have a high density of tumor infiltrating CD8+ T cells³³. In fact, it has been shown that neo-antigens derived from frameshift mutations can elicit cytotoxic T cell responses^{32,34,33}. A recent study demonstrated that a high load of frameshift indels or other mutation types correlates with response to checkpoint inhibitors³⁵.

Binding affinity to MHC class-I molecules was systematically predicted for frameshift indel and point mutations derived neoantigens³⁵. Based on this analysis, neoantigens derived from frameshifts indels result in 3 times more high-affinity MHC binders compared to point mutation derived neoantigens, consistent with earlier work³¹. Almost all frameshift derived neoantigens are so-called mutant-specific binders, which means that cells with reactive T cell receptors for those frameshift neoantigens are (likely) not cleared by immune tolerance mechanisms³⁵. These data are all in favour of neo-peptides from frameshift being superior antigens.

Here we report that frame shift mutations, which are also mostly unique among patients and tumors, nevertheless converge to neo open reading frame peptides (NOPs) from their translation products that surprisingly result in common neoantigens in large groups of cancer patients. The disclosure is based, in part, on the identification of common, tumor specific novel open reading frames resulting from frame shift mutations. Accordingly, the present disclosure provides novel tumor neoantigens and vaccines for the treatment of cancer. In some embodiments, multiple neoantigens corresponding to multiple NOPs can be combined, preferably within a single peptide or a nucleic acid molecule encoding such single peptide. This has the advantage that a large percentage of the patients can be treated with a single vaccine.

While not wishing to be bound by theory, the surprisingly high number of frame shift induced novel open reading frames shared by cancer patients can be explained, at least in part, as follows. Firstly, on the molecular level, different frame shift mutations can lead to the generation of shared novel open reading frames (or sharing at least part of a novel open reading frame). Secondly, the data presented herein suggests that frame shift mutations are strong loss-of-function mutations. This is illustrated in FIG. 2A, where it can be seen that the SNVs in the TCGA database are clustered within the p53 gene, presumably because mutations elsewhere in the gene do not inactive gene function. In contrast, frame shift mutations occur throughout the p53 gene (FIG. 2B). This suggests that frame shift mutations virtually anywhere in the p53 ORF reduce function (splice variants possibly excluded), while not all point mutations in p53 are expected to reduce function. Finally, the process of tumorigenesis naturally selects for loss of function mutations in genes that may suppress tumorigenesis. Interestingly, the present disclosure identifies frame shift mutations in genes that were not previously known as classic tumor suppressors, or that apparently do so only in some tissue tumor types (see, e.g., FIG. 8). These three factors are likely to contribute to the surprisingly high number of frame shift induced novel open reading frames shared by cancer patients; in particular, while frame shift mutations generally represent less than 10% of the mutations in cancer cells, their contribution to neoantigens and potential as vaccines is much higher. The high immunogenic potential of peptides resulting from frameshifts is to a large part attributable to their unique sequence, which is not part of any native protein sequence in humans, and would therefore not be recognised as ‘self’ by the immune system, which would lead to immune tolerance effects. The high immunogenic potential of out-of-frame peptides has been demonstrated in several recent papers.

Neoantigens are antigens that have at least one alteration that makes them distinct from the corresponding wild-type, parental antigen, e.g., via mutation in a tumor cell. A neoantigen can include a polypeptide sequence or a nucleotide sequence

As used herein the term “ORF” refers to an open reading frame. As used herein the term “neoORF” is a tumor-specific ORF (i.e., neoantigen) arising from a frame shift mutation. Peptides arising from such neo ORFs are also referred to herein as neo open reading frame peptides (NOPs) and neoantigens.

A “frame shift mutation” is a mutation causing a change in the frame of the protein, for example as the consequence of an insertion or deletion mutation (other than insertion or deletion of 3 nucleotides, or multitudes thereof). Such frameshift mutations result in new amino acid sequences in the C-terminal part of the protein. These new amino acid sequences generally do not exist in the absence of the frameshift mutation and thus only exist in cells having the mutation (e.g., in tumor cells and pre-malignant progenitor cells).

Novel 3′ neo open reading frame peptides (i.e., NOPs) of TP53, ARID1A, PTEN, KMT2D, KMT2B, and CDKN2A are depicted in table 1. The NOPs, are defined as the amino acid sequences encoded by the longest neo open reading frame sequence identified. Sequences of these NOPs are represented in table 1 as follows:

TP53: Sequences 1-28; more preferably sequences 1-28.

ARID1A: Sequences 29-129; more preferably sequences 29-88.

CDKN2A: Sequences 130-156; more preferably sequences 130-136.

KMT2B: Sequences 157-272, more preferably sequences 157-172.

KMT2D: Sequences 273-527, more preferably sequences 273-306.

PTEN: Sequences 528-558, more preferably sequences 528-544.

The most preferred neoantigens are TP53 frameshift mutation peptides, followed by ARID1A frameshift mutation peptides, followed by KMT2D frameshift mutation peptides, followed by PTEN frameshift mutation peptides, followed by KMT2B frameshift mutation peptides, followed by CDKN2A frameshift mutation peptides. The preference for individual neoantigens directly correlates with the frequency of their occurrence in cancer patients, with TP53 frameshift mutation peptides covering up to 4% of cancer patients, ARID1A frameshift mutation peptides covering up to 3% of cancer patients, KMT2D frameshift mutation peptides covering up to 2.14% of cancer patients, PTEN frameshift mutation peptides covering up to 1.3% of cancer patients, KMT2B frameshift mutation peptides covering up to 1.1% of cancer patients, CDKN2A frameshift mutation peptides covering up to 0.6% of cancer patients.

TABLE 1
Library of NOP sequences
Sequences of NOPs including the percentage of cancer patients
identified in the present study with each NOP. The sequences
referred to herein correspond to the sequence numbering in
the table below. Different predicted alternative splice
forms are indicated as “alt splice x”.

| | | % | | |
|---|---|---|---|---|
| Sequence | Peptide ID | patients | Peptide Sequence | Gene |
| 1 | pNOP36301 | 0.88 | TGGPSSPSSHWKTPVVIYWDGTALRCVFVPV | TP53 |
| | alt | | LGETGAQRKRISARKGSLTTSCPQGALSEHC | |
| | splice a | | PTTPAPLPSQRRNHWMENISPFRSVGVSASR | |
| | | | CSES | |
| 2 | pNOP31232 | 0.83 | TGGPSSPSSHWKTPVVIYWDGTALRCVFVPV | TP53 |
| | alt | | LGETGAQRKRISARKGSLTTSCPQGALSEHC | |
| | splice a | | PTTPAPLPSQRRNHWMENISPFRTRPAFKKK | |
| | | | IVKESMKMVL | |
| 3 | pNOP38141 | 0.83 | TGGPSSPSSHWKTPVVIYWDGTALRCVFVPV | TP53 |
| | alt | | LGETGAQRKRISARKGSLTTSCPQGALSEHC | |
| | splice a | | PTTPAPLPSQRRNHWMENISPFRCYLTYDGV | |
| | | | TS | |
| 4 | pNOP59073 | 0.76 | CCPRTILNNGSLKTQVQMKLPECQRLLPPWP | TP53 |
| | | | LHQQLLHRRPLHQPPPGPCHLLSLPRKPTRA | |
| | | | ATVSVWASCILGQPSL | |
| 5 | pNOP49591 | 0.65 | SSQNARGCSPRGPCTSSSYTGGPCTSPLLAP | TP53 |
| | | | VIFCPFPENLPGQLRFPSGLLAFWDSQVCDL | |
| | | | HVLPCPQQDVLPTGQDLPCAAVG | |
| 6 | pNOP70126 | 0.58 | GAAPTMSAAQIAMVWPLLSILSEWKEICVWS | TP53 |
| | | | IWMTETLFDIVWWCPMSRLRLALTVPPSTTT | |
| | | | TCVTVPAWAA | |
| 7 | pNOP224126 | 0.46 | CFANWPRPALCSCGLIPHPRPAPASAPWPST | TP53 |
| | | | SSHST | |
| 8 | pNOP272502 | 0.23 | FHTPARHPRPRHGHLQAVTAHDGGCEALPPP | TP53 |
| 9 | pNOP316190 | 0.17 | VRKHFQTYGNYFLKTTFCPPCRPKQWMI | TP53 |
| 10 | pNOP193414 | 0.15 | ASTAQQHQLLSPAKEETTGWRIFHPSGPDQL | TP53 |
| | alt | | SKRKLLKRA | |
| | splice b | | | |
| 11 | pNOP158914 | 0.12 | LARTPLPSTRCFANWPRPALCSCGLIPHPRP | TP53 |
| | | | APASAPWPSTSSHST | |
| 12 | pNOP281999 | 0.11 | ASTAQQHQLLSPAKEETTGWRIFHPSDPWA | TP53 |
| | alt | | | |
| | splice b | | | |
| 13 | pNOP293143 | 0.11 | ASTAQQHQLLSPAKEETTGWRIFHPSDAT | TP53 |
| | alt | | | |
| | splice b | | | |
| 14 | pNOP252394 | 0.11 | GACLCLSWERPAHRGRESPQERGASPRAA | TP53 |
| | | | PREH | |
| 15 | pNOP136003 | 0.10 | SPKRVSLPPAIKNSCSRQKGLTQTDILHF | TP53 |
| | | | LFPTDSLPPPSLPPLPFWVLGL | |
| 16 | pNOP385655 | 0.09 | QFLHGRHEPEAHPHHHHTGRLQW | TP53 |
| 17 | pNOP405064 | 0.07 | RWSGPSSASYPSGRKFACGVFG | TP53 |
| 18 | pNOP539666 | 0.05 | DVLPTGQDLPCAAVG | TP53 |
| 19 | pNOP59708 | 0.03 | LRLTFSTSCSPLTASHPHLSLPCHFGFWVF | TP53 |
| | | | EPLLAIGVRQKHPGLPFALSRGSTEQVGLH | |
| | | | WCFVVG | |
| | | | RRMGSRTYQLRF | |
| 20 | pNOP367554 | 0.03 | MRPWNSRMPRLGRSQGGAGLTPAT | TP53 |
| 21 | pNOP703537 | 0.02 | LYHHPLQLHV | TP53 |
| 22 | pNOP602122 | 0.02 | KQRSVPLAVPSNG | TP53 |
| 23 | pNOP243169 | 0.01 | GLGTQGCPGWEGARGEQGSLQPPEVQKGSV | TP53 |
| | | | YLPP | |
| 24 | pNOP483390 | <0.01 | RRAPSESGNIFRPMETTS | TP53 |
| 25 | pNOP433152 | <0.01 | HGHLQAVTAHDGGCEALPPP | TP53 |
| 26 | pNOP445026 | <0.01 | TRRKLKILSVGVSASRCSES | TP53 |
| 27 | pNOP604680 | <0.01 | LTMVLLPDKLVVS | TP53 |
| 28 | pNOP619453 | <0.01 | WRSRSQILASSPLRSYRRMIHLWWTAQISL | TP53 |
| | | | GVCRSLTVACCTGGLVGGTPLSISRPTSRA | |
| | | | RQSCCLPGLTHPAHQPLG | |
| 29 | pNOP82315 | 0.23 | SMALGPHSRISCLPTQTRGCILLAATPRSS | ARID1A |
| | | | SSSSSNDMIPMAISSPPKAPLLAAPSPASR | |
| | | | LQCINSNSRIT | |
| 30 | pNOP6110 | 0.21 | SGQWMAHMALLPSGTKGRCTACHTALGRGS | ARID1A |
| | alt | | LSSSSCPQPSPSLPASNKLPSLPLSKMYTT | |
| | splice a | | SMAMPILPLPQLLLSADQQAAPRTNFHSSL | |
| | | | AETVSLHPLAPMPSKTCHHKFWPHPPSAAW | |
| | | | RSCIALWCASSVTERTRCAGRWLWYCWPTW | |
| | | | LRGTAWQLVPLQCRRAVSATS | |
| 31 | pNOP88606 | 0.18 | WASTNQALPKIEVICRGTPRCPSTVPPSPA | ARID1A |
| | | | QPYLRVSLPEDRYTQAWAPTSRTPWGAMVP | |
| | | | RGVSMAH | |
| 32 | pNOP43369 | 0.18 | KVATPGSQTIMPCPMPTTPVQAWLEA | ARID1A |
| | | | PCRAGRRVPWAASLIHSRFLLMDNKAPAGM | |
| | | | VNRARLHITTSKVLTLSSSSHPTPSNHRPR | |
| | | | PLMPNLRISSSHSLNHHSSSPLSLHTPSSH | |
| | | | PSLHISSPRLHTPPSSRRHSSTPRASPPTH | |
| | | | SHRLSLLTSSSNLS | |
| 33 | pNOP5538 | 0.18 | SQHPRRSPSRLRILSPSLSSPSKLPIPSS | ARID1A |
| | | | ASLHRRSYLKIHLGLRHPQPPQPHGAARR | |
| | | | RRWRQQRWGGGASSLSRGRLAAPSLRLRA | |
| | | | TLRPEPVCRRRRRGRRLPPTTWRTTKPWP | |
| | | | GSAAERRRRGPGALRGAPAELSRPRLPQP | |
| | | | PVQLLLPQPQRLPPARPGLRAELPERWHS | |
| | | | GLRRGGGCRLQAASLLQRLRLLVVFVLRS | |
| | | | AALRGHGGRRPLRGRRGNSPAHRHPHPQP | |
| | | | TAHVAQLGPGLPGLPRGRLQWRAPGRGRR | |
| | | | QGPGGHGLAVLGGCGGGSCGGGRLGRGPT | |
| | | | KEPPRAHEPREQR | |
| 34 | pNOP1299 | 0.17 | RRGAAARPDPSAIQSNGSDGQDETSAIWR | ARID1A |
| | | | DAPREVALRAPARRRLPAPSRLPPPAPPP | |
| | | | PRRLRPSLSSASGPWGEAAPPRPAGELPS | |
| | | | PPPPPPSTNCSRRPARPGATRATPGATTV | |
| | | | AGPRTGAPARARRTWPRSVGGLRRRQLRR | |
| | | | RPPREGPNKGATTR | |
| 35 | pNOP16341 | 0.16 | PPILAATGTSVRTAARTWVPRAAIRVPDP | ARID1A |
| | | | AAVPDDHAGPGAECHGRPLLYTADSSLWT | |
| | | | TRPQRVWSTGPDSILQPAKSSPSAAAATL | |
| | | | LPATTVPDPSCPTFVSAAATVSTTTAPVL | |
| | | | SASILPAAIPASTSAVPGSIPLPAVDDTA | |
| | | | APPEPAPLLTATGSVSLPAAATSAASTLD | |
| | | | ALPAGCVSSAPVSAVPANCLFPAAL | |
| 36 | pNOP3000 | 0.15 | PSTAGAISRFIWVSGILSPLNDLQALGPH | ARID1A |
| | | | SRISCLPTQTRGCILLAATPRSSSSSSSN | |
| | | | DMIPMAISSPPKAPLLAAPSPASRLQCIN | |
| | | | SNSRY | |
| 37 | pNOP39264 | 0.15 | PALLPCPGQWRTAPLLASLHSCTLGSSSV | ARID1A |
| | alt | | SFLSSYLPSPAWHPRPFPVPCWLSRQCCS | |
| | splice a | | VSLRTTLACCSARQPDATSATQWPVGQHH | |
| | | | ASFHEPIKHCPRSRLYAEEPPDAPVQFPP | |
| | | | ARLSLISASAFRRTDTHRHGLLPAELHGE | |
| | | | LWSPGGSVWPT | |
| 38 | pNOP13360 | 0.13 | RWLPQAAKL | ARID1A |
| 39 | pNOP323677 | 0.08 | LRSTRTKNGGNLQPTSMWAHQAVLPAP | ARID1A |
| 40 | pNOP81513 | 0.08 | KSSISSVSMPLNARLNGEKTLPQTSLQLL | ARID1A |
| | | | IPRSPSPRSSLPLLRDQDLCRGPRLPSQP | |
| | | | AVPWQKEET | |
| 41 | pNOP109934 | 0.07 | ETSGPLSPLCVCEGDWWIDSGaQEQKMAG | ARID1A |
| | | | TCNQPQCGHIKQCCQLLEKAVYPVSLCL | |
| 42 | pNOP141882 | 0.07 | CGHDAAGCPRAACLGQGGREPLRVYSVRI | ARID1A |
| | | | TAVGHLGITVDELIGFTSHLHGRAGRPRR | |
| | | | RQQPGQPAAAAALGAEESRAAAAGGGGGR | |
| | | | GGGGGSGRARGNEGSRRAGKRG | |
| 43 | pNOP26533 | 0.07 | PRRGAAAAAGKGAAGRGREQWGWRRRRSR | ARID1A |
| | | | QRRRARRGAGPEELERERGPAATKWSGGG | |
| | | | TAWRCSGKTPWLHSPTSRGSWTYLHTPRA | |
| | | | FACLSWTDSYTGQFALQLKPRTPFP | |
| 44 | pNOP40276 | 0.05 | PWAPMPSFPRRDWSWKPSANSASRTTMWT | ARID1A |
| 45 | pNOP57388 | 0.05 | AHQGFPAAKESRVIQLSLLSLLIPPL | ARID1A |
| | | | TCLASEALPRPLLALPPVLLSLAQDH | |
| | | | SRLLQCQATRCHLGHPVASRTASCILP | |
| 46 | pNOP22341 | 0.05 | TITSRSRPAAAVAAAAMGWGRLLTQP | ARID1A |
| | | | RPPCRPQPTASGNPTAGARLPSP | |
| | | | PPRPPSSTNNMADNKALAWQ | |
| | | | RCRAAAAGAWSPTRGPSRTLTTTA | |
| | | | SPTTSTTPTTPTAAPTPRPPRPTR | |
| 47 | pNOP232518 | 0.05 | CGGLPARCIPWPRWTRTTQSLLCTNHGCWT | ARID1A |
| | | | SRYHR | |
| 48 | pNOP86506 | 0.04 | KGGGTGPRGELQQSGVVVGLLGDAPGKHLG | ARID1A |
| | | | YTRQHLGAVGPISIPREHLPACPGRTPTLG | |
| | | | SLPFS | |
| 49 | pNOP266437 | 0.04 | PRMELRVQRPSRRAASFHLALAQHRATGTS | ARID1A |
| | | | RS | |
| 50 | pNOP317526 | 0.04 | APGAAAAGGSRSPGPLSHPVQWIRWAR | ARID1A |
| 51 | pNOP91542 | 0.04 | HGQYATSGWVRDVSPTRGHEPENPRNCCRH | ARID1A |
| | | | ACCCQLYPKQAARLPQYESRGHDGNWTSLW | |
| | | | TRD | |
| 52 | pNOP160041 | 0.03 | QGPLHLTTSPHQACRITFLRYPALLPCPGQW | ARID1A |
| | | | RTAPLLASLHSCTLG | |
| 53 | pNOP205126 | 0.03 | QQQRVHQGQQTRRGPHLMDLQKNGSQPLWMT | ARID1A |
| | | | CCLLGLAP | |
| 54 | pNOP78127 | 0.03 | YGWHDQPSGTPIFHGWNHGQQFCRDGSQPRD | ARID1A |
| | | | DGPWGCKVNSSHQNEQQGRWDTQDRIQIQEI | |
| | | | QFFYYNQ | |
| 55 | pNOP204073 | 0.03 | NAAHRSEGQPRRLVAFPWHTPAPIWSLCPCA | ARID1A |
| | | | PHDKAPSI | |
| 56 | pNOP578746 | 0.03 | PLPPAAAAAAAATT | ARID1A |
| 57 | pNOP108335 | 0.03 | RTNPTVRMRPHCVPFWTGRILLPSAASVCPI | ARID1A |
| | | | PFEACHLCQAMTLRCPNTQGCCSSWAS | |
| 58 | pNOP140600 | 0.03 | SPGPLFHPGPQCRPFPAETGLGNPQQTQHP | ARID1A |
| | | | GQQCGPDSGHTPLQPPGEW | |
| 59 | pNOP162214 | 0.03 | APTSRRPPEPISIPVWPRPCLCTPWHQCPAK | ARID1A |
| | | | HATTNDGRPHTGISCTVFDWPVMTAVGHLPP | |
| | | | PCVCACVENLETDCCPLFMQNHLRIQFTLCC | |
| | | | PASPLGKSLSCFSLLLPP | |
| 60 | pNOP28463 | 0.03 | PLPPSPHAFLFLVLTLLPSGPYPTLFEKTKL | ARID1A |
| | | | CLHRRLFLF | |
| 61 | pNOP28543 | 0.03 | FLWQSVLHPRHPFWQPLPQPADYNVSTATAEL | ARID1A |
| | alt | | QAANGWHIWPSCQAARRGDVQRAIQHWAGAAS | |
| | splice b | | AAAVAPSPAPACQPATSCPAFPSARCIQPVWQ | |
| | | | CLSCHCHSCY | |
| 62 | pNOP342491 | 0.03 | STLRDPHIPWVEPWPTILQGWQPAQR | ARID1A |
| 63 | pNOP382230 | 0.03 | LCQQAEHGLCPPGPRLSWREPNR | ARID1A |
| | | | PKEPGVPGDGCGTAGQPGSGGQPGSSCHCSA | |
| | | | EGQYRQPPGLPRGQPCRHTVPAEPGQPPPHA | |
| 64 | pNOP84384 | 0.03 | EPTL | ARID1A |
| 65 | pNOP171474 | 0.02 | QVSIPALWDENAEGRSPSTCLAHSTCPCAAPHD | ARID1A |
| | | | SAGYHLPTWLC | |
| 66 | pNOP251638 | 0.02 | DPTVYPSGLAGFSCQALRLCVQYHSKPVICARQ | ARID1A |
| | | | FQEVPAQDPASLSCGIRIYAGAPDSPVNQQFHG | |
| | | | RRRRLKATNSSIHTTQSDPPIARHEQEQFSWD | |
| 67 | pNOP76377 | 0.02 | PGCL | ARID1A |
| 68 | pNOP115908 | 0.02 | TTRQMGHPRQNPNPRNPVLLLQPMRRSPSCMSW | ARID1A |
| | | | VVSLRGRCGWTVIWPSLRRRPWA | |
| 69 | pNOP145255 | 0.02 | SHTACVEAEEAAHNERHWNPGGMAGNDVPQVWS | ARID1A |
| | | | PGREHMGIRYHQHPAV | |
| 70 | pNOP157058 | 0.02 | AYPDPLREQDRAAAFPASRTLPTSPSEACDNSR | ARID1A |
| | | | GYTRDNRPGGAPT | |
| 71 | pNOP221454 | 0.02 | RSMRWVTQDRERYWILGGSARCLVQLPWRVGKK | ARID1A |
| | | | KKNF | |
| 72 | pNOP222331 | 0.02 | TEQMKCCTQIRGPTTKARGLPMAHASPHMVPLP | ARID1A |
| | | | LCPP | |
| 73 | pNOP272985 | 0.02 | GKLQGVIPSCPQGRAPTAGWVTPTWLPALG | ARID1A |
| 74 | pNOP289760 | 0.02 | RTALPPHSSSRARPASSTCRTHPLSQLVWT | ARID1A |
| 75 | pNOP329083 | 0.02 | TGKPKKLLSPCMLLPTLSKTGRQATPI | ARID1A |
| 76 | pNOP120573 | 0.01 | CLAQCQLPQCRHGWRHKPHGCRRSNAWTAWHPTL | ARID1A |
| | | | WHTPSREDESRLHGQPALWP | |
| 77 | pNOP419746 | 0.01 | PIIMPTGRARALPPRAPPIMA | ARID1A |
| 78 | pNOP472965 | 0.01 | GRARRYEPEPSVKTLQLA | ARID1A |
| 79 | pNOP144966 | 0.01 | RQPPGRKARAPPWGRRSRWERSCRTGPRAMGVAA | ARID1A |
| | | | AAEPAAAAGPARSRT | |
| 80 | pNOP271959 | 0.01 | DVQTPRAAAHPGQADPAAPQAPRTEAGTTNL | ARID1A |
| 81 | pNOP280686 | 0.01 | VTPPWATGLMALTWPICHLRLGQGCVPHQGA | ARID1A |
| 82 | pNOP325333 | 0.01 | PLQSCCRPWARKCGDGTTTALSLWRSL | ARID1A |
| 83 | pNOP339133 | 0.01 | PPHGDRRSSESWSEHIRDFQQPRRAE | ARID1A |
| 84 | pNOP460168 | 0.01 | QICLLWVGNLWTSIASMCL | ARID1A |
| 85 | pNOP471545 | 0.01 | FGGISPSHLALLKPHSLC | ARID1A |
| 86 | pNOP484623 | 0.01 | SHQLQHPHHTVRSPHCQA | ARID1A |
| 87 | pNOP526697 | 0.01 | PRTENATGSWEVQaGV | ARID1A |
| 88 | pNOP568326 | 0.01 | GDSLFRQGQASFRE | ARID1A |
| 89 | pNOP187097 | <0.01 | DLSHMAGLTHTRSNRDLRQDRSKDMGTQGSHTGPR | ARID1A |
| | | | PRSGTR | |
| 90 | pNOP286473 | <0.01 | LPAPTKHAESHSSGIQPCSPAPANGEPHLS | ARID1A |
| 91 | pNOP345053 | <0.01 | AGAIQLGSRMPLMMEVTPHSRSGIP | ARID1A |
| 92 | pNOP355250 | <0.01 | RKPSSSSGRRRGARRRRRQRPSAGK | ARID1A |
| 93 | pNOP357957 | <0.01 | TPWVPEVKCMDSLASHLMAHSLQGG | ARID1A |
| 94 | pNOP399373 | <0.01 | LHIPEAEFHDSKPWVSAQYEYL | ARID1A |
| 95 | pNOP450666 | <0.01 | EMWRWDHDSTIPMEVLMTE | ARID1A |
| 96 | pNOP503306 | <0.01 | PSTEPPEHQDPRGRTPQ | ARID1A |
| 97 | pNOP525902 | <0.01 | PFQARTSQLQRIVRRS | ARID1A |
| 98 | pNOP583798 | <0.01 | SCCTTSTQNGSRHH | ARID1A |
| 99 | pNOP584557 | <0.01 | SLHVLRAGPQRRDG | ARID1A |
| 100 | pNOP600191 | <0.01 | IPSTSCCMMTTAS | ARID1A |
| 101 | pNOP667279 | <0.01 | LMKRRRNRTKG | ARID1A |
| 102 | pNOP152466 | <0.01 | FLWQSVLHPRHPFWQPLPQPADYNVSTAT | ARID1A |
| | alt splice b | | AGIQPCSPAPANGEPHLS | |
| 103 | pNOP326245 | <0.01 | QQHHDLQPQSAPRVARAPCRIFPTMPD | ARID1A |
| 104 | pNOP363287 | <0.01 | GKHEHWGPTAESHAFQPRLGDVFS | ARID1A |
| 105 | pNOP366177 | <0.01 | LASHDSRGTPPPPVCVCVCGELRN | ARID1A |
| 106 | pNOP390796 | <0.01 | WAAPYRHQLRLLSKAPCGRGVMT | ARID1A |
| 107 | pNOP391130 | <0.01 | WPRRSPPPPPAAWATRRRRRPRS | ARID1A |
| 108 | pNOP532250 | <0.01 | SSSHGGWGRRRRTSRS | ARID1A |
| 109 | pNOP535077 | <0.01 | WELDLLMDKGLIVWLA | ARID1A |
| 110 | pNOP536697 | <0.01 | AFSQDPPACLIYLVQ | ARID1A |
| 111 | pNOP539995 | <0.01 | EFRGHQGEQQVSIWH | ARID1A |
| 112 | pNOP561120 | <0.01 | WGACPMSQIRILMAA | ARID1A |
| 113 | pNOP564630 | <0.01 | CPSSLVSWQRAHGH | ARID1A |
| 114 | pNOP580855 | <0.01 | QWPAALADWWGGHH | ARID1A |
| 115 | pNOP596649 | <0.01 | GEGHGHDKSACCG | ARID1A |
| 116 | pNOP600818 | <0.01 | KCRRQVPQYLPRT | ARID1A |
| 117 | pNOP616167 | <0.01 | TGRRPSPRHLCSC | ARID1A |
| 118 | pNOP616285 | <0.01 | THWFHKSFVMYCF | ARID1A |
| 119 | pNOP624639 | <0.01 | EEDVGGPLSGLH | ARID1A |
| 120 | pNOP628397 | <0.01 | GSLWQHEESSRE | ARID1A |
| 121 | pNOP643975 | <0.01 | RTRTGTRALGPP | ARID1A |
| 122 | pNOP650952 | <0.01 | WTSRKTDHSHYG | ARID1A |
| 123 | pNOP658966 | <0.01 | GCSARHHVAGA | ARID1A |
| 124 | pNOP700714 | <0.01 | KTLEPRRHGG | ARID1A |
| 125 | pNOP704301 | <0.01 | MTSPWGQKEL | ARID1A |
| 126 | pNOP708028 | <0.01 | PSTSVSSQGC | ARID1A |
| 127 | pNOP708425 | <0.01 | QASSKDRTEE | ARID1A |
| 128 | pNOP709605 | <0.01 | QSEDGAWNRA | ARID1A |
| 129 | pNOP718154 | <0.01 | TRRGRRRGSS | ARID1A |
| | | | WAAPEWRSCCCSTARSPTAPTPPLSPDPCT | |
| | | | TLPGRASWTRWWCCTGPGRGWTCAMPGAVC | |
| | | | P | |
| 130 | pNOP42370 | 0.43 | WTWLRSWAIAMSHGTCARLRGAPEAVTMPA | CDKN2A |
| | | | LRSEADPGHDDGQRPSGGAAAAPRRGAQLR | |
| | | | RPRHSHPTRARRCPGGLPGHAGGAAPGRGA | |
| | | | AG | |
| 131 | pNOP64888 | 0.28 | RARCLGPSARGPGGSSRFLEDQVMMMGSAR | CDKN2A |
| | | | VAELLLLHGAEPNCADPATLTRPVHDAARE | |
| | | | GFLDTLVVLHRAGARL | |
| 132 | pNOP23100 | 0.19 | DVRDAWGRLPVDLAEELGHRDVARYLRAAA | CDKN2A |
| | | | GGTRGSNHARIDAAEGPSDIPD | |
| 133 | pNOP340964 | 0.06 | RRCGRCWRRGRCPTHRIVTVGGRSRS | CDKN2A |
| | alt | | | |
| | splice a | | | |
| 134 | pNOP309800 | 0.04 | LAGHGRGPGSGRGGAGAAGGGGAAQRTE | CDKN2A |
| 135 | pNOP159351 | 0.03 | MPRKVPQTSPIERTREALRNLGKLRSSVTE | CDKN2A |
| | alt | | GPTGPQLPPPQPTPLS | |
| | splice b | | | |
| 136 | pNOP374903 | 0.02 | WSRRRGAAWSLRLTGWPRPRPGVG | CDKN2A |
| 137 | pNOP412936 | <0.01 | GAGPSRCRTVPARGCGGHQRQ | CDKN2A |
| 138 | pNOP103788 | <0.01 | KQACVGKLRDSAEERQSLRRPLVIASW | CDKN2A |
| | | | LAHSAPGAKDAWGCGKGKATSSRIRAW | |
| | | | HYIPD | |
| 139 | pNOP149155 | <0.01 | PCPHRCRGRSLSWLDQPQDFQTQLCVA | CDKN2A |
| | alt | | SSGDLSISALLHNSTNLTLLS | |
| | splice c | | | |
| 140 | pNOP219511 | <0.01 | MPRKVPQLAGPTSGFPNPIVRGIIWRSL | CDKN2A |
| | alt | | DLGSSAQLN | |
| | splice b | | | |
| | | <0.01 | | |
| 141 | pNOP255336 | | MPRKVPQLQLASRSREVKKETSAPVTAS | CDKN2A |
| | alt | | IRVPI | |
| | splice b | | | |
| 142 | pNOP258500 | <0.01 | SQTSSWRRPGGLGSQGRGMRSHARTDLS | CDKN2A |
| | | | NAEKI | |
| 143 | pNOP267771 | <0.01 | RRRLRLQLQLASGSRFRRSCQLQRGSRA | CDKN2A |
| | | | EQKA | |
| | | <0.01 | RRCGRCWRRGRCPTHRIVTVGGRSRWVE | |
| | | | GLQREQGMAGDSGGRSLQGNWNQVALRF | |
| | | | SGKR | |
| 144 | pNOP31901 | | GGFLGSFQKGFVITDLLLATPWGLGKPR | CDKN2A |
| | alt | | KRNEEPRAYRSLEC | |
| | splice a | | | |
| 145 | pNOP334099 | <0.01 | GFSWFTSRGSRGSGQRQGRPPLWPSC | CDKN2A |
| 146 | pNOP371501 | <0.01 | RVCSGSRGWRATLEDEVCRGIGIR | CDKN2A |
| 147 | pNOP401561 | <0.01 | PCPHRCRGRSLRHPRLKEPERL | CDKN2A |
| | alt | | | |
| | splice c | | | |
| 148 | pNOP419434 | <0.01 | PCPHRCRGRSLRNDRKPFVGL | CDKN2A |
| | alt | | | |
| | splice c | | | |
| 149 | pNOP461083 | <0.01 | RFDSPEKGEASWGVFRRGL | CDKN2A |
| 150 | pNOP578182 | <0.01 | PCPHRCRGRSLSYS | CDKN2A |
| | alt | | | |
| | splice c | | | |
| 151 | pNOP598590 | <0.01 | HGAGGGEQHGAFG | CDKN2A |
| 152 | pNOP605842 | <0.01 | NGAGGGEQHGAFG | CDKN2A |
| 153 | pNOP639300 | <0.01 | PSGFGARAARRE | CDKN2A |
| 154 | pNOP67306 | <0.01 | SETICGFVEAGMRREATGFRRGAPEPEAPFGY | CDKN2A |
| | | | RKLAGSLRTRCKRCLGMREGKGHIFTPSRLAL | |
| | | | HPRLKEPERL | |
| 155 | pNOP81258 | <0.01 | HDDGQRPSGGAAAAPRRGAQLRRPRHSHPTRA | CDKN2A |
| | | | RRCPGGLPGHAGGAAPGRGAAGRARCLGPSAR | |
| | | | GPG | |
| 156 | pNOP97211 | <0.01 | HGAQVLGDPPDSARVRPAASEGFRGSHPAAHG | CDKN2A |
| | | | GVGSARGARRCGPRADATEEPASRAAAASRGL | |
| | | | NPMPSTCSLVPSALTPWVLCLISRTARDGSSP | |
| | | | LATSAPVCTGAQWMLGGAAGIGAEFWSIGHGG | |
| | | | RGKSQLTWRLQRRTRPLCTAPPLPQSPQVVRT | |
| | | | PHWTQMFLSLELLSATRPFRTWTLHCGQI | |
| 157 | pNOP6876 | 0.20 | QAAPLLQPPVLFRGLESKCPTTRHPGGPWGVS | KMT2B |
| | | | PLAPCPPLEVHLH | |
| 158 | pNOP339832 | 0.12 | QMWLLPPQRPLPGNGVRKAQNGWCRHRRCCP | KMT2B |
| | | | GIPMNLLRPPLVLQAHAGGRELGGPGRRWWP | |
| | | | TQGPRSRTPSCSASQLGAASNSDPPMISSR | |
| | | | IRMTRSPGAPLLLGVGPPEKMSCHCQNLRS | |
| | | | RAGPANLPCSLCCSSRPEGAWTRMLWPLAPL | |
| 159 | pNOP9663 | 0.10 | | KMT2B |
| | | | LLFPMAGLESRSLPMVCTASVWILRRIVIVP | |
| | | | APPVSSRHPGDLWMKTPPNPQRWRSHLSCDL | |
| | | | PLPPPHLFPRSQHQSPLHHVPQLLHLPQFH | |
| 160 | pNOP73574 | 0.07 | SLRRDGPSVCSPLCQGAPRWCACCVPAKDST | KMT2B |
| | | | SWCSVKSAVTHSTHSAWRRPSGPCPSITTPG | |
| | | | AAVAANSATSVDAKWDPSTSWSASAAAMHTT | |
| | | | RPVWGPAIQPGPRANGATGSVQPVCAVRAVG | |
| | | | QLQARTGT | |
| 161 | pNOP8413 | 0.07 | SSGLEITASAPGAPSYMRKETTARSVHAAMKT | KMT2B |
| | | | TTMRAR | |
| 162 | pNOP212366 | 0.06 | PTTSPQWETRTSQLPPDVPVVPALWLPGRLHHG | KMT2B |
| | | | GPPLLRLRDPFRTARLGAVHLRTVCWGSAAPLA | |
| | | | RGPERGPPGGPAPGAPGPAELQGGGPTAALHPV | |
| | | | WARWEATAPRTLRPASCESALRGWPLQVCAQLH | |
| | | | GGHGGHPHAALGGGRDPGPPGWRPDEGAPAEAA | |
| | | | RICVRLVRRPRPQVLATEYPAAKRSPSQCGVAP | |
| | | | IPGSCLCAVETAGTRDPRIRAASRGSLSSIPGQ | |
| | | | GSGCLLTPGGPPSVCTLPQIRGCRLQGGGAALV | |
| | | | HRAERVDTRQLCHLVGGSLRGERRLPQECA | |
| 163 | pNOP1023 | 0.03 | | KMT2B |
| | | | CCCGPREADALRALPEAWRHGGLLPVLLPQQLPL | |
| | | | HVCPGQLLHLPG | |
| 164 | pNOP284432 | 0.03 | GVLGMEVLALERSHSPRRLPWLMAASPPKA | KMT2B |
| 165 | pNOP149964 | 0.02 | RPPQTPKGGGLTCPATSHYHLPTCSPGASTSPLS | KMT2B |
| | | | TTCPNSSIYPSSTP | |
| 166 | pNOP170320 | 0.02 | LNFSGGPRHPKHPGAGHVSPPPPGGLGDGPQDGQ | KMT2B |
| | | | QAPAGGSSKQWTPRCMAMPPASSTTPVSPTASLG | |
| | | | SSTWRARNTLLSSPCAASCVVRSSPTTTSSPSRM | |
| | | | PATSCPA | |
| 167 | pNOP35490 | 0.02 | TVAPSAAVGSLTEAVAAHHDPSHLLLPSLPSCP | KMT2B |
| 168 | pNOP536795 | 0.02 | AGPSRGACARCSRAC | KMT2B |
| 169 | pNOP27215 | 0.02 | IPMGLLGQRSISGSAPLTCSTSWPPSTGCSLRGPPVMR | KMT2B |
| | alt | | KRMRCSSGQPDVPPAWSCPWPCVFVTLRRRPKKLWVST | |
| | splice a | | DQPSTGEACSVSATSTRGRWSSSTLALSSARC | |
| 170 | pNOP346473 | 0.02 | DDPPSSSSPSRCGSYPPKDPCPETGALEGRWRRWPGLS | KMT2B |
| | | | SRSPTEALSGLKMSRWKLRESGPQVPSPLCKVPASNMS | |
| | | | AVMLLWPWVRPGPWCLKMSLASVPSLSGIGRTSPQRIH | |
| | | | HRRPRLRVSRHGPGGERWRQQALGENQSPQVLEGPW | |
| 171 | pNOP8126 | 0.02 | PTHPGAHCPPITARRCAWLDVDTVGAAYVCRTVGPVS | KMT2B |
| | | | TA | |
| 172 | pNOP81603 | 0.02 | LLQPLHLLHPSHPLRHLLHPHSALHHHPQCPHHLYHPL | KMT2B |
| | | | HRLLPKRSRRNPLLLWSQLRAPGRGAGLP | |
| 173 | pNOP102672 | 0.01 | AVGQPARPARPSASRGCPLSPAGPRQHLPHTKPPGWM | KMT2B |
| | alt | | KMERPQRIPLRFQGLAVAGLAV | |
| | splice b | | | |
| 174 | pNOP113418 | 0.01 | GAEPAPQTYPAACVAAQGPKAPGQGCFGPWPLCFFSQW | KMT2B |
| | | | LDWKAEVSRWCAPRPCGF | |
| 175 | pNOP129859 | 0.01 | KPPLSSGCPLLPQSSQPSHLPQGSWLPLARPHLHHPLK | KMT2B |
| | | | TWAQTSRTWRWCQD | |
| 176 | pNOP139147 | 0.01 | LWCPPLVWPPALPLEPPALNSWTAWTTALTVRLRRCSS | KMT2B |
| | | | LGARARLLRGQE | |
| 177 | pNOP142719 | 0.01 | GLPWSSRPTPGGGSWGAPGGGGGPPRARGAGLPPAAQV | KMT2B |
| | | | SSALRQTATLLGRGVPSRGSSSEQRATDTGSATAAPAG | |
| | | | LANPAPAPGTTATTATAAATAVTTADASPGKSPDCGRG | |
| | | | FLAAVWGRGEDVQPPQESQSAAIQDRSAAAAEGGSFHA | |
| | | | AEPWRADGGGGRGCQADLRQRP | |
| 178 | pNOP17169 | 0.01 | CPV | KMT2B |
| 179 | pNOP172961 | 0.01 | VGRDSWASTMMLSSSWPSSSPEPSVASTISSVTTSRER | KMT2B |
| | | | ARRSRPLCGAAVARRGRAEPSPGRTRPCSVCWGSAGACA | |
| | | | GSAACGPARGSSGAGDGVGAGAGARVEAA | |
| 180 | pNOP20643 | 0.01 | CRRRRAVTGNPTRRSFRVFIQMKMWPPVPCALRSDPSEV | KMT2B |
| | | | ERPEVGVASIRRPPFLLLA | |
| 181 | pNOP233428 | 0.01 | ERAALRSRVPCARSPHQTCLPSCCCGPGSGPGHGA | KMT2B |
| 182 | pNOP283728 | 0.01 | GAHLRLQVPHRGCQQQAALQLWRQALPSVP | KMT2B |
| 183 | pNOP306682 | 0.01 | ELWGNSRQELGRRVVWRLQPLPQVHPAI | KMT2B |
| 184 | pNOP392368 | 0.01 | AQHRRGGDGHRVLWHCHPLGVD | KMT2B |
| 185 | pNOP443670 | 0.01 | SRKCKRPEGMPDSDISPLVE | KMT2B |
| 186 | pNOP482268 | 0.01 | REPGPKTDWPTSALRDQQ | KMT2B |
| 187 | pNOP499276 | 0.01 | LGARGPPCSSASDPPRKAPTSCGSSET | KMT2B |
| | | | SDWQLEMQGGARSRTWDPQAWRTVKPW | |
| | | | RPWRQGPRPRWWAPLCDQVCFK | |
| 188 | pNOP54281 | 0.01 | GQKSKDGTIVLGTRIRSRSRST | KMT2B |
| 189 | pNOP569191 | 0.01 | GPPTGHRCSCPWSSRWDNCPWDSNQVK | KMT2B |
| | | | VKVNMRKVGRMSPKEELDLDREGALAG | |
| | | | KSRNRSWMTRKKRRKKKKKKT | |
| 190 | pNOP73224 | 0.01 | RREKRRKKEL | KMT2B |
| 191 | pNOP109317 | <0.01 | ALPGRDCSRWGHGEQPRGPGGQLRGGV | KMT2B |
| | | | QPHLPLHPLPCDCGVRPWSGPQRYPWS | |
| | | | PPH | |
| 192 | pNOP12376 | <0.01 | AVGQPARPARPSASRGCPLSPAGPRQH | KMT2B |
| | alt | | LPHTKPPGWMKMERPQRIPLRFQGLAV | |
| | splice b | | AGPSRNGPLCCHFRKMVLPRSPMVPQT | |
| | | | CCLSPSGTTIQVRLRALRKSLHPQMIK | |
| | | | RTRPQNGLAHICASRSAVRMGSALRQR | |
| | | | AWRGRGEL | |
| 193 | pNOP12501 | <0.01 | | KMT2B |
| | | | NLRSAGSTPTTPSTGDGVPGCQTESFP | |
| | | | MRCCPHPWIMSMRSGDSRNQRPQNQGS | |
| | | | LQGIPQQHSRARIRLPSHTWRTPVSVH | |
| | | | SASNTGMQTPRRRGGSCTSGRTSGHTS | |
| | | | TVPSGRRKSSRRTTAPSR | |
| 194 | pNOP137356 | <0.01 | CSAHSAITGCMPSARGSQMKTTRSFQD | KMT2B |
| | | | CQTRCCTPADRVLGQRSPAGERP | |
| 195 | pNOP14051 | <0.01 | APLAHSEPGPSTAARFRQRPSSSPPFF | KMT2B |
| | | | FGGSNQSAQLLAIPEALGGCLLWPPAL | |
| | | | PWKSIFTDPPHPHSGRPGLPSSPQTFP | |
| | | | SSQPFGSQAASITVGLPSSKNLPSAQG | |
| | | | APSYLSRHSPHTYLRGAGSPWPGPIST | |
| | | | TP | |
| 196 | pNOP145287 | <0.01 | SLAPRWAAACPPASATSTSCVPGPATA | KMT2B |
| | | | SSRMTRKSSARNTLISWMARKL | |
| 197 | pNOP159086 | <0.01 | LPASGRSGKLLGQGQRAPLLPLQPPAP | KMT2B |
| | | | PREALRKTVPPWPPKAPPS | |
| 198 | pNOP160746 | <0.01 | RWRGLRGYPSGSRAWQWRAPPGTVPFA | KMT2B |
| | alt | | ATSGRWSSPGPRWSPRPAA | |
| | splice c | | | |
| 199 | pNOP170722 | <0.01 | NIRLAAGNARRGPVQDLGPPGVEDSQA | KMT2B |
| | | | VEAVEAGAAAEVVGSPL | |
| 200 | pNOP170957 | <0.01 | PGSCPLLPQPLHLPRPPPHPLLLPPP | KMT2B |
| | | | PGGPYSFGPLSLPQAKPT | |
| 201 | pNOP172435 | <0.01 | SSHLCPPPFPPRLPPPGLCPQAPSSAC | KMT2B |
| | | | CPWSEWSALPRPRHPLP | |
| 202 | pNOP173362 | <0.01 | WRRRRAAAVAPGLAPRGAASRAGRGAP | KMT2B |
| | | | AGAGAAADGATGPKECG | |
| 203 | pNOP181020 | <0.01 | FRERVADGGPECAHLCARGPPDGV | KMT2B |
| | | | LAVCQQRTPRAGVLSSLL | |
| 204 | pNOP183367 | <0.01 | PGSAWGARWGRKSWAPPGTVPFAAT | KMT2B |
| | | | SGRWSSPGPRWSPRPAA | |
| 205 | pNOP199665 | <0.01 | VSASRMATTSLCTASWRTWWASSCG | KMT2B |
| | | | TRRRERPRTAGLEAR | |
| 206 | pNOP207889 | <0.01 | ALHPPAVSGTAPRTASRPLQEEAAS | KMT2B |
| | | | SSGGRSSCDNPQT | |
| 207 | pNOP2249 | <0.01 | VPLPPAGRGPGGAAPESPWGCSGRGL | KMT2B |
| | alt splice d | | SPLCLQQYIPPSPAATCRKCTFDMFN | |
| | | | FLASQHRVLPEGATCDEEEDEVQLRS | |
| | | | TRRATSLELPMAMRFRHLKKTSKEAV | |
| | | | GVYRSAIHGRGLFCKRNIDAGEMVIE | |
| | | | YSGIVIRSVLTDKREKFYDGKGIGCY | |
| | | | MFRMDDFDVVDATMHGNAARFINHSC | |
| | | | EPNCFSRVIHVEGQKHIVIFALRRIL | |
| | | | RGEELTYDYKFPIEDASNKLPCNCGA | |
| | | | KRCRRFLN | |
| 208 | pNOP23566 | <0.01 | DGGGGGRRQLPRAWLRAGPLPGPAAG | KMT2B |
| | | | RRRGRGPRRTGQRGRKSAGSSAARRW | |
| | | | RDGAGRSRARGGHGPAPFAGAPPGPA | |
| | | | PAPPPVGRPAGPAGPGTGSGPGLGPE | |
| | | | SRLRAGGGEQ | |
| 209 | pNOP23765 | <0.01 | NGGGGGRRQLPRAWLRAGPLPGPAAG | KMT2B |
| | | | RRRGRGPRRTGQRGRKSAGSSAARRW | |
| | | | RDGAGRSRARGGHGPAPFAGAPPGPA | |
| | | | PAPPPVGRPAGPAGPGTGSGPGLGPE | |
| | | | SRLRAGGGEQ | |
| 210 | pNOP252560 | <0.01 | GGAAASGPGHASFGARSSPGRGPWGC | KMT2B |
| | | | RGQGPASKPPQCVGSLTWIGLGSPLG | |
| | | | KKVLGPSRNGPLCCHFRKMVLPRSPM | |
| | | | VPQTCCLSPSGTTIQVRLRA | |
| 211 | pNOP25410 | <0.01 | LRKSLHPQMIKRTRPQNGLAHICASR | KMT2B |
| | | | SAVRMGSALRQRAWRGRGEL | |
| 212 | pNOP263780 | <0.01 | IPMGLLGQRSISALSSTVYSSFPCCH | KMT2B |
| | alt | | LQEVHL | |
| | splice a | | | |
| 213 | pNOP269620 | <0.01 | VPLPPAGRGPGGAAPESPWGCSGRGL | KMT2B |
| | alt | | SPEVHL | |
| | splice d | | | |
| 214 | pNOP278498 | <0.01 | RRRCSASSREPKCSYSRSISSSSRR | KMT2B |
| | | | WQLPCR | |
| 215 | pNOP281826 | <0.01 | APRWWAHCCSAPSVGQMGSNCTQDP | KMT2B |
| | | | AACKL | |
| 216 | pNOP287880 | <0.01 | PLGPWGAATGARGTAPRRSPAPPPA | KMT2B |
| | | | TSTSL | |
| 217 | pNOP295363 | <0.01 | GKLAGCPPKKSWIWTGREPLLEKAG | KMT2B |
| | | | TEAG | |
| 218 | pNOP295589 | <0.01 | GRELGGGVENSDRESARGPRACPTQ | KMT2B |
| | | | TSLL | |
| 219 | pNOP317592 | <0.01 | AQLLLSGHPRGGPETHCYLRPAPHP | KMT2B |
| | | | AW | |
| 220 | pNOP323657 | <0.01 | LRPWLPTTTPHTSCCRRCHLAPSLG | KMT2B |
| | | | AP | |
| 221 | pNOP326541 | <0.01 | RCPSPQCPPSPGSAGPRHRGYIIGVRD | KMT2B |
| 222 | pNOP328068 | <0.01 | SGQGSLGLQGTGPGLLRTCHRKLWILC | KMT2B |
| 223 | pNOP331404 | <0.01 | ALALPLSPPNPPHPKSYLSTSWGKYL | KMT2B |
| 224 | pNOP331561 | <0.01 | APQTRHIQNHTCQQAGASICEDGWGG | KMT2B |
| 225 | pNOP340189 | <0.01 | RCGPQFPALCAPIPARSSAPRSGSQA | KMT2B |
| 226 | pNOP363468 | <0.01 | GPAIGNCGFCVEEPRGSWGWRCWP | KMT2B |
| 227 | pNOP367137 | <0.01 | LTSGRSSTMGRASGAICSAWMTLM | KMT2B |
| 228 | pNOP370489 | <0.01 | RGRREERRRRKRQGGRREGRKSCS | KMT2B |
| 229 | pNOP373366 | <0.01 | TPMVLMFSAESMWTSRASTSSGSS | KMT2B |
| 230 | pNOP376070 | <0.01 | ASGSGPHQPPQPASIRPCGHHSC | KMT2B |
| 231 | pNOP378678 | <0.01 | GAAQVNQTCHQPGAAHGHAFSSP | KMT2B |
| 232 | pNOP384879 | <0.01 | PHPHICLAPRGPRGPGVKPWPCP | KMT2B |
| 233 | pNOP393358 | <0.01 | CSPPSLCGLRGHQLQAEVLDGA | KMT2B |
| 234 | pNOP394645 | <0.01 | EQDDAVRTVRSLGACQVRGALR | KMT2B |
| 235 | pNOP402065 | <0.01 | PPAQLTPPAHLPGSQGPQGSGC | KMT2B |
| 236 | pNOP407306 | <0.01 | TSPSLGALTPRSSAVYTGSVTK | KMT2B |
| 237 | pNOP411745 | <0.01 | EDVQRSCGCLQISHPRARPVL | KMT2B |
| 238 | pNOP41189 | <0.01 | TCPTPSEAATFAPHHFPHGSHLLDS | KMT2B |
| | | | APRPPPRRAARGRSGPPCPAPATPS | |
| | | | PDAGAEQWASQPAPPGHPRQEGVHF | |
| | | | LRPVPASTSPIQSPPAG | |
| 239 | pNOP426146 | <0.01 | VLLTWTSRPACWGLSPSRKRL | KMT2B |
| 240 | pNOP459923 | <0.01 | QAGEVLRWEGHRVLYVPHG | KMT2B |
| 241 | pNOP462749 | <0.01 | RWRGLRGYPSGSRAWQWRV | KMT2B |
| | alt | | | |
| | splice c | | | |
| 242 | pNOP468831 | <0.01 | CCHLPGRAAPRSPALPAL | KMT2B |
| 243 | pNOP469462 | <0.01 | CSGRHDAWQCRPLHQPLL | KMT2B |
| 244 | pNOP483192 | <0.01 | RPGPRLRGHGGGVRTECC | KMT2B |
| 245 | pNOP533725 | <0.01 | TSPAGPGTPSTPEPGM | KMT2B |
| 246 | pNOP538448 | <0.01 | CQLRKRKRQSCHHRL | KMT2B |
| 247 | pNOP546704 | <0.01 | KRPDDSEDAVALGFR | KMT2B |
| 248 | pNOP56683 | <0.01 | PIPPILPGGGRAAPAPASRHLVLPSLQI | KMT2B |
| | | | LPRLWTQRSWIQAPPGVRALPPCIPPGL | |
| | | | SGAQLSNPGHAQTAPLDLFSLCAL | |
| 249 | pNOP581470 | <0.01 | RGIRRGGVSGFSFR | KMT2B |
| 250 | pNOP582085 | <0.01 | RLGRWNDWLKKAGR | KMT2B |
| 251 | pNOP599417 | <0.01 | HVQLPGLPAPGAP | KMT2B |
| 252 | pNOP607050 | <0.01 | PCEDENPHSAWGP | KMT2B |
| 253 | pNOP60902 | <0.01 | ECPVTVPAGKGGGSRPWGRIRAHRFWRD | KMT2B |
| | | | PGPHTPALTALPSRQEDAHGSMWTLSGL | |
| | | | PTCAGLWVLCQLPRQAQVWGP | |
| 254 | pNOP609760 | <0.01 | QSPNLSPHLLWFQ | KMT2B |
| 255 | pNOP614494 | <0.01 | SPGWQGNCEPRWF | KMT2B |
| 256 | pNOP616888 | <0.01 | TRCHQRAHWFHPH | KMT2B |
| 257 | pNOP619315 | <0.01 | WQPALPRPDRQPS | KMT2B |
| 258 | pNOP625450 | <0.01 | ERKLLPDLYTLL | KMT2B |
| 259 | pNOP62604 | <0.01 | EETVHPKGTHISLDLTDPGAAP5SPSPS | KMT2B |
| | | | TSPGPLPTPCSCHLLPEAPTPSGPSVYP | |
| | | | KRSPPEDLRIGAYSSSSWGS | |
| 260 | pNOP644158 | <0.01 | RWLGRVNLSHPQ | KMT2B |
| 261 | pNOP650472 | <0.01 | WNEWGETPGHPP | KMT2B |
| 262 | pNOP660324 | <0.01 | GRHRTDGAGTD | KMT2B |
| 263 | pNOP661817 | <0.01 | HQEAVLCIPEV | KMT2B |
| 264 | pNOP673600 | <0.01 | QNRGSEDGTTG | KMT2B |
| 265 | pNOP675110 | <0.01 | RGVTPPGASPG | KMT2B |
| 266 | pNOP706730 | <0.01 | PGLRGQPAGD | KMT2B |
| 267 | pNOP711022 | <0.01 | RISGSLLCLW | KMT2B |
| 268 | pNOP71226 | <0.01 | SLGLRGTALPHWLPVLPSVLEHSGCS | KMT2B |
| | | | EALLVSVPNSGVSAMGAEGRASSPGG | |
| | | | CRGEPDHCAQPRPFLRAPRW | |
| 269 | pNOP720871 | <0.01 | WNDWLKKAGR | KMT2B |
| 270 | pNOP82310 | <0.01 | RSTNRCLLLLLLGLLKPLSQSLLLPMT | KMT2B |
| | | | LQLSLSLGQWAAPTTSACLDSPLWSPL | |
| | | | LLRPRCPLTGLQL | |
| 271 | pNOP8822 | <0.01 | GDDASCGKGRGKAATTASDSSSPFTSS | KMT2B |
| | | | TPPTPFDISSTPTLPSTTTPSVPTTST | |
| | | | IPSTASCPRGAGGIPSSCGPSYVLQEE | |
| | | | GPASPDSQPAGGAGSCSGRARGHLSSH | |
| | | | SNPQHRHGRPSGRQSHRGPQKHHLPEE | |
| | | | YPAVYYACGECPLLPCHQDTPAIYG | |
| 272 | pNOP99414 | <0.01 | ATGHRHRLSYCSPCRPCKPSSCPRHYRH | KMT2B |
| | | | HSHSCSHRRHHSRCLPWKKPGLRAWVPC | |
| | | | RCLGTRRCHCCPHLRSHPCPHHLRNHPR | |
| | | | PHHLRHHACHHHLRNCPHPHFLRHCTCP | |
| | | | GRWRNRPSLRRLRSLLCLPHLNHHLFLH | |
| | | | WRSRPCLHRKSHPHLLHLRRLYPHHLKH | |
| | | | RPCPHHLKNLLCPRHLRNCPLPRHLKHL | |
| | | | ACLHHLRSHPCPLHLKSHPCLHHRRHLV | |
| | | | CSHHLKSLLCPLHLRSLPFPHHLRHHAC | |
| | | | PHHLRTRLCPHHLKNHLCPPHLRYRAYP | |
| | | | PCLWCHACLHRLRNLPCPHRLRSLPRPL | |
| | | | HLRLHASPHHLRTPPHPHHLRTHLLPHH | |
| | | | RRTRSCPCRWRSHPCCHYLRSRNSAPGP | |
| | | | RGRTCHPGLRSRTCPPGLRSHTYLRRLR | |
| | | | SHTCPPSLRSHAYALCLRSHTCPPRLRD | |
| | | | HICPLSLRNCTCPPRLRSRTCLLCLRSH | |
| | | | ACPPNLRNHTCPPSLRSHACPPGLRNRI | |
| | | | CPLSLRSHPCPLGLKSPLRSQANALHLR | |
| | | | SCPCSLPLGNHPYLPCLE | |
| 273 | pNOP134 | 0.3 | SQPCLSLGNHLCPLCPRSCRCPHLGSHP | KMT2D |
| | | | CRLS | |
| 274 | pNOP234091 | 0.2 | GPRSHPLPRLWHLLLQVTQTSFALAPTL | KMT2D |
| | | | THMLSPH | |
| | | | ARVMPVPVFLAQSPSWALQTRRGVAPCP | |
| | | | WSWGSLRMLVQPEMRAPYGSVLTHCQRL | |
| | | | MTHYC | |
| 275 | pNOP21934 | 0.12 | AMLGQLSAEAKLRGRRGGGAAPQPVPAS | KMT2D |
| | | | NRVAAAVSQEDAGLVEEPMEDVVEDGPG | |
| 276 | pNOP111349 | 0.08 | PTLRWGLGGSQQPCPRGQQVSSMPRSQV | KMT2D |
| | | | GSPPILSGPLGRVHLWAPPLPCVSLSLR | |
| | | | Q | |
| 277 | pNOP170800 | 0.06 | NRLMRRLNGRPCCGGWSQDPWALRSALP | KMT2D |
| | | | LLLMPLNPAWHLCSLRCCSRAGWWSVLC | |
| | | | VRCVARPPTPHACCSVMTVILATTHTAW | |
| | | | TPHCSPSPRAAGSASGVCPVCSV | |
| 278 | pNOP44838 | 0.06 | GLLPLASTVNGRIVTHTVGPVPAWPCHH | KMT2D |
| | | | CTSGANGEDGLASQARQDWRVLSPQMPL | |
| | | | ALMTRRMGTWTPMSCSRVKVVWSTWSAK | |
| 279 | pNOP22159 | 0.05 | LNWRAPSALMWSLAKRRPRKAKNASVNH | KMT2D |
| | | | IGLALVVSWCDSGNPTHARKRGLLHRRR | |
| | | | C | |
| 280 | pNOP118654 | 0.04 | PGSSPHQQGAEARGTGQPAPRCCPHHFHWQP | KMT2D |
| | alt | | HYPRRLVYLCGRVPEAAGGLGAWP | |
| | splice a | | | |
| 281 | pNOP70346 | 0.04 | HHAEYRGSLLQHRQICPNAGHVCGMWQLWPG | KMT2D |
| | | | GRGPPPCLFAVLSVLSPLLCQQQDHQGDAA | |
| | | | QGLALCGVYCV | |
| 282 | pNOP8757 | 0.04 | SSGERFQQLTKPPTCKRPKITGQLTASTRCR | KMT2D |
| | alt | | SQGHWAARPPLLPPPFSLAAPLPPPACLPLR | |
| | splice b | | TGS | |
| 283 | pNOP129784 | 0.03 | KHCSCYAQSTVRGLHIWRRLAVQCVRGQGSCV | KMT2D |
| | | | TCSSVPAVGITITGPAWTLLWTARSWLVRIKI | |
| | | | QNRQLMDLQLLRTQVPLSQTCPTHMWERSLSL | |
| | | | VLGVPGFRRLLRTAVGVRCG | |
| 284 | pNOP17440 | 0.03 | VVLSVTAGSPVYTGSGSYGALSCHLIGPGVQW | KMT2D |
| | | | CPLGGAQGPMRQCCPVRTYHRLVSLRALHLPT | |
| 285 | pNOP257632 | 0.03 | RRKSLGHPLLAMGPQTWALLTHPPQAPTWVAWS | KMT2D |
| 286 | pNOP69709 | 0.03 | ACPPYDPSPISRLPSGAGFSHPDGAPSSSVFAT | KMT2D |
| | | | PSAFPGSPKLPSFPVLSSCPTTVRSLPVESHRE | |
| | | | GSGGLR | |
| 287 | pNOP16127 | 0.02 | KAAVRHCRGPFFKVDSLWAICPPAAQWTPTQASA | KMT2D |
| | alt | | SPRSWILGSAGASLARNPVSPTAPGRAQVAPRPP | |
| | splice c | | PPQPPPRRVRATDSPITSGVFSAGRRMRSWASCP | |
| | | | PSHLCSMPTLIFLISSKTTQTGQAVANKS | |
| 288 | pNOP189145 | 0.02 | LLGPNLRPLRAAVLCPLAHCPPTLSPECLPVLSP | KMT2D |
| | | | SPAPSLH | |
| 289 | pNOP21288 | 0.02 | SRRRARCLALTRLVSSSSSSHPRCPPKCLRRTPL | KMT2D |
| | | | DWPLPIPWSPASPRHRPPIPPILVLRGPLRSPRC | |
| | | | WAPHLVLGLASQGNSTLPHLAPPDTSPPHLTHSS | |
| | | | NPAAPRWITWLCLRALG | |
| 290 | pNOP23772 | 0.02 | NRRAPPQSHPLSTAIPTMSPIWMCDSSRPHLLKN | KMT2D |
| | | | PPRPLPPWHLLLPVPLLSPWLNFPPNPWLSHPSP | |
| | | | HLCHWPHPLNQPDPSPVPGPLKKVKIPVLLASRN | |
| | | | GKECAGSGFGCC | |
| 291 | pNOP269687 | 0.02 | VRTPTDWLLKGFGAWRYQVFPHRNPQPHRPLN | KMT2D |
| 292 | pNOP29324 | 0.02 | GQGLDLRAHPGSLPHQEPYLQDQSLALSIPHLHH | KMT2D |
| | | | PALKSQRDLHNYLPPAPSFPLRPSSLPPIQGPPN | |
| | | | LRGQPWSRLLGGSHLLLPSLQIPCLARVWDLGIP | |
| | | | QTT | |
| 293 | pNOP58594 | 0.02 | SKSLASFSGENGCTCSVWGALCSTPSDSCCLTRW | KMT2D |
| | | | LTFIVPLPSIPWATRPRASIGASAPTIVAAAIA | |
| | | | VLLVRTTGGRSL | |
| 294 | pNOP62730 | 0.02 | GIPTQHQAGTSGRAMCPGSPVSEEGGQWGANRGT | KMT2D |
| | | | RNQQPPPAGRPSLRSWASALAEATPGKE | |
| | | | CATQHWAGVRGAAS | |
| 295 | pNOP8118 | 0.02 | YRATTSQTRTCPPVWAGSAWGWNHAYGGSASSTA | KMT2D |
| | | | PRSPGQKPTAAALKSSAAAAATGTPHAA | |
| | | | AAAAESGSTPDPTLPGAWDPDLSPPGPPGLPTST | |
| | | | WGLPWTTDRPPPGARGRASTSGPTPAPCPTRSLI | |
| | | | YRTSPWPCPSHTSTIQPSRAKETFTITFPQLPAS | |
| | | | H | |
| 296 | pNOP106859 | 0.02 | HPGLCLLKLFAHHPLPLASSPLTLILAHPHALSPVT | KMT2D |
| | | | HLPHCISHPDPSPLKLPLRLGLAPCQGPKWAAPQFC | |
| | | | PVPWDGCICGHPLSHAFHFPSGSRGAFPKAPCPSAW | |
| | | | SPATPWDQQPFWARPHLGQASKHKLHSSHRELPPIG | |
| | | | QPPGAQQRVHRGELWAVPTTPSVGSATTCTRRIPPL | |
| | | | PVP | |
| 297 | pNOP11179 | 0.02 | WSLTAIRHHLSCRKARRPRDWNG | KMT2D |
| 298 | pNOP188940 | 0.02 | KTWRPMTPTWMTCSMETSLTCWHILILSWTLGTRR | KMT2D |
| | | | ISSMST | |
| 299 | pNOP243509 | 0.02 | GVSHAHSLCCCSQEPEWRDGGSGGAAEHEDPQLLPQG | KMT2D |
| | | | TSTHRAAPWGPAAGPQGRAMGCPHYALRRFCHHLHPT | |
| | | | DPSPTCPMEPHSDQASPLLSKSE | |
| 300 | pNOP28077 | 0.02 | KTQGLEWVALWRQLNSQVPRTQACPALAKQSWRSNG | KMT2D |
| | | | SASDYESC | |
| 301 | pNOP363905 | 0.02 | GWVSSPHFAGGWGVPSSPARGASR | KMT2D |
| 302 | pNOP36658 | 0.02 | GPYTCPPRRTWRVLLGSPLVCCMVGRRMGAGGPRTMW | KMT2D |
| | | | CGQGHLLRDLTALLPLHQARCLHPL | |
| | | | PLTWMSTALPLPLRDCQRFLPIHENTAAAMPRAQ | |
| 303 | pNOP390234 | 0.02 | VEARPPLLGHRTRAALWGCPQAS | KMT2D |
| 304 | pNOP493996 | 0.02 | GAATLPPVRGAAPVTPA | KMT2D |
| 305 | pNOP61039 | 0.02 | GHQEPATTSCWQALAQKLGICSCRSYSGQRMCNSALG | KMT2D |
| | | | GGPRGCELRSTGTLTASWLGWSRNYRVPPATRRMQQQ | |
| | | | GSL | |
| 306 | pNOP96015 | 0.02 | VLSSSSSYRHSSCSGSCSRVRQYARPHPTRSLGPRPLP | KMT2D |
| | | | SRASWAANLNLGASLDHRQAPSRS | |
| 307 | pNOP102126 | 0.01 | TTVFIQHPTPRVLPCQLVWSWSTGPRRALSLAAPILW | KMT2D |
| | | | PWKLGSCPVRIPSWMTILMPTRPFKAFTGKAAAAAAA | |
| | | | TYAAGPETAAAAAAATAAAAPSRTGGNPAATAAGSWS | |
| | | | TDKPSSGSQAPGPYASQQPPRPPGPAAVPSTTPGAPG | |
| | | | HAGPCPGGCVAAAAPWSGPPGPSQTGAYDPVPGAQFP | |
| | | | PAGTAGSGPYGTQAGHSPAAAAATTAPTARVHGRAVP | |
| | | | SSAESDVTQWAAQTERSAHGLFTAASAAAAAATATAT | |
| | | | SAAAAAAATTATATSAATASTAATAAAASTTAAATAS | |
| | | | TAATAATTATATTTAA | |
| 308 | pNOP1069 | 0.01 | VSTAAATAADGPFKPESNFTVSSATTAAASGTWPWHA | KMT2D |
| | | | SKASSTLF | |
| 309 | pNOP108932 | 0.01 | VPRWREFPPVCQALVSQCLVQLVLPSSLSCGTMYRKD | KMT2D |
| | | | WDLGALRFLVRAHLRDPVFTL | |
| 310 | pNOP110054 | 0.01 | GEAQGGGGWTPPFSLPIHHCYPQGRARTCCQFPWPGA | KMT2D |
| | | | KARTEHDGQPGYPDGHRAIF | |
| 311 | pNOP114830 | 0.01 | PSAPCASELVPPAAAIACVAPMSTILLVPSVPSACSS | KMT2D |
| | | | RTRPCCVQCIRSRGPVSKS | |
| 312 | pNOP127724 | 0.01 | TRTASGLWNPWPRRQPYATAEALSSRWTPFGQSA | KMT2D |
| | | | LQQPNGLLPRPLPVPVPGF | |
| 313 | pNOP137298 | 0.01 | CLQSPPDPSGISGRAPEPGLGPKAPGATPCPGFG | KMT2D |
| | | | TFSSKSPRHLSPWLLH | |
| 314 | pNOP139704 | 0.01 | PSPGCSVPPSWHSRVRALWDTGWSQPSSSSSNNS | KMT2D |
| | | | TNSKGPWQGCPIFSRV | |
| 315 | pNOP154481 | 0.01 | PLWRSTPNASRQQGRAHHVKNRKSHVHRWPPHHP | KMT2D |
| | | | LSSNPTSLTRSLI | |
| 316 | pNOP155302 | 0.01 | RSPTPMRCCSQRAPPGQALSQRRGKLRVLVGRKR | KMT2D |
| | | | VWKARAQTLALIG | |
| 317 | pNOP172213 | 0.01 | SHCKGQDGGFERHQESDGSGQHWGGTWYEQTASV | KMT2D |
| | | | SASPEALGGT | |
| 318 | pNOP178870 | 0.01 | TISAWHWWFHGATAEIPHTHEKGACCTGGGVEWG | KMT2D |
| | alt | | WAARRGDTC | |
| | splice d | | | |
| 319 | pNOP179906 | 0.01 | ALPQAPTPGARPSAFAGPLWTGPCLSPGAPLPHG | KMT2D |
| | | | TAHLSPLS | |
| 320 | pNOP182619 | 0.01 | LPANVLAGSALNAKCAKPAGNLGMTLRCWFVRRVT | KMT2D |
| | | | KDTILSA | |
| 321 | pNOP187538 | 0.01 | FGSRSSATPCGRRRKQLQQLQEQWGLQAAGVLSPA | KMT2D |
| | | | ALPLSS | |
| 322 | pNOP18835 | 0.01 | KAAVRHCRGPFFKVDSLWAICPPAAQWTPTQASASP | KMT2D |
| | alt | | RSWILARNPVSPTAPGRAQVAPRPPPPQPPPRRVRA | |
| | splice c | | TDSPITSGVFSAGRRMRSWASCPPSHLCSMPTLIFL | |
| | | | ISSKTTQTGQAVANKS | |
| 323 | pNOP193752 | 0.01 | CRTCVWYVAALAGGQRATSLPVRSALSAITLTVSTA | KMT2D |
| | | | RSPRGLFSQFGWVPTAAFPGSCRCPTARFAPATDAH | |
| | | | PATSSCPPATPGSIHGYGVQSRAYAKWAAWR | |
| 324 | pNOP20115 | 0.01 | AGRLGTPAELTASAITEAHGHHATFHVHEAAAIGNA | KMT2D |
| | | | AAAGKQLLPRYRPGQICCRRYH | |
| 325 | pNOP201536 | 0.01 | ELLCSAPSLTALRPFLPSACQSSVPVQLPVSTDTPA | KMT2D |
| | | | SVCTCWLPCLHPLTIRLRMSGWRVMRIAILLTALCQ | |
| | | | LHPLRASWGRRPLVSLIWAQAGGSKRTGPSPL | |
| 326 | pNOP20393 | 0.01 | SSPSFLGPASQSSQIPNLMGPLAWRSLESCLSQLGKR | KMT2D |
| | | | AKEVRCQSCSQSLLLQPRT | |
| 327 | pNOP209010 | 0.01 | EPWGRGRQSFRAPALAPTFWGVPEGPRGEEGRAWGILS | KMT2D |
| 328 | pNOP209424 | 0.01 | GGEGAAAQLPSPFPHQTGSQQQFPRKTPASWRSPWRTW | KMT2D |
| 329 | pNOP211152 | 0.01 | LPHILPGPPTAHRPQGRLEVQWCVLYAVWGCFPWLPL | KMT2D |
| 330 | pNOP224854 | 0.01 | EEEATAARAQEEQTGGHVPCLLAGSLLWEGAAGPEP | KMT2D |
| 331 | pNOP245157 | 0.01 | LLTLIALPVRRRRKKMMTPCRIPWFSSPTQTNLS | KMT2D |
| 332 | pNOP257396 | 0.01 | RLPCAPGPRGAGPCDPYGGLPRMQADSRAGLTM | KMT2D |
| 333 | pNOP264714 | 0.01 | LHTLWALCQPGDLPYLSCSLRRRGPTNPVPPL | KMT2D |
| 334 | pNOP284778 | 0.01 | HHSAGRTAAHVPCGGPCVPRHRTAAASPDG | KMT2D |
| 335 | pNOP287872 | 0.01 | PLCPLWQWLPSQWAEPAEGGLWKWGAAHWP | KMT2D |
| 336 | pNOP298931 | 0.01 | NHPWRNCLLTLGSARRAGCAGPVGRAQQN | KMT2D |
| 337 | pNOP303477 | 0.01 | VAPSWGQGPSLAMTDSPGHLHQPRLPLWM | KMT2D |
| 338 | pNOP310713 | 0.01 | MDRWCLRHPNSASSRNLGKSHVPWEPSQ | KMT2D |
| 339 | pNOP318057 | 0.01 | CHQIPFLLHSHPSSQLRPHRPCLLWGS | KMT2D |
| 340 | pNOP324899 | 0.01 | PADTTLVAAPHPTPIGAAEDGEWRHPI | KMT2D |
| 341 | pNOP334374 | 0.01 | GLTCFPTTGGLAHVPAAGGVTPVATT | KMT2D |
| 342 | pNOP336175 | 0.01 | KGTEGYFRGEESRPAGCLAYTPSQSD | KMT2D |
| 343 | pNOP352206 | 0.01 | MASPHLKSWGSTPRMLPLPGIVKGH | KMT2D |
| 344 | pNOP376012 | 0.01 | ARQPLDGLRWHHALHPHNPHHGG | KMT2D |
| 345 | pNOP408074 | 0.01 | VTRRHHPRRCPPPHPHRCSRRW | KMT2D |
| 346 | pNOP412059 | 0.01 | ELLSLSPLSQSPGRSDYPLRC | KMT2D |
| | | | ALSPWALYSSFSSSSSCNSNSNFSSSS | |
| | | | SSSYNSNSNFSSNSFNSSNSSSSFNNS | |
| | | | SSNSFNSSNSSYNS | |
| 347 | pNOP44778 | 0.01 | NSNNNSSSFNSSSNSSRWAF | KMT2D |
| 348 | pNOP465144 | 0.01 | TQPFLQRPLRGPLHIREGR | KMT2D |
| 349 | pNOP483870 | 0.01 | RTLPAPFPLGTFSCQSPY | KMT2D |
| 350 | pNOP487229 | 0.01 | VAQEDPPCWKSLSSRVGL | KMT2D |
| 351 | pNOP490058 | 0.01 | APVGGPPKRGDATAAPT | KMT2D |
| 352 | pNOP513338 | 0.01 | AVRPFLQLGWAGQALD | KMT2D |
| 353 | pNOP548811 | 0.01 | LTIVRCWDSYQRRQS | KMT2D |
| 354 | pNOP558727 | 0.01 | TGGPAAGGGARTLGP | KMT2D |
| | | | DRWQSSSNSSRVLEYRQTKLWVPSPRAL | |
| | | | CLPAATKASWSSSCPLNHPRGPRACWAL | |
| | | | PRWLCCSS | |
| 355 | pNOP56040 | 0.01 | STLELWAPRALTDRCL | KMT2D |
| 356 | pNOP608986 | 0.01 | QGTARHASLLFLS | KMT2D |
| 357 | pNOP85659 | 0.01 | AWGTTSVPSARGAAWPIWGAILVASADATRS | KMT2D |
| | | | PSSSTLTHHHSCGPTGPVSFGGVRVPLWCQR | |
| | | | GQ | |
| 358 | pNOP109806 | <0.01 | EAPKLSISEHPILGPCPYSSNSNNCGSNNRQ | KMT2D |
| | | | QQQPPCDLPCQLAFHQLLDLNLAAKP | |
| 359 | pNOP116135 | <0.01 | WGSQMRLSCTRWRLRKFQNLNAQPWNPVPPV | KMT2D |
| | | | LSLPQWGTFPAPPPALPQPWMTSLA | |
| 360 | pNOP118804 | <0.01 | PSRRAVGGRRMSGKWQSLWSSLAQPCDLTRY | KMT2D |
| | | | RETCVAAVSVMRRVTGPLMGLPVC | |
| 361 | pNOP118816 | <0.01 | PTGPTSPHSPAARGTGQPAPRCCPHHFHWQP | KMT2D |
| | | | HYPRRLVYLCGRVPEAAGGLGAWP | |
| 362 | pNOP127343 | <0.01 | SGPCKIIQGHNLPNQDLSSSLGRVCLGLESC | KMT2D |
| | | | LRWVSFEHSSKESWPKTHSCGT | |
| 363 | pNOP137386 | <0.01 | CSVAWLYPEEPTRHLEPPETGEPRPRATHSA | KMT2D |
| | | | QLYLQCLQSGCATALGPTS | |
| 364 | pNOP142770 | <0.01 | GPQKPREMEAQKGRNSPHRRKEMMVQILQMK | KMT2D |
| | | | NPVASRAKPIHQDLRMGA | |
| 365 | pNOP143S20 | <0.01 | LCLLPALRGKACGACCTSRAGAHEGERARAP | KMT2D |
| | | | VLSLRRCVADRNWHGLAA | |
| 366 | pNOP144316 | <0.01 | PNRAGEATAAPATTRAADSAADPAQHPAAGE | KMT2D |
| | | | GNSCSSCRSSGASRQLGC | |
| 367 | pNOP144483 | <0.01 | PVRLTDRPYISAFPRSQGHWAARPPLLPPPF | KMT2D |
| | | | SLAAPLPPPACLPLRTGS | |
| 368 | pNOP152835 | <0.01 | GRSAQDPLPLWSLELSEMDELRSFEATRQGS | KMT2D |
| | | | PPTHNLFPERDEGEER | |
| 369 | pNOP161094 | <0.01 | SSGERFQQLTKPPTCKRPKITGQLTASTRCR | KMT2D |
| | alt | | SRLRARSTSRPRWAT | |
| | splice b | | | |
| 370 | pNOP165656 | <0.01 | QRIPYFLPKTTHGGTACSLLEVQGVPGVPGL | KMT2D |
| | | | WGGLSRTESQLGW | |
| 371 | pNOP169094 | <0.01 | GKTQPLWMGLMLRVHSQSLDRPLAVWLVNLK | KMT2D |
| | | | APLCSWTPRSWPL | |
| 372 | pNOP172370 | <0.01 | SQLLLPLRLWLLTLIALPVRRRRKKMMTPCR | KMT2D |
| | alt | | IPWFSSPTQTNLS | |
| | splice e | | | |
| 373 | pNOP172794 | <0.01 | TRRGKALTLWGLTTPACPTPAPASAQLSAAA | KMT2D |
| | | | ATSEASRTTAAAS | |
| 374 | pNOP17361 | <0.01 | RSRLVYTASPGRLCVPSSALPKKLAVSSQKLM | KMT2D |
| | | | LRSSSWLQSSRARSRNNWIRSGNSRRSTLISW | |
| | | | QNIGTSSSNNSSSSSNNSNSTQLCWLSALPRV | |
| | | | PGCSPSSLVSCSLAMGCSHHRGLRVGKPEVFA | |
| 375 | pNOP174645 | <0.01 | EEGAAEEAAAFSTVAACPAAAATAAAAFPTVC | KMT2D |
| | | | TRPCPGHVFAT | |
| 376 | pNOP175361 | <0.01 | GVAVPYPAAPTDAAEGARGADWCTPQVPEGSV | KMT2D |
| | | | CQAAHCQKSWP | |
| 377 | pNOP183568 | <0.01 | PRGSRGDLAVICRTMWQLGVARSGVLVIPPSL | KMT2D |
| | | | VPTRPLLLRE | |
| 378 | pNOP185368 | <0.01 | TRVELYCLLSNNSSSKWHLALACQQSLFNTFL | KMT2D |
| | | | ALEPWVQPSS | |
| 379 | pNOP191904 | <0.01 | STPLVPKGTVTLSHRWLPPSWRHPSALHQK | KMT2D |
| | alt | | LTALTLSLSPL | |
| | splice f | | | |
| 380 | pNOP194798 | <0.01 | GLICAPPAGSALCFLRGSAWVHDPEPSGPP | KMT2D |
| | | | TAHARAAHAK | |
| 381 | pNOP198849 | <0.01 | SRSNWQCSSSWQTASSQIQTWTNLLQKISL | KMT2D |
| | | | IPLQRPRWWL | |
| 382 | pNOP198864 | <0.01 | SSAATVNGGCMQAVRASSQRTMWSRQPMKA | KMT2D |
| | | | LTVSPASPTW | |
| 383 | pNOP199023 | <0.01 | SYGGPCAAPDAGRLISSWGWPARGIPHYP | KMT2D |
| | | | TWHPQTPALHT | |
| 384 | pNOP199159 | <0.01 | TISAWHWWFHGATAEIPHTHEKGACCTGGG | KMT2D |
| | alt | | VEWGWAARRG | |
| | splice d | | | |
| 385 | pNOP211037 | <0.01 | LKGMRRRSNSGEGARRANWRTCSLLTCRKP | KMT2D |
| | | | SLGRSCWT | |
| 386 | pNOP214330 | <0.01 | TGFPQKNCPRWNPRTCSSSSRMFWALNENS | KMT2D |
| | | | IWVVEPLA | |
| 387 | pNOP215253 | <0.01 | WSPFLLSVRHSFSIPWFPKTPLLPSALLLP | KMT2D |
| | | | YHCPFPPR | |
| 388 | pNOP215460 | <0.01 | AAESRPDPLCWDTGQEQPCGVAPKQAEWPH | KMT2D |
| | | | PGARVLP | |
| 389 | pNOP217529 | <0.01 | GPAPSHPSRDPQTSGANLGAASWEGLTCCC | KMT2D |
| | | | PACRYLV | |
| 390 | pNOP217538 | <0.01 | GPFCSWGGPAKLWTRDPKSQGRWRLRKEGT | KMT2D |
| | | | PHIAERR | |
| 391 | pNOP218359 | <0.01 | ITARGGELSKLFIPLWAPPPYGAATHDQPH | KMT2D |
| | | | WLCPIRA | |
| 392 | pNOP218743 | <0.01 | KSTQWLSSTLAPSFGTRWPTGGRKSTKSRI | KMT2D |
| | | | EASTCSE | |
| 393 | pNOP220563 | <0.01 | QGSGTLGSPRQPSRNPEARAEQPGTWASGPG | KMT2D |
| | | | EWTGGA | |
| 394 | pNOP223482 | <0.01 | YSSGPTAATATFWWGWIPGWPFRGLLPWQPC | KMT2D |
| | | | SSKPRT | |
| 395 | pNOP240334 | <0.01 | WAAG1PGWAQGHFLAVGTQLRRPPLGPREDH | KMT2D |
| | | | QLTC | |
| 396 | pNOP248474 | <0.01 | SPLSLSLVSRHPMGSTAILGPAPPWASLKAQ | KMT2D |
| | | | TTQ | |
| 397 | pNOP251217 | <0.01 | CQCQFSWLRAPPGLSRPGGGWLPVHGVGGLY | KMT2D |
| | | | GC | |
| 398 | pNOP257143 | <0.01 | RFPSSSPQEMERSALEAASAAADHPEGQWAA | KMT2D |
| | | | GG | |
| 399 | pNOP258695 | <0.01 | STPLAVPDQSLKSSHTTNAFSHPLSHLILTT | KMT2D |
| | alt | | TL | |
| | splice f | | | |
| 400 | pNOP259446 | <0.01 | VGSMEGRQAWYPSRAHSQCYHRSPWAPCHLP | KMT2D |
| | | | CA | |
| 401 | pNOP261027 | <0.01 | CHCPLSRGLRGHAHLLEPPHQQSSLLLSLFYW | KMT2D |
| 402 | pNOP261872 | <0.01 | EGLLWGHGRTTSSPADPQPTEWPRRILPAGKV | KMT2D |
| 403 | pNOP270434 | <0.01 | AAAQCTERTGTWGHSVSWSGPTSETPFLPCK | KMT2D |
| 404 | pNOP276046 | <0.01 | MPSLGTQCHQSSPFPNGGPFLPRPQPCPSPG | KMT2D |
| 405 | pNOP277209 | <0.01 | PVLLYQLWASLSRGLPGHCSDCPQTCWLAVP | KMT2D |
| 406 | pNOP277754 | <0.01 | RARCSVRCMPRAAKGWARDLYATQGTRAPAM | KMT2D |
| 407 | pNOP279143 | <0.01 | SKSSSRAWRTWSSLTPLPRPCGIASLSLWLP | KMT2D |
| 408 | pNOP285042 | <0.01 | IEQQSSSNTPHQGSYPANWFGAGQPAPVEH | KMT2D |
| 409 | pNOP302234 | <0.01 | SPHSLGTHNSCLSNPSPSLSPALCSCSHL | KMT2D |
| 410 | pNOP318220 | <0.01 | CPPSHQLMPSSNAWLHPWLWCPIKGIC | KMT2D |
| 411 | pNOP318964 | <0.01 | eaqagyraaeqdpettgsgpetaegah | KMT2D |
| 412 | pNOP323435 | <0.01 | LNHCPGWRAVKTIYSAMGATPLWSCHS | KMT2D |
| 413 | pNOP323658 | <0.01 | LRQDFHRRTAQDGIQGPAAALQGCSGL | KMT2D |
| 414 | pNOP325001 | <0.01 | PDHVTTAQAAPTARTAWPPRRGRIGGF | KMT2D |
| 415 | pNOP325387 | <0.01 | PMTISLILRTISTRSPATVEPGIVGNG | KMT2D |
| 416 | pNOP325875 | <0.01 | PWSPGSNPPPDGQGTKHRRPSRFFRGH | KMT2D |
| 417 | pNOP341158 | <0.01 | RSLLSPPILASLPPLAVAAQSMGRAS | KMT2D |
| 418 | pNOP343442 | <0.01 | TWTWTCGCTSTVPFGPRRCMRPRAGH | KMT2D |
| 419 | pNOP344075 | <0.01 | WACPSAEPGPGPVGAPQLCPLVHGGV | KMT2D |
| 420 | pNOP356926 | <0.01 | SQARLPRLVKPLQTNHEALEKGSSS | KMT2D |
| 421 | pNOP362881 | <0.01 | FWESQASGDSSGLQWGSGAALCSL | KMT2D |
| 422 | pNOP363170 | <0.01 | GGPLEVGRCPLALTTIPSCLPRIT | KMT2D |
| 423 | pNOP364735 | <0.01 | IITFFSTGGVALVSTGRVTPISCT | KMT2D |
| 424 | pNOP370861 | <0.01 | RMMKSLLTWVWVWMWPRVMMNLAP | KMT2D |
| 425 | pNOP37587 | <0.01 | GISEHLHRRDQHPLQQAVCALQVISVPAAAHRM | KMT2D |
| | | | EEQRVPGSLPYPGPGALCSQGPRKAHNGYRVHW | |
| | | | HHHSERGGQPAGENLRRAESRHLHVPNKQ | |
| 426 | pNOP378675 | <0.01 | GAALVPSPWGTILISLAWRASPV | KMT2D |
| 427 | pNOP378896 | <0.01 | GFQDNSSSKLACSTQQVEEAMGS | KMT2D |
| 428 | pNOP386633 | <0.01 | RHPQCPVTLRSQAPQVKGCLALT | KMT2D |
| 429 | pNOP388467 | <0.01 | SMKLTSGSMRSGCSIPSSSYRCS | KMT2D |
| 430 | pNOP394670 | <0.01 | EQRAAGVCNQSHRAGPGGPGLH | KMT2D |
| 431 | pNOP404863 | <0.01 | RTGRATCTGGPHTTHSHQIRHR | KMT2D |
| 432 | pNOP405923 | <0.01 | SPRWRRVDATLLLANSPLLPPR | KMT2D |
| 433 | pNOP406378 | <0.01 | STPLAVPDQSLKSSHTTNGPIP | KMT2D |
| | alt | | | |
| | splice f | | | |
| 434 | pNOP410165 | <0.01 | AVDHLLRPHLCPTCWLSPLFP | KMT2D |
| 435 | pNOP413106 | <0.01 | GEAKLPSPCSRPHLLGSPGRP | KMT2D |
| 436 | pNOP414691 | <0.01 | HLTKRTKSSSSPAGESPKERS | KMT2D |
| 437 | pNOP421083 | <0.01 | QRGQNHHHLQPANPQRRGANL | KMT2D |
| 438 | pNOP421373 | <0.01 | RASG PGGIRSSPTETLSPTGP | KMT2D |
| 439 | pNOP425823 | <0.01 | TWPPSPRFPVGGNFHPSARPW | KMT2D |
| 440 | pNOP43053 | <0.01 | PLGVWHYLDSLVAPSLIQLWPNSSNSNILVGL | KMT2D |
| | | | DPWLALQGASSLATLLFEASDLIQGFYRKGSC | |
| | | | SCSSNVCSWPRNCSSSSSSNSSSSTF | |
| 441 | pNOP438522 | <0.01 | PAALPGTLTIPVPLTVWPKS | KMT2D |
| 442 | pNOP458695 | <0.01 | PAPHSRWRKPWAARQWIIF | KMT2D |
| 443 | pNOP466225 | <0.01 | VSEGRGALWADGACRASHS | KMT2D |
| 444 | pNOP46646 | <0.01 | PASYPCSLRTCWSMRRRSCRRSSSFQHSCSLPS | KMT2D |
| | | | SSSNSSSSIPYCLHQALPRPCLCHMRALLPVWL | |
| | | | GPNSSFPWVLQVPDSQVCPSH | |
| 445 | pNOP468251 | <0.01 | APERSCGRRTGSGPARPC | KMT2D |
| 446 | pNOP473253 | <0.01 | GSWWEGKGSGRQEPRHWP | KMT2D |
| 447 | pNOP481442 | <0.01 | QKPRSQSRAAWYLGIWTR | KMT2D |
| 448 | pNOP487911 | <0.01 | VTVGCPHPGDTHQPSTRS | KMT2D |
| 449 | pNOP490152 | <0.01 | AREWGFDLAWWTCSIWG | KMT2D |
| 450 | pNOP490194 | <0.01 | ARQDGELTGSQRVTPAH | KMT2D |
| 451 | pNOP494542 | <0.01 | GIAPIPPACGVTPVSTA | KMT2D |
| | alt | | | |
| | splice g | | | |
| 452 | pNOP494543 | <0.01 | GIAPVPAAGGIAPLSAA | KMT2D |
| | alt | | | |
| | splice g | | | |
| 453 | pNOP501743 | <0.01 | NPHTLQTAPYPEQHQHV | KMT2D |
| | alt | | | |
| | splice h | | | |
| 454 | pNOP502714 | <0.01 | PLCNPRNQGPCNVKPNH | KMT2D |
| 455 | pNOP506673 | <0.01 | RVTHVSTTGGISSVPTI | KMT2D |
| 456 | pNOP507548 | <0.01 | SLPASSQPAHFCSGSDQ | KMT2D |
| 457 | pNOP508277 | <0.01 | SSQQPYEAPYPEQHQHV | KMT2D |
| 458 | pNOP512482 | <0.01 | AGSGRVYGAAWHSLAT | KMT2D |
| 459 | pNOP513379 | <0.01 | AWPPQSSGPGSWEVAL | KMT2D |
| 460 | pNOP513605 | <0.01 | CGAWQRGDRGKQKTQA | KMT2D |
| 461 | pNOP514247 | <0.01 | CSGFTARAWTDPWQFG | KMT2D |
| 462 | pNOP517078 | <0.01 | GALYTSGRAVSNRNYP | KMT2D |
| 463 | pNOP518512 | <0.01 | GVGPAVHHLTCALCQH | KMT2D |
| 464 | pNOP522295 | <0.01 | LAPVSSGVPWGEPRAQ | KMT2D |
| 465 | pNOP523824 | <0.01 | LTLLRHPPGWPGVKDTSHGRISE | KMT2D |
| | | | QAAATTAAAAATTATALSCAGSQ | |
| | | | PFPESPAAHQAPWSAAPWPWAAA | |
| | | | TTGASGWAS | |
| 466 | pNOP52423 | <0.01 | RRSSPDPWGYGTTWTAWWPLP | KMT2D |
| 467 | pNOP526117 | <0.01 | PICSAPIDSSAPTSAP | KMT2D |
| 468 | pNOP530549 | <0.01 | SAEPCGSWEWPGAECW | KMT2D |
| 469 | pNOP530881 | <0.01 | SFPHLQAPQWGRLLPS | KMT2D |
| 470 | pNOP537026 | <0.01 | ALLLSSGGSTLSGTR | KMT2D |
| 471 | pNOP548556 | <0.01 | LRGAQSTRAAGATAL | KMT2D |
| 472 | pNOP550374 | <0.01 | NPHTLQTRFHIHYLI | KMT2D |
| | alt | | QQAGWAGAETTGYPQQQ | |
| | splice h | | GGCSSKEAFDTEAQAGT | |
| | | | EGKRQVGELPKEAAEGG | |
| | | | RGQGQRGLAE | |
| 473 | pNOP55230 | <0.01 | TAETGAVPAAPNGACYH | KMT2D |
| | | | RQF | |
| 474 | pNOP563434 | <0.01 | ARAELFCCLPAGLH | KMT2D |
| 475 | pNOP566785 | <0.01 | EPDQQADQGGRHSP | KMT2D |
| 476 | pNOP568806 | <0.01 | GKQGSNLSPSWRPP | KMT2D |
| 477 | pNOP569843 | <0.01 | GVWPGLRPLTPAAL | KMT2D |
| 478 | pNOP570795 | <0.01 | HRSPSGYRRQATGW | KMT2D |
| 479 | pNOP573651 | <0.01 | KSQSPSTFASKVCG | KMT2D |
| 480 | pNOP575068 | <0.01 | LLWPRGRHSPSGWD | KMT2D |
| 481 | pNOP580906 | <0.01 | RACSPGSGCGCGQG | KMT2D |
| 482 | pNOP580931 | <0.01 | RAGGAPQGCCLCPG | KMT2D |
| 483 | pNOP581766 | <0.01 | RIPWPRGQSRYTRT | KMT2D |
| 484 | pNOP584053 | <0.01 | SFLPITRYPSLPVP | KMT2D |
| 485 | pNOP588394 | <0.01 | VRPAQPTCGRGLCP | KMT2D |
| 486 | pNOP589969 | <0.01 | YLLTCLQRAPWSRA | KMT2D |
| 487 | pNOP591792 | <0.01 | ATRPLTSATGLIP | KMT2D |
| 488 | pNOP594808 | <0.01 | EKRLTCCDSSLSI | KMT2D |
| 489 | pNOP594895 | <0.01 | ELPLSQWPLNQER | KMT2D |
| 490 | pNOP595078 | <0.01 | EPLHRGRCGAGSR | KMT2D |
| 491 | pNOP596763 | <0.01 | GGCISGGGSLCSV | KMT2D |
| 515 | pNOP684498 | <0.01 | WLRAALGWHLV | KMT2D |
| 516 | pNOP68935 | <0.01 | PTLPATSTSHAFLYGCEQPATG | KMT2D |
| | | | RRLPSFLSASTLSWVPALTAAT | |
| | | | ATTVAATTGNSSNLHAICHVSS | |
| | | | LSINSWT | |
| 517 | pNOP704364 | <0.01 | MWRLPCTEDC | KMT2D |
| 518 | pNOP706242 | <0.01 | PAESSALGEG | KMT2D |
| 519 | pNOP708910 | <0.01 | QKLAWPCCVT | KMT2D |
| 520 | pNOP709657 | <0.01 | QSPLPAKGQR | KMT2D |
| 521 | pNOP713389 | <0.01 | RWCGAHGVRN | KMT2D |
| 522 | pNOP715424 | <0.01 | SQLLLPLRLW | KMT2D |
| | alt | | | |
| | splice e | | | |
| 523 | pNOP718753 | <0.01 | TWHLRKPGDQ | KMT2D |
| 524 | pNOP78569 | <0.01 | EHLGGGGPSFPSSGLRPVGARGP | KMT2D |
| | | | GPLPCHPPHSSGQHPSLPRYQT | |
| | | | LWGPWPGGPWKAACHNLGKGQRK | |
| 525 | pNOP81414 | <0.01 | IPTRSGLRTTLSVTAVTKPREVRL | KMT2D |
| | | | SAPLLSSIPRCVADFHPQSLAIPP | |
| | | | LTSPMLCTLHAKGSQRVGT | |
| 526 | pNOP85855 | <0.01 | DPGRGTDECGGCPAPRTANQVLPV | KMT2D |
| | | | PANWCHQQLQSHALPQCLPFCLCH | |
| | | | PCQVHVLQGQDHAVSNA | |
| 527 | pNOP98767 | <0.01 | TAPACLRHIRAPSQARPTPPTASS | KMT2D |
| | | | LCTPSHLSTGGCAPNGRTTCTWLA | |
| | | | PVSRAWGSMQPRT | |
| 528 | pNOP402895 | 0.23 | QKMILTKQIKTKPTDTFLQILR | PTEN |
| 529 | pNOP173513 | 0.16 | YQSRVLPQTEQDAKKGQNVSLLGK | PTEN |
| | | | YILHTRTRGNLRKSRKWKSM | |
| 530 | pNOP127569 | 0.14 | SWKGTNWCNDMCIFITSGQIFKGT | PTEN |
| | | | RGPRFLWGSKDQRQKGSNYSQSEA | |
| | | | LCVLL | |
| 531 | pNOP175050 | 0.07 | GFWIQSIKTITRYTIFVLKDIMTP | PTEN |
| | | | PNLIAELHNILLKTITHHS | |
| 532 | pNOP268063 | 0.07 | RYIPPIQDPHDGKTSSCTLSSLSR | PTEN |
| | | | YLCVVISK | |
| 533 | pNOP266820 | 0.04 | QKQKEISRGWIRLRLDLYLSKHYC | PTEN |
| | | | YGISCRKT | |
| 534 | pNOP421008 | 0.04 | QPSSKRSLAETKGDIKRMDST | PTEN |
| 535 | pNOP197013 | 0.04 | NYSNVQWRNLQSSVCGLPAKGEDIF | PTEN |
| | | | LQFRTHTTGRQVHVL | |
| 536 | pNOP325196 | 0.04 | PIFIQTLLLWDFLQKDLKAYTGTILMM | PTEN |
| 537 | pNOP546300 | 0.03 | KMEVYVIKKSIAFAV | PTEN |
| 538 | pNOP410561 | 0.03 | CLKLFQCSVAELAILSLWSAS | PTEN |
| 539 | pNOP547556 | 0.03 | LFPVRGAMCIIIATC | PTEN |
| 540 | pNOP554260 | 0.02 | RIIWIIDQWHCCFTR | PTEN |
| 541 | pNOP143081 | 0.02 | HQMLVTMNLIIIDILTPLTLIQRMNLLM | PTEN |
| | | | KISIHKLQKSEFFFIKRDKTP | |
| 542 | pNOP606239 | 0.02 | NLSNPFVKILTNG | PTEN |
| 543 | pNOP699983 | 0.01 | KPLQDIQSLC | PTEN |
| 544 | pNOP494212 | 0.01 | GEAVLHKNSRGAVKSRG | PTEN |
| 545 | pNOP445691 | <0.01 | VKMTIMLQQFTVKLERDELV | PTEN |
| 546 | pNOP571289 | <0.01 | IHSSYQDQRKPQKK | PTEN |
| 547 | pNOP682176 | <0.01 | TSGTVVSQDDV | PTEN |
| 548 | pNOP102380 | <0.01 | WSGGEKRRRRRPRRLQLQGGGLSRLSPFPGL | PTEN |
| | | | GTPESWSLPFYCLQHGGGGGGTSRDPGRF | |
| 549 | pNOP25104 | <0.01 | TSRPPPPHPPWPGLRRPPAEAAVRRIIRLLP | PTEN |
| | | | IPLPPLPGLWLLRRSRPSRCNHPAAAAAAIT | |
| | | | RLRSRAKRRQSEGHQLPPSPEPFPSCRRSPA | |
| | | | TSSFCHLSPPFSSATGSQT | |
| 550 | pNOP341110 | <0.01 | RSAYTNYKSLNFFLSRGIKHHENKLE | PTEN |
| 551 | pNOP401700 | <0.01 | PGAGGRSGGGGGRGGCSSREGV | PTEN |
| 552 | pNOP55619 | <0.01 | VACHHFQGWERRRVGLSPSTASNTAAAAAAH | PTEN |
| | | | PGTRAGFKPPVRRRRTPRGPGSGGRRRRQPF | |
| | | | GGLFVFSPFRCRRCQASGC | |
| 553 | pNOP61010 | <0.01 | GEAGPVAATIQQPPQQPLPGCGPEPSGGRAR | PTEN |
| | | | GISYRQVQSHFHPAEEAPPPAASAISLLLFL | |
| | | | QPQAPRHDSHHQRDR | |
| 554 | pNOP612548 | <0.01 | RSRQIQRLAVQLL | PTEN |
| 555 | pNOP672549 | <0.01 | PTTARTYQTLL | PTEN |
| 556 | pNOP673116 | <0.01 | QGISSTYFNKK | PTEN |
| 557 | pNOP676378 | <0.01 | RQSQPILFSKF | PTEN |
| 558 | pNOP685797 | <0.01 | YVHIYYIGANF | PTEN |

In a preferred embodiment the disclosure provides one or more frameshift-mutation peptides (also referred to herein as ‘neoantigens’) comprising an amino acid sequence selected from the groups:

(i) Sequences 29-129, an amino acid sequence having 90% identity to Sequences 29-129, or a fragment thereof comprising at least 10 consecutive amino acids of Sequences 29-129;

(ii) Sequences 130-156, an amino acid sequence having 90% identity to Sequences 130-156, or a fragment thereof comprising at least 10 consecutive amino acids of Sequences 130-156;

(iii) Sequences 157-272, an amino acid sequence having 90% identity to Sequences 157-272, or a fragment thereof comprising at least 10 consecutive amino acids of Sequences 157-272;

(iv) Sequences 273-527, an amino acid sequence having 90% identity to Sequences 273-527, or a fragment thereof comprising at least 10 consecutive amino acids of Sequences 273-527;

(v) Sequences 528-558, an amino acid sequence having 90% identity to Sequences 528-558, or a fragment thereof comprising at least 10 consecutive amino acids of Sequences 528-558, and

(vi) Sequences 1-28, an amino acid sequence having 90% identity to Sequences 1-28, or a fragment thereof comprising at least 10 consecutive amino acids of Sequences 1-28.

As will be clear to a skilled person, the preferred amino acid sequences may also be provided as a collection of tiled sequences, wherein such a collection comprises two or more peptides that have an overlapping sequence. Such ‘tiled’ peptides have the advantage that several peptides can be easily synthetically produced, while still covering a large portion of the NOP. In an exemplary embodiment, a collection comprising at least 3, 4, 5, 6, 10, or more tiled peptides each having between 10-50, preferably 12-45, more preferably 15-35 amino acids, is provided. As described further herein, such tiled peptides are preferably directed to the C-terminus of a pNOP. As will be clear to a skilled person, a collection of tiled peptides comprising an amino acid sequence of Sequence X, indicates that when aligning the tiled peptides and removing the overlapping sequences, the resulting tiled peptides provide the amino acid sequence of Sequence X, albeit present on separate peptides. As is also clear to a skilled person, a collection of tiled peptides comprising a fragment of 10 consecutive amino acids of Sequence X, indicates that when aligning the tiled peptides and removing the overlapping sequences, the resulting tiled peptides provide the amino acid sequence of the fragment, albeit present on separate peptides. When providing tiled peptides, the fragment preferably comprises at least 20 consecutive amino acids of a sequence as disclosed herein.

Specific NOP sequences cover a large percentage of cancer patients. Preferred NOP sequences, or subsequences of NOP sequences, are those that target the largest percentage of cancer patients. Preferred sequences are, preferably in this order of preference, Sequence 1 (0.9% of cancer patients) and Sequences 2-4 (0.8% of cancer patients), Sequence 5 (covering 0.7% of cancer patients), 6 (covering 0.6% of cancer patients), Sequence 7 (covering 0.5% of cancer patients), Sequence 130 (covering 0.4% of cancer patients), Sequences 273, 131 (covering 0.3% of cancer patients), Sequences 8-10, 30-37, 132, 157, 274, 528, 529 (each covering 0.2% of cancer patients), Sequences 11-18, 38-47, 133, 158-162, 275-279, 530-532 (each covering 0.1% of cancer patients), Sequences 48-51, 134, 280-282, 533-536 (each covering 0.04% of cancer patients), Sequences 19-20, 52-64, 135, 163-164, 283-286, 537-539 (each covering 0.03% of cancer patients), Sequences 21,22, 65-75, 136, 165-172, 287-306, 540-542 (each covering 0.02% of cancer patients), Sequences 23, 76-88, 173-190, 307-357, 543-544 (each covering 0.01% of cancer patients), and all other Sequences listed in Table 1 and not mentioned in this paragraph (each covering <0.01% of cancer patients).

As discussed further herein, neoantigens also include the nucleic acid molecules (such as DNA and RNA) encoding said amino acid sequences. The preferred sequences listed above are also the preferred sequences for the embodiments described further herein.

Preferably, the neoantigens and vaccines disclosed herein induce an immune response, or rather the neoantigens are immunogenic. Preferably, the neoantigens bind to an antibody or a T-cell receptor. In preferred embodiments, the neoantigens comprise an MHCI or MHCII ligand.

The major histocompatibility complex (MHC) is a set of cell surface molecules encoded by a large gene family in vertebrates. In humans, MHC is also referred to as human leukocyte antigen (HLA). An MHC molecule displays an antigen and presents it to the immune system of the vertebrate. Antigens (also referred to herein as ‘MHC ligands’) bind MHC molecules via a binding motif specific for the MHC molecule. Such binding motifs have been characterized and can be identified in proteins. See for a review Meydan et al. 2013 BMC Bioinformatics 14:S13.

MHC-class I molecules typically present the antigen to CD8 positive T-cells whereas MHC-class II molecules present the antigen to CD4 positive T-cells. The terms “cellular immune response” and “cellular response” or similar terms refer to an immune response directed to cells characterized by presentation of an antigen with class I or class II MHC involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

In preferred embodiments, the present disclosure involves the stimulation of an anti-tumor CTL response against tumor cells expressing one or more tumor-expressed antigens (i.e., NOPs) and preferably presenting such tumor-expressed antigens with class I MHC.

In some embodiments, an entire NOP (e.g., Sequence 1) may be provided as the neoantigen (i.e., peptide). The length of the NOPs identified herein vary from around 10 to around 140 amino acids. Preferred NOPs are at least 20 amino acids in length, more preferably at least 30 amino acids, and most preferably at least 50 amino acids in length. While not wishing to be bound by theory, it is believed that neoantigens longer than 10 amino acids can be processed into shorter peptides, e.g., by antigen presenting cells, which then bind to MHC molecules.

In some embodiments, fragments of a NOP can also be presented as the neoantigen. The fragments comprise at least 8 consecutive amino acids of the NOP, preferably at least 10 consecutive amino acids, and more preferably at least 20 consecutive amino acids, and most preferably at least 30 amino acids. In some embodiments, the fragments can be about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, or about 120 amino acids or greater. Preferably, the fragment is between 8-50, between 8-30, or between 10-20 amino acids. As will be understood by the skilled person, fragments greater than about 10 amino acids can be processed to shorter peptides, e.g., by antigen presenting cells.

The specific mutations resulting in the generation of a neo open reading frame may differ between individuals resulting in differing NOP lengths. However, as depicted in, e.g., FIG. 2, such individuals share common NOP sequences, in particular at the C-terminus of an NOP. While suitable fragments for use as neoantigens may be located at any position along the length of an NOP, fragments located near the C-terminus are preferred as they are expected to benefit a larger number of patients. Preferably, fragments of a NOP correspond to the C-terminal (3′) portion of the NOP, preferably the C-terminal 10 consecutive amino acids, more preferably the C-terminal 20 consecutive amino acids, more preferably the C-terminal 30 consecutive amino acids, more preferably the C-terminal 40 consecutive amino acids, more preferably the C-terminal 50 consecutive amino acids, more preferably the C-terminal 60 consecutive amino acids, more preferably the C-terminal 70 consecutive amino acids, more preferably the C-terminal 80 consecutive amino acids, more preferably the C-terminal 90 consecutive amino acids, and most preferably the C-terminal 100 or more consecutive amino acids. As is clear to a skilled person, the C-terminal amino acids need not include the, e.g., 1-5 most C-terminal amino acids. In some embodiments a subsequence of the preferred C-terminal portion of the NOP may be highly preferred for reasons of manufacturability, solubility and MHC binding strength.

Suitable fragments for use as neoantigens can be readily determined. The NOPs disclosed herein may be analysed by known means in the art in order to identify potential MHC binding peptides (i.e., MHC ligands). Suitable methods are described herein in the examples and include in silico prediction methods (e.g., ANNPRED, BIMAS, EPIMHC, HLABIND, IEDB, KISS, MULTIPRED, NetMHC, PEPVAC, POPI, PREDEP, RANKPEP, SVMHC, SVRMHC, and SYFFPEITHI, see Lundegaard 2010 130:309-318 for a review). MHC binding predictions depend on HLA genotypes, furthermore it is well known in the art that different MHC binding prediction programs predict different MHC affinities for a given epitope. While not wishing to be limited by such predictions, at least 60% of NOP sequences as defined herein, contain one or more predicted high affinity MHC class I binding epitope of 10 amino acids, based on allele HLA-A0201 and using NetMHC4.0.

A skilled person will appreciate that natural variations may occur in the genome resulting in variations in the sequence of an NOP. Accordingly, a neoantigen of the disclosure may comprise minor sequence variations, including, e.g., conservative amino acid substitutions. Conservative substitutions are well known in the art and refer to the substitution of one or more amino acids by similar amino acids. For example, a conservative substitution can be the substitution of an amino acid for another amino acid within the same general class (e.g., an acidic amino acid, a basic amino acid, or a neutral amino acid). A skilled person can readily determine whether such variants retain their immunogenicity, e.g., by determining their ability to bind MHC molecules.

Preferably, a neoantigen has at least 90% sequence identity to the NOPs disclosed herein. Preferably, the neoantigen has at least 95% or 98% sequence identity. The term “% sequence identity” is defined herein as the percentage of nucleotides in a nucleic acid sequence, or amino acids in an amino acid sequence, that are identical with the nucleotides, resp amino acids, in a nucleic acid or amino acid sequence of interest, after aligning the sequences and optionally introducing gaps, if necessary, to achieve the maximum percent sequence identity. The skilled person understands that consecutive amino acid residues in one amino acid sequence are compared to consecutive amino acid residues in another amino acid sequence. Methods and computer programs for alignments are well known in the art. Sequence identity is calculated over substantially the whole length, preferably the whole (full) length, of a sequence of interest.

The disclosure also provides at least two frameshift-mutation derived peptides (i.e., neoantigens), also referred to herein as a ‘collection’ of peptides. Preferably the collection comprises at least 3, at least 4, at least 5, at least 10, at least 15, or at least 20, or at least 50 neoantigens. In some embodiments, the collections comprise less than 20, preferably less than 15 neoantigens. Preferably, the collections comprise the top 20, more preferably the top 15 most frequently occurring neoantigens in cancer patients. The neoantigens are selected from

(i) Sequences 29-129, an amino acid sequence having 90% identity to Sequences 29-129, or a fragment thereof comprising at least 10 consecutive amino acids of Sequences 29-129;

(ii) Sequences 130-156, an amino acid sequence having 90% identity to Sequences 130-156, or a fragment thereof comprising at least 10 consecutive amino acids of Sequences 130-156;

(iii) Sequences 157-272, an amino acid sequence having 90% identity to Sequences 157-272, or a fragment thereof comprising at least 10 consecutive amino acids of Sequences 157-272;

(iv) Sequences 273-527, an amino acid sequence having 90% identity to Sequences 273-527, or a fragment thereof comprising at least 10 consecutive amino acids of Sequences 273-527;

(v) Sequences 528-558, an amino acid sequence having 90% identity to Sequences 528-558, or a fragment thereof comprising at least 10 consecutive amino acids of Sequences 528-558 and

(vi) Sequences 1-28, an amino acid sequence having 90% identity to Sequences 1-28, or a fragment thereof comprising at least 10 consecutive amino acids of Sequences 1-28.

Preferably, the collection comprises at least two frameshift-mutation derived peptides corresponding to the same gene. Preferably, a collection is provided comprising:

(i) a peptide, or a collection of tiled peptides, having the amino acid sequence selected from Sequence 29, an amino acid sequence having 90% identity to Sequence 29, or a fragment thereof comprising at least 10 consecutive amino acids of Sequence 29; and

a peptide, or a collection of tiled peptides, having the amino acid sequence selected from Sequence 30, an amino acid sequence having 90% identity to Sequence 30, or a fragment thereof comprising at least 10 consecutive amino acids of Sequence 30; preferably also comprising

a peptide, or a collection of tiled peptides, having the amino acid sequence selected from Sequences 31-33, an amino acid sequence having 90% identity to Sequences 31-33, or a fragment thereof comprising at least 10 consecutive amino acids of Sequences 31-33;

(ii) a peptide, or a collection of tiled peptides, having the amino acid sequence selected from Sequence 130, an amino acid sequence having 90% identity to Sequence 130, or a fragment thereof comprising at least 10 consecutive amino acids of Sequence 130; and

a peptide, or a collection of tiled peptides, having the amino acid sequence selected from Sequence 131, an amino acid sequence having 90% identity to Sequence, or a fragment thereof comprising at least 10 consecutive amino acids of Sequence,

(iii) a peptide, or a collection of tiled peptides, having the amino acid sequence selected from Sequence 157, an amino acid sequence having 90% identity to Sequence 157, or a fragment thereof comprising at least 10 consecutive amino acids of Sequence 157; and

a peptide, or a collection of tiled peptides, having the amino acid sequence selected from Sequence 158, an amino acid sequence having 90% identity to Sequence 158, or a fragment thereof comprising at least 10 consecutive amino acids of Sequence 158;

(iv) a peptide, or a collection of tiled peptides, having the amino acid sequence selected from Sequence 273, an amino acid sequence having 90% identity to Sequence 273, or a fragment thereof comprising at least 10 consecutive amino acids of Sequence 273; and

a peptide, or a collection of tiled peptides, having the amino acid sequence selected from Sequence 274, an amino acid sequence having 90% identity to Sequence 274, or a fragment thereof comprising at least 10 consecutive amino acids of Sequence 274;

(v) a peptide, or a collection of tiled peptides, having the amino acid sequence selected from Sequence 528, an amino acid sequence having 90% identity to Sequence 528, or a fragment thereof comprising at least 10 consecutive amino acids of Sequence 528; and

a peptide, or a collection of tiled peptides, having the amino acid sequence selected from Sequence 529, an amino acid sequence having 90% identity to Sequence 529, or a fragment thereof comprising at least 10 consecutive amino acids of Sequence 529 and/or

(vi) at least two peptides, wherein each peptide, or a collection of tiled peptides, comprises a different amino acid sequence selected from Sequences 1-3, an amino acid sequence having 90% identity to Sequences 1-3, or a fragment thereof comprising at least 10 consecutive amino acids of Sequences 1-3, preferably also comprising
a peptide, or a collection of tiled peptides, having the amino acid sequence selected from Sequence 4-15, an amino acid sequence having 90% identity to Sequence 4-15, or a fragment thereof comprising at least 10 consecutive amino acids of Sequence 4-15.

In some embodiments, the collection comprises two or more neoantigens corresponding to the same NOP. For example, the collection may comprise two (or more) fragments of Sequence 29 or the collection may comprise a peptide having Sequence 29 and a peptide having 95% identity to Sequence 29. For example, the collection may comprise two (or more) fragments of Sequence 1 or the collection may comprise a peptide having Sequence 1 and a peptide having 95% identity to Sequence 1.

Preferably, the collection comprises two or more neoantigens corresponding to different NOPs. In some embodiments, the collection comprises two or more neoantigens corresponding to different NOPs of the same gene. For example the peptide may comprise the amino acid sequence of Sequence 29 (or a fragment or collection of tiled fragments thereof) and the amino acid sequence of Sequence 30 (or a fragment or collection of tiled fragments thereof). For example the peptide may comprise the amino acid sequence of Sequence 1 (or a fragment or collection of tiled fragments thereof) and the amino acid sequence of Sequence 4 (or a fragment or collection of tiled fragments thereof).

Preferably, the collection comprises Sequences 29-129, preferably 29-88, more preferably 29-33 (or a fragment or collection of tiled fragments thereof).

Preferably, the collection comprises Sequences 130-156, preferably 130-136, more preferably 130-133 (or a fragment or collection of tiled fragments thereof).

Preferably, the collection comprises Sequences 157-272, preferably 157-172, more preferably 157-159 (or a fragment or collection of tiled fragments thereof).

Preferably, the collection comprises Sequences 273-527, preferably 273-306, more preferably 273-275 (or a fragment or collection of tiled fragments thereof).

Preferably, the collection comprises Sequences 528-558, preferably 528-544, more preferably 528-530 (or a fragment or collection of tiled fragments thereof).

Preferably, the collection comprises Sequences 528-558, preferably 528-544, more preferably 528-530 (or a fragment or collection of tiled fragments thereof).

In a preferred embodiment, the collections disclosed herein include
a peptide, or a collection of tiled peptides, comprising an amino acid sequence selected from Sequences 1-3, an amino acid sequence having 90% identity to Sequences 1-3, or a fragment thereof comprising at least 10 consecutive amino acids of Sequences 1-3, and
a peptide, or a collection of tiled peptides, comprising an amino acid sequence selected from Sequence 4, an amino acid sequence having 90% identity to Sequence 4, or a fragment thereof comprising at least 10 consecutive amino acids of Sequence 4, preferably also comprising
a peptide, or a collection of tiled peptides, comprising an amino acid sequence selected from Sequence 5, an amino acid sequence having 90% identity to Sequence 5, or a fragment thereof comprising at least 10 consecutive amino acids of Sequence 5,
a peptide, or a collection of tiled peptides, comprising an amino acid sequence selected from Sequence 6, an amino acid sequence having 90% identity to Sequence 6, or a fragment thereof comprising at least 10 consecutive amino acids of Sequence 6,
a peptide, or a collection of tiled peptides, comprising an amino acid sequence selected from Sequence 7, an amino acid sequence having 90% identity to Sequence 7, or a fragment thereof comprising at least 10 consecutive amino acids of Sequence 7,
a peptide, or a collection of tiled peptides, comprising an amino acid sequence selected from Sequence 8, an amino acid sequence having 90% identity to Sequence 8, or a fragment thereof comprising at least 10 consecutive amino acids of Sequence 8,
a peptide, or a collection of tiled peptides, comprising an amino acid sequence selected from Sequence 9, an amino acid sequence having 90% identity to Sequence 9, or a fragment thereof comprising at least 10 consecutive amino acids of Sequence 9,
a peptide, or a collection of tiled peptides, comprising an amino acid sequence selected from Sequence 10, an amino acid sequence having 90% identity to Sequence 10, or a fragment thereof comprising at least 10 consecutive amino acids of Sequence 10, and/or
a peptide, or a collection of tiled peptides, comprising an amino acid sequence selected from Sequence 11, an amino acid sequence having 90% identity to Sequence 11, or a fragment thereof comprising at least 10 consecutive amino acids of Sequence 11.

Preferably, the collection further comprises all of Sequences 1-28, preferably 1-23 (or a variant or fragment or collection of tiled fragments thereof as disclosed herein).

In some embodiments, the collection comprises two or more neoantigens corresponding to different NOPs of different genes. For example the collection may comprise a peptide having the amino acid sequence of Sequence 29 (or a fragment or collection of tiled fragments thereof) and a peptide having the amino acid sequence of Sequence 130 (or a fragment or collection of tiled fragments thereof). Preferably, the collection comprises at least one neoantigen from group (i) and at least one neoantigen from group (ii); at least one neoantigen from group (i) and at least one neoantigen from group (iii); at least one neoantigen from group (i) and at least one neoantigen from group (iv); at least one neoantigen from group (i) and at least one neoantigen from group (v); at least one neoantigen from group (ii) and at least one neoantigen from group (iii); at least one neoantigen from group (ii) and at least one neoantigen from group (iv); at least one neoantigen from group (ii) and at least one neoantigen from group (v); or at least one neoantigen from group (iii) and at least one neoantigen from group (iv). Preferably, the collection comprises at least one neoantigen from group (i), at least one neoantigen from group (ii), and at least one neoantigen from group (iii). Preferably, the collection comprises at least one neoantigen from each of groups (i) to (iv). Preferably, the collection comprises at least one neoantigen from each of groups (i) to (v).

Preferably, the collection comprises at least one neoantigen from group (i) and at least one neoantigen from group (vi); at least one neoantigen from group (ii) and at least one neoantigen from group (vi); at least one neoantigen from group (iii) and at least one neoantigen from group (vi); at least one neoantigen from group (iv) and at least one neoantigen from group (vi); at least one neoantigen from group (v) and at least one neoantigen from group (vi); Preferably, the collection comprises at least one neoantigen from group (i), at least one neoantigen from group (ii), and at least one neoantigen from group (vi). Preferably, the collection comprises at least one neoantigen from each of groups (i) to (vi).

In preferred embodiments, the collection includes Sequence 130 and one or both of Sequences 273, 131 (or a variant or fragment or collection of tiled fragments thereof as disclosed herein). In a preferred embodiment, the collections disclosed herein include Sequence 1 (or a variant or fragment or collection of tiled fragments thereof as disclosed herein). In preferred embodiments, the collection even further includes one or more of Sequences 30-37, 132, 157, 274, 528, 529 (or a variant or fragment or collection of tiled fragments thereof as disclosed herein). In preferred embodiments, the collection even further includes one or more of Sequences 38-47, 133, 158-162, 275-279, 530-532 (or a variant or fragment or collection of tiled fragments thereof as disclosed herein). In preferred embodiments, the collection even further includes one or more of Sequences 48-51, 134, 280-282, 533-536 (or a variant or fragment or collection of tiled fragments thereof as disclosed herein). In preferred embodiments, the collection even further includes one or more of Sequences 52-64, 135, 163-164, 283-286, 537-539 (or a variant or fragment or collection of tiled fragments thereof as disclosed herein). In preferred embodiments, the collection even further includes one or more of Sequences 65-75, 136, 165-172, 287-306, 540-542 (or a variant or fragment or collection of tiled fragments thereof as disclosed herein). In preferred embodiments, the collection even further includes one or more of Sequences 76-88, 173-190, 307-357, 543-544 (or a variant or fragment or collection of tiled fragments thereof as disclosed herein). In preferred embodiments, the collection even further includes all other Sequences listed in Table 1 and not mentioned in this paragraph (or a variant or fragment or collection of tiled fragments thereof as disclosed herein).

In a preferred embodiment, the collections disclosed herein include two or all of Sequence 1-3 (or a variant or fragment or collection of tiled fragments thereof as disclosed herein). In some embodiments, the collection further includes Sequence 4 (or a variant or fragment or collection of tiled fragments thereof as disclosed herein). In some embodiments, the collection further includes one or both of Sequence 5 and 6 (or a variant or fragment or collection of tiled fragments thereof as disclosed herein). In some embodiments, the collection further includes one or both of Sequence 7, 8 (or a variant or fragment or collection of tiled fragments thereof as disclosed herein). In some embodiments, the collection further includes one or more, preferably all of Sequence 9-24 (or a variant or fragment or collection of tiled fragments thereof as disclosed herein). In some embodiments, the collection further includes one or more, preferably all of Sequence 25-28 (or a variant or fragment or collection of tiled fragments thereof as disclosed herein).

In a preferred embodiment, the collections disclosed herein include Sequence 130 (or a variant or fragment or collection of tiled fragments thereof as disclosed herein). Preferably, the collection includes Sequence 130 (or a variant or fragment or collection of tiled fragments thereof as disclosed herein) and one or more sequences selected from 1-23, 29-88, 130-136, 157-172, 273-306, 528-544 (or a variant or fragment or collection of tiled fragments thereof as disclosed herein).

Such collections comprising multiple neoantigens have the advantage that a single collection (e.g, when used as a vaccine) can benefit a larger group of patients having different frameshift mutations. This makes it feasible to construct and/or test the vaccine in advance and have the vaccine available for off-the-shelf use. This also greatly reduces the time from screening a tumor from a patient to administering a potential vaccine for said tumor to the patient, as it eliminates the time of production, testing and approval. In addition, a single collection consisting of multiple neoantigens corresponding to different genes will limit possible resistance mechanisms of the tumor, e.g. by losing one or more of the targeted neoantigens.

In some embodiments, the collection of frameshift mutation peptides may further include one or more TP53 frameshift-mutation peptides. Suitable TP53 frameshift-mutation peptides include sequences 1-28, preferably sequences 1-18 (or fragment or collection of tiled fragments thereof as disclosed herein). In a preferred embodiment, the collections disclosed herein include Sequence 1 (or a variant or fragment or collection of tiled fragments thereof as disclosed herein). In preferred embodiments, the collection further includes one, two or more of Sequences 2-4 (or a variant or fragment or collection of tiled fragments thereof as disclosed herein). In preferred embodiments, the collection further includes Sequence 5 (or a variant or fragment or collection of tiled fragments thereof as disclosed herein). In preferred embodiments, the collection even further includes Sequence 6 (or a variant or fragment or collection of tiled fragments thereof as disclosed herein). In preferred embodiments, the collection even further includes Sequence 7 (or a variant or fragment or collection of tiled fragments thereof as disclosed herein).

In some embodiments, the collection of TP53 frameshift-mutation peptides further comprises one or more ARID1A frameshift-mutation peptides as disclosed herein, one or more CDKN2A frameshift-mutation peptides as disclosed herein, one or more KMT2B frameshift-mutation peptides as disclosed herein, one or more KMT2D frameshift-mutation peptides as disclosed herein, and/or one or more PTEN frameshift-mutation peptides as disclosed herein.

Suitable ARID1A frameshift-mutation peptides to be combined with TP53 frameshift-mutation peptides, include sequences 29-129 (or a fragment or collection of tiled fragments thereof), preferably sequences 29-38. Suitable CDKN2A frameshift-mutation peptides to be combined with TP53 frameshift-mutation peptides, include sequences 130-156 (or a fragment or collection of tiled fragments thereof), preferably sequences 130-136. Suitable KMT2B frameshift-mutation peptides to be combined with TP53 frameshift-mutation peptides, include sequences 157-272 (or a fragment or collection of tiled fragments thereof), preferably sequences 157-164. Suitable KMT2D frameshift-mutation peptides to be combined with TP53 frameshift-mutation peptides, include sequences 273-527 (or a fragment or collection of tiled fragments thereof), preferably sequences 273-286. Suitable PTEN frameshift-mutation peptides to be combined with TP53 frameshift-mutation peptides, include sequences 528-558 (or a fragment or collection of tiled fragments thereof), preferably sequences 528-542. Preferably, the collections comprise TP53 frameshift-mutation peptides, ARID1A frameshift-mutation peptides, and CDKN2A frameshift-mutation peptides.

In preferred embodiments, the neoantigens (i.e., peptides) are directly linked. Preferably, the neoantigens are linked by peptide bonds, or rather, the neoantigens are present in a single polypeptide. Accordingly, the disclosure provides polypeptides comprising at least two peptides (i.e., neoantigens) as disclosed herein. In some embodiments, the polypeptide comprises 3, 4, 5, 6, 7, 8, 9, 10 or more peptides as disclosed herein (i.e., neoantigens). Such polypeptides are also referred to herein as ‘polyNOPs’. A collection of peptides can have one or more peptides and one or more polypeptides comprising the respective neoantigens.

In an exemplary embodiment, a polypeptide of the disclosure may comprise 10 different neoantigens, each neoantigen having between 10-400 amino acids. Thus, the polypeptide of the disclosure may comprise between 100-4000 amino acids, or more. As is clear to a skilled person, the final length of the polypeptide is determined by the number of neoantigens selected and their respective lengths. A collection may comprise two or more polypeptides comprising the neoantigens which can be used to reduce the size of each of the polypeptides.

In some embodiments, the amino acid sequences of the neoantigens are located directly adjacent to each other in the polypeptide. For example, a nucleic acid molecule may be provided that encodes multiple neoantigens in the same reading frame. In some embodiments, a linker amino acid sequence may be present. Preferably a linker has a length of 1, 2, 3, 4 or 5, or more amino acids. The use of linker may be beneficial, for example for introducing, among others, signal peptides or cleavage sites. In some embodiments at least one, preferably all of the linker amino acid sequences have the amino acid sequence VDD.

As will be appreciated by the skilled person, the peptides and polypeptides disclosed herein may contain additional amino acids, for example at the N- or C-terminus. Such additional amino acids include, e.g., purification or affinity tags or hydrophilic amino acids in order to decrease the hydrophobicity of the peptide. In some embodiments, the neoantigens may comprise amino acids corresponding to the adjacent, wild-type amino acid sequences of the relevant gene, i.e., amino acid sequences located 5′ to the frame shift mutation that results in the neo open reading frame. Preferably, each neoantigen comprises no more than 20, more preferably no more than 10, and most preferably no more than 5 of such wild-type amino acid sequences.

In preferred embodiments, the peptides and polypeptides disclosed herein have a sequence depicted as follows:

A-B-C-(D-E)ₙ, wherein
A, C, and E are independently 0-100 amino acids
B and D are amino acid sequences as disclosed herein and selected from sequences 29-558, or an amino acid sequence having 90% identity to Sequences 29-558, or a fragment thereof comprising at least 10 consecutive amino acids of Sequences 29-558,
n is an integer from 0 to 500.

Preferably, B and D are different amino acid sequences. Preferably, n is an integer from 0-200. Preferably A, C, and E are independently 0-50 amino acids, more preferably independently 0-20 amino acids.

The peptides and polypeptides disclosed herein can be produced by any method known to a skilled person. In some embodiments, the peptides and polypeptide are chemically synthesized. The peptides and polypeptide can also be produced using molecular genetic techniques, such as by inserting a nucleic acid into an expression vector, introducing the expression vector into a host cell, and expressing the peptide. Preferably, such peptides and polypeptide are isolated, or rather, substantially isolated from other polypeptides, cellular components, or impurities. The peptide and polypeptide can be isolated from other (poly)peptides as a result of solid phase protein synthesis, for example. Alternatively, the peptides and polypeptide can be substantially isolated from other proteins after cell lysis from recombinant production (e.g., using HPLC).

The disclosure further provides nucleic acid molecules encoding the peptides and polypeptide disclosed herein. Based on the genetic code, a skilled person can determine the nucleic acid sequences which encode the (poly)peptides disclosed herein. Based on the degeneracy of the genetic code, sixty-four codons may be used to encode twenty amino acids and translation termination signal.

In a preferred embodiment, the nucleic acid molecules are codon optimized. As is known to a skilled person, codon usage bias in different organisms can effect gene expression level. Various computational tools are available to the skilled person in order to optimize codon usage depending on which organism the desired nucleic acid will be expressed. Preferably, the nucleic acid molecules are optimized for expression in mammalian cells, preferably in human cells. Table 2 lists for each acid amino acid (and the stop codon) the most frequently used codon as encountered in the human exome.

TABLE 2
most frequently used
codon for each amino acid
and most frequently used
stop codon.

| | |
|---|---|
| A | GCC |
| C | TGC |
| D | GAC |
| E | GAG |
| F | TTC |
| G | GGC |
| H | CAC |
| I | ATC |
| K | AAG |
| L | CTG |
| M | ATG |
| N | AAC |
| P | CCC |
| Q | CAG |
| R | CGG |
| S | AGC |
| T | ACC |
| V | GTG |
| W | TGG |
| Y | TAC |
| Stop | TGA |

In preferred embodiments, at least 50%, 60%, 70%, 80%, 90%, or 100% of the amino acids are encoded by a codon corresponding to a codon presented in Table 2.

In preferred embodiments, the nucleic acid molecule encodes for a linker amino acid sequence in the peptide. Preferably, the nucleic acid sequence encoding the linker comprises at least one codon triplet that codes for a stop codon when a frameshift occurs. Preferably, said codon triplet is chosen from the group consisting of: ATA, CTA, GTA, TTA, ATG, CTG, GTG, TTG, AAA, AAC, AAG, AAT, AGA, AGC, AGG, AGT, GAA, GAC, GAG, and GAT. These codons do not code for a stop codon, but could create a stop codon in case of a frame shift, such as when read in the +1, +2, +4, +, 5, etc. reading frame. For example, two amino acid encoding sequences are linked by a linker amino acid encoding sequence as follows (linker amino acid encoding sequence in bold):

CTATACAGGCGAATGAGATTATG

Resulting in the following amino acid sequence (amino acid linker sequence in bold): LYRRMRL

In case of a +1 frame shift, the following sequence is encoded:

YTGE [stop] DY

This embodiment has the advantage that if a frame shift occurs in the nucleotide sequence encoding the peptide, the nucleic acid sequence encoding the linker will terminate translation, thereby preventing expression of (part of) the native protein sequence for the gene related to peptide sequence encoded by the nucleotide sequence.

In some preferred embodiments, the linker amino acid sequences are encoded by the nucleotide sequence GTAGATGAC. This linker has the advantage that it contains two out of frame stop codons (TAG and TGA), one in the +1 and one in the −1 reading frame. The amino acid sequence encoded by this nucleotide sequence is VDD. The added advantage of using a nucleotide sequence encoding for this linker amino acid sequence is that any frame shift will result in a stop codon.

The disclosure also provides binding molecules and a collection of binding molecules that bind the neoantigens disclosed herein and or a neoantigen/MHC complex. In some embodiments the binding molecule is an antibody, a T-cell receptor, or an antigen binding fragment thereof. In some embodiments the binding molecule is a chimeric antigen receptor comprising i) a T cell activation molecule; ii) a transmembrane region; and iii) an antigen recognition moiety; wherein said antigen recognition moieties bind the neoantigens disclosed herein and or a neoantigen/MHC complex.

The term “antibody” as used herein refers to an immunoglobulin molecule that is typically composed of two identical pairs of polypeptide chains, each pair of chains consisting of one “heavy” chain with one “light” chain. The human light chains are classified as kappa and lambda. The heavy chains comprise different classes namely: mu, delta, gamma, alpha or epsilon. These classes define the isotype of the antibody, such as IgM, IgD, IgG IgA and IgE, respectively. These classes are important for the function of the antibody and help to regulate the immune response. Both the heavy chain and the light chain comprise a variable domain and a constant region. Each heavy chain variable region (VH) and light chain variable region (VL) comprises complementary determining regions (CDR) interspersed by framework regions (FR). The variable region has in total four FRs and three CDRs. These are arranged from the amino- to the carboxyl-terminus as follows: FR1. CDR1, FR2, CDR2, FR3, CDR3, FR4. The variable regions of the light and heavy chain together form the antibody binding site and define the specificity for the epitope.

The term “antibody” encompasses murine, humanized, deimmunized, human, and chimeric antibodies, and an antibody that is a multimeric form of antibodies, such as dimers, trimers, or higher-order multimers of monomeric antibodies. The term antibody also encompasses monospecific, bispecific or multi-specific antibodies, and any other modified configuration of the immunoglobulin molecule that comprises an antigen recognition site of the required specificity.

Preferably, an antibody or antigen binding fragment thereof as disclosed herein is a humanized antibody or antigen binding fragment thereof. The term “humanized antibody” refers to an antibody that contains some or all of the CDRs from a non-human animal antibody while the framework and constant regions of the antibody contain amino acid residues derived from human antibody sequences. Humanized antibodies are typically produced by grafting CDRs from a mouse antibody into human framework sequences followed by back substitution of certain human framework residues for the corresponding mouse residues from the source antibody. The term “deimmunized antibody” also refers to an antibody of non-human origin in which, typically in one or more variable regions, one or more epitopes have been removed, that have a high propensity of constituting a human T-cell and/or B-cell epitope, for purposes of reducing immunogenicity. The amino acid sequence of the epitope can be removed in full or in part. However, typically the amino acid sequence is altered by substituting one or more of the amino acids constituting the epitope for one or more other amino acids, thereby changing the amino acid sequence into a sequence that does not constitute a human T-cell and/or B-cell epitope. The amino acids are substituted by amino acids that are present at the corresponding position(s) in a corresponding human variable heavy or variable light chain as the case may be.

In some embodiments, an antibody or antigen binding fragment thereof as disclosed herein is a human antibody or antigen binding fragment thereof. The term “human antibody” refers to an antibody consisting of amino acid sequences of human immunoglobulin sequences only. Human antibodies may be prepared in a variety of ways known in the art.

As used herein, antigen-binding fragments include Fab, F(ab′), F(ab′)2, complementarity determining region (CDR) fragments, single-chain antibodies (scFv), bivalent single-chain antibodies, and other antigen recognizing immunoglobulin fragments.

In some embodiments, the antibody or antigen binding fragment thereof is an isolated antibody or antigen binding fragment thereof. The term “isolated” as used herein refer to material which is substantially or essentially free from components which normally accompany it in nature.

In some embodiments, the antibody or antigen binding fragment thereof is linked or attached to a non-antibody moiety. In preferred embodiments, the non-antibody moiety is a cytotoxic moiety such as auristatins, maytanasines, calicheasmicins, duocarymycins, a-amanitin, doxorubicin, and centanamycin. Other suitable cytotoxins and methods for preparing such antibody drug conjugates are known in the art; see, e.g., WO2013085925A1 and WO2016133927A1.

Antibodies which bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e g, affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, B. B., et al., Selected Methods In Cellular Immunology, (W.H. Freeman, ed.) San Francisco (1980)). Peptides corresponding to the neoantiens disclosed herein may be used for immunization in order to produce antibodies which recognize a particular epitope. Screening for recognition of the epitope can be performed using standard immunoassay methods including ELISA techniques, radioimmunoassays, immunofluorescence, immunohistochemistry, and Western blotting. See, Short Protocols in Molecular Biology, Chapter 11, Green Publishing Associates and John Wiley & Sons, Edited by Ausubel, F. M et al., 1992. In vitro methods of antibody selection, such as antibody phage display, may also be used to generate antibodies recognizing the neoantigens disclosed herein (see, e.g., Schirrmann et al. Molecules 2011 16:412-426).

T-cell receptors (TCRs) are expressed on the surface of T-cells and consist of an α chain and a β chain. TCRs recognize antigens bound to MHC molecules expressed on the surface of antigen-presenting cells. The T-cell receptor (TCR) is a heterodimeric protein, in the majority of cases (95%) consisting of a variable alpha (α) and beta (β) chain, and is expressed on the plasma membrane of T-cells. The TCR is subdivided in three domains: an extracellular domain, a transmembrane domain and a short intracellular domain. The extracellular domain of both α and β chains have an immunoglobulin-like structure, containing a variable and a constant region. The variable region recognizes processed peptides, among which neoantigens, presented by major histocompatibility complex (MHC) molecules, and is highly variable. The intracellular domain of the TCR is very short, and needs to interact with CD3ζ to allow for signal propagation upon ligation of the extracellular domain.

With the focus of cancer treatment shifted towards more targeted therapies, among which immunotherapy, the potential of therapeutic application of tumor-directed T-cells is increasingly explored. One such application is adoptive T-cell therapy (ATCT) using genetically modified T-cells that carry chimeric antigen receptors (CARs) recognizing a particular epitope (Ref Gomes-Silva 2018). The extracellular domain of the CAR is commonly formed by the antigen-specific subunit of (scFv) of a monoclonal antibody that recognizes a tumor-antigen (Ref Abate-Daga 2016). This enables the CAR T-cell to recognize epitopes independent of MHC-molecules, thus widely applicable, as their functionality is not restricted to individuals expressing the specific MHC-molecule recognized by the TCR. Methods for engineering TCRs that bind a particular epitope are known to a skilled person. See, for example, US20100009863A1, which describes methods of modifying one or more structural loop regions. The intracellular domain of the CAR can be a TCR intracellular domain or a modified peptide to enable induction of a signaling cascade without the need for interaction with accessory proteins. This is accomplished by inclusion of the CD3-signalling domain, often in combination with one or more co-stimulatory domains, such as CD28 and 4-1BB, which further enhance CAR T-cell functioning and persistence (Ref Abate-Daga 2016).

The engineering of the extracellular domain towards an scFv limits CAR T-cell to the recognition of molecules that are expressed on the cell-surface. Peptides derived from proteins that are expressed intracellularly can be recognized upon their presentation on the plasma membrane by MHC molecules, of which human form is called human leukocyte antigen (HLA). The HLA-haplotype generally differs among individuals, but some HLA types, like HLA-A*02:01, are globally common. Engineering of CAR T-cell extracellular domains recognizing tumor-derived peptides or neoantigens presented by a commonly shared HLA molecule enables recognition of tumor antigens that remain intracellular. Indeed CAR T-cells expressing a CAR with a TCR-like extracellular domain have been shown to be able to recognize tumor-derived antigens in the context of HLA-A*02:01 (Refs Zhang 2014, Ma 2016, Liu 2017).

In some embodiments, the binding molecules are monospecific, or rather they bind one of the neoantigens disclosed herein. In some embodiments, the binding molecules are bispecific, e.g., bispecific antibodies and bispecific chimeric antigen receptors.

In some embodiments, the disclosure provides a first antigen binding domain that binds a first neoantigen described herein and a second antigen binding domain that binds a second neoantigen described herein. The first and second antigen binding domains may be part of a single molecule, e.g., as a bispecific, antibody or bispecific chimeric antigen receptor or they may be provided on separate molecules, e.g., as a collection of antibodies, T-cell receptors, or chimeric antigen receptors. In some embodiments, 3, 4, 5 or more antigen binding domains are provided each binding a different neoantigen disclosed herein. As used herein, an antigen binding domain includes the variable (antigen binding) domain of a T-cell receptor and the variable domain of an antibody (e.g., comprising a light chain variable region and a heavy chain variable region).

The disclosure further provides nucleic acid molecules encoding the antibodies, TCRs, and CARs disclosed herein. In a preferred embodiment, the nucleic acid molecules are codon optimized as disclosed herein.

The disclosure further provides vectors comprising the nucleic acids molecules disclosed herein. A “vector” is a recombinant nucleic acid construct, such as plasmid, phase genome, virus genome, cosmid, or artificial chromosome, to which another nucleic acid segment may be attached. The term “vector” includes both viral and non-viral means for introducing the nucleic acid into a cell in vitro, ex vivo or in vivo. The disclosure contemplates both DNA and RNA vectors. The disclosure further includes self-replicating RNA with (virus-derived) replicons, including but not limited to mRNA molecules derived from mRNA molecules from alphavirus genomes, such as the Sindbis, Semliki Forest and Venezuelan equine encephalitis viruses.

Vectors, including plasmid vectors, eukaryotic viral vectors and expression vectors are known to the skilled person. Vectors may be used to express a recombinant gene construct in eukaryotic cells depending on the preference and judgment of the skilled practitioner (see, for example, Sambrook et al., Chapter 16). For example, many viral vectors are known in the art including, for example, retroviruses, adeno-associated viruses, and adenoviruses. Other viruses useful for introduction of a gene into a cell include, but a not limited to, arenavirus, herpes virus, mumps virus, poliovirus, Sindbis virus, and vaccinia virus, such as, canary pox virus. The methods for producing replication-deficient viral particles and for manipulating the viral genomes are well known. In preferred embodiments, the vaccine comprises an attenuated or inactivated viral vector comprising a nucleic acid disclosed herein.

Preferred vectors are expression vectors. It is within the purview of a skilled person to prepare suitable expression vectors for expressing the inhibitors disclosed hereon. An “expression vector” is generally a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. Suitable regulatory sequences including enhancers, promoters, translation initiation signals, and polyadenylation signals may be included. Additionally, depending on the host cell chosen and the vector employed, other sequences, such as an origin of replication, additional DNA restriction sites, enhancers, and sequences conferring inducibility of transcription may be incorporated into the expression vector. The expression vectors may also contain a selectable marker gene which facilitates the selection of host cells transformed or transfected. Examples of selectable marker genes are genes encoding a protein such as G418 and hygromycin which confer resistance to certain drugs, 6-galactosidase, chloramphenicol acetyltransferase, and firefly luciferase.

The expression vector can also be an RNA element that contains the sequences required to initiate translation in the desired reading frame, and possibly additional elements that are known to stabilize or contribute to replicate the RNA molecules after administration. Therefore when used herein the term DNA when referring to an isolated nucleic acid encoding the peptide according to the invention should be interpreted as referring to DNA from which the peptide can be transcribed or RNA molecules from which the peptide can be translated.

Also provided for is a host cell comprising a nucleic acid molecule or a vector as disclosed herein. The nucleic acid molecule may be introduced into a cell (prokaryotic or eukaryotic) by standard methods. As used herein, the terms “transformation” and “transfection” are intended to refer to a variety of art recognized techniques to introduce a DNA into a host cell. Such methods include, for example, transfection, including, but not limited to, liposome-polybrene, DEAE dextran-mediated transfection, electroporation, calcium phosphate precipitation, microinjection, or velocity driven microprojectiles (“biolistics”). Such techniques are well known by one skilled in the art. See, Sambrook et al. (1989) Molecular Cloning: A Laboratory Manaual (2 ed. Cold Spring Harbor Lab Press, Plainview, N.Y.). Alternatively, one could use a system that delivers the DNA construct in a gene delivery vehicle. The gene delivery vehicle may be viral or chemical. Various viral gene delivery vehicles can be used with the present invention. In general, viral vectors are composed of viral particles derived from naturally occurring viruses. The naturally occurring virus has been genetically modified to be replication defective and does not generate additional infectious viruses, or it may be a virus that is known to be attenuated and does not have unacceptable side effects.

Preferably, the host cell is a mammalian cell, such as MRCS cells (human cell line derived from lung tissue), HuH7 cells (human liver cell line), CHO-cells (Chinese Hamster Ovary), COS-cells (derived from monkey kidney (African green monkey), Vero-cells (kidney epithelial cells extracted from African green monkey), Hela-cells (human cell line), BHK-cells (baby hamster kidney cells, HEK-cells (Human Embryonic Kidney), NSO-cells (Murine myeloma cell line), C127-cells (nontumorigenic mouse cell line), PerC6®-cells (human cell line, Crucell), and Madin-Darby Canine Kidney (MDCK) cells. In some embodiments, the disclosure comprises an in vitro cell culture of mammalian cells expressing the neoantigens disclosed herein. Such cultures are useful, for example, in the production of cell-based vaccines, such as viral vectors expressing the neoantigens disclosed herein.

In some embodiments the host cells express the antibodies, TCRs, or CARs as disclosed herein. As will be clear to a skilled person, individual polypeptide chains (e.g., immunoglobulin heavy and light chains) may be provided on the same or different nucleic acid molecules and expressed by the same or different vectors. For example, in some embodiments, a host cell is transfected with a nucleic acid encoding an α-TCR polypeptide chain and a nucleic acid encoding a β-polypeptide chain.

In preferred embodiments, the disclosure provides T-cells expressing a TCR or CAR as disclosed herein. T cells may be obtained from, e.g., peripheral blood mononuclear cells, bone marrow, lymph node tissue, cord blood, thymus tissue, spleen tissue, and tumors. Preferably, the T-cells are obtained from the individual to be treated (autologous T-cells). T-cells may also be obtained from healthy donors (allogenic T-cells). Isolated T-cells are expanded in vitro using established methods, such as stimulation with cytokines (IL-2). Methods for obtaining and expanding T-cells for adoptive therapy are well known in the art and are also described, e.g., in EP2872533A1.

The disclosure also provides vaccines comprising one or more neoantigens as disclosed herein. In particular, the vaccine comprises one or more (poly)peptides, antibodies or antigen binding fragments thereof, TCRs, CARS, nucleic acid molecules, vectors, or cells (or cell cultures) as disclosed herein.

The vaccine may be prepared so that the selection, number and/or amount of neoantigens (e.g., peptides or nucleic acids encoding said peptides) present in the composition is patient-specific. Selection of one or more neoantigens may be based on sequencing information from the tumor of the patient. For any frame shift mutation found, a corresponding NOP is selected. Preferably, the vaccine comprises more than one neoantigen corresponding to the NOP selected. In case multiple frame shift mutations (multiple NOPs) are found, multiple neoantigens corresponding to each NOP may be selected for the vaccine.

The selection may also be dependent on the specific type of cancer, the status of the disease, earlier treatment regimens, the immune status of the patient, and, HLA-haplotype of the patient. Furthermore, the vaccine can contain individualized components, according to personal needs of the particular patient.

As is clear to a skilled person, if multiple neoantigens are used, they may be provided in a single vaccine composition or in several different vaccines to make up a vaccine collection. The disclosure thus provides vaccine collections comprising a collection of tiled peptides, collection of peptides as disclosed herein, as well as nucleic acid molecules, vectors, or host cells as disclosed herein. As is clear to a skilled person, such vaccine collections may be administered to an individual simultaneously or consecutively (e.g., on the same day) or they may be administered several days or weeks apart.

Various known methods may be used to administer the vaccines to an individual in need thereof. For instance, one or more neoantigens can be provided as a nucleic acid molecule directly, as “naked DNA”. Neoantigens can also be expressed by attenuated viral hosts, such as vaccinia or fowlpox. This approach involves the use of a virus as a vector to express nucleotide sequences that encode the neoantigen. Upon introduction into the individual, the recombinant virus expresses the neoantigen peptide, and thereby elicits a host CTL response. Vaccination using viral vectors is well-known to a skilled person and vaccinia vectors and methods useful in immunization protocols are described in, e.g., U.S. Pat. No. 4,722,848. Another vector is BCG (Bacille Calmette Guerin) as described in Stover et al. (Nature 351:456-460 (1991)).

Preferably, the vaccine comprises a pharmaceutically acceptable excipient and/or an adjuvant. The compositions may contain pharmaceutically acceptable auxiliary substances as required to approximate physiological conditions, such as pH adjusting and buffering agents, tonicity adjusting agents, wetting agents and the like. Suitable adjuvants are well-known in the art and include, aluminum (or a salt thereof, e.g., aluminium phosphate and aluminium hydroxide), monophosphoryl lipid A, squalene MF59), and cytosine phosphoguanine (CpG), montanide, liposomes (e.g. CAF adjuvants, cationic adjuvant formulations and variations thereof), lipoprotein conjugates (e.g. Amplivant), Resiquimod, Iscomatrix, hiltonol, poly-ICLC (polyriboinosinic-polyribocytidylic acid-polylysine carboxymethylcellulose). A skilled person is able to determine the appropriate adjuvant, if necessary, and an immune-effective amount thereof. As used herein, an immune-effective amount of adjuvant refers to the amount needed to increase the vaccine's immunogenicity in order to achieve the desired effect.

The disclosure also provides the use of the neoantigens disclosed herein for the treatment of disease, in particular for the treatment of cancer in an individual. It is within the purview of a skilled person to diagnose an individual with as having cancer.

As used herein, the terms “treatment,” “treat,” and “treating” refer to reversing, alleviating, or inhibiting the progress of a disease, or reversing, alleviating, delaying the onset of, or inhibiting one or more symptoms thereof. Treatment includes, e.g., slowing the growth of a tumor, reducing the size of a tumor, and/or slowing or preventing tumor metastasis.

The term ‘individual’ includes mammals, both humans and non-humans and includes but is not limited to humans, non-human primates, canines, felines, murines, bovines, equines, and porcines. Preferably, the human is a mammal.

As used herein, administration or administering in the context of treatment or therapy of a subject is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners.

The optimum amount of each neoantigen to be included in the vaccine composition and the optimum dosing regimen can be determined by one skilled in the art without undue experimentation. The composition may be prepared for injection of the peptide, nucleic acid molecule encoding the peptide, or any other carrier comprising such (such as a virus or liposomes). For example, doses of between 1 and 500 mg 50 μg and 1.5 mg, preferably 125 μg to 500 μg, of peptide or DNA may be given and will depend from the respective peptide or DNA. Other methods of administration are known to the skilled person. Preferably, the vaccines may be administered parenterally, e.g., intravenously, subcutaneously, intradermally, intramuscularly, or otherwise.

For therapeutic use, administration may begin at or shortly after the surgical removal of tumors. This can be followed by boosting doses until at least symptoms are substantially abated and for a period thereafter.

In some embodiments, the vaccines may be provided as a neoadjuvant therapy, e.g., prior to the removal of tumors or prior to treatment with radiation or chemotherapy. Neoadjuvant therapy is intended to reduce the size of the tumor before more radical treatment is used. For that reason being able to provide the vaccine off-the-shelf or in a short period of time is very important.

Also disclosed herein, the vaccine is capable of initiating a specific T-cell response. It is within the purview of a skilled person to measure such T-cell responses either in vivo or in vitro, e.g. by analyzing IFN-γ production or tumor killing by T-cells. In therapeutic applications, vaccines are administered to a patient in an amount sufficient to elicit an effective CTL response to the tumor antigen and to cure or at least partially arrest symptoms and/or complications.

The vaccine disclosed herein can be administered alone or in combination with other therapeutic agents. The therapeutic agent is for example, a chemotherapeutic agent, radiation, or immunotherapy, including but not limited to checkpoint inhibitors, such as nivolumab, ipilimumab, pembrolizumab, or the like. Any suitable therapeutic treatment for a particular, cancer may be administered.

The term “chemotherapeutic agent” refers to a compound that inhibits or prevents the viability and/or function of cells, and/or causes destruction of cells (cell death), and/or exerts anti-tumor/anti-proliferative effects. The term also includes agents that cause a cytostatic effect only and not a mere cytotoxic effect. Examples of chemotherapeutic agents include, but are not limited to bleomycin, capecitabine, carboplatin, cisplatin, cyclophosphamide, docetaxel, doxorubicin, etoposide, interferon alpha, irinotecan, lansoprazole, levamisole, methotrexate, metoclopramide, mitomycin, omeprazole, ondansetron, paclitaxel, pilocarpine, rituxitnab, tamoxifen, taxol, trastuzumab, vinblastine, and vinorelbine tartrate.

Preferably, the other therapeutic agent is an anti-immunosuppressive/immunostimulatory agent, such as anti-CTLA antibody or anti-PD-1 or anti-PD-L1. Blockade of CTLA-4 or PD-L1 by antibodies can enhance the immune response to cancerous cells. In particular, CTLA-4 blockade has been shown effective when following a vaccination protocol.

As is understood by a skilled person the vaccine and other therapeutic agents may be provided simultaneously, separately, or sequentially. In some embodiments, the vaccine may be provided several days or several weeks prior to or following treatment with one or more other therapeutic agents. The combination therapy may result in an additive or synergistic therapeutic effect.

As disclosed herein, the present disclosure provides vaccines which can be prepared as off-the-shelf vaccines. As used herein “off-the-shelf” means a vaccine as disclosed herein that is available and ready for administration to a patient. For example, when a certain frame shift mutation is identified in a patient, the term “off-the-shelf” would refer to a vaccine according to the disclosure that is ready for use in the treatment of the patient, meaning that, if the vaccine is peptide based, the corresponding polyNOP peptide may, for example already be expressed and for example stored with the required excipients and stored appropriately, for example at −20° C. or −80° C. Preferably the term “off-the-shelf” also means that the vaccine has been tested, for example for safety or toxicity. More preferably the term also means that the vaccine has also been approved for use in the treatment or prevention in a patient. Accordingly, the disclosure also provides a storage facility for storing the vaccines disclosed herein. Depending on the final formulation, the vaccines may be stored frozen or at room temperature, e.g., as dried preparations. Preferably, the storage facility stores at least 20 or at least 50 different vaccines, each recognizing a neoantigen disclosed herein.

The present disclosure also contemplates methods which include determining the presence of NOPs in a tumor sample. In a preferred embodiment, a tumor of a patient can be screened for the presence of frame shift mutations and an NOP can be identified that results from such a frame shift mutation. Based on the NOP(s) identified in the tumor, a vaccine comprising the relevant NOP(s) can be provided to immunize the patient, so the immune system of the patient will target the tumor cells expressing the neoantigen. An exemplary workflow for providing a neoantigen as disclosed herein is as follows. When a patient is diagnosed with a cancer, a biopsy may be taken from the tumor or a sample set is taken of the tumor after resection. The genome, exome and/or transcriptome is sequenced by any method known to a skilled person. The outcome is compared, for example using a web interface or software, to the library of NOPs disclosed herein. A patient whose tumor expresses one of the NOPs disclosed herein is thus a candidate for a vaccine comprising the NOP (or a fragment thereof).

Accordingly, the disclosure provides a method for determining a therapeutic treatment for an individual afflicted with cancer, said method comprising determining the presence of a frame shift mutation which results in the expression of an NOP selected from sequences 29-558. Identification of the expression of an NOP indicates that said individual should be treated with a vaccine corresponding to the identified NOP. For example, if it is determined that tumor cells from an individual express Sequence 29, then a vaccine comprising Sequence 29 or a fragment thereof is indicated as a treatment for said individual.

Accordingly, the disclosure provides a method for determining a therapeutic treatment for an individual afflicted with cancer, said method comprising determining the presence of a frame shift mutation which results in the expression of an NOP selected from sequences 1-28. Identification of the expression of an NOP indicates that said individual should be treated with a vaccine corresponding to the identified NOP. For example, if it is determined that tumor cells from an individual express Sequence 1, then a vaccine comprising Sequence 1 or a fragment thereof is indicated as a treatment for said individual. In some embodiments, the method further comprises determining the presence of a frame shift mutation which results in the expression of an NOP selected from sequences 29-558.

Accordingly, the disclosure provides a method for determining a therapeutic treatment for an individual afflicted with cancer, said method comprising

a. performing complete, targeted or partial genome, exome, ORFeome, or transcriptome sequencing of at least one tumor sample obtained from the individual to obtain a set of sequences of the subject-specific tumor genome, exome, ORFeome, or transcriptome;
b. comparing at least one sequence or portion thereof from the set of sequences with one or more sequences selected from: Sequences 29-558;
c. identifying a match between the at least one sequence or portion thereof from the set of sequences and a sequence from groups (i) to (v) when the sequences have a string in common representative of at least 8 amino acids to identify a neoantigen encoded by a frameshift mutation;

wherein a match indicates that said individual is to be treated with the vaccine as disclosed herein.

Accordingly, the disclosure provides a method for determining a therapeutic treatment for an individual afflicted with cancer, said method comprising

a. performing complete, targeted or partial genome, exome, ORFeome, or transcriptome sequencing of at least one tumor sample obtained from the individual to obtain a set of sequences of the subject-specific tumor genome, exome, ORFeome, or transcriptome;
b. comparing at least one sequence or portion thereof from the set of sequences with one or more sequences selected from: Sequences 1-28 and optionally, one or more sequences selected from 29-558;
c. identifying a match between the at least one sequence or portion thereof from the set of sequences and a sequence from groups (i) to (v) when the sequences have a string in common representative of at least 8 amino acids to identify a neoantigen encoded by a frameshift mutation;

wherein a match indicates that said individual is to be treated with the vaccine as disclosed herein.

As used herein the term “sequence” can refer to a peptide sequence, DNA sequence or RNA sequence. The term “sequence” will be understood by the skilled person to mean either or any of these, and will be clear in the context provided. For example, when comparing sequences to identify a match, the comparison may be between DNA sequences, RNA sequences or peptide sequences, but also between DNA sequences and peptide sequences. In the latter case the skilled person is capable of first converting such DNA sequence or such peptide sequence into, respectively, a peptide sequence and a DNA sequence in order to make the comparison and to identify the match. As is clear to a skilled person, when sequences are obtained from the genome or exome, the DNA sequences are preferably converted to the predicted peptide sequences. In this way, neo open reading frame peptides are identified.

As used herein the term “exome” is a subset of the genome that codes for proteins. An exome can be the collective exons of a genome, or also refer to a subset of the exons in a genome, for example all exons of known cancer genes.

As used herein the term “transcriptome” is the set of all RNA molecules is a cell or population of cells. In a preferred embodiment the transcriptome refers to all mRNA.

In some preferred embodiments the genome is sequenced. In some preferred embodiments the exome is sequenced. In some preferred embodiments the transcriptome is sequenced. In some preferred embodiments a panel of genes is sequenced, for example ARID1A, PTEN, KMT2D, KMT2B, and/or CDKN2A. In some preferred embodiments a single gene is sequenced. In some preferred embodiments TP53 is sequenced. In some embodiments additional genes are sequenced, for example ARID1A, PTEN, KMT2D, KMT2B, and CDKN2A. Preferably the transcriptome is sequenced, in particular the mRNA present in a sample from a tumor of the patient. The transcriptome is representative of genes and neo open reading frame peptides as defined herein being expressed in the tumor in the patient.

As used herein the term “sample” can include a single cell or multiple cells or fragments of cells or an aliquot of body fluid, taken from an individual, by means including venipuncture, excretion, ejaculation, massage, biopsy, needle aspirate, lavage sample, scraping, surgical incision, or intervention or other means known in the art. The DNA and/or RNA for sequencing is preferably obtained by taking a sample from a tumor of the patient. The skilled person knowns how to obtain samples from a tumor of a patient and depending on the nature, for example location or size, of the tumor. Preferably the sample is obtained from the patient by biopsy or resection. The sample is obtained in such manner that is allows for sequencing of the genetic material obtained therein. In order to prevent a less accurate identification of at least one antigen, preferably the sequence of the tumor sample obtained from the patient is compared to the sequence of other non-tumor tissue of the patient, usually blood, obtained by known techniques (e.g. venipuncture).

Identification of frame shift mutations can be done by sequencing of RNA or DNA using methods known to the skilled person. Sequencing of the genome, exome, ORFeome, or transcriptome may be complete, targeted or partial. In some embodiments the sequencing is complete (whole sequencing). In some embodiments the sequencing is targeted. With targeted sequencing is meant that purposively certain region or portion of the genome, exome, ORFeome or transcriptome are sequenced. For example targeted sequencing may be directed to only sequencing for sequences in the set of sequences obtained from the cancer patient that would provide for a match with one or more of the sequences in the sequence listing, for example by using specific primers. In some embodiment only portion of the genome, exome, ORFeome or transcriptome is sequenced. The skilled person is well-aware of methods that allow for whole, targeted or partial sequencing of the genome, exome, ORFeome or transcriptome of a tumor sample of a patient. For example any suitable sequencing-by-synthesis platform can be used including the Genome Sequencers from Illumina/Solexa, the Ion Torrent system from Applied BioSystems, and the RSII or Sequel systems from Pacific Biosciences. Alternatively Nanopore sequencing may be used, such as the MinION, GridION or PromethION platform offered by Oxford Nanopore Technologies. The method of sequencing the genome, exome, ORFeome or transcriptome is not in particular limited within the context of the present invention.

Sequence comparison can be performed by any suitable means available to the skilled person. Indeed the skilled person is well equipped with methods to perform such comparison, for example using software tools like BLAST and the like, or specific software to align short or long sequence reads, accurate or noisy sequence reads to a reference genome, e.g. the human reference genome GRCh37 or GRCh38. A match is identified when a sequence identified in the patients material and a sequence as disclosed herein have a string, i.e. a peptide sequence (or RNA or DNA sequence encoding such peptide (sequence) in case the comparison is on the level of RNA or DNA) in common representative of at least 8, preferably at least 10 adjacent amino acids. Furthermore, sequence reads derived from a patients cancer genome (or transcriptome) can partially match the genomic DNA sequences encoding the amino acid sequences as disclosed herein, for example if such sequence reads are derived from exon/intron boundaries or exon/exon junctions, or if part of the sequence aligns upstream (to the 5′ end of the gene) of the position of a frameshift mutation. Analysis of sequence reads and identification of frameshift mutations will occur through standard methods in the field. For sequence alignment, aligners specific for short or long reads can be used, e.g. BWA (Li and Durbin, Bioinformatics. 2009 Jul. 15; 25(14):1754-60) or Minimap2 (Li, Bioinformatics. 2018 Sep. 15; 34(18):3094-3100). Subsequently, frameshift mutations can be derived from the read alignments and their comparison to a reference genome sequence (e.g. the human reference genome (RCh37) using variant calling tools, for example Genome Analysis ToolKit (GATK), and the like (McKenna et al. Genome Res. 2010 Sep.; 20(9):1297-303).

A match between an individual patient's tumor sample genome or transcriptome sequence and one or more NOPs disclosed herein indicates that said tumor expresses said NOP and that said patient would likely benefit from treatment with a vaccine comprising said NOP (or a fragment thereof). More specifically, a match occurs if a frameshift mutation is identified in said patient's tumor genome sequence and said frameshift leads to a novel reading frame (+1 or −1 with respect to the native reading from of a gene). In such instance, the predicted out-of-frame peptide derived from the frameshift mutation matches any of the sequences 1-352 as disclosed herein. In some embodiments, said patient is administered said NOP (e.g., by administering the peptides, nucleic acid molecules, vectors, host cells or vaccines as disclosed herein).

In some embodiments, the methods further comprise sequencing the genome, exome, ORFeome, or transcriptome (or a part thereof) from a normal, non-tumor sample from said individual and determining whether there is a match with one or more NOPs identified in the tumor sample. Although the neoantigens disclosed herein appear to be specific to tumors, such methods may be employed to confirm that the neoantigen is tumor specific and not, e.g., a germline mutation.

The disclosure further provides the use of the neoantigens and vaccines disclosed herein in prophylactic methods from preventing or delaying the onset of cancer. Approximately 38% of individuals will develop cancer and the neo open reading frames disclosed herein occur in up to 8.2% of cancer patients. Prophylactic vaccination based on frameshift resulting peptides disclosed herein would thus provide protection to approximately 3.1% of the general population. The vaccine may be specifically used in a prophylactic setting for individuals having an increased risk of developing cancer. For example, prophylactic vaccination is expected to provide possible protection to around 8.2% of all individuals at risk for cancer and who would develop cancer as a result of this risk factor. In some embodiments, the prophylactic methods are useful for individuals who are genetically related to individuals afflicted with cancer. In some embodiments, the prophylactic methods are useful for the general population.

In some embodiments, the individual is at risk of developing cancer. It is understood to a skilled person that being at risk of developing cancer indicates that the individual has a higher risk of developing cancer than the general population; or rather the individual has an increased risk over the average of developing cancer. Such risk factors are known to a skilled person and include
the genetic background of said individual, in particular predisposing germline mutations, preferably the mutation is in one of the mismatch repair genes (Lynch disease) and/or a mutation in TP53, BRCA1, BRCA2, CHEK2, MLH1, MSH2, MSH6, PMS1, PMS2, ERCC1, CDKN2A, XPA, FANCG, BAP1, POLD1, EPCAM, MAP2K2, SH2B3, PRDM9, PTCH1, RAD51D, PRF1, PTEN, PALB2, ERCC4, DIS3L2, TRIM37, NTHL1, FANCC, BRIP1, NBN, ERCC2, FANCD2, SDHA, UROD, DROSHA, ATM, DICER1, WRN, BRCA2, APC, ATR, ABCB11, SUFU, RAD51C, POLE, RET, MPL, XPC, SMARCA4, FH, HMBS, NF1, POT1, FAH, GJB2, CBL, RECQL, FANCM, KIT, RECQL4, MUTYH, DOCK8, RB1, ERCC3, EXT1, ERCC5, SDHB, FANCA, BUB1B, KRAS, ALK, SOS1, CDC73, COL7A1, TMEM127, CYLD, BLM, TSC1, SLC25A13, ITK, FANCI, FANCF, RHBDF2, HFE, SBDS, GBA, FANCL, FLCN;
previous history of cancer in said individual, for example, an individual that was treated for cancer and is in remission;
increased age of said individual, in some embodiments the risk of developing cancer increases above the age of 40, above the age of 50 and even more so above the age of 60;
exposure of said individual to carcinogens, for example, tobacco, radon, asbestos, formaldehyde, ultraviolet rays, ionizing radiation, alcohol, processed meat, engine exhaust, pollution, paint chemicals, wood dust, etc.; and/or
lifestyle factors associated with cancer development including poor diet or a diet high in red meat and/or processed meat, limited physical activity, obesity, smoking, drinking alcohol.

In some embodiments, said individual has a germline mutation in a gene that increases the chance that the individual will develop cancer, preferably the mutation is in one or more of the following genes: TP53, BRCA1, BRCA2, CHEK2, MLH1, MSH2, MSH6, PMS1, PMS2, ERCC1, CDKN2A, XPA, FANCG, BAP1, POLD1, EPCAM, MAP2K2, SH2B3, PRDM9, PTCH1, RAD51D, PRF1, PTEN, PALB2, ERCC4, DIS3L2, TRIM37, NTHL1, FANCC, BRIP1, NBN, ERCC2, FANCD2, SDHA, UROD, DROSHA, ATM, DICER1, WRN, BRCA2, APC, ATR, ABCB11, SUFU, RAD51C, POLE, RET, MPL, XPC, SMARCA4, FH, HMBS, NF1, POT1, FAH, GJB2, CBL, RECQL, FANCM, KIT, RECQL4, MUTYH, DOCK8, RB1, ERCC3, EXT1, ERCC5, SDHB, FANCA, BUB1B, KRAS, ALK, SOS1, CDC73, COL7A1, TMEM127, CYLD, BLM, TSC1, SLC25A13, ITK, FANCI, FANCF, RHBDF2, HFE, SBDS, GBA, FANCL, and FLCN.

In some embodiments, prophylactic methods are provided which include a step of determining whether an individual is at risk of developing cancer, in particular whether they have germline mutation in one or more of the following genes: TP53, BRCA1, BRCA2, CHEK2, MLH1, MSH2, MSH6, PMS1, PMS2, ERCC1, CDKN2A, XPA, FANCG, BAP1, POLD1, EPCAM, MAP2K2, SH2B3, PRDM9, PTCH1, RAD51D, PRF1, PTEN, PALB2, ERCC4, DIS3L2, TRIM37, NTHL1, FANCC, BRIP1, NBN, ERCC2, FANCD2, SDHA, UROD, DROSHA, ATM, DICER1, WRN, BRCA2, APC, ATR, ABCB11, SUFU, RAD51C, POLE, RET, MPL, XPC, SMARCA4, FH, HMBS, NF1, POT1, FAH, GJB2, CBL, RECQL, FANCM, KIT, RECQL4, MUTYH, DOCK8, RB1, ERCC3, EXT1, ERCC5, SDHB, FANCA, BUB1B, KRAS, ALK, SOS1, CDC73, COL7A1, TMEM127, CYLD, BLM, TSC1, SLC25A13, ITK, FANCI, FANCF, RHBDF2, HFE, SBDS, GBA, FANCL, and FLCN.

The disclosure further provides a method of immunizing an individual at risk of developing cancer comprising identifying whether said individual has a risk factor for developing cancer. Cancer risk factors are known to a skilled person and include those disclosed above. The methods further comprise selecting novel open reading frames associated with an identified risk factor or associated with cancer. See, e.g., FIG. 8 which demonstrates the association of novel open reading frames in particular genes with particular cancers. The methods further comprise immunizing said individual having a risk factor for developing cancer. The individual can be immunized with
one or more peptides comprising the amino acid sequence of one or more novel open reading frame peptides,
a collection of tiled peptides comprising said amino acid sequences,
peptide fragments comprising at least 10 consecutive amino acids of said sequences, and/or
one or more nucleic acid molecules encoding said peptides, collection of tiled peptides, or peptide fragments. The peptides and nucleic acid molecules can be prepared in a vaccine formulation as described herein. Preferred novel open reading frames include those depicted as sequences 29-558 as well as sequences 1-28.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

All patent and literature references cited in the present specification are hereby incorporated by reference in their entirety. For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1 Frame shift initiated translation in the TCGA (n=10,186) cohort is of sufficient size for immune presentation. A. Peptide length distribution of frame shift mutation initiated translation up to the first encountered stop codon. Dark shades are unique peptide sequences derived from frameshift mutations, light shade indicates the total sum (unique peptides derived from frameshifts multiplied by number of patients containing that frameshift). B. Gene distribution of peptides with length 10 or longer and encountered in up to 10 patients.

FIG. 2 Neo open reading frame peptides (TOGA cohort) converge on common peptide sequences. Graphical representation in an isoform of TP53, where amino acids are colored distinctly. A. somatic single nucleotide variants, B. positions of frame shift mutations on the −1 and the +1 frame. C. amino acid sequence of TP53. D. Peptide (10aa) library (n=1,000) selection. Peptides belonging to −1 or +1 frame are separated vertically E,F pNOPs for the different frames followed by all encountered frame shift mutations (rows), translated to a stop codon (lines) colored by amino acid.

FIG. 3 A recurrent peptide selection procedure can generate a ‘fixed’ library to cover up to 50% of the TOGA cohort. Graph depicts the number of unique patients from the TCGA cohort (10,186 patients) accommodated by a growing library of 10-mer peptides, picked in descending order of the number patients with that sequence in their NOPs. A peptide is only added if it adds a new patient from the TCGA cohort. The dark blue line shows that an increasing number of 10-mer peptides covers an increasing number of patients from the TCGA cohort (up to 50% if using 3000 unique 10-mer peptides). Light shaded blue line depicts the number of patients containing the peptide that was included (right Y-axis). The best peptide covers 89 additional patients from the TCGA cohort (left side of the blue line), the worst peptide includes only 1 additional patient (right side of the blue line).

FIG. 4 For some cancers up to 70% of patients contain a recurrent NOP. TCGA cohort ratio of patients separated by tumor type that could be ‘helped’ using optimally selected peptides for genes encountered most often within a cancer. Coloring represents the ratio, using 1, 2 . . . 10 genes, or using all encountered genes (lightest shade)

FIG. 5 Examples of NOPs. Selection of genes containing NOPs of 10 or more amino acids.

FIG. 6 Frame shift presence in mRNA from 58 CCLE colorectal cancer cell lines.

a. Cumulative counting of RNAseq allele frequency (Samtools mpileup (XO:1/all)) at the genomic position of DNA detected frame shift mutations.
b. IGV examples of frame shift mutations in the BAM files of CCLE cell lines.

FIG. 7 Example of normal isoforms, using shifted frame.

Genome model of CDKN2A with the different isoforms are shown on the minus strand of the genome. Zoom of the middle exon depicts the 2 reading frames that are encountered in the different isoforms.

FIG. 8 Gene prevalence vs Cancer type.

Percentage of frameshift mutations (resulting in peptides of 10 aa or longer), assessed by the type of cancer in the TCGA cohort. Genes where 50% or more of the frameshifts occur within a single tumor type are indicated in bold.. Cancer type abbreviations are as follows:

### LAML Acute Myeloid Leukemia

ACC Adrenocortical carcinoma

### BLCA Bladder Urothelial Carcinoma

### LGG Brain Lower Grade Glioma

BRCA Breast invasive carcinoma
CESC Cervical squamous cell carcinoma and endocervical adenocarcinoma

### CHOL Cholangiocarcinoma

### LCML Chronic Myelogenous Leukemia

COAD Colon adenocarcinoma

### CNTL Controls

ESCA Esophageal carcinoma
GBM Glioblastoma multiforme
HNSC Head and Neck squamous cell carcinoma

### KICH Kidney Chromophobe

KIRC Kidney renal clear cell carcinoma
KIRP Kidney renal papillary cell carcinoma
LIHC Liver hepatocellular carcinoma
LUAD Lung adenocarcinoma
LUSC Lung squamous cell carcinoma

### DLBC Lymphoid Neoplasm Diffuse Large B-cell Lymphoma

### MESO Mesothelioma

### MISC, Miscellaneous

OV Ovarian serous cystadenocarcinoma
PAAD Pancreatic adenocarcinoma

### PCPG Pheochromocytoma and Paraganglioma

PRAD Prostate adenocarcinoma
READ Rectum adenocarcinoma

### SARC, Sarcoma

### SKCM Skin Cutaneous Melanoma

STAD Stomach adenocarcinoma

### TGCT Testicular Germ Cell Tumors

### THYM Thymoma

THCA Thyroid carcinoma

### UCS Uterine Carcinosarcoma

### UCEC Uterine Corpus Endometrial Carcinoma

### UVM Uveal Melanoma

FIG. 9 NOPs in the MSK-IMPACT study

Frame shift analysis in the targeted sequencing panel of the MSK-IMPACT study, covering up to 410 genes in more 10,129 patients (with at least 1 somatic mutation). a. FS peptide length distribution, b. Gene count of patients containing NOPs of 10 or more amino acids. c. Ratio of patients separated by tumor type that possess a neo epitope using optimally selected peptides for genes encountered most often within a cancer. Coloring represents the ratio, using 1, 2 . . . 10 genes, or using all encountered genes (lightest shade) d. Examples of NOPs for 4 genes.

FIGS. 10-15 Out-of-frame peptide sequences based on frameshift mutations in cancer patients, for FIG. 10 (KMT2B), FIG. 11 (KMT2D), FIG. 12 (CDKN2A), FIG. 13 (PTEN), FIG. 14 (ARID1A), FIG. 15 (TP53).

### EXAMPLES

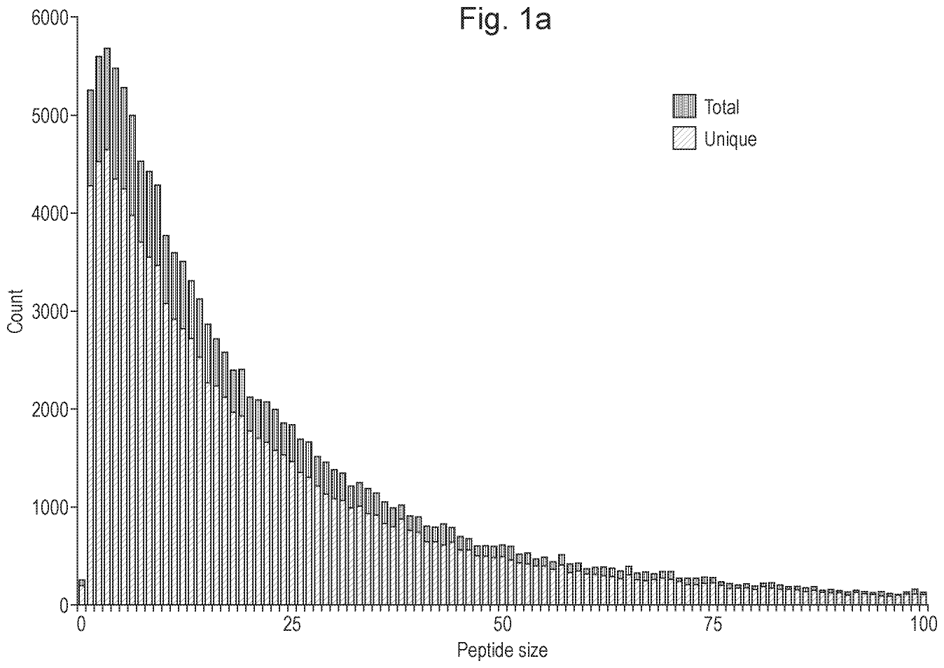

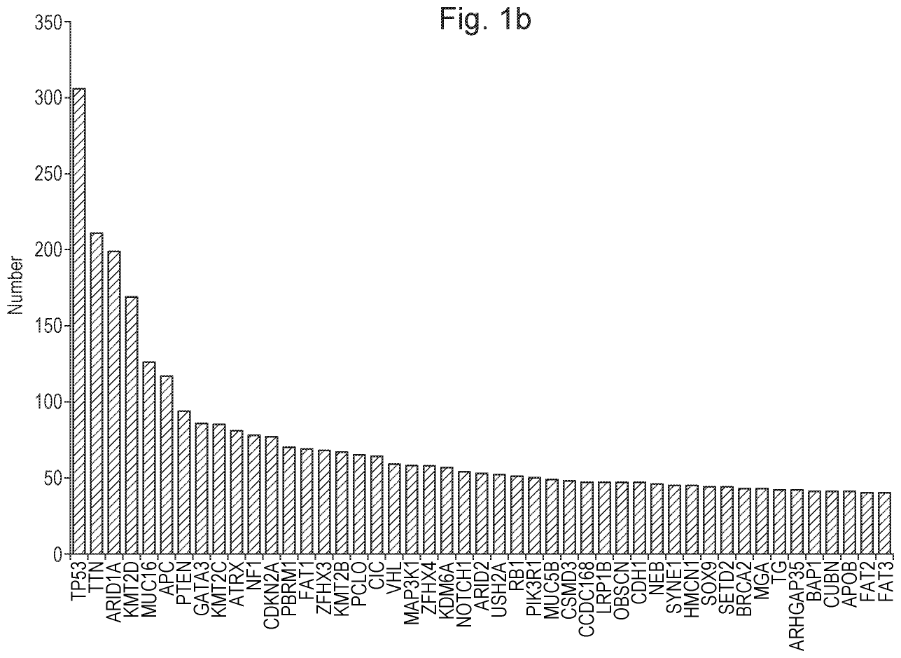

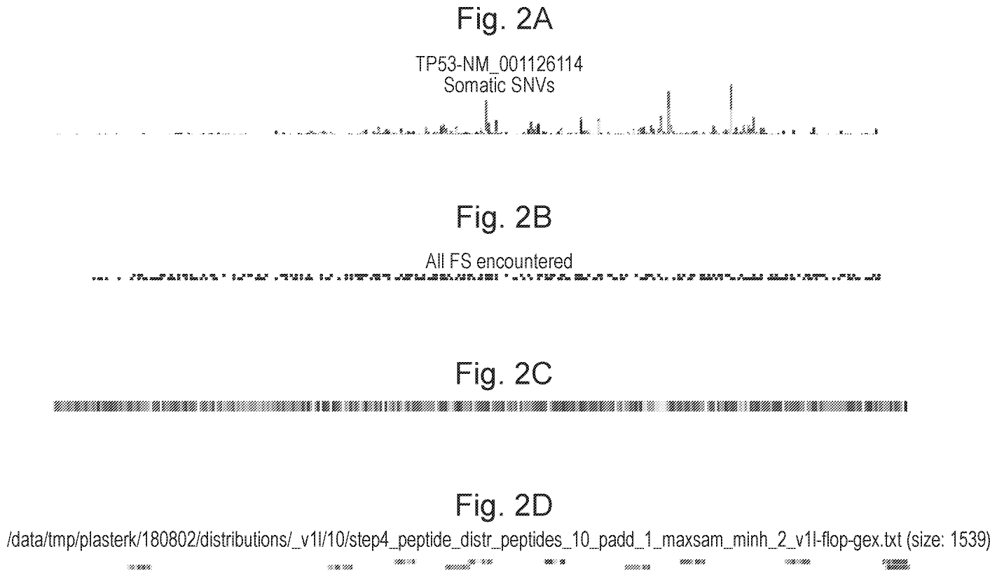

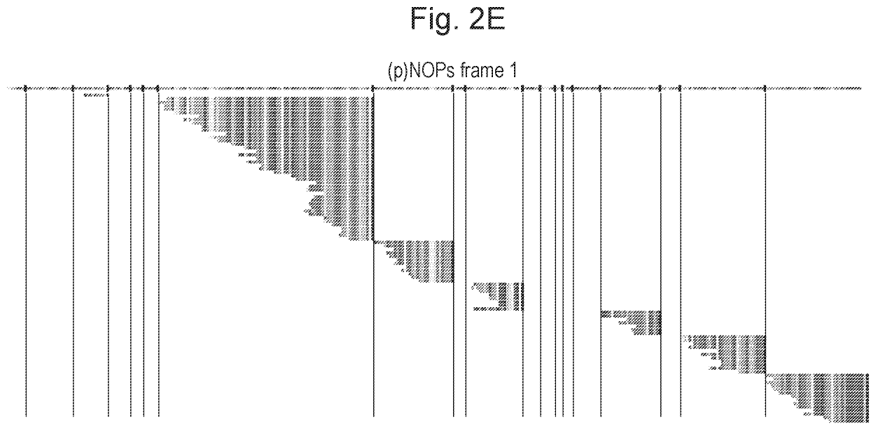

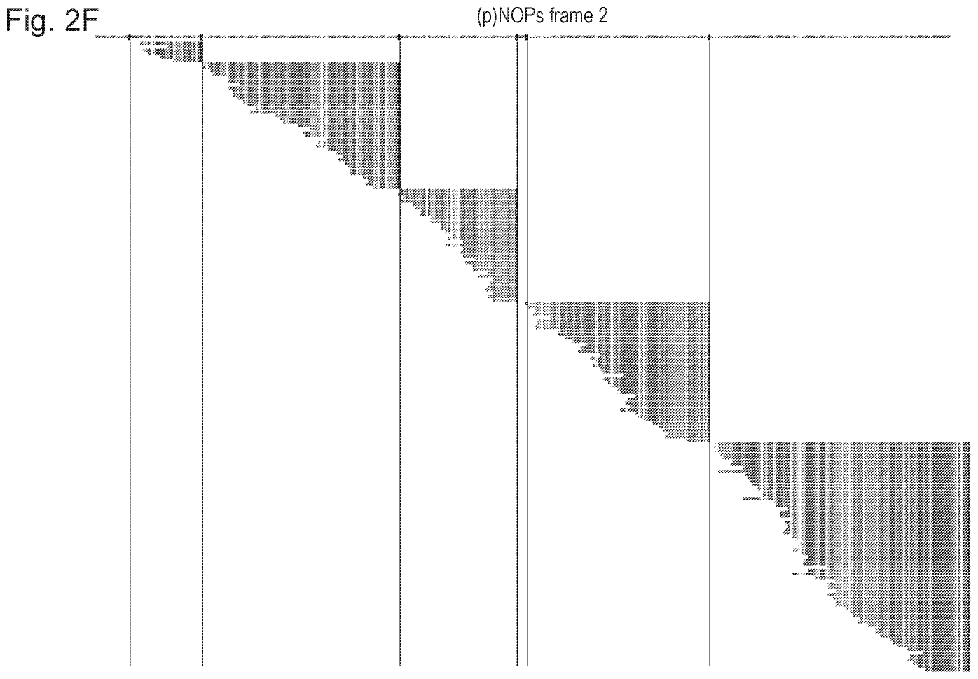

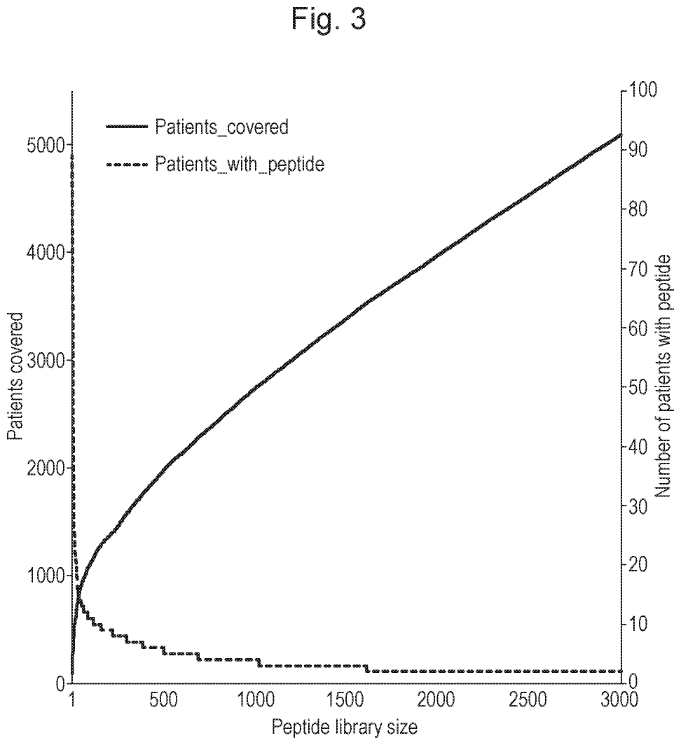

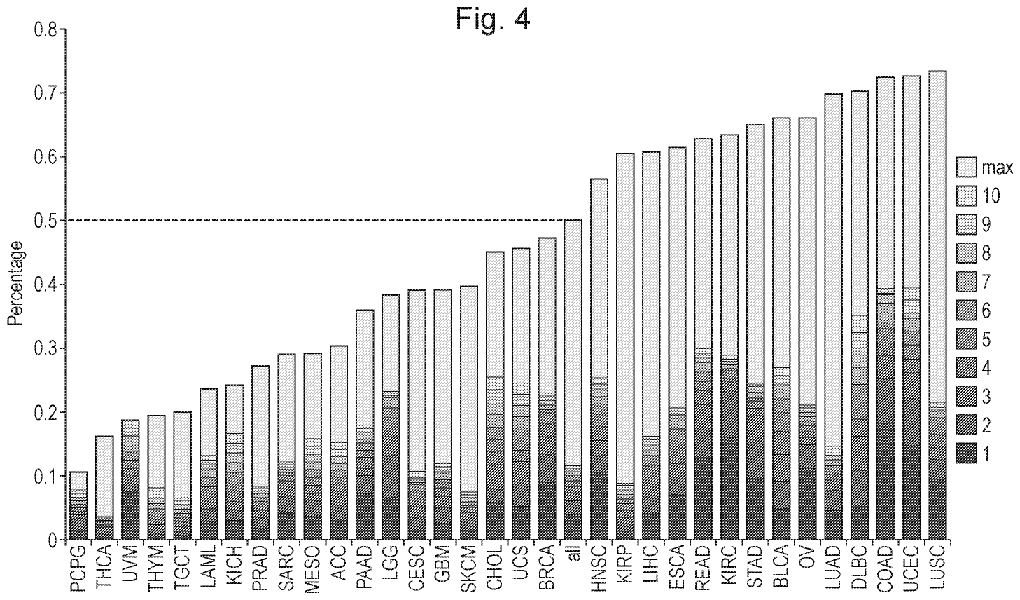

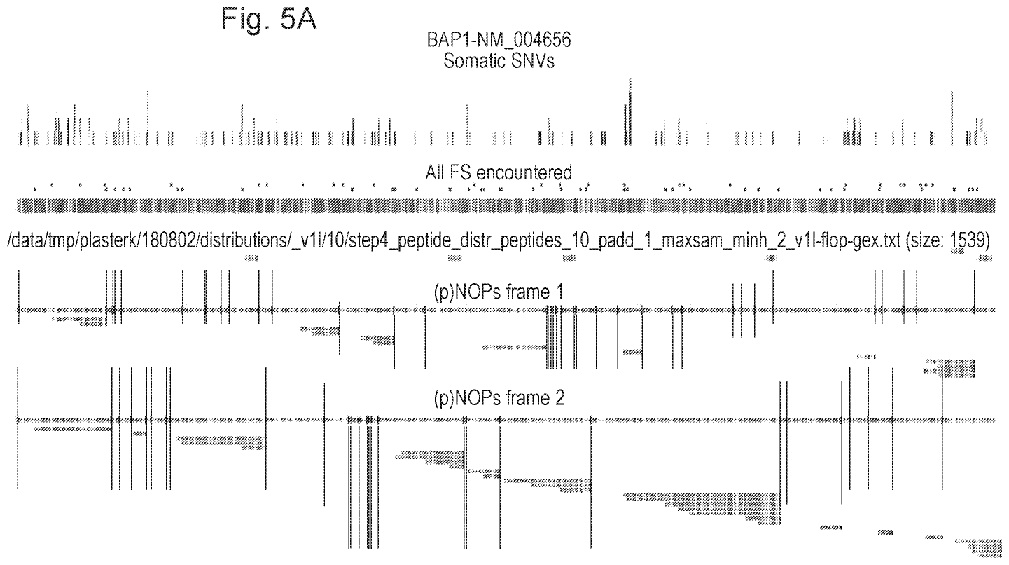

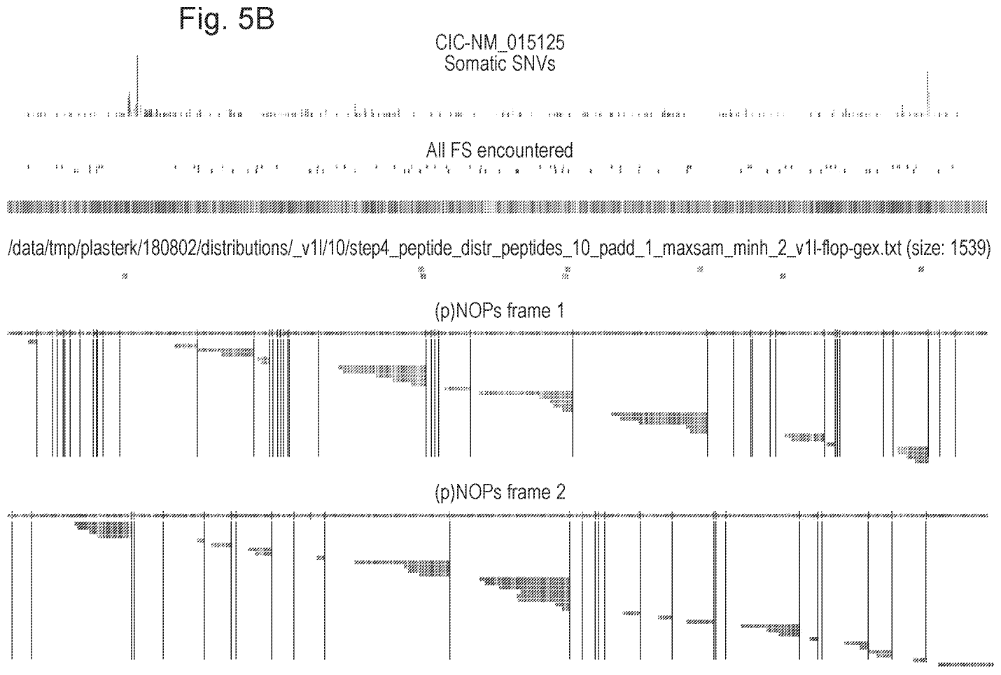

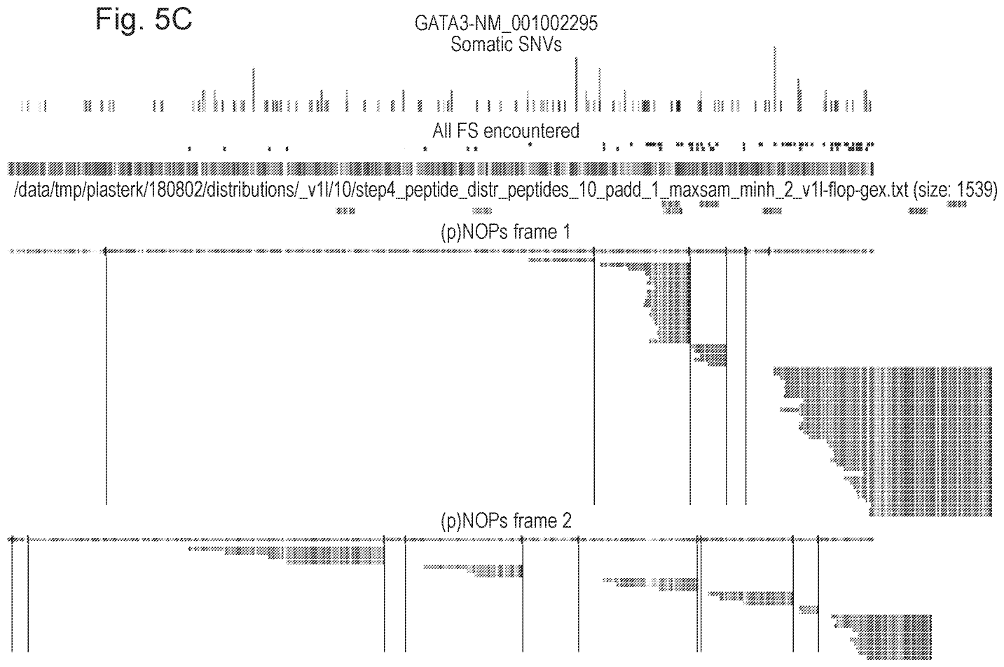

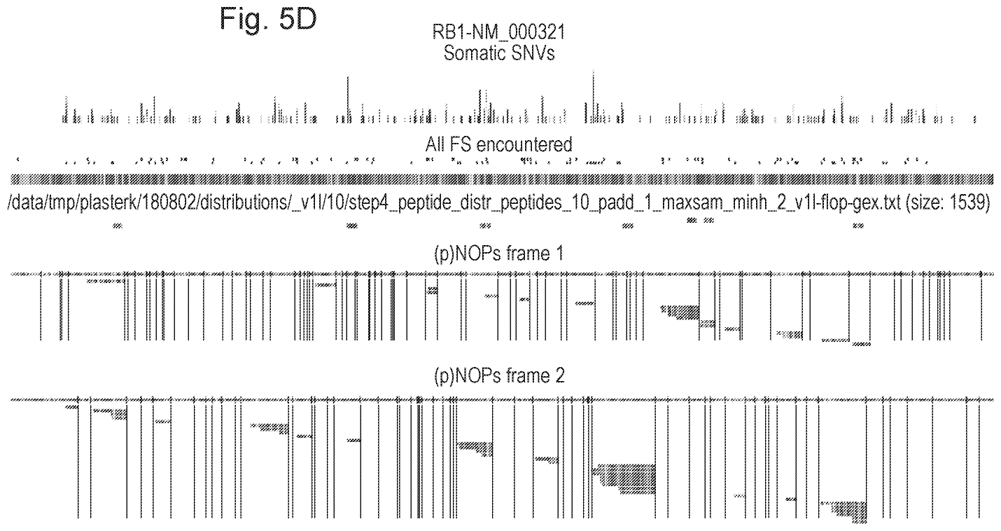

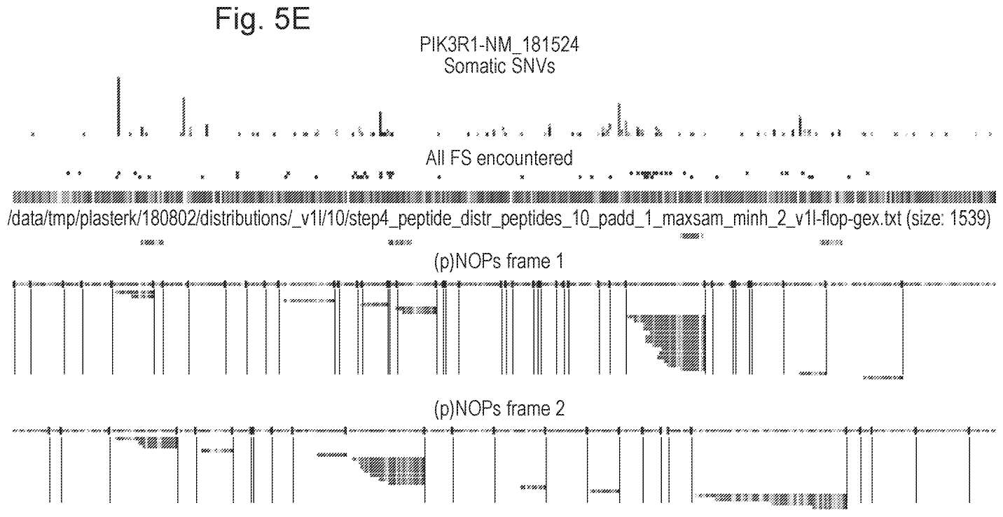

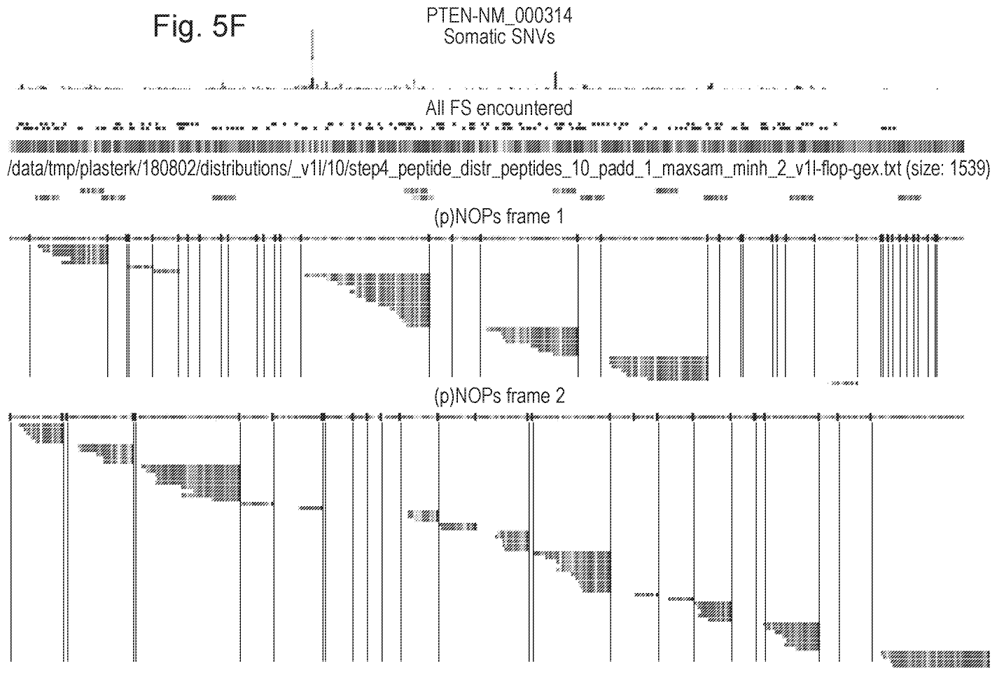

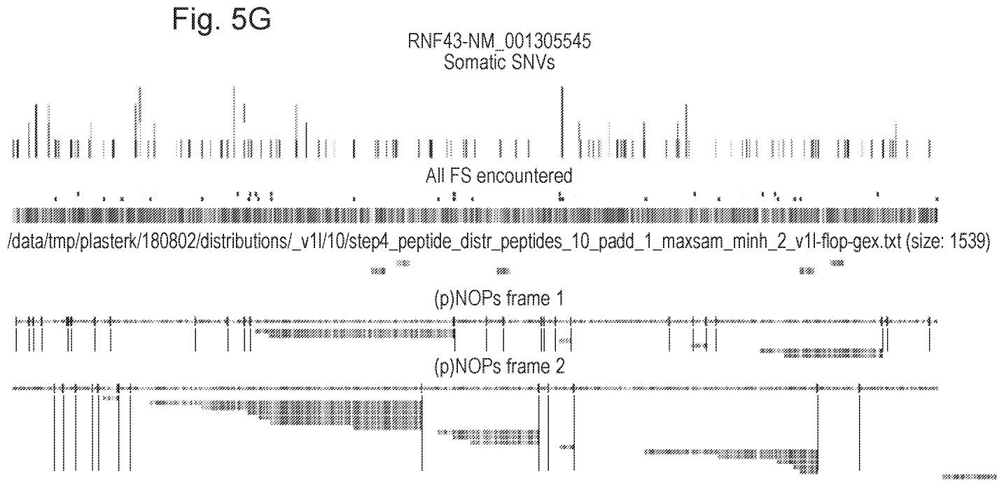

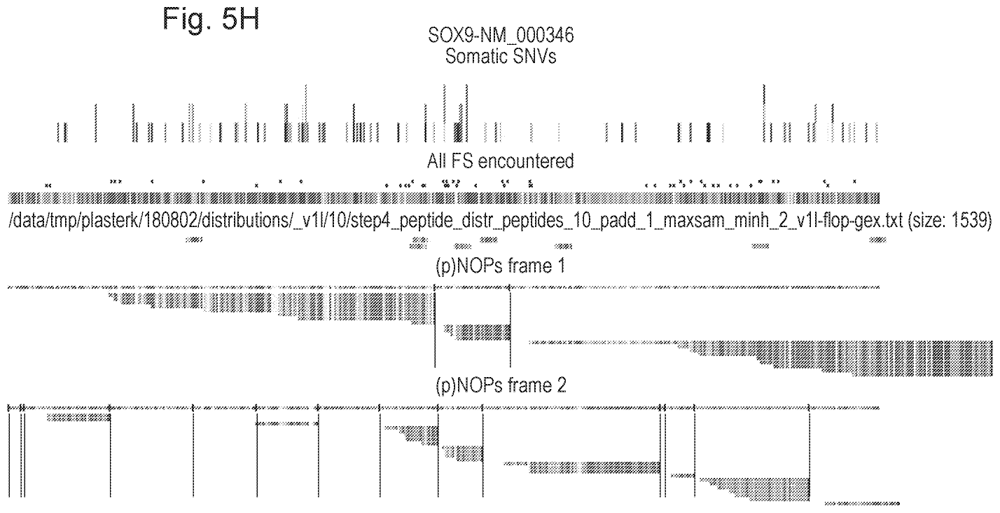

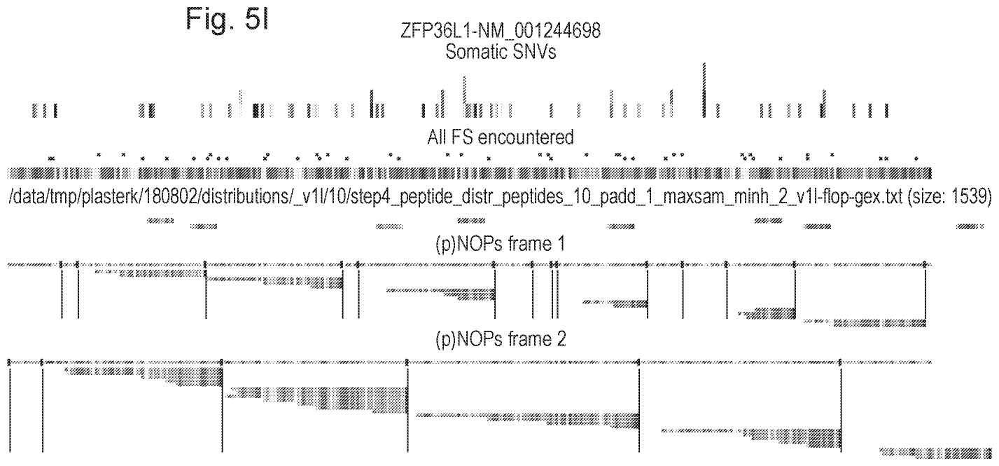

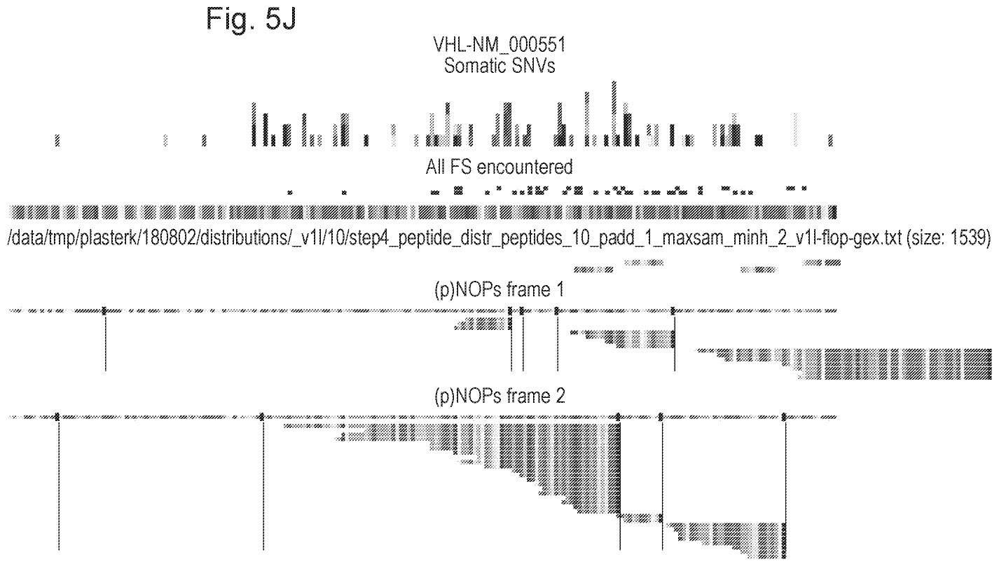

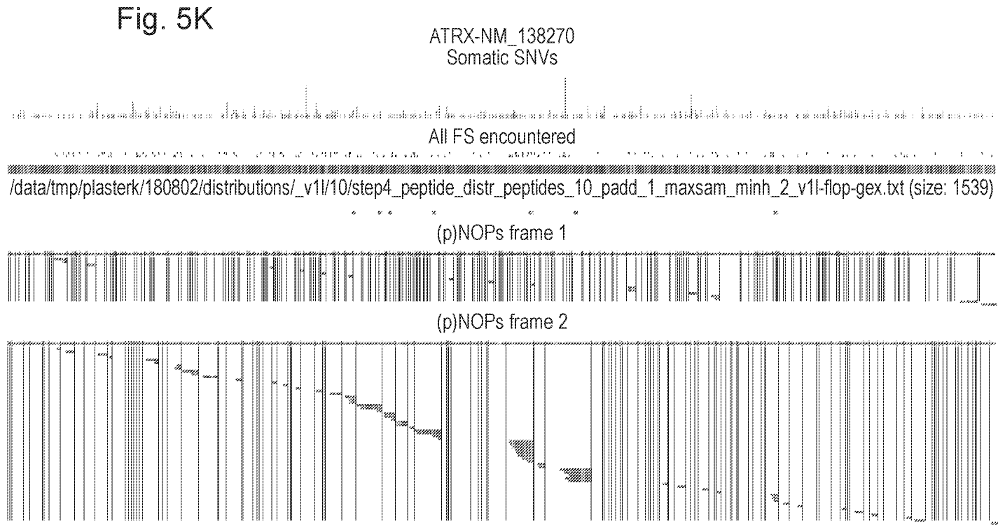

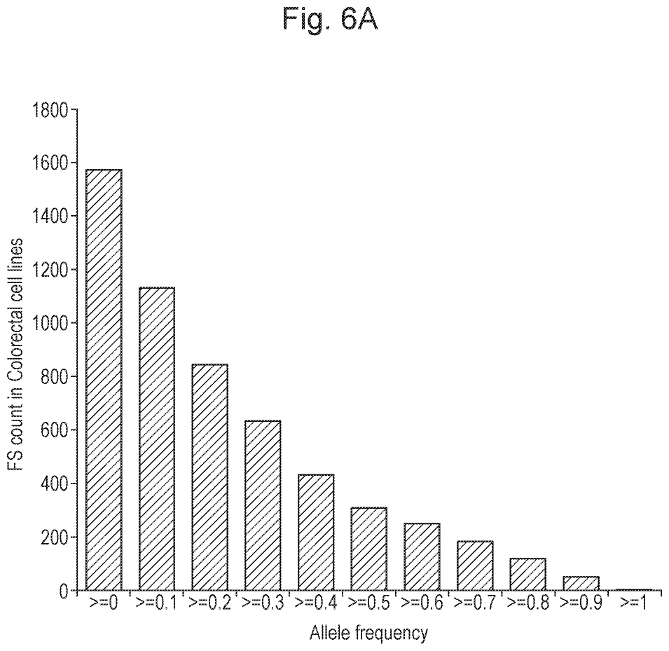

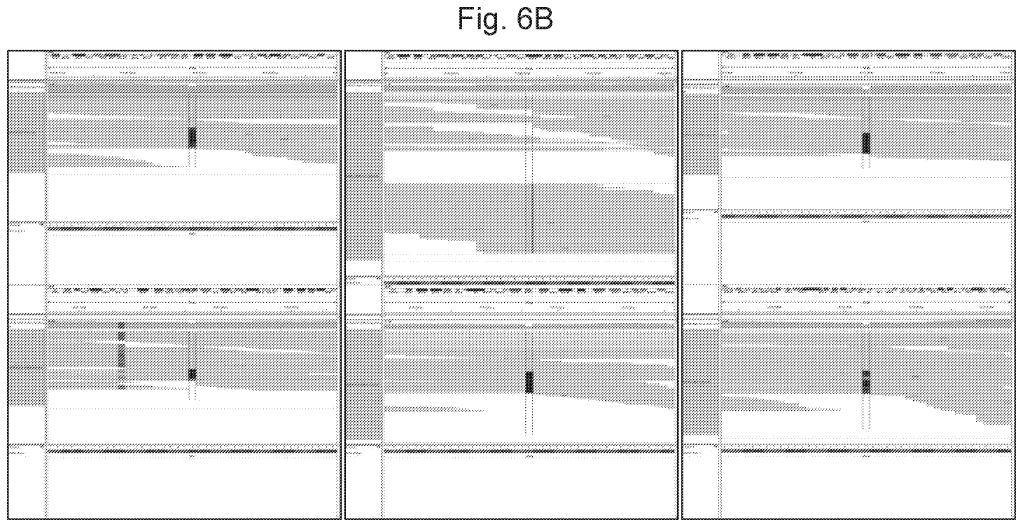

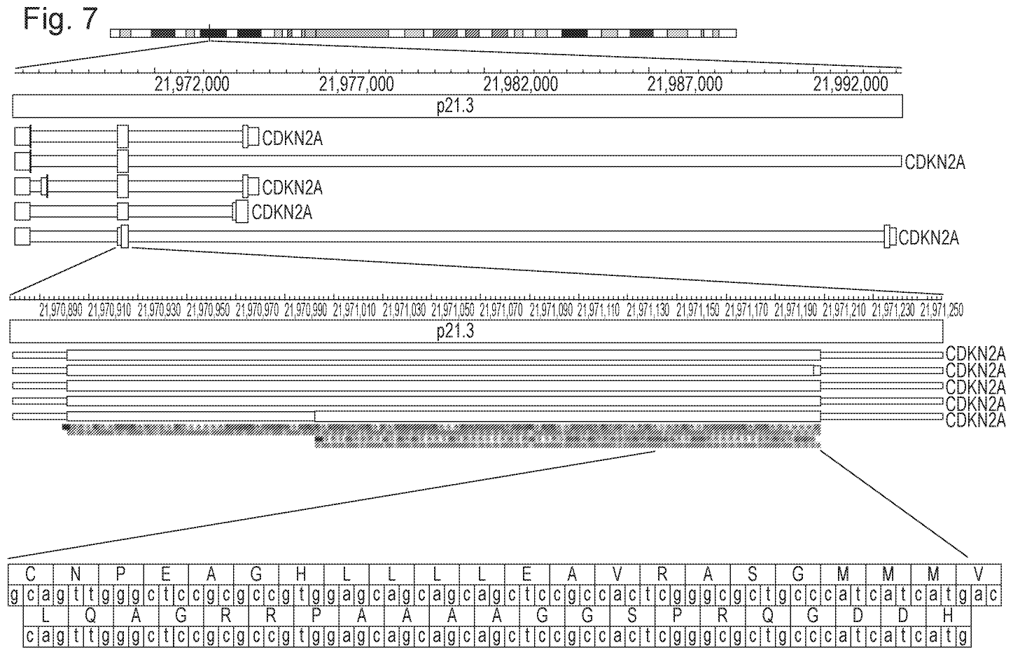

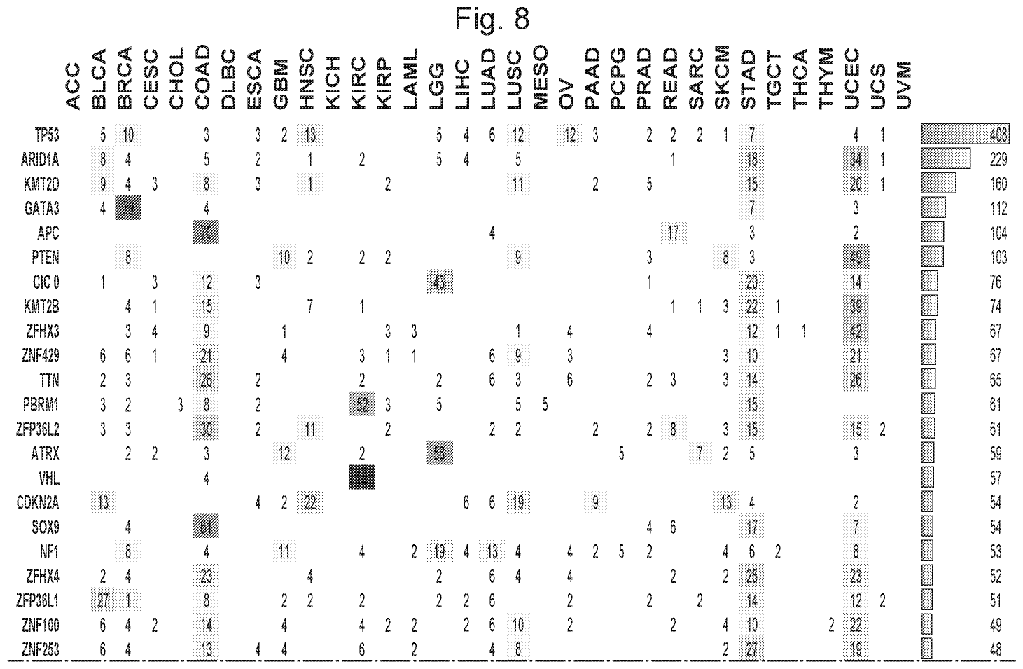

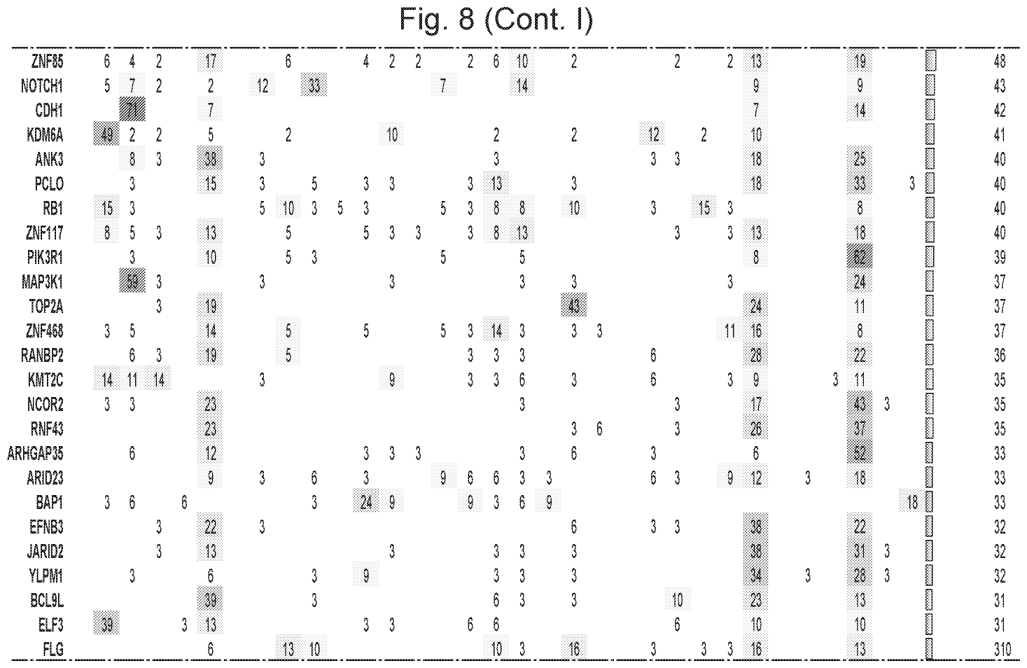

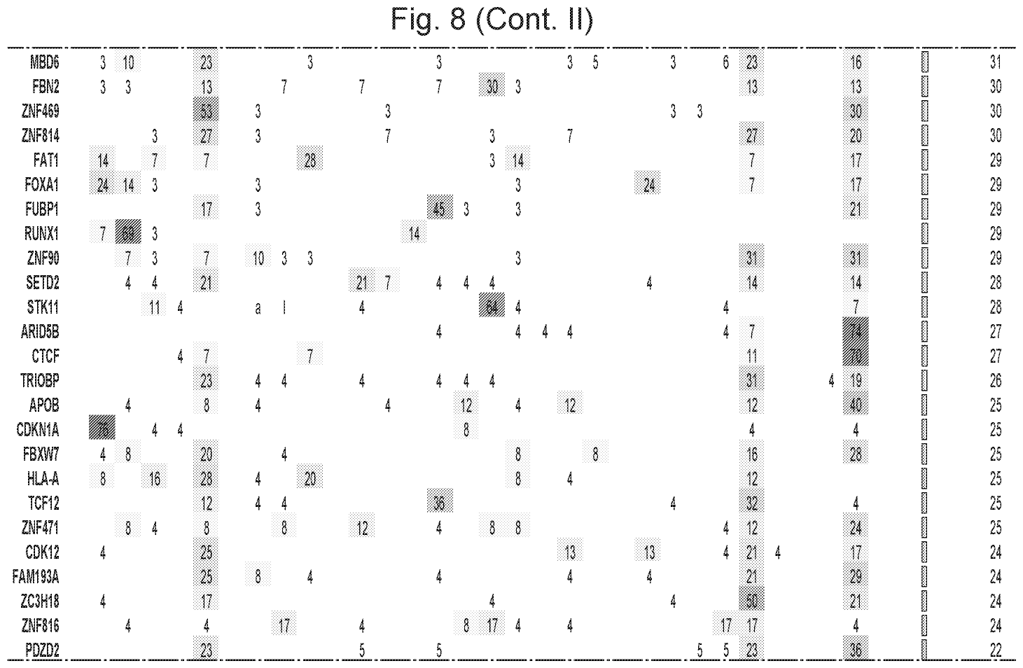

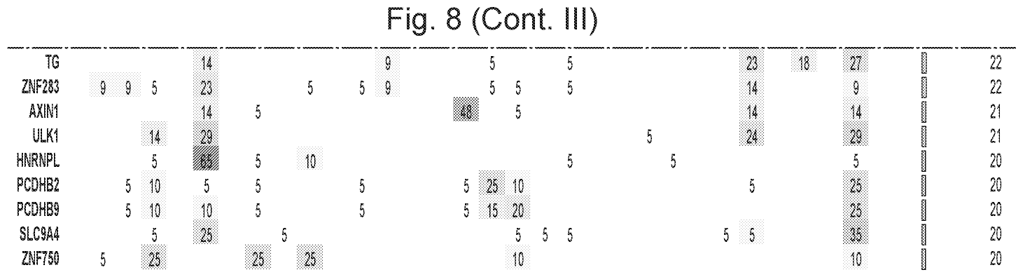

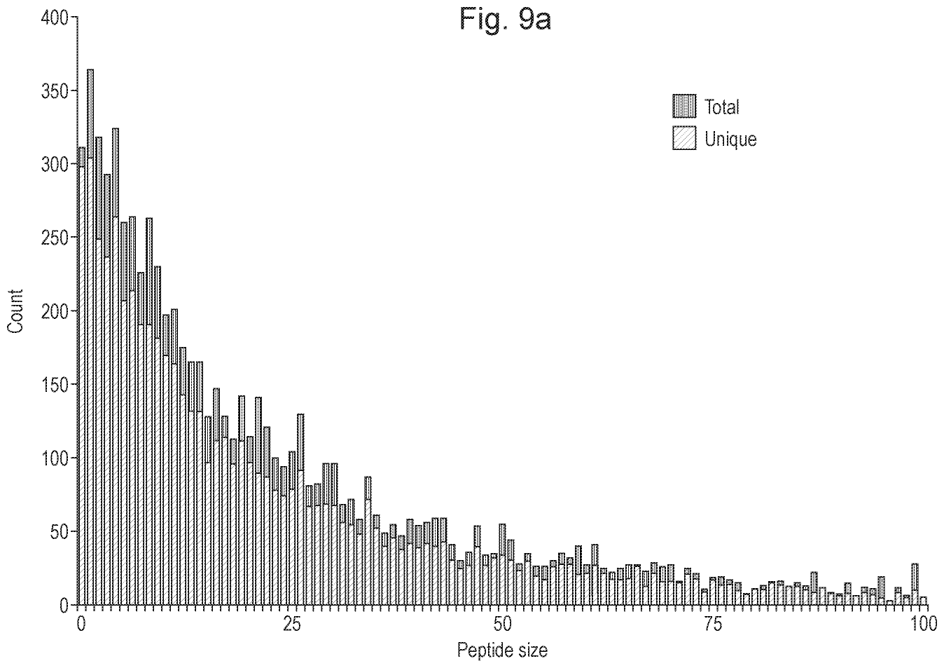

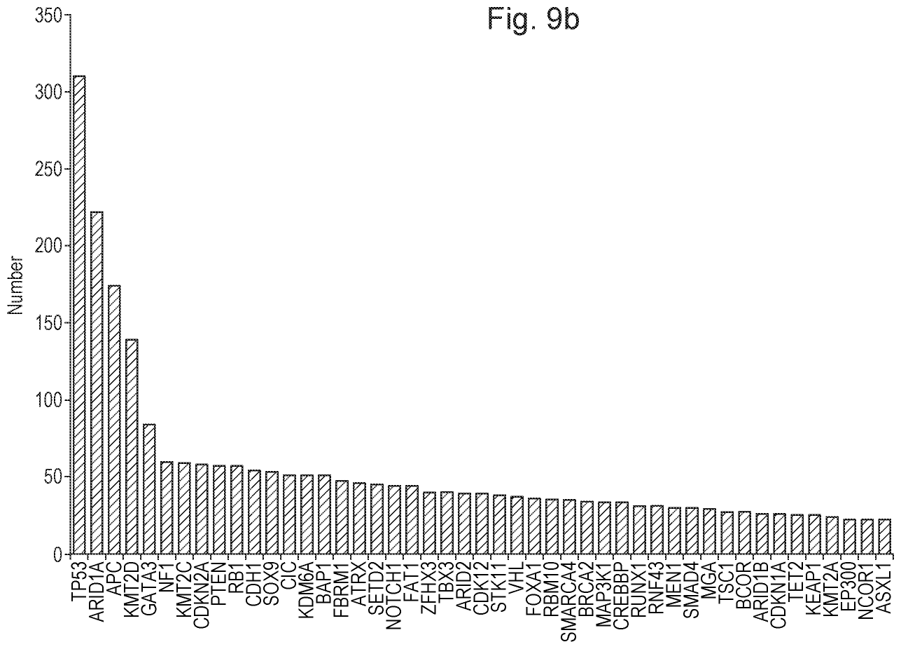

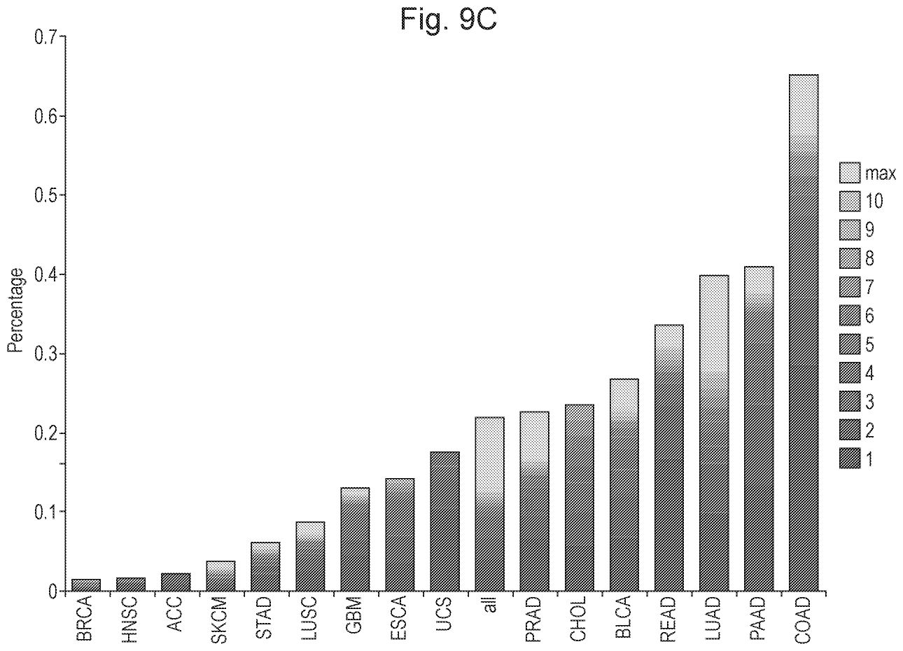

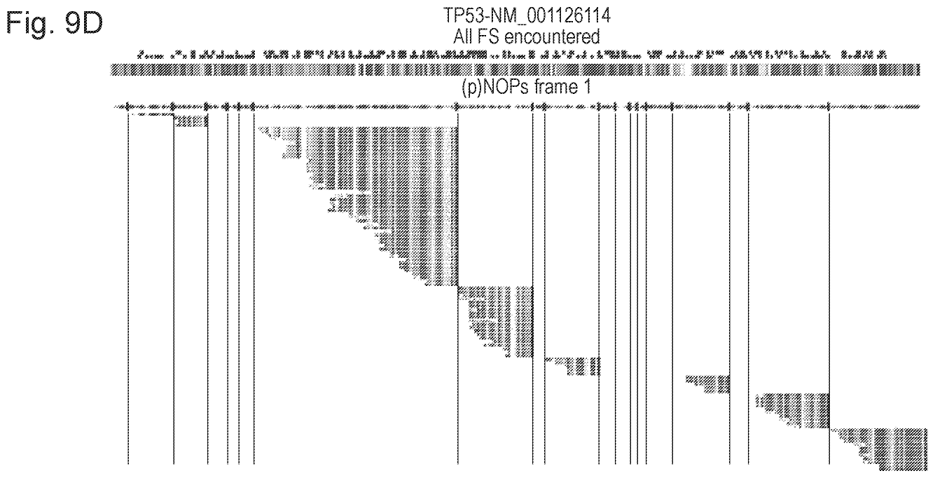

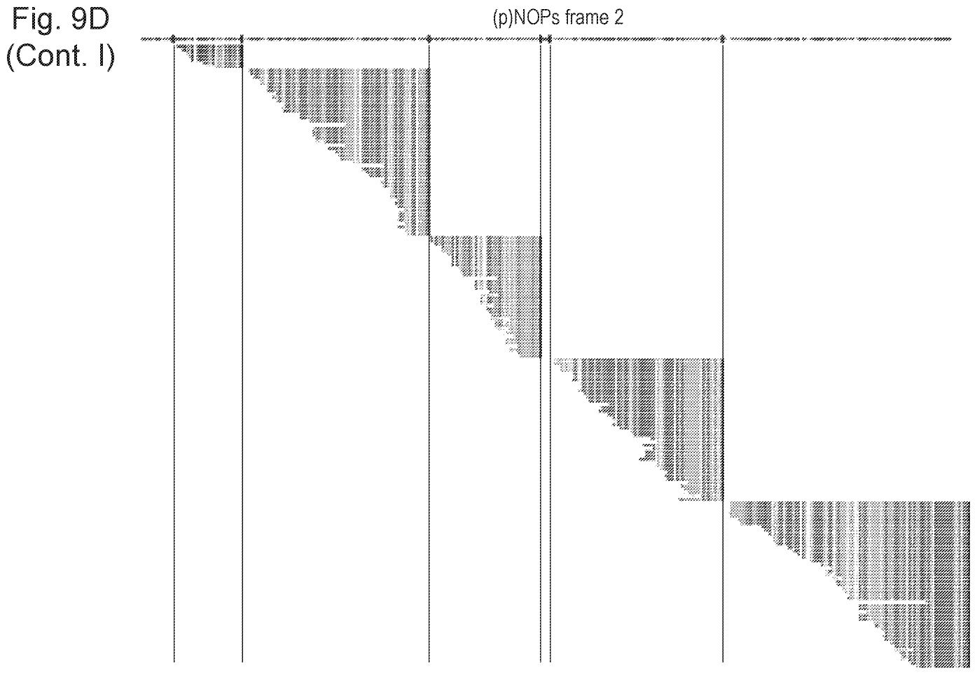

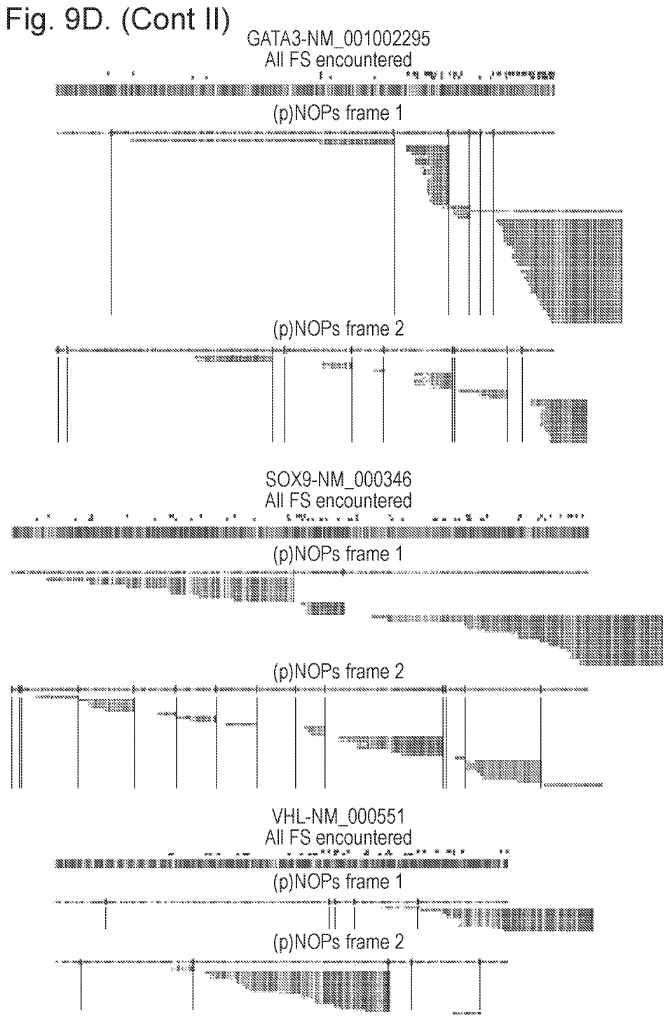

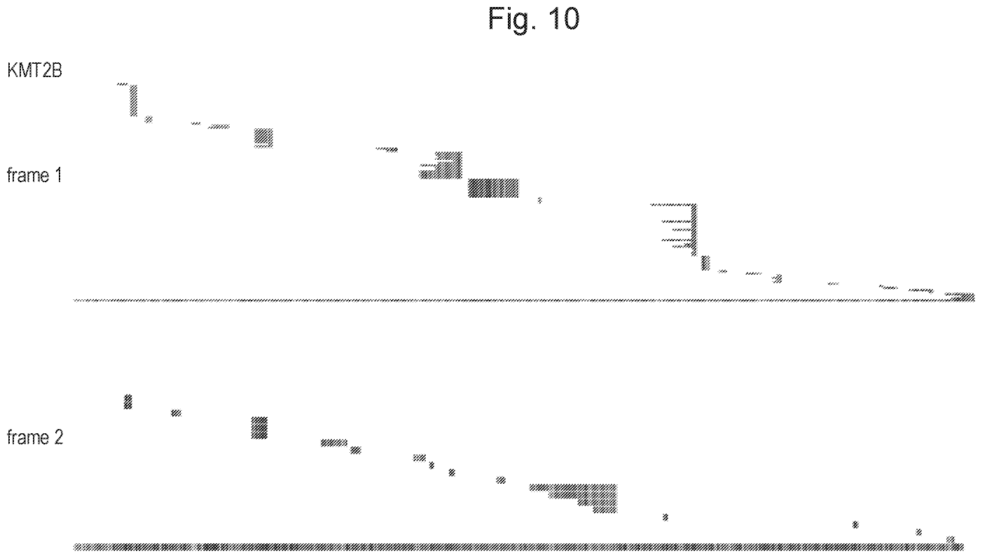

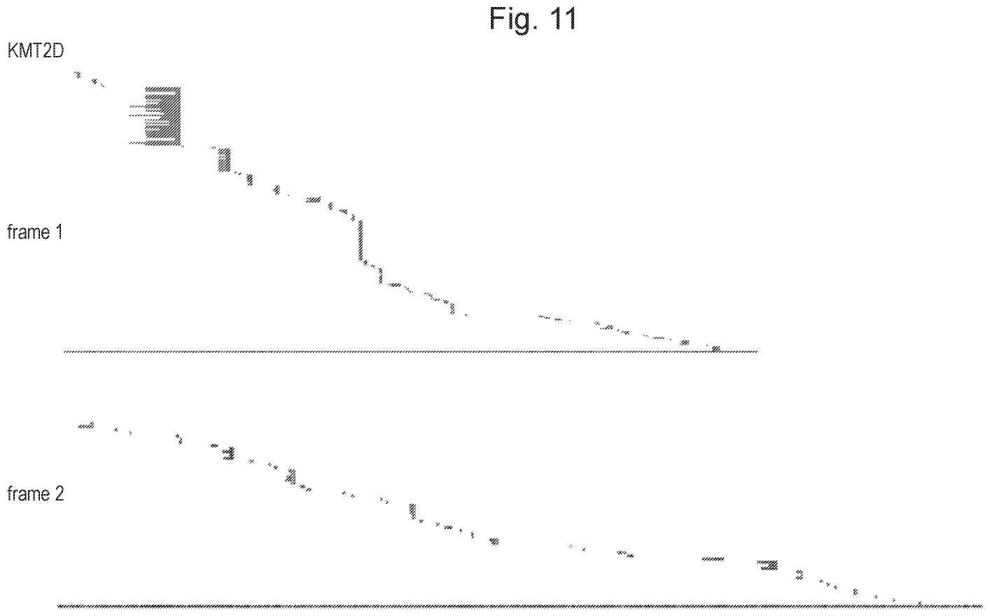

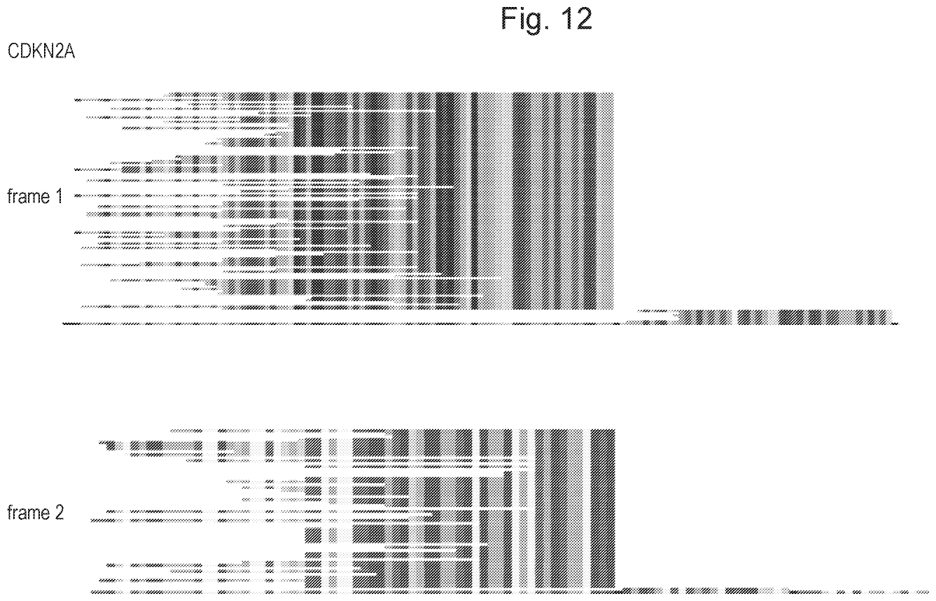

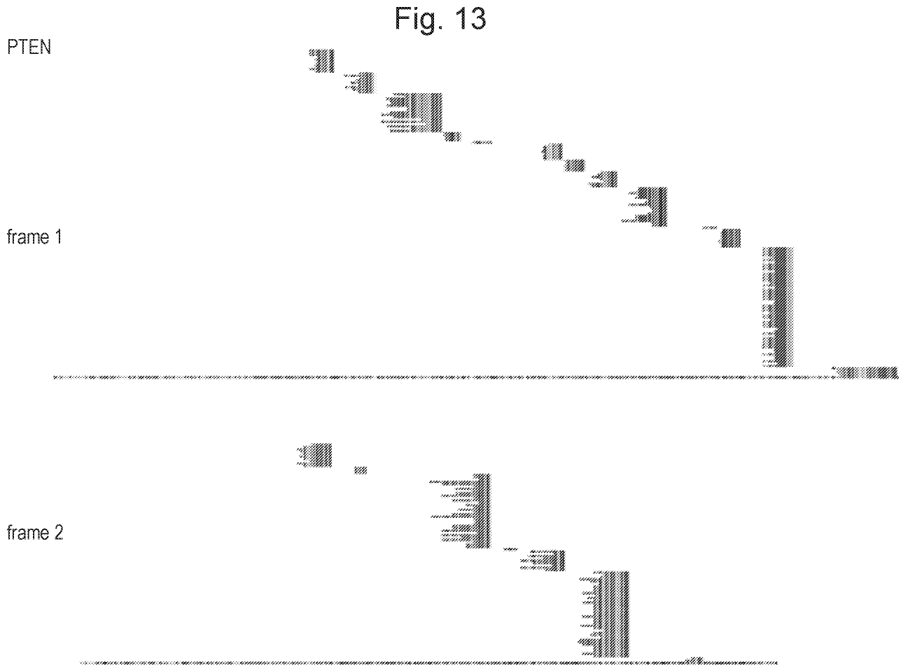

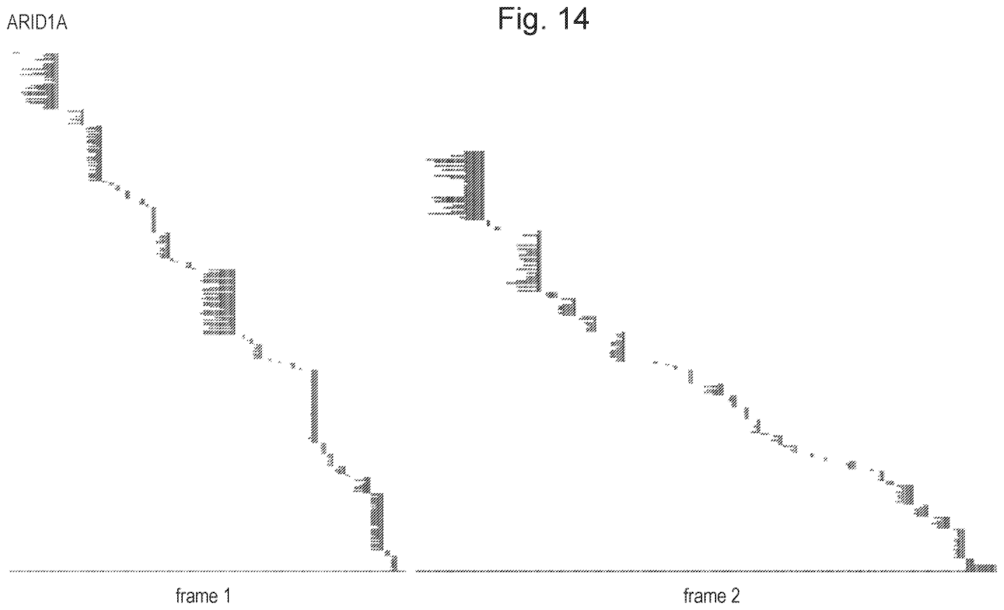

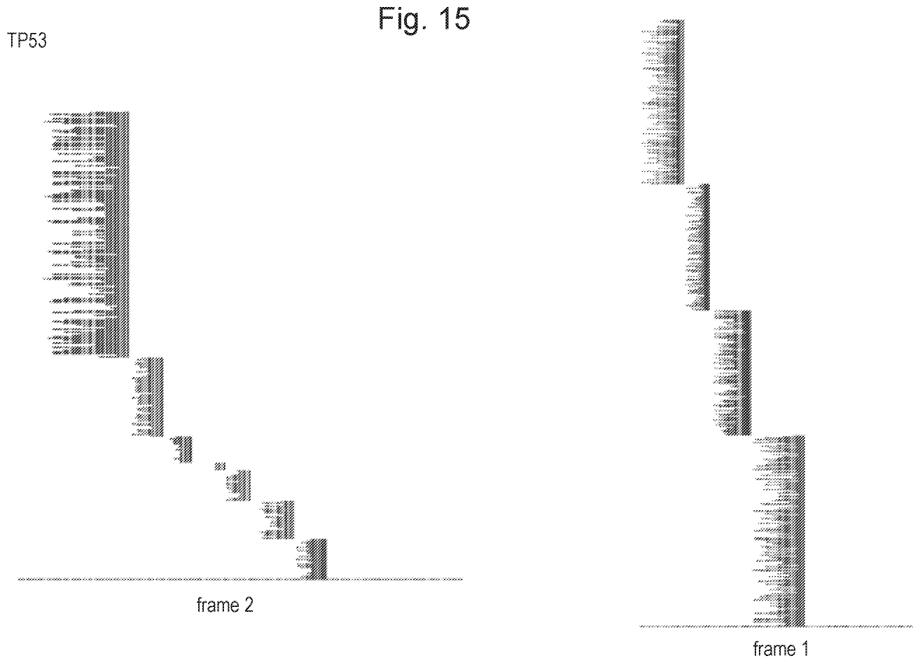

We have analyzed 10,186 cancer genomes from 33 tumor types of the 40 TCGA (The Cancer Genome Atlas²²) and focused on the 143,444 frame shift mutations represented in this cohort. Translation of these mutations after re-annotation to a RefSeq annotation, starting in the protein reading frame, can lead to 70,439 unique peptides that are 10 or more amino acids in length (a cut off we have set at a size sufficient to shape a distinct epitope in the context of MHC (FIG. 1a). The list of genes most commonly represented in the cohort and containing such frame shift mutations is headed nearly exclusively by tumor driver genes, such as NF1, RB, BRCA2 (FIG. 1b) whose whole or partial loss of function apparently contributes to tumorigenesis. Note that a priori frame shift mutations are expected to result in loss of gene function more than a random SNV, and more independent of the precise position. NOPs initiated from a frameshift mutation and of a significant size are prevalent in tumors, and are enriched in cancer driver genes. Alignment of the translated NOP products onto the protein sequence reveals that a wide array of different frame shift mutations translate in a common downstream stretch of neo open reading frame peptides (NOPs′), as dictated by the −1 and +1 alternative reading frames. While we initially screened for NOPs of ten or more amino acids, their open reading frame in the out-of-frame genome often extends far beyond that search window. As a result we see (FIG. 2) that hundreds of different frame shift mutations all at different sites in the gene nevertheless converge on only a handful of NOPs. Similar patterns are found in other common driver genes (FIG. 5). FIG. 2 illustrates that the precise location of a frame shift does not seem to matter much; the more or less straight slope of the series of mutations found in these 10,186 tumors indicates that it is not relevant for the biological effect (presumably reduction/loss of gene function) where the precise frame shift is, as long as translation stalls in the gene before the downstream remainder of the protein is expressed. As can also be seen in FIG. 2, all frame shift mutations alter the reading frame to one of the two alternative frames. Therefore, for potential immunogenicity the relevant information is the sequence of the alternative ORFs and more precisely, the encoded peptide sequence between 2 stop codons. We term these peptides ‘proto Neo Open Reading Frame peptides’ or pNOPs, and generated a full list of all thus defined out of frame protein encoding regions in the human genome, of 10 amino acids or longer. We refer to the total sum of all Neo-ORFs as the Neo-ORFeome. The Neo-ORFeome contains all the peptide potential that the human genome can generate after simple frame-shift induced mutations. The size of the Neo-ORFeome is 46.6 Mb. To investigate whether or not Nonsense Mediated Decay would wipe out frame shift mRNAs, we turned to a public repository containing read coverage for a large collection of cell lines (CCLE). We processed the data in a similar fashion as for the TCGA, identified the locations of frame shifts and subsequently found that, in line with the previous literature²³⁻²⁵, at least a large proportion of expressed genes also contained the frame shift mutation within the expressed mRNAs (FIG. 6). On the mRNA level, NOPs can be detected in RNAseq data. We next investigated how the number of patients relates to the number of NOPs. We sorted 10-mer peptides from NOPs by the number of new patients that contain the queried peptide. Assessed per tumor type, frame shift mutations in genes with very low to absent mRNA expression were removed to avoid overestimation. Of note NOP sequences are sometimes also encountered in the normal ORFeome, presumably as result of naturally occurring isoforms (e.g., FIG. 7). Also these peptides were excluded. We can create a library of possible ‘vaccines’ that is optimally geared towards covering the TCGA cohort, a cohort large enough that, also looking at the data presented here, it is representative of future patients (FIG. 10). Using this strategy 30% of all patients can be covered with a fixed collection of only 1,244 peptides of length 10 (FIG. 3). Since tumors will regularly have more than 1 frame shift mutation, one can use a ‘cocktail’ of different NOPs to optimally attack a tumor. Indeed, given a library of 1,244 peptides, 27% of the covered TCGA patients contain 2 or more ‘vaccine’ candidates. In conclusion, using a limited pool with optimal patient inclusion of vaccines, a large proportion of patients is covered. Strikingly, using only 6 genes (TP53, ARID1A, KMT2D, GATA3, APC, PTEN), already 10% of the complete TCGA cohort is covered. Separating this by the various tumor types, we find that for some cancers (like Pheochromocytoma and Paraganglioma (PCPG) or Thyroid carcinoma (THCA)) the hit rate is low, while for others up to 39% can be covered even with only 10 genes (Colon adenocarcinoma (COAD) using 60 peptides, Uterine Corpus Endometrial Carcinoma (UCEC) using 90 peptides), FIG. 4. At saturation (using all peptides encountered more than once) 50% of TCGA is covered and more than 70% can be achieved for specific cancer types (COAD, UCEC, Lung squamous cell carcinoma (LUSC) 72%, 73%, 73% respectively). As could be expected, these roughly follow the mutational load in the respective cancer types. In addition some frame shifted genes are highly enriched in specific tumor types (e.g. VHL, GATA3. FIG. 8). We conclude that at saturating peptide coverage, using only very limited set of genes, a large cohort of patients can be provided with off the shelf vaccines. To validate the presence of NOPs, we used the targeted sequencing data on 10,129 patients from the MSK-IMPACT cohort 26. For the 341-410 genes assessed in this cohort, we obtained strikingly similar results in terms of genes frequently affected by frame shifts and the NOPs that they create (FIG. 9). Even within this limited set of genes, 86% of the library peptides (in genes targeted by MSK-IMPACT) were encountered in the patient set. Since some cancers, like glioblastoma or pancreatic cancer, show survival expectancies after diagnosis measured in months rather than years (e.g. see 27), it is of importance to move as much of the work load and time line to the moment before diagnosis. Since the time of whole exome sequencing after biopsy is currently technically days, and since the scan of a resulting sequence against a public database describing these NOPs takes seconds, and the shipment of a peptide of choice days, a vaccination can be done theoretically within days and practically within a few weeks after biopsy. This makes it attractive to generate a stored and quality controlled peptide vaccine library based on the data presented here, possibly with replicates stored on several locations in the world. The synthesis in advance will—by economics of scale—reduce costs, allow for proper regulatory oversight, and can be quality certified, in addition to saving the patient time and thus provide chances. The present invention will likely not replace other therapies, but be an additional option in the treatment repertoire. The advantages of scale also apply to other means of vaccination against these common neoantigens, by RNA- or DNA-based approaches (e.g. 28), or recombinant bacteria (e.g. 29). The present invention also provides neoantigen directed application of the CAR-T therapy (For recent review see 30, and references therein), where the T-cells are directed not against a cell-type specific antigens (such as CD19 or CD20), but against a tumor specific neoantigen as provided herein. E.g. once one functional T-cell against any of the common p53 NOPs (FIG. 2) is identified, the recognition domains can be engineered into T-cells for any future patient with such a NOP, and the constructs could similarly be deposited in an off-the-shelf library. In the present invention, we have identified that various frame shift mutations can result in a source for common neo open reading frame peptides, suitable as pre-synthesized vaccines. This may be combined with immune response stimulating measures such as but not limited checkpoint inhibition to help instruct our own immune system to defeat cancer.

### Methods:

TCGA frameshift mutations—Frame shift mutations were retrieved from Varscan and mutect files per tumor type via https://portal.gdc.cancer.gov/. Frame shift mutations contained within these files were extracted using custom perl scripts and used for the further processing steps using HG38 as reference genome build.

CCLE frameshift mutations—For the CCLE cell line cohort, somatic mutations were retrieved from http://www.broadinstitute.org/ecle/data/browseData?conversationPropagation=begin

(CCLE_hybrid_capture1650_hg19_NoCommonSNPs_NoNeutralVariants_CDS_201 2.02.20.maf). Frame shift mutations were extracted using custom perl scripts using hg19 as reference genome.

Refseq annotation—To have full control over the sequences used within our analyses, we downloaded the reference sequences from the NCBI website (2018-02-27) and extracted mRNA and coding sequences from the gbff files using custom perl scripts. Subsequently, mRNA and every exon defined within the mRNA sequences were aligned to the genome (hg19 and hg38) using the BLAT suite. The best mapping locations from the psl files were subsequently used to place every mRNA on the genome, using the separate exons to perform fine placement of the exonic borders. Using this procedure we also keep track of the offsets to enable placement of the amino acid sequences onto the genome.

Mapping genome coordinate onto Refseq—To assess the effect of every mentioned frame shift mutation within the cohorts (CCLE or TCGA), we used the genome coordinates of the frameshifts to obtain the exact protein position on our reference sequence database, which were aligned to the genome builds. This step was performed using custom perl scripts taking into account the codon offsets and strand orientation, necessary for the translation step described below.

Translation of FS peptides—Using the reference sequence annotation and the positions on the genome where a frame shift mutation was identified, the frame shift mutations were used to translate peptides until a stop codon was encountered. The NOP sequences were recorded and used in downstream analyses as described in the text.

Verification of FS mRNA expression in the CCLE colorectal cancer cell lines—For a set of 59 colorectal cancer cell lines, the HG19 mapped ham files were downloaded from https://portal.gdc.cancer.gov/. Furthermore, the locations of FS mutations were retrieved from CCLE_hybrid_capture1650_hg19_NoCommonSNPs_NoNeutralVariants_CDS_201 2.02.20.maf

(http://www.broadinstitute.org/cele/data/browseDateconversationPropagation=beg in), by selection only frameshift entries. Entries were processed similarly to the TCGA data, but this time based on a HG19 reference genome. To get a rough indication that a particular location in the genome indeed contains an indel in the RNAseq data, we first extracted the count at the location of a frameshift by making use of the pileup function in samtools. Next we used the special tag XO:1 to isolate reads that contain an indel in it. On those bam files we again used the pileup function to count the number of reads containing an indel (assuming that the indel would primarily be found at the frameshift instructed location). Comparison of those 2 values can then be interpreted as a percentage of indel at that particular location. To reduce spurious results, at least 10 reads needed to be detected at the FS location in the original bam file.

Defining peptide library—To define peptide libraries that are maximized on performance (covering as many patients with the least amount of peptides) we followed the following procedure. From the complete TCGA cohort, FS translated peptides of size 10 or more (up to the encountering of a stop codon) were cut to produce any possible 10-mer. Then in descending order of patients containing a 10-mer, a library was constructed. A new peptide was added only if an additional patient in the cohort was included. peptides were only considered if they were seen 2 or more times in the TCGA cohort, if they were not filtered for low expression (see Filtering for low expression section), and if the peptide was not encountered in the orfeome (see Filtering for peptide presence orfeome). In addition, since we expect frame shift mutations to occur randomly and be composed of a large array of events (insertions and deletions of any non triplet combination), frame shift mutations being encountered in more than 10 patients were omitted to avoid focusing on potential artefacts. Manual inspection indicated that these were cases with e.g. long stretches of Cs, where sequencing errors are common.

Filtering for low expression—Frameshift mutations within genes that are not expressed are not likely to result in the expression of a peptide. To take this into account we calculated the average expression of all genes per TCGA entity and arbitrarily defined a cutoff of 2 log 2 units as a minimal expression. Any frameshift mutation where the average expression within that particular entity was below the cutoff was excluded from the library. This strategy was followed, since mRNA gene expression data was not available for every TCGA sample that was represented in the sequencing data set. Expression data (RNASEQ v2) was pooled and downloaded from the R2 platform (http://r2.amc.nl). In current sequencing of new tumors with the goal of neoantigen identification such mRNA expression studies are routine and allow routine verification of presence of mutant alleles in the mRNA pool.

Filtering for peptide presence orfeome—Since for a small percentage of genes, different isoforms can actually make use of the shifted reading frame, or by chance a 10-mer could be present in any other gene, we verified the absence of any picked peptide from peptides that can be defined in any entry of the reference sequence collection, once converted to a collection of tiled 10-mers.

Generation of cohort coverage by all peptides per gene To generate overviews of the proportion of patients harboring exhaustive FS peptides starting from the most mentioned gene, we first pooled all peptides of size 10 by gene and recorded the largest group of patients per tumor entity. Subsequently we picked peptides identified in the largest set of patients and kept on adding a new peptide in descending order, but only when at least 1 new patient was added. Once all patients containing a peptide in the first gene was covered, we progressed to the next gene and repeated the procedure until no patient with FS mutations leading to a peptide of size 10 was left.

proto-NOP (pNOP) and Neo-ORFeome proto—NOPs are those peptide products that result from the translation of the gene products when the reading frame is shifted by −1 or +1 base (so out of frame). Collectively, these pNOPs form the Neo-Orfeome. As such we generated a pNOP reference base of any peptide with length of 10 or more amino acids, from the RefSeq collection of sequences. Two notes: the minimal length of 10 amino acids is a choice; if one were to set the minimal window at 8 amino acids the total numbers go up a bit, e.g. the 30% patient covery of the library goes up. On a second note: we limited our definition to ORFs that can become in frame after a single insertion deletion on that location; this includes obviously also longer insertion or deletion stretches than +1 or −1. The definition has not taken account more complex events that get an out-of-frame ORF in frame, such as mutations creating or deleting splice sites, or a combination of two frame shifts at different sites that result in bypass of a natural stop codon; these events may and will occur, but counting those in will make the definition of the Neo-ORFeome less well defined. For the magnitude of the numbers these rare events do not matter much.

Visualizing flops—Visualization of the nops was performed using custom perl scripts, which were assembled such that they can accept all the necessary input data structures such as protein sequence, frameshifted protein sequences, somatic mutation data, library definitions, and the peptide products from frameshift translations.

Detection of frameshift resulting neopeptides in cancer patients with cancer predisposition mutations—Somatic and germline mutation data were downloaded from the supplementary files attached to the manuscript posted here:

https://www.biorxiv.org/content/biorxiv/early/2019/01/16/415133.full.pdf.

Frameshift mutations were selected from the somatic mutation files and out-of-frame peptides were predicted using custom Perl and Python scripts, based on the human reference genome GRCh37. Out-of-frame peptides were selected based on their length (>=10 amino acids) and mapped against out of frame peptide sequences for each possible alternative transcript for genes present in the human genome, based on Ensembl annotation (ensembl.org).

### REFERENCES

1 Schumacher T. N., & Schreiber R. D. Neoantigens in cancer immunotherapy. Science. 348, 69-74 (2015).
2 Gubin M. M., Artyomov M. N., Mardis E. R., & Schreiber R. D. Tumor neoantigens: building a framework for personalized cancer immunotherapy. J Clin Invest. 125, 3413-21 (2015).
3 Ward J. P., Gubin M. M., & Schreiber R. D. The Role of Neoantigens in Naturally Occurring and Therapeutically Induced Immune Responses to Cancer. Adv Immunol. 130, 25-74 (2016).
4 DeWeerdt S. Calling cancer's bluff with neoantigen vaccines. Nature. 552, S76-S77 (2017).
5 Guo C., et al. Therapeutic cancer vaccines: past, present, and future. Adv Cancer Res. 119, 421-75 (2013).
6 Overwijk W. W., Wang E., Marincola F. M., Rammensee H. G., & Restifo N. P. Mining the mutanome: developing highly personalized Immunotherapies based on mutational analysis of tumors. J Immunother Cancer. 1, 11 (2013).
7 Yamada A., Sasada T., Noguchi M., & Itoh K. Next-generation peptide vaccines for advanced cancer. Cancer Sci. 104, 15-21 (2013).
8 Ott P. A., et al. An immunogenic personal neoantigen vaccine for patients with melanoma. Nature. 547, 217-221 (2017).
9 Wirth T. C., & Kuhnel F. Neoantigen Targeting-Dawn of a New Era in Cancer Immunotherapy? Front Immunol. 8, 1848 (2017).
10 Yarchoan M., Hopkins A., & Jaffee E. M. Tumor Mutational Burden and Response Rate to PD-1 Inhibition. N Engl J Med. 377, 2500-2501 (2017).
11 Sahin U., et al. Personalized RNA mutanome vaccines mobilize poly-specific therapeutic immunity against cancer. Nature. 547, 222-226 (2017).
12 Linnebacher M., et al. Frameshift peptide-derived T-cell epitopes: a source of novel tumor-specific antigens. Int J Cancer. 93, 6-11 (2001).
13 Sonntag K., et al. Immune monitoring and TCR sequencing of CD4 T cells in a long term responsive patient with metastasized pancreatic ductal carcinoma treated with individualized, neoepitope derived multipeptide vaccines: a case report. et al. J Transl Med. 16, 23 (2018).
14 MacArthur D. G., et al. A systematic survey of loss-of-function variants in human protein-coding genes. Science. 335, 823-8 (2012).
15 Turajlic S., et al. Insertion-and-deletion-derived tumour-specific neoantigens and the immunogenic phenotype: a pan-cancer analysis. Lancet Oncol. 18, 1009-1021 (2017).
16 Rammensee H., Bachmann J., Emmerich N. P., Bachor O. A., & Stevanovic S. SYFPEITHI: database for MHC ligands and peptide motifs. Immunogenetics. 50, 213-9 (1999).
17 Alvarez B., Barra C., Nielsen M., & Andreatta M. Computational Tools for the Identification and Interpretation of Sequence Motifs in Immunopeptidomes. Proteomics. 18, e1700252 (2018).
18 Andreatta M., et al. Accurate pan-specific prediction of peptide-MHC class II binding affinity with improved binding core identification. Immunogenetics. 67, 641-50 (2015).
19 Rizvi N. A., et al. Cancer immunology. Mutational landscape determines sensitivity to PD-1 blockade in non-small cell lung cancer. Science. 348, 124-8 (2015).
20 Prickett T. D., et al. Durable Complete Response from Metastatic Melanoma after Transfer of Autologous T Cells Recognizing 10 Mutated Tumor Antigens. Cancer Immunol Res. 4, 669-78 (2016).
21 Liu R., et al. H7N9 T-cell epitopes that mimic human sequences are less immunogenic and may induce Treg-mediated tolerance. Hum Vaccin Immunother. 11, 2241-52 (2015).
22 Weinstein J. N., et al. The Cancer Genome Atlas Pan-Cancer analysis project. Nat Genet. 45, 1113-20 (2013).
23 Lindeboom R. G., Supek F., & Lehner B. The rules and impact of nonsense-mediated mRNA decay in human cancers. Nat Genet. 48, 1112-8 (2016).
24 Longman D., Plasterk R. H., Johnstone I. L., & Caceres J. F. Mechanistic insights and identification of two novel factors in the C. elegans NMD pathway. Genes Dec. 21, 1075-85 (2007).
25 Nguyen L. S., Wilkinson M. F., & Gecz J. Nonsense-mediated mRNA decay: inter-individual variability and human disease. Neurosci Biobehav Reu. 46 Pt 2, 175-86 (2014).
26 Zehir A., et al. Mutational landscape of metastatic cancer revealed from prospective clinical sequencing of 10,000 patients. Nat Med. 23, 703-713 (2017).
27 Fest J., et al. Underestimation of pancreatic cancer in the national cancer registry Eur J Cancer. 72, 186-191 (2017).
28 Boisguerin V., et al. Translation of genomics-guided RNA-based personalised cancer vaccines: towards the bedside. Br J Cancer. 111, 1469-75 (2014).
29 Keenan B. P., et al. A Listeria vaccine and depletion of T-regulatory cells activate immunity against early stage pancreatic intraepithelial neoplasms and prolong survival of mice. Gastroenterology. 146, 1784-94.e6 (2014).
30 Ramello M. C., Haura E. B., & Abate-Daga D. CAR-T cells and combination therapies: What's next in the immunotherapy revolution? Pharmacol Res. 129, 194-203 (2018).
31 Giannakis, Marios, et al. “Genomic Correlates of Immune-Cell Infiltrates in Colorectal Carcinoma.” Cell Reports, vol. 17, no. 4, October 2016, p. 1206.
32 Linnebacher, M., et al. “Frameshift Peptide-Derived T-Cell Epitopes: A Source of Novel Tumor-Specific Antigens.” International Journal of Cancer. Journal International Du Cancer, vol. 93, no. 1, July 2001, pp. 6-11.
33 Maby, Pauline, et al. “Correlation between Density of CD8+ T-Cell Infiltrate in Microsatellite Unstable Colorectal Cancers and Frameshift Mutations: A Rationale for Personalized Immunotherapy.” Cancer Research, vol. 75, no. 17, September 2015, pp. 3446-55.
34 Saeterdal, I., et al. “A TGF betaRII Frameshift-Mutation-Derived CTL Epitope Recognised by HLA-A2-Restricted CD8+ T Cells.” Cancer Immunology, Immunotherapy: CII, vol. 50, no. 9, November 2001, pp. 469-76.
35 Turajlic, Samra, et al. “Insertion-and-Deletion-Derived Tumour-Specific Neoantigens and the Immunogenic Phenotype: A Pan-Cancer Analysis.” The Lancet Oncology, vol. 18, no. 8, August 2017, pp. 1009-21.
36 Williams, David S., et al. “Nonsense Mediated Decay Resistant Mutations Are a Source of Expressed Mutant Proteins in Colon Cancer Cell Lines with Microsatellite Instability.” PloS One, vol. 5, no. 12, December 2010, p. e16012.

1.-23. (canceled)

24. A vaccine or collection of vaccines for treating cancer comprising:
(i) a peptide, or a collection of tiled peptides, having the amino acid sequence selected from SEQ ID NO:29, an amino acid sequence having 90% identity to SEQ ID NO:29, a fragment thereof comprising at least 10 consecutive amino acids of SEQ ID NO:29, or one or more nucleic acids encoding said peptide or collection of tiled peptides; and
a peptide, or a collection of tiled peptides, having the amino acid sequence selected from SEQ ID NO:30, an amino acid sequence having 90% identity to SEQ ID NO:30, a fragment thereof comprising at least 10 consecutive amino acids of SEQ ID NO:30, or one or more nucleic acids encoding said peptide or collection of tiled peptides;
optionally also comprising a peptide, or a collection of tiled peptides, having the amino acid sequence selected from any of SEQ ID NOS:31-33, an amino acid sequence having 90% identity to any of SEQ ID NOS:31-33, a fragment thereof comprising at least 10 consecutive amino acids of any of SEQ ID NOS:31-33, or one or more nucleic acids encoding said peptide or collection of tiled peptides;
(ii) a peptide, or a collection of tiled peptides, having the amino acid sequence selected from SEQ ID NO:130, an amino acid sequence having 90% identity to SEQ ID NO:130, a fragment thereof comprising at least 10 consecutive amino acids of SEQ ID NO:130, or one or more nucleic acids encoding said peptide or collection of tiled peptides; and
a peptide, or a collection of tiled peptides, having the amino acid sequence selected from SEQ ID NO:131, an amino acid sequence having 90% identity to SEQ ID NO:131, a fragment thereof comprising at least 10 consecutive amino acids of SEQ ID NO:131 or one or more nucleic acids encoding said peptide or collection of tiled peptides,
(iii) a peptide, or a collection of tiled peptides, having the amino acid sequence selected from SEQ ID NO:157, an amino acid sequence having 90% identity to SEQ ID NO:157, a fragment thereof comprising at least 10 consecutive amino acids of SEQ ID NO:157, or one or more nucleic acids encoding said peptide or collection of tiled peptides; and
a peptide, or a collection of tiled peptides, having the amino acid sequence selected from SEQ ID NO:158, an amino acid sequence having 90% identity to SEQ ID NO:158, a fragment thereof comprising at least 10 consecutive amino acids of SEQ ID NO:158, or one or more nucleic acids encoding said peptide or collection of tiled peptides;
(iv) a peptide, or a collection of tiled peptides, having the amino acid sequence selected from SEQ ID NO:273, an amino acid sequence having 90% identity to SEQ ID NO:273, a fragment thereof comprising at least 10 consecutive amino acids of SEQ ID NO:273, or one or more nucleic acids encoding said peptide or collection of tiled peptides; and
a peptide, or a collection of tiled peptides, having the amino acid sequence selected from SEQ ID NO:274, an amino acid sequence having 90% identity to SEQ ID NO:274, a fragment thereof comprising at least 10 consecutive amino acids of SEQ ID NO:274, or one or more nucleic acids encoding said peptide or collection of tiled peptides;
(v) a peptide, or a collection of tiled peptides, having the amino acid sequence selected from SEQ ID NO:528, an amino acid sequence having 90% identity to SEQ ID NO:528, a fragment thereof comprising at least 10 consecutive amino acids of SEQ ID NO:528, or one or more nucleic acids encoding said peptide or collection of tiled peptides; and
a peptide, or a collection of tiled peptides, having the amino acid sequence selected from SEQ ID NO:529, an amino acid sequence having 90% identity to SEQ ID NO:529, a fragment thereof comprising at least 10 consecutive amino acids of SEQ ID NO:529, or one or more nucleic acids encoding said peptide or collection of tiled peptides; and/or
(vi) at least two peptides, wherein each peptide, or a collection of tiled peptides, comprises a different amino acid sequence selected from any of SEQ ID NOS:1-3, an amino acid sequence having 90% identity to any of SEQ ID NOS:1-3, a fragment thereof comprising at least 10 consecutive amino acids of any of SEQ ID NOS:1-3, or one or more nucleic acids encoding said peptide or collection of tiled peptides,
optionally also comprising a peptide, or a collection of tiled peptides, having the amino acid sequence selected from any of SEQ ID NOS:4-15, an amino acid sequence having 90% identity to any of SEQ ID NOS:4-15, a fragment thereof comprising at least 10 consecutive amino acids of any of SEQ ID NOS:4-15, or one or more nucleic acids encoding said peptide or collection of tiled peptides.

25. The vaccine or collection of vaccines of claim 24, wherein at least two of said peptides are linked.

26. The vaccine or collection of vaccines of 25, wherein said peptides are comprised within the same polypeptide.

27. The vaccine or collection of vaccines of claim 24, wherein the nucleic acid molecules encoding said peptides are comprised in one or more vectors.

28. The vaccine or collection of vaccines of claim 27, wherein said vector is a viral vector.

29. The vaccine or collection of vaccines of claim 24, further comprising a pharmaceutically acceptable excipient, an adjuvant, or a therapeutic agent.

30. The vaccine or collection of vaccines of claim 29, wherein the therapeutic agent is a checkpoint inhibitor, a chemotherapeutic agent, or an antibody.

31. The vaccine or collection of vaccines of claim 24, further comprising an immune-effective amount of adjuvant.

32. The vaccine or collection of vaccines of claim 24, comprising (i), (ii), (iii), (iv), (v), and (vi).

33. A method for providing a vaccine for immunizing a patient against a cancer in said patient comprising determining the sequence of ARID1A, CDKN2A, KMT2B, KMT2D, TP53, and/or PTEN in cancer cells of said cancer and when the determined sequence comprises a frameshift mutation that produces a neoantigen of any of SEQ ID NOS:1-352 or a fragment thereof, providing a vaccine selected from the vaccine or collection of vaccines of claim 24.

34. A method of treating an individual for cancer or reducing the risk of developing said cancer, the method comprising administering to the individual in need thereof a vaccine selected from the vaccine or collection of vaccines of claim 24.


SEQUENCE LISTING (583 sequences)

SEQ ID NO: 1 - protein, 97 aa, Artificial Sequence
  pNOP36301 alt splice a
TGGPSSPSSHWKTPVVIYWDGTALRCVFVPVLGETGAQRKRISARKGSLTTSCPQGALSEHCPTTPAPLPSQRRNHWMENISPFRSVGVSASRCSES

SEQ ID NO: 2 - protein, 103 aa, Artificial Sequence
  pNOP31232 alt splice a
TGGPSSPSSHWKTPVVIYWDGTALRCVFVPVLGETGAQRKRISARKGSLTTSCPQGALSEHCPTTPAPLPSQRRNHWMENISPFRTRPAFKKKIVKESMKMVL

SEQ ID NO: 3 - protein, 95 aa, Artificial Sequence
  pNOP38141 alt splice a
TGGPSSPSSHWKTPVVIYWDGTALRCVFVPVLGETGAQRKRISARKGSLTTSCPQGALSEHCPTTPAPLPSQRRNHWMENISPFRCYLTYDGVTS

SEQ ID NO: 4 - protein, 78 aa, Artificial Sequence
  pNOP59073
CCPRTILNNGSLKTQVQMKLPECQRLLPPWPLHQQLLHRRPLHQPPPGPCHLLSLPRKPTRAATVSVWASCILGQPSL

SEQ ID NO: 5 - protein, 85 aa, Artificial Sequence
  pNOP49591
SSQNARGCSPRGPCTSSSYTGGPCTSPLLAPVIFCPFPENLPGQLRFPSGLLAFWDSQVCDLHVLPCPQQDVLPTGQDLPCAAVG

SEQ ID NO: 6 - protein, 72 aa, Artificial Sequence
  pNOP70126
GAAPTMSAAQIAMVWPLLSILSEWKEICVWSIWMTETLFDIVWWCPMSRLRLALTVPPSTTTTCVTVPAWAA

SEQ ID NO: 7 - protein, 36 aa, Artificial Sequence
  pNOP224126
CFANWPRPALCSCGLIPHPRPAPASAPWPSTSSHST

SEQ ID NO: 8 - protein, 31 aa, Artificial Sequence
  pNOP272502
FHTPARHPRPRHGHLQAVTAHDGGCEALPPP

SEQ ID NO: 9 - protein, 28 aa, Artificial Sequence
  pNOP316190
VRKHFQTYGNYFLKTTFCPPCRPKQWMI

SEQ ID NO: 10 - protein, 40 aa, Artificial Sequence
  pNOP193414 alt splice b
ASTAQQHQLLSPAKEETTGWRIFHPSGPDQLSKRKLLKRA

SEQ ID NO: 11 - protein, 46 aa, Artificial Sequence
  pNOP158914
LARTPLPSTRCFANWPRPALCSCGLIPHPRPAPASAPWPSTSSHST

SEQ ID NO: 12 - protein, 30 aa, Artificial Sequence
  pNOP281999 alt splice b
ASTAQQHQLLSPAKEETTGWRIFHPSDPWA

SEQ ID NO: 13 - protein, 29 aa, Artificial Sequence
  pNOP293143 alt splice b
ASTAQQHQLLSPAKEETTGWRIFHPSDAT

SEQ ID NO: 14 - protein, 33 aa, Artificial Sequence
  pNOP252394
GACLCLSWERPAHRGRESPQERGASPRAAPREH

SEQ ID NO: 15 - protein, 51 aa, Artificial Sequence
  pNOP136003
SPKRVSLPPAIKNSCSRQKGLTQTDILHFLFPTDSLPPPSLPPLPFWVLGL

SEQ ID NO: 16 - protein, 23 aa, Artificial Sequence
  pNOP385655
QFLHGRHEPEAHPHHHHTGRLQW

SEQ ID NO: 17 - protein, 22 aa, Artificial Sequence
  pNOP405064
RWSGPSSASYPSGRKFACGVFG

SEQ ID NO: 18 - protein, 15 aa, Artificial Sequence
  pNOP539666
DVLPTGQDLPCAAVG

SEQ ID NO: 19 - protein, 78 aa, Artificial Sequence
  pNOP59708
LRLTFSTSCSPLTASHPHLSLPCHFGFWVFEPLLAIGVRQKHPGLPFALSRGSTEQVGLHWCFVVGRRMGSRTYQLRF

SEQ ID NO: 20 - protein, 24 aa, Artificial Sequence
  pNOP367554
MRPWNSRMPRLGRSQGGAGLTPAT

SEQ ID NO: 21 - protein, 10 aa, Artificial Sequence
  pNOP703537
LYHHPLQLHV

SEQ ID NO: 22 - protein, 13 aa, Artificial Sequence
  pNOP602122
KQRSVPLAVPSNG

SEQ ID NO: 23 - protein, 34 aa, Artificial Sequence
  pNOP243169
GLGTQGCPGWEGARGEQGSLQPPEVQKGSVYLPP

SEQ ID NO: 24 - protein, 18 aa, Artificial Sequence
  pNOP483390
RRAPSESGNIFRPMETTS

SEQ ID NO: 25 - protein, 20 aa, Artificial Sequence
  pNOP433152
HGHLQAVTAHDGGCEALPPP

SEQ ID NO: 26 - protein, 20 aa, Artificial Sequence
  pNOP445026
TRRKLKILSVGVSASRCSES

SEQ ID NO: 27 - protein, 13 aa, Artificial Sequence
  pNOP604680
LTMVLLPDKLVVS

SEQ ID NO: 28 - protein, 13 aa, Artificial Sequence
  pNOP619453
WRSRSQILASSPL

SEQ ID NO: 29 - protein, 67 aa, Artificial Sequence
  pNOP82315
RSYRRMIHLWWTAQISLGVCRSLTVACCTGGLVGGTPLSISRPTSRARQSCCLPGLTHPAHQPLGSM

SEQ ID NO: 30 - protein, 179 aa, Artificial Sequence
  pNOP6110 alt splice a
ALGPHSRISCLPTQTRGCILLAATPRSSSSSSSNDMIPMAISSPPKAPLLAAPSPASRLQCINSNSRITSGQWMAHMALLPSGTKGRCTACHTALGRGSLSSSSCPQPSPSLPASNKLPSLPLSKMYTTSMAMPILPLPQLLLSADQQAAPRTNFHSSLAETVSLHPLAPMPSKTCHHK

SEQ ID NO: 31 - protein, 64 aa, Artificial Sequence
  pNOP88606
FWPHPPSAAWRSCIALWCASSVTERTRCAGRWLWYCWPTWLRGTAWQLVPLQCRRAVSATSWAS

SEQ ID NO: 32 - protein, 90 aa, Artificial Sequence
  pNOP43369
TNQALPKIEVICRGTPRCPSTVPPSPAQPYLRVSLPEDRYTQAWAPTSRTPWGAMVPRGVSMAHKVATPGSQTIMPCPMPTTPVQAWLEA

SEQ ID NO: 33 - protein, 185 aa, Artificial Sequence
  pNOP5538
PCRAGRRVPWAASLIHSRFLLMDNKAPAGMVNRARLHITTSKVLTLSSSSHPTPSNHRPRPLMPNLRISSSHSLNHHSSSPLSLHTPSSHPSLHISSPRLHTPPSSRRHSSTPRASPPTHSHRLSLLTSSSNLSSQHPRRSPSRLRILSPSLSSPSKLPIPSSASLHRRSYLKIHLGLRHPQPPQ

SEQ ID NO: 34 - protein, 282 aa, Artificial Sequence
  pNOP1299
PHGAARRRRWRQQRWGGGASSLSRGRLAAPSLRLRATLRPEPVCRRRRRGRRLPPTTWRTTKPWPGSAAERRRRGPGALRGAPAELSRPRLPQPPVQLLLPQPQRLPPARPGLRAELPERWHSGLRRGGGCRLQAASLLQRLRLLVVFVLRSAALRGHGGRRPLRGRRGNSPAHRHPHPQPTAHVAQLGPGLPGLPRGRLQWRAPGRGRRQGPGGHGLAVLGGCGGGSCGGGRLGRGPTKEPPRAHEPREQRRRGAAARPDPSAIQSNGSDGQDETSAIWRD

SEQ ID NO: 35 - protein, 130 aa, Artificial Sequence
  pNOP16341
APREVALRAPARRRLPAPSRLPPPAPPPPRRLRPSLSSASGPWGEAAPPRPAGELPSPPPPPPSTNCSRRPARPGATRATPGATTVAGPRTGAPARARRTWPRSVGGLRRRQLRRRPPREGPNKGATTRP

SEQ ID NO: 36 - protein, 222 aa, Artificial Sequence
  pNOP3000
PILAATGTSVRTAARTWVPRAAIRVPDPAAVPDDHAGPGAECHGRPLLYTADSSLWTTRPQRVWSTGPDSILQPAKSSPSAAAATLLPATTVPDPSCPTFVSAAATVSTTTAPVLSASILPAAIPASTSAVPGSIPLPAVDDTAAPPEPAPLLTATGSVSLPAAATSAASTLDALPAGCVSSAPVSAVPANCLFPAALPSTAGAISRFIWVSGILSPLNDLQ

SEQ ID NO: 37 - protein, 93 aa, Artificial Sequence
  pNOP39264 alt splice a
ALGPHSRISCLPTQTRGCILLAATPRSSSSSSSNDMIPMAISSPPKAPLLAAPSPASRLQCINSNSRYPALLPCPGQWRTAPLLASLHSCTLG

SEQ ID NO: 38 - protein, 140 aa, Artificial Sequence
  pNOP13360
SSSVSFLSSYLPSPAWHPRPFPVPCWLSRQCCSVSLRTTLACCSARQPDATSATQWPVGQHHASFHEPIKHCPRSRLYAEEPPDAPVQFPPARLSLISASAFRRTDTHRHGLLPAELHGELWSPGGSVWPTRWLPQAAKL

SEQ ID NO: 39 - protein, 27 aa, Artificial Sequence
  pNOP323677
LRSTRTKNGGNLQPTSMWAHQAVLPAP

SEQ ID NO: 40 - protein, 67 aa, Artificial Sequence
  pNOP81513
KSSISSVSMPLNARLNGEKTLPQTSLQLLIPRSPSPRSSLPLLRDQDLCRGPRLPSQPAVPWQKEET

SEQ ID NO: 41 - protein, 57 aa, Artificial Sequence
  pNOP109934
ETSGPLSPLCVCEGDWWIDSGQQEQKMAGTCNQPQCGHIKQCCQLLEKAVYPVSLCL

SEQ ID NO: 42 - protein, 49 aa, Artificial Sequence
  pNOP141882
CGHDAAGCPRAACLGQGGREPLRVYSVRITAVGHLGITVDELIGFTSHL

SEQ ID NO: 43 - protein, 109 aa, Artificial Sequence
  pNOP26533
HGRAGRPRRRQQPGQPAAAAALGAEESRAAAAGGGGGRGGGGGSGRARGNEGSRRAGKRGPRRGAAAAAGKGAAGRGREQWGWRRRRSRQRRRARRGAGPEELERERGP

SEQ ID NO: 44 - protein, 92 aa, Artificial Sequence
  pNOP40276
AATKWSGGGTAWRCSGKTPWLHSPTSRGSWTYLHTPRAFACLSWTDSYTGQFALQLKPRTPFPPWAPMPSFPRRDWSWKPSANSASRTTMWT

SEQ ID NO: 45 - protein, 79 aa, Artificial Sequence
  pNOP57388
AHQGFPAAKESRVIQLSLLSLLIPPLTCLASEALPRPLLALPPVLLSLAQDHSRLLQCQATRCHLGHPVASRTASCILP

SEQ ID NO: 46 - protein, 117 aa, Artificial Sequence
  pNOP22341
TITSRSRPAAAVAAAAMGWGRLLTQPRPPCRPQPTASGNPTAGARLPSPPPRPPSSTNNMADNKALAWQRCRAAAAGAWSPTRGPSRTLTTTASPTTSTTPTTPTAAPTPRPPRPTR

SEQ ID NO: 47 - protein, 35 aa, Artificial Sequence
  pNOP232518
CGGLPARCLPWPRWTRTTQSLLCTNHGCWTSRYHR

SEQ ID NO: 48 - protein, 65 aa, Artificial Sequence
  pNOP86506
KGGGTGPRGELQQSGVVVGLLGDAPGKHLGYTRQHLGAVGPISIPREHLPACPGRTPTLGSLPFS

SEQ ID NO: 49 - protein, 32 aa, Artificial Sequence
  pNOP266437
PRMELRVQRPSRRAASFHLALAQHRATGTSRS

SEQ ID NO: 50 - protein, 27 aa, Artificial Sequence
  pNOP317526
APGAAAAGGSRSPGPLSHPVQWIRWAR

SEQ ID NO: 51 - protein, 63 aa, Artificial Sequence
  pNOP91542
HGQYATSGWVRDVSPTRGHEPENPRNCCRHACCCQLYPKQAARLPQYESRGHDGNWTSLWTRD

SEQ ID NO: 52 - protein, 46 aa, Artificial Sequence
  pNOP160041
QGPLHLTTSPHQACRITFLRYPALLPCPGQWRTAPLLASLHSCTLG

SEQ ID NO: 53 - protein, 39 aa, Artificial Sequence
  pNOP205126
QQQRVHQGQQTRRGPHLMDLQKNGSQPLWMTCCLLGLAP

SEQ ID NO: 54 - protein, 69 aa, Artificial Sequence
  pNOP78127
YGWHDQPSGTPIFHGWNHGQQFCRDGSQPRDDGPWGCKVNSSHQNEQQGRWDTQDRIQIQEIQFFYYNQ

SEQ ID NO: 55 - protein, 39 aa, Artificial Sequence
  pNOP204073
NAAHRSEGQPRRLVAFPWHTPAPIWSLCPCAPHDKAPSI

SEQ ID NO: 56 - protein, 14 aa, Artificial Sequence
  pNOP578746
PLPPAAAAAAAATT

SEQ ID NO: 57 - protein, 58 aa, Artificial Sequence
  pNOP108335
RTNPTVRMRPHCVPFWTGRILLPSAASVCPIPFEACHLCQAMTLRCPNTQGCCSSWAS

SEQ ID NO: 58 - protein, 50 aa, Artificial Sequence
  pNOP140600
SPGPLFHPGPQCRPFPAETGLGNPQQTQHPGQQCGPDSGHTPLQPPGEVV

SEQ ID NO: 59 - protein, 45 aa, Artificial Sequence
  pNOP162214
APTSRRPPEPISIPVWPRPCLCTPWHQCPAKHATTNDGRPHTGIS

SEQ ID NO: 60 - protein, 106 aa, Artificial Sequence
  pNOP28463
CTVFDWPVMTAVGHLPPPCVCACVENLETDCCPLFMQNHLRIQFTLCCPASPLGKSLSCFSLLLPPPLPPSPHAFLFLVLTLLPSGPYPTLFEKTKLCLHRRLFLF

SEQ ID NO: 61 - protein, 106 aa, Artificial Sequence
  pNOP28543 alt splice b
FLWQSVLHPRHPFWQPLPQPADYNVSTATAELQAANGWHIWPSCQAARRGDVQRAIQHWAGAASAAAVAPSPAPACQPATSCPAFPSARCIQPVWQCLSCHCHSCY

SEQ ID NO: 62 - protein, 26 aa, Artificial Sequence
  pNOP342491
STLRDPHIPWVEPWPTILQGWQPAQR

SEQ ID NO: 63 - protein, 23 aa, Artificial Sequence
  pNOP382230
LCQQAEHGLCPPGPRLSWREPNR

SEQ ID NO: 64 - protein, 66 aa, Artificial Sequence
  pNOP84384
PKEPGVPGDGCGTAGQPGSGGQPGSSCHCSAEGQYRQPPGLPRGQPCRHTVPAEPGQPPPHAEPTL

SEQ ID NO: 65 - protein, 44 aa, Artificial Sequence
  pNOP171474
QVSIPALWDENAEGRSPSTCLAHSTCPCAAPHDSAGYHLPTWLC

SEQ ID NO: 66 - protein, 33 aa, Artificial Sequence
  pNOP251638
DPTVYPSGLAGFSCQALRLCVQYHSKPVICARQ

SEQ ID NO: 67 - protein, 69 aa, Artificial Sequence
  pNOP76377
FQEVPAQDPASLSCGIRIYAGAPDSPVNQQFHGRRRRLKATNSSIHTTQSDPPIARHEQEQFSWDPGCL

SEQ ID NO: 68 - protein, 56 aa, Artificial Sequence
  pNOP115908
TTRQMGHPRQNPNPRNPVLLLQPMRRSPSCMSWVVSLRGRCGWTVIWPSLRRRPWA

SEQ ID NO: 69 - protein, 49 aa, Artificial Sequence
  pNOP145255
SHTACVEAEEAAHNERHWNPGGMAGNDVPQVWSPGREHMGIRYHQHPAV

SEQ ID NO: 70 - protein, 46 aa, Artificial Sequence
  pNOP157058
AYPDPLREQDRAAAFPASRTLPTSPSEACDNSRGYTRDNRPGGAPT

SEQ ID NO: 71 - protein, 37 aa, Artificial Sequence
  pNOP221454
RSMRWVTQDRERYWILGGSARCLVQLPWRVGKKKKNF

SEQ ID NO: 72 - protein, 37 aa, Artificial Sequence
  pNOP222331
TEQMKCCTQIRGPTTKARGLPMAHASPHMVPLPLCPP

SEQ ID NO: 73 - protein, 31 aa, Artificial Sequence
  pNOP272985
GKLQGVIPSCPQGRAPTAGWVTPTVVLPALG

SEQ ID NO: 74 - protein, 30 aa, Artificial Sequence
  pNOP289760
RTALPPHSSSRARPASSTCRTHPLSQLVWT

SEQ ID NO: 75 - protein, 27 aa, Artificial Sequence
  pNOP329083
TGKPKKLLSPCMLLPTLSKTGRQATPI

SEQ ID NO: 76 - protein, 54 aa, Artificial Sequence
  pNOP120573
CLAQCQLPQCRHGWRHKPHGCRRSNAWTAWHPTLWHTPSREDESRLHGQPALWP

SEQ ID NO: 77 - protein, 21 aa, Artificial Sequence
  pNOP419746
PIIMPTGRARALPPRAPPIMA

SEQ ID NO: 78 - protein, 18 aa, Artificial Sequence
  pNOP472965
GRARRYEPEPSVKTLQLA

SEQ ID NO: 79 - protein, 49 aa, Artificial Sequence
  pNOP144966
RQPPGRKARAPPWGRRSRWERSCRTGPRAMGVAAAAEPAAAAGPARSRT

SEQ ID NO: 80 - protein, 31 aa, Artificial Sequence
  pNOP271959
DVQTPRAAAHPGQADPAAPQAPRTEAGTTNL

SEQ ID NO: 81 - protein, 31 aa, Artificial Sequence
  pNOP280686
VTPPWATGLMALTWPICHLRLGQGCVPHQGA

SEQ ID NO: 82 - protein, 27 aa, Artificial Sequence
  pNOP325333
PLQSCCRPWARKCGDGTTTALSLWRSL

SEQ ID NO: 83 - protein, 26 aa, Artificial Sequence
  pNOP339133
PPHGDRRSSESWSEHIRDFQQPRRAE

SEQ ID NO: 84 - protein, 19 aa, Artificial Sequence
  pNOP460168
QICLLWVGNLWTSIASMCL

SEQ ID NO: 85 - protein, 18 aa, Artificial Sequence
  pNOP471545
FGGISPSHLALLKPHSLC

SEQ ID NO: 86 - protein, 18 aa, Artificial Sequence
  pNOP484623
SHQLQHPHHTVRSPHCQA

SEQ ID NO: 87 - protein, 16 aa, Artificial Sequence
  pNOP526697
PRTENATGSWEVQQGV

SEQ ID NO: 88 - protein, 14 aa, Artificial Sequence
  pNOP568326
GDSLFRQGQASFRE

SEQ ID NO: 89 - protein, 41 aa, Artificial Sequence
  pNOP187097
DLSHMAGLTHTRSNRDLRQDRSKDMGTQGSHTGPRPRSGTR

SEQ ID NO: 90 - protein, 30 aa, Artificial Sequence
  pNOP286473
LPAPTKHAESHSSGIQPCSPAPANGEPHLS

SEQ ID NO: 91 - protein, 25 aa, Artificial Sequence
  pNOP345053
AGAIQLGSRMPLMMEVTPHSRSGIP

SEQ ID NO: 92 - protein, 25 aa, Artificial Sequence
  pNOP355250
RKPSSSSGRRRGARRRRRQRPSAGK

SEQ ID NO: 93 - protein, 25 aa, Artificial Sequence
  pNOP357957
TPWVPEVKCMDSLASHLMAHSLQGG

SEQ ID NO: 94 - protein, 22 aa, Artificial Sequence
  pNOP399373
LHIPEAEFHDSKPWVSAQYEYL

SEQ ID NO: 95 - protein, 19 aa, Artificial Sequence
  pNOP450666
EMWRWDHDSTIPMEVLMTE

SEQ ID NO: 96 - protein, 17 aa, Artificial Sequence
  pNOP503306
PSTEPPEHQDPRGRTPQ

SEQ ID NO: 97 - protein, 16 aa, Artificial Sequence
  pNOP525902
PFQARTSQLQRIVRRS

SEQ ID NO: 98 - protein, 14 aa, Artificial Sequence
  pNOP583798
SCCTTSTQNGSRHH

SEQ ID NO: 99 - protein, 14 aa, Artificial Sequence
  pNOP584557
SLHVLRAGPQRRDG

SEQ ID NO: 100 - protein, 13 aa, Artificial Sequence
  pNOP600191
IPSTSCCMMTTAS

SEQ ID NO: 101 - protein, 11 aa, Artificial Sequence
  pNOP667279
LMKRRRNRTKG

SEQ ID NO: 102 - protein, 47 aa, Artificial Sequence
  pNOP152466 alt splice b
FLWQSVLHPRHPFWQPLPQPADYNVSTATAGIQPCSPAPANGEPHLS

SEQ ID NO: 103 - protein, 27 aa, Artificial Sequence
  pNOP326245
QQHHDLQPQSAPRVARAPCRIFPTMPD

SEQ ID NO: 104 - protein, 24 aa, Artificial Sequence
  pNOP363287
GKHEHWGPTAESHAFQPRLGDVFS

SEQ ID NO: 105 - protein, 24 aa, Artificial Sequence
  pNOP366177
LASHDSRGTPPPPVCVCVCGELRN

SEQ ID NO: 106 - protein, 23 aa, Artificial Sequence
  pNOP390796
WAAPYRHQLRLLSKAPCGRGVMT

SEQ ID NO: 107 - protein, 23 aa, Artificial Sequence
  pNOP391130
WPRRSPPPPPAAWATRRRRRPRS

SEQ ID NO: 108 - protein, 16 aa, Artificial Sequence
  pNOP532250
SSSHGGWGRRRRTSRS

SEQ ID NO: 109 - protein, 16 aa, Artificial Sequence
  pNOP535077
WELDLLMDKGLIVWLA

SEQ ID NO: 110 - protein, 15 aa, Artificial Sequence
  pNOP536697
AFSQDPPACLIYLVQ

SEQ ID NO: 111 - protein, 15 aa, Artificial Sequence
  pNOP539995
EFRGHQGEQQVSIWH

SEQ ID NO: 112 - protein, 15 aa, Artificial Sequence
  pNOP561120
WGACPMSQIRILMAA

SEQ ID NO: 113 - protein, 14 aa, Artificial Sequence
  pNOP564630
CPSSLVSWQRAHGH

SEQ ID NO: 114 - protein, 14 aa, Artificial Sequence
  pNOP580855
QWPAALADWWGGHH

SEQ ID NO: 115 - protein, 13 aa, Artificial Sequence
  pNOP596649
GEGHGHDKSACCG

SEQ ID NO: 116 - protein, 13 aa, Artificial Sequence
  pNOP600818
KCRRQVPQYLPRT

SEQ ID NO: 117 - protein, 13 aa, Artificial Sequence
  pNOP616167
TGRRPSPRHLCSC

SEQ ID NO: 118 - protein, 13 aa, Artificial Sequence
  pNOP616285
THWFHKSFVMYCF

SEQ ID NO: 119 - protein, 12 aa, Artificial Sequence
  pNOP624639
EEDVGGPLSGLH

SEQ ID NO: 120 - protein, 12 aa, Artificial Sequence
  pNOP628397
GSLWQHEESSRE

SEQ ID NO: 121 - protein, 12 aa, Artificial Sequence
  pNOP643975
RTRTGTRALGPP

SEQ ID NO: 122 - protein, 12 aa, Artificial Sequence
  pNOP650952
WTSRKTDHSHYG

SEQ ID NO: 123 - protein, 11 aa, Artificial Sequence
  pNOP658966
GCSARHHVAGA

SEQ ID NO: 124 - protein, 10 aa, Artificial Sequence
  pNOP700714
KTLEPRRHGG

SEQ ID NO: 125 - protein, 10 aa, Artificial Sequence
  pNOP704301
MTSPWGQKEL

SEQ ID NO: 126 - protein, 10 aa, Artificial Sequence
  pNOP708028
PSTSVSSQGC

SEQ ID NO: 127 - protein, 10 aa, Artificial Sequence
  pNOP708425
QASSKDRTEE

SEQ ID NO: 128 - protein, 10 aa, Artificial Sequence
  pNOP709605
QSEDGAWNRA

SEQ ID NO: 129 - protein, 10 aa, Artificial Sequence
  pNOP718154
TRRGRRRGSS

SEQ ID NO: 130 - protein, 91 aa, Artificial Sequence
  pNOP42370
WAAPEWRSCCCSTARSPTAPTPPLSPDPCTTLPGRASWTRWWCCTGPGRGWTCAMPGAVCPWTWLRSWAIAMSHGTCARLRGAPEAVTMPA

SEQ ID NO: 131 - protein, 75 aa, Artificial Sequence
  pNOP64888
LRSEADPGHDDGQRPSGGAAAAPRRGAQLRRPRHSHPTRARRCPGGLPGHAGGAAPGRGAAGRARCLGPSARGPG

SEQ ID NO: 132 - protein, 115 aa, Artificial Sequence
  pNOP23100
GSSRFLEDQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 133 - protein, 26 aa, Artificial Sequence
  pNOP340964 alt splice a
RRCGRCWRRGRCPTHRIVTVGGRSRS

SEQ ID NO: 134 - protein, 28 aa, Artificial Sequence
  pNOP309800
LAGHGRGPGSGRGGAGAAGGGGAAQRTE

SEQ ID NO: 135 - protein, 46 aa, Artificial Sequence
  pNOP159351 alt splice b
MPRKVPQTSPIERTREALRNLGKLRSSVTEGPTGPQLPPPQPTPLS

SEQ ID NO: 136 - protein, 24 aa, Artificial Sequence
  pNOP374903
WSRRRGAAWSLRLTGWPRPRPGVG

SEQ ID NO: 137 - protein, 21 aa, Artificial Sequence
  pNOP412936
GAGPSRCRTVPARGCGGHQRQ

SEQ ID NO: 138 - protein, 59 aa, Artificial Sequence
  pNOP103788
KQACVGKLRDSAEERQSLRRPLVIASWLAHSAPGAKDAWGCGKGKATSSRLRAWHYIPD

SEQ ID NO: 139 - protein, 48 aa, Artificial Sequence
  pNOP149155 alt splice c
PCPHRCRGRSLSWLDQPQDFQTQLCVASSGDLSISALLHNSTNLTLLS

SEQ ID NO: 140 - protein, 37 aa, Artificial Sequence
  pNOP219511 alt splice b
MPRKVPQLAGPTSGFPNPIVRGIIWRSLDLGSSAQLN

SEQ ID NO: 141 - protein, 33 aa, Artificial Sequence
  pNOP255336 alt splice b
MPRKVPQLQLASRSREVKKETSAPVTASIRVPI

SEQ ID NO: 142 - protein, 33 aa, Artificial Sequence
  pNOP258500
SQTSSWRRPGGLGSQGRGMRSHARTDLSNAEKI

SEQ ID NO: 143 - protein, 32 aa, Artificial Sequence
  pNOP267771
RRRLRLQLQLASGSRFRRSCQLQRGSRAEQKA

SEQ ID NO: 144 - protein, 102 aa, Artificial Sequence
  pNOP31901 alt splice a
RRCGRCWRRGRCPTHRIVTVGGRSRWVEGLQREQGMAGDSGGRSLQGNWNQVALRFSGKRGGFLGSFQKGFVITDLLLATPWGLGKPRKRNEEPRAYRSLEC

SEQ ID NO: 145 - protein, 26 aa, Artificial Sequence
  pNOP334099
GFSWFTSRGSRGSGQRQGRPPLWPSC

SEQ ID NO: 146 - protein, 24 aa, Artificial Sequence
  pNOP371501
RVCSGSRGWRATLEDEVCRGIGIR

SEQ ID NO: 147 - protein, 22 aa, Artificial Sequence
  pNOP401561 alt splice c
PCPHRCRGRSLRHPRLKEPERL

SEQ ID NO: 148 - protein, 21 aa, Artificial Sequence
  pNOP419434 alt splice c
PCPHRCRGRSLRNDRKPFVGL

SEQ ID NO: 149 - protein, 19 aa, Artificial Sequence
  pNOP461083
RFDSPEKGEASWGVFRRGL

SEQ ID NO: 150 - protein, 14 aa, Artificial Sequence
  pNOP578182 alt splice c
PCPHRCRGRSLSYS

SEQ ID NO: 151 - protein, 13 aa, Artificial Sequence
  pNOP598590
HGAGGGEQHGAFG

SEQ ID NO: 152 - protein, 13 aa, Artificial Sequence
  pNOP605842
NGAGGGEQHGAFG

SEQ ID NO: 153 - protein, 12 aa, Artificial Sequence
  pNOP639300
PSGFGARAARRE

SEQ ID NO: 154 - protein, 74 aa, Artificial Sequence
  pNOP67306
SETICGFVEAGMRREATGFRRGAPEPEAPFGYRKLAGSLRTRCKRCLGMREGKGHIFTPSRLALHPRLKEPERL

SEQ ID NO: 155 - protein, 67 aa, Artificial Sequence
  pNOP81258
HDDGQRPSGGAAAAPRRGAQLRRPRHSHPTRARRCPGGLPGHAGGAAPGRGAAGRARCLGPSARGPG

SEQ ID NO: 156 - protein, 61 aa, Artificial Sequence
  pNOP97211
HGAQVLGDPPDSARVRPAASEGFRGSHPAAHGGVGSARGARRCGPRADATEEPASRAAAAS

SEQ ID NO: 157 - protein, 173 aa, Artificial Sequence
  pNOP6876
RGLNPMPSTCSLVPSALTPWVLCLISRTARDGSSPLATSAPVCTGAQWMLGGAAGIGAEFWSIGHGGRGKSQLTWRLQRRTRPLCTAPPLPQSPQVVRTPHWTQMFLSLELLSATRPFRTWTLHCGQIQAAPLLQPPVLFRGLESKCPTTRHPGGPWGVSPLAPCPPLEVHLH

SEQ ID NO: 158 - protein, 26 aa, Artificial Sequence
  pNOP339832
QMWLLPPQRPLPGNGVRKAQNGWCRH

SEQ ID NO: 159 - protein, 156 aa, Artificial Sequence
  pNOP9663
RRCCPGIPMNLLRPPLVLQAHAGGRELGGPGRRWWPTQGPRSRTPSCSASQLGAASNSDPPMISSRIRMTRSPGAPLLLGVGPPEKMSCHCQNLRSRAGPANLPCSLCCSSRPEGAWTRMLWPLAPLLLFPMAGLESRSLPMVCTASVWILRRIVI

SEQ ID NO: 160 - protein, 71 aa, Artificial Sequence
  pNOP73574
VPAPPVSSRHPGDLWMKTPPNPQRWRSHLSCDLPLPPPHLFPRSQHQSPLHHVPQLLHLPQFHSLRRDGPS

SEQ ID NO: 161 - protein, 163 aa, Artificial Sequence
  pNOP8413
VCSPLCQGAPRWCACCVPAKDSTSWCSVKSAVTHSTHSAWRRPSGPCPSITTPGAAVAANSATSVDAKVVDPSTSWSASAAAMHTTRPVWGPAIQPGPRANGATGSVQPVCAVRAVGQLQARTGTSSGLEITASAPGAPSYMRKETTARSVHAAMKTTTMRAR

SEQ ID NO: 162 - protein, 38 aa, Artificial Sequence
  pNOP212366
PTTSPQWETRTSQLPPDVPVVPALWLPGRLHHGGPPLL

SEQ ID NO: 163 - protein, 302 aa, Artificial Sequence
  pNOP1023
RLRDPFRTARLGAVHLRTVCWGSAAPLARGPERGPPGGPAPGAPGPAELQGGGPTAALHPVWARWEATAPRTLRPASCESALRGWPLQVCAQLHGGHGGHPHAALGGGRDPGPPGWRPDEGAPAEAARICVRLVRRPRPQVLATEYPAAKRSPSQCGVAPIPGSCLCAVETAGTRDPRIRAASRGSLSSIPGQGSGCLLTPGGPPSVCTLPQIRGCRLQGGGAALVHRAERVDTRQLCHLVGGSLRGERRLPQECACCCGPREADALRALPEAWRHGGLLPVLLPQQLPLHVCPGQLLHLPG

SEQ ID NO: 164 - protein, 30 aa, Artificial Sequence
  pNOP284432
GVLGMEVLALERSHSPRRLPWLMAASPPKA

SEQ ID NO: 165 - protein, 48 aa, Artificial Sequence
  pNOP149964
RPPQTPKGGGLTCPATSHYHLPTCSPGASTSPLSTTCPNSSIYPSSTP

SEQ ID NO: 166 - protein, 44 aa, Artificial Sequence
  pNOP170320
LNFSGGPRHPKHPGAGHVSPPPPGGLGDGPQDGQQAPAGGSSKQ

SEQ ID NO: 167 - protein, 98 aa, Artificial Sequence
  pNOP35490
WTPRCMAMPPASSTTPVSPTASLGSSTWRARNTLLSSPCAASCVVRSSPTTTSSPSRMPATSCPATVAPSAAVGSLTEAVAAHHDPSHLLLPSLPSCP

SEQ ID NO: 168 - protein, 15 aa, Artificial Sequence
  pNOP536795
AGPSRGACARCSRAC

SEQ ID NO: 169 - protein, 108 aa, Artificial Sequence
  pNOP27215 alt splice a
IPMGLLGQRSISGSAPLTCSTSWPPSTGCSLRGPPVMRKRMRCSSGQPDVPPAWSCPWPCVFVTLRRRPKKLWVSTDQPSTGEACSVSATSTRGRWSSSTLALSSARC

SEQ ID NO: 170 - protein, 25 aa, Artificial Sequence
  pNOP346473
DDPPSSSSPSRCGSYPPKDPCPETG

SEQ ID NO: 171 - protein, 164 aa, Artificial Sequence
  pNOP8126
ALEGRWRRWPGLSSRSPTEALSGLKMSRWKLRESGPQVPSPLCKVPASNMSAVMLLWPWVRPGPWCLKMSLASVPSLSGIGRTSPQRIHHRRPRLRVSRHGPGGERWRQQALGENQSPQVLEGPWPTHPGAHCPPITARRCAWLDVDTVGAAYVCRTVGPVSTA

SEQ ID NO: 172 - protein, 67 aa, Artificial Sequence
  pNOP81603
LLQPLHLLHPSHPLRHLLHPHSALHHHPQCPHHLYHPLHRLLPKRSRRNPLLLWSQLRAPGRGAGLP

SEQ ID NO: 173 - protein, 59 aa, Artificial Sequence
  pNOP102672 alt splice b
AVGQPARPARPSASRGCPLSPAGPRQHLPHTKPPGWMKMERPQRIPLRFQGLAVAGLAV

SEQ ID NO: 174 - protein, 56 aa, Artificial Sequence
  pNOP113418
GAEPAPQTYPAACVAAQGPKAPGQGCFGPWPLCFFSQWLDWKAEVSRWCAPRPCGF

SEQ ID NO: 175 - protein, 52 aa, Artificial Sequence
  pNOP129859
KPPLSSGCPLLPQSSQPSHLPQGSWLPLARPHLHHPLKTWAQTSRTWRWCQD

SEQ ID NO: 176 - protein, 50 aa, Artificial Sequence
  pNOP139147
LWCPPLVWPPALPLEPPALNSWTAWTTALTVRLRRCSSLGARARLLRGQE

SEQ ID NO: 177 - protein, 49 aa, Artificial Sequence
  pNOP142719
GLPWSSRPTPGGGSWGAPGGGGGPPRARGAGLPPAAQVSSALRQTATLL

SEQ ID NO: 178 - protein, 128 aa, Artificial Sequence
  pNOP17169
GRGVPSRGSSSEQRATDTGSATAAPAGLANPAPAPGTTATTATAAATAVTTADASPGKSPDCGRGFLAAVWGRGEDVQPPQESQSAAIQDRSAAAAEGGSFHAAEPWRADGGGGRGCQADLRQRPCPV

SEQ ID NO: 179 - protein, 44 aa, Artificial Sequence
  pNOP172961
VGRDSWASTMMLSSSWPSSSPEPSVASTISSVTTSRERARRSRP

SEQ ID NO: 180 - protein, 120 aa, Artificial Sequence
  pNOP20643
LCGAAVARRGRAEPSPGRTRPCSVCWGSAGACAGSAACGPARGSSGAGDGVGAGAGARVEAACRRRRAVTGNPTRRSFRVFIQMKMWPPVPCALRSDPSEVERPEVGVASIRRPPFLLLA

SEQ ID NO: 181 - protein, 35 aa, Artificial Sequence
  pNOP233428
ERAALRSRVPCARSPHQTCLPSCCCGPGSGPGHGA

SEQ ID NO: 182 - protein, 30 aa, Artificial Sequence
  pNOP283728
GAHLRLQVPHRGCQQQAALQLWRQALPSVP

SEQ ID NO: 183 - protein, 28 aa, Artificial Sequence
  pNOP306682
ELWGNSRQELGRRVVWRLQPLPQVHPAI

SEQ ID NO: 184 - protein, 22 aa, Artificial Sequence
  pNOP392368
AQHRRGGDGHRVLWHCHPLGVD

SEQ ID NO: 185 - protein, 20 aa, Artificial Sequence
  pNOP443670
SRKCKRPEGMPDSDISPLVE

SEQ ID NO: 186 - protein, 18 aa, Artificial Sequence
  pNOP482268
REPGPKTDWPTSALRDQQ

SEQ ID NO: 187 - protein, 17 aa, Artificial Sequence
  pNOP499276
LGARGPPCSSASDPPRK

SEQ ID NO: 188 - protein, 81 aa, Artificial Sequence
  pNOP54281
APTSCGSSETSDWQLEMQGGARSRTWDPQAWRTVKPWRPWRQGPRPRWWAPLCDQVCFKGQKSKDGTIVLGTRIRSRSRST

SEQ ID NO: 189 - protein, 14 aa, Artificial Sequence
  pNOP569191
GPPTGHRCSCPWSS

SEQ ID NO: 190 - protein, 71 aa, Artificial Sequence
  pNOP73224
RWDNCPWDSNQVKVKVNMRKVGRMSPKEELDLDREGALAGKSRNRSWMTRKKRRKKKKKKTRREKRRKKEL

SEQ ID NO: 191 - protein, 57 aa, Artificial Sequence
  pNOP109317
ALPGRDCSRWGHGEQPRGPGGQLRGGVQPHLPLHPLPCDCGVRPWSGPQRYPWSPPH

SEQ ID NO: 192 - protein, 143 aa, Artificial Sequence
  pNOP12376 alt splice b
AVGQPARPARPSASRGCPLSPAGPRQHLPHTKPPGWMKMERPQRIPLRFQGLAVAGPSRNGPLCCHFRKMVLPRSPMVPQTCCLSPSGTTIQVRLRALRKSLHPQMIKRTRPQNGLAHICASRSAVRMGSALRQRAWRGRGEL

SEQ ID NO: 193 - protein, 143 aa, Artificial Sequence
  pNOP12501
NLRSAGSTPTTPSTGDGVPGCQTESFPMRCCPHPWIMSMRSGDSRNQRPQNQGSLQGIPQQHSRARIRLPSHTWRTPVSVHSASNTGMQTPRRRGGSCTSGRTSGHTSTVPSGRRKSSRRTTAPSRMCMLLWPEGGRCAASSA

SEQ ID NO: 194 - protein, 50 aa, Artificial Sequence
  pNOP137356
CSAHSAITGCMPSARGSQMKTTRSFQDCQTRCCTPADRVLGQRSPAGERP

SEQ ID NO: 195 - protein, 137 aa, Artificial Sequence
  pNOP14051
APLAHSEPGPSTAARFRQRPSSSPPFFFGGSNQSAQLLAIPEALGGCLLWPPALPWKSIFTDPPHPHSGRPGLPSSPQTFPSSQPFGSQAASITVGLPSSKNLPSAQGAPSYLSRHSPHTYLRGAGSPWPGPISTTP

SEQ ID NO: 196 - protein, 49 aa, Artificial Sequence
  pNOP145287
SLAPRWAAACPPASATSTSCVPGPATASSRMTRKSSARNTLISWMARKL

SEQ ID NO: 197 - protein, 46 aa, Artificial Sequence
  pNOP159086
LPASGRSGKLLGQGQRAPLLPLQPPAPPREALRKTVPPWPPKAPPS

SEQ ID NO: 198 - protein, 46 aa, Artificial Sequence
  pNOP160746 alt splice c
RWRGLRGYPSGSRAWQWRAPPGTVPFAATSGRWSSPGPRWSPRPAA

SEQ ID NO: 199 - protein, 44 aa, Artificial Sequence
  pNOP170722
NIRLAAGNARRGPVQDLGPPGVEDSQAVEAVEAGAAAEVVGSPL

SEQ ID NO: 200 - protein, 44 aa, Artificial Sequence
  pNOP170957
PGSCPLLPQPLHLPRPPPHPLLLPPPPGGPYSFGPLSLPQAKPT

SEQ ID NO: 201 - protein, 44 aa, Artificial Sequence
  pNOP172435
SSHLCPPPFPPRLPPPGLCPQAPSSACCPWSEWSALPRPRHPLP

SEQ ID NO: 202 - protein, 44 aa, Artificial Sequence
  pNOP173362
WRRRRAAAVAPGLAPRGAASRAGRGAPAGAGAAADGATGPKECG

SEQ ID NO: 203 - protein, 42 aa, Artificial Sequence
  pNOP181020
FRERVADGGPECAHLCARGPPDGVLAVCQQRTPRAGVLSSLL

SEQ ID NO: 204 - protein, 42 aa, Artificial Sequence
  pNOP183367
PGSAWGARWGRKSWAPPGTVPFAATSGRWSSPGPRWSPRPAA

SEQ ID NO: 205 - protein, 40 aa, Artificial Sequence
  pNOP199665
VSASRMATTSLCTASWRTWWASSCGTRRRERPRTAGLEAR

SEQ ID NO: 206 - protein, 38 aa, Artificial Sequence
  pNOP207889
ALHPPAVSGTAPRTASRPLQEEAASSSGGRSSCDNPQT

SEQ ID NO: 207 - protein, 242 aa, Artificial Sequence
  pNOP2249 alt splice d
VPLPPAGRGPGGAAPESPWGCSGRGLSPLCLQQYIPPSPAATCRKCTFDMFNFLASQHRVLPEGATCDEEEDEVQLRSTRRATSLELPMAMRFRHLKKTSKEAVGVYRSAIHGRGLFCKRNIDAGEMVIEYSGIVIRSVLTDKREKFYDGKGIGCYMFRMDDFDVVDATMHGNAARFINHSCEPNCFSRVIHVEGQKHIVIFALRRILRGEELTYDYKFPIEDASNKLPCNCGAKRCRRFLN

SEQ ID NO: 208 - protein, 114 aa, Artificial Sequence
  pNOP23566
DGGGGGRRQLPRAWLRAGPLPGPAAGRRRGRGPRRTGQRGRKSAGSSAARRWRDGAGRSRARGGHGPAPFAGAPPGPAPAPPPVGRPAGPAGPGTGSGPGLGPESRLRAGGGEQ

SEQ ID NO: 209 - protein, 114 aa, Artificial Sequence
  pNOP23765
NGGGGGRRQLPRAWLRAGPLPGPAAGRRRGRGPRRTGQRGRKSAGSSAARRWRDGAGRSRARGGHGPAPFAGAPPGPAPAPPPVGRPAGPAGPGTGSGPGLGPESRLRAGGGEQ

SEQ ID NO: 210 - protein, 33 aa, Artificial Sequence
  pNOP252560
GGAAASGPGHASFGARSSPGRGPWGCRGQGPAS

SEQ ID NO: 211 - protein, 111 aa, Artificial Sequence
  pNOP25410
KPPQCVGSLTWIGLGSPLGKKVLGPSRNGPLCCHFRKMVLPRSPMVPQTCCLSPSGTTIQVRLRALRKSLHPQMIKRTRPQNGLAHICASRSAVRMGSALRQRAWRGRGEL

SEQ ID NO: 212 - protein, 32 aa, Artificial Sequence
  pNOP263780 alt splice a
IPMGLLGQRSISALSSTVYSSFPCCHLQEVHL

SEQ ID NO: 213 - protein, 32 aa, Artificial Sequence
  pNOP269620 alt splice d
VPLPPAGRGPGGAAPESPWGCSGRGLSPEVHL

SEQ ID NO: 214 - protein, 31 aa, Artificial Sequence
  pNOP278498
RRRCSASSREPKCSYSRSISSSSRRWQLPCR

SEQ ID NO: 215 - protein, 30 aa, Artificial Sequence
  pNOP281826
APRWWAHCCSAPSVGQMGSNCTQDPAACKL

SEQ ID NO: 216 - protein, 30 aa, Artificial Sequence
  pNOP287880
PLGPWGAATGARGTAPRRSPAPPPATSTSL

SEQ ID NO: 217 - protein, 29 aa, Artificial Sequence
  pNOP295363
GKLAGCPPKKSWIWTGREPLLEKAGTEAG

SEQ ID NO: 218 - protein, 29 aa, Artificial Sequence
  pNOP295589
GRELGGGVENSDRESARGPRACPTQTSLL

SEQ ID NO: 219 - protein, 27 aa, Artificial Sequence
  pNOP317592
AQLLLSGHPRGGPETHCYLRPAPHPAW

SEQ ID NO: 220 - protein, 27 aa, Artificial Sequence
  pNOP323657
LRPWLPTTTPHTSCCRRCHLAPSLGAP

SEQ ID NO: 221 - protein, 27 aa, Artificial Sequence
  pNOP326541
RCPSPQCPPSPGSAGPRHRGYIIGVRD

SEQ ID NO: 222 - protein, 27 aa, Artificial Sequence
  pNOP328068
SGQGSLGLQGTGPGLLRTCHRKLWILC

SEQ ID NO: 223 - protein, 26 aa, Artificial Sequence
  pNOP331404
ALALPLSPPNPPHPKSYLSTSWGKYL

SEQ ID NO: 224 - protein, 26 aa, Artificial Sequence
  pNOP331561
APQTRHIQNHTCQQAGASICEDGWGG

SEQ ID NO: 225 - protein, 26 aa, Artificial Sequence
  pNOP340189
RCGPQFPALCAPIPARSSAPRSGSQA

SEQ ID NO: 226 - protein, 24 aa, Artificial Sequence
  pNOP363468
GPAIGNCGFCVEEPRGSWGWRCWP

SEQ ID NO: 227 - protein, 24 aa, Artificial Sequence
  pNOP367137
LTSGRSSTMGRASGAICSAWMTLM

SEQ ID NO: 228 - protein, 24 aa, Artificial Sequence
  pNOP370489
RGRREERRRRKRQGGRREGRKSCS

SEQ ID NO: 229 - protein, 24 aa, Artificial Sequence
  pNOP373366
TPMVLMFSAESMWTSRASTSSGSS

SEQ ID NO: 230 - protein, 23 aa, Artificial Sequence
  pNOP376070
ASGSGPHQPPQPASIRPCGHHSC

SEQ ID NO: 231 - protein, 23 aa, Artificial Sequence
  pNOP378678
GAAQVNQTCHQPGAAHGHAFSSP

SEQ ID NO: 232 - protein, 23 aa, Artificial Sequence
  pNOP384879
PHPHICLAPRGPRGPGVKPWPCP

SEQ ID NO: 233 - protein, 22 aa, Artificial Sequence
  pNOP393358
CSPPSLCGLRGHQLQAEVLDGA

SEQ ID NO: 234 - protein, 22 aa, Artificial Sequence
  pNOP394645
EQDDAVRTVRSLGACQVRGALR

SEQ ID NO: 235 - protein, 22 aa, Artificial Sequence
  pNOP402065
PPAQLTPPAHLPGSQGPQGSGC

SEQ ID NO: 236 - protein, 22 aa, Artificial Sequence
  pNOP407306
TSPSLGALTPRSSAVYTGSVTK

SEQ ID NO: 237 - protein, 21 aa, Artificial Sequence
  pNOP411745
EDVQRSCGCLQISHPRARPVL

SEQ ID NO: 238 - protein, 92 aa, Artificial Sequence
  pNOP41189
TCPTPSEAATFAPHHFPHGSHLLDSAPRPPPRRAARGRSGPPCPAPATPSPDAGAEQWASQPAPPGHPRQEGVHFLRPVPASTSPIQSPPAG

SEQ ID NO: 239 - protein, 21 aa, Artificial Sequence
  pNOP426146
VLLTWTSRPACWGLSPSRKRL

SEQ ID NO: 240 - protein, 19 aa, Artificial Sequence
  pNOP459923
QAGEVLRWEGHRVLYVPHG

SEQ ID NO: 241 - protein, 19 aa, Artificial Sequence
  pNOP462749 alt splice c
RWRGLRGYPSGSRAWQWRV

SEQ ID NO: 242 - protein, 18 aa, Artificial Sequence
  pNOP468831
CCHLPGRAAPRSPALPAL

SEQ ID NO: 243 - protein, 18 aa, Artificial Sequence
  pNOP469462
CSGRHDAWQCRPLHQPLL

SEQ ID NO: 244 - protein, 18 aa, Artificial Sequence
  pNOP483192
RPGPRLRGHGGGVRTECC

SEQ ID NO: 245 - protein, 16 aa, Artificial Sequence
  pNOP533725
TSPAGPGTPSTPEPGM

SEQ ID NO: 246 - protein, 15 aa, Artificial Sequence
  pNOP538448
CQLRKRKRQSCHHRL

SEQ ID NO: 247 - protein, 15 aa, Artificial Sequence
  pNOP546704
KRPDDSEDAVALGFR

SEQ ID NO: 248 - protein, 80 aa, Artificial Sequence
  pNOP56683
PIPPILPGGGRAAPAPASRHLVLPSLQILPRLWTQRSWIQAPPGVRALPPCIPPGLSGAQLSNPGHAQTAPLDLFSLCAL

SEQ ID NO: 249 - protein, 14 aa, Artificial Sequence
  pNOP581470
RGIRRGGVSGFSFR

SEQ ID NO: 250 - protein, 14 aa, Artificial Sequence
  pNOP582085
RLGRWNDWLKKAGR

SEQ ID NO: 251 - protein, 13 aa, Artificial Sequence
  pNOP599417
HVQLPGLPAPGAP

SEQ ID NO: 252 - protein, 13 aa, Artificial Sequence
  pNOP607050
PCEDENPHSAWGP

SEQ ID NO: 253 - protein, 77 aa, Artificial Sequence
  pNOP60902
ECPVTVPAGKGGGSRPWGRIRAHRFWRDPGPHTPALTALPSRQEDAHGSMWTLSGLPTCAGLWVLCQLPRQAQVWGP

SEQ ID NO: 254 - protein, 13 aa, Artificial Sequence
  pNOP609760
QSPNLSPHLLWFQ

SEQ ID NO: 255 - protein, 13 aa, Artificial Sequence
  pNOP614494
SPGWQGNCEPRWF

SEQ ID NO: 256 - protein, 13 aa, Artificial Sequence
  pNOP616888
TRCHQRAHWFHPH

SEQ ID NO: 257 - protein, 13 aa, Artificial Sequence
  pNOP619315
WQPALPRPDRQPS

SEQ ID NO: 258 - protein, 12 aa, Artificial Sequence
  pNOP625450
ERKLLPDLYTLL

SEQ ID NO: 259 - protein, 76 aa, Artificial Sequence
  pNOP62604
EETVHPKGTHISLDLTDPGAAPSSPSPSTSPGPLPTPCSCHLLPEAPTPSGPSVYPKRSPPEDLRIGAYSSSSWGS

SEQ ID NO: 260 - protein, 12 aa, Artificial Sequence
  pNOP644158
RWLGRVNLSHPQ

SEQ ID NO: 261 - protein, 12 aa, Artificial Sequence
  pNOP650472
WNEWGETPGHPP

SEQ ID NO: 262 - protein, 11 aa, Artificial Sequence
  pNOP660324
GRHRTDGAGTD

SEQ ID NO: 263 - protein, 11 aa, Artificial Sequence
  pNOP661817
HQEAVLCIPEV

SEQ ID NO: 264 - protein, 11 aa, Artificial Sequence
  pNOP673600
QNRGSEDGTTG

SEQ ID NO: 265 - protein, 11 aa, Artificial Sequence
  pNOP675110
RGVTPPGASPG

SEQ ID NO: 266 - protein, 10 aa, Artificial Sequence
  pNOP706730
PGLRGQPAGD

SEQ ID NO: 267 - protein, 10 aa, Artificial Sequence
  pNOP711022
RISGSLLCLW

SEQ ID NO: 268 - protein, 72 aa, Artificial Sequence
  pNOP71226
SLGLRGTALPHWLPVLPSVLEHSGCSEALLVSVPNSGVSAMGAEGRASSPGGCRGEPDHCAQPRPFLRAPRW

SEQ ID NO: 269 - protein, 10 aa, Artificial Sequence
  pNOP720871
WNDWLKKAGR

SEQ ID NO: 270 - protein, 67 aa, Artificial Sequence
  pNOP82310
RSTNRCLLLLLLGLLKPLSQSLLLPMTLQLSLSLGQWAAPTTSACLDSPLWSPLLLRPRCPLTGLQL

SEQ ID NO: 271 - protein, 160 aa, Artificial Sequence
  pNOP8822
GDDASCGKGRGKAATTASDSSSPFTSSTPPTPFDISSTPTLPSTTTPSVPTTSTIPSTASCPRGAGGIPSSCGPSYVLQEEGPASPDSQPAGGAGSCSGRARGHLSSHSNPQHRHGRPSGRQSHRGPQKHHLPEEYPAVYYACGECPLLPCHQDTPAIYG

SEQ ID NO: 272 - protein, 60 aa, Artificial Sequence
  pNOP99414
ATGHRHRLSYCSPCRPCKPSSCPRHYRHHSHSCSHRRHHSRCLPWKKPGLRAWVPCRCLG

SEQ ID NO: 273 - protein, 494 aa, Artificial Sequence
  pNOP134
TRRCHCCPHLRSHPCPHHLRNHPRPHHLRHHACHHHLRNCPHPHFLRHCTCPGRWRNRPSLRRLRSLLCLPHLNHHLFLHWRSRPCLHRKSHPHLLHLRRLYPHHLKHRPCPHHLKNLLCPRHLRNCPLPRHLKHLACLHHLRSHPCPLHLKSHPCLHHRRHLVCSHHLKSLLCPLHLRSLPFPHHLRHHACPHHLRTRLCPHHLKNHLCPPHLRYRAYPPCLWCHACLHRLRNLPCPHRLRSLPRPLHLRLHASPHHLRTPPHPHHLRTHLLPHHRRTRSCPCRWRSHPCCHYLRSRNSAPGPRGRTCHPGLRSRTCPPGLRSHTYLRRLRSHTCPPSLRSHAYALCLRSHTCPPRLRDHICPLSLRNCTCPPRLRSRTCLLCLRSHACPPNLRNHTCPPSLRSHACPPGLRNRICPLSLRSHPCPLGLKSPLRSQANALHLRSCPCSLPLGNHPYLPCLESQPCLSLGNHLCPLCPRSCRCPHLGSHPCRLS

SEQ ID NO: 274 - protein, 35 aa, Artificial Sequence
  pNOP234091
GPRSHPLPRLWHLLLQVTQTSFALAPTLTHMLSPH

SEQ ID NO: 275 - protein, 117 aa, Artificial Sequence
  pNOP21934
ARVMPVPVFLAQSPSWALQTRRGVAPCPWSWGSLRMLVQPEMRAPYGSVLTHCQRLMTHYCAMLGQLSAEAKLRGRRGGGAAPQPVPASNRVAAAVSQEDAGLVEEPMEDVVEDGPG

SEQ ID NO: 276 - protein, 57 aa, Artificial Sequence
  pNOP111349
PTLRWGLGGSQQPCPRGQQVSSMPRSQVGSPPILSGPLGRVHLWAPPLPCVSLSLRQ

SEQ ID NO: 277 - protein, 44 aa, Artificial Sequence
  pNOP170800
NRLMRRLNGRPCCGGWSQDPWALRSALPLLLMPLNPAWHLCSLR

SEQ ID NO: 278 - protein, 88 aa, Artificial Sequence
  pNOP44838
CCSRAGVVWSVLCVRCVARPPTPHACCSVMTVILATTHTAWTPHCSPSPRAAGSASGVCPVCSVGLLPLASTVNGRIVTHTVGPVPAW

SEQ ID NO: 279 - protein, 117 aa, Artificial Sequence
  pNOP22159
PCHHCTSGANGEDGLASQARQDWRVLSPQMPLALMTRRMGTWTPMSCSRVKVVWSTWSAKLNWRAPSALMWSLAKRRPRKAKNASVNHIGLALVVSWCDSGNPTHARKRGLLHRRRC

SEQ ID NO: 280 - protein, 55 aa, Artificial Sequence
  pNOP118654 alt splice a
PGSSPHQQGAEARGTGQPAPRCCPHHFHWQPHYPRRLVYLCGRVPEAAGGLGAWP

SEQ ID NO: 281 - protein, 72 aa, Artificial Sequence
  pNOP70346
HHAEYRGSLLQHRQICPNAGHVCGMWQLWPGGRGPPPCLFAVLSVLSPLLCQQQDHQGDAAQGLALCGVYCV

SEQ ID NO: 282 - protein, 65 aa, Artificial Sequence
  pNOP8757 alt splice b
SSGERFQQLTKPPTCKRPKITGQLTASTRCRSQGHWAARPPLLPPPFSLAAPLPPPACLPLRTGS

SEQ ID NO: 283 - protein, 52 aa, Artificial Sequence
  pNOP129784
KHCSCYAQSTVRGLHIWRRLAVQCVRGQGSCVTCSSVPAVGITITGPAWTLL

SEQ ID NO: 284 - protein, 128 aa, Artificial Sequence
  pNOP17440
WTARSWLVRIKIQNRQLMDLQLLRTQVPLSQTCPTHMWERSLSLVLGVPGFRRLLRTAVGVRCGVVLSVTAGSPVYTGSGSYGALSCHLIGPGVQWCPLGGAQGPMRQCCPVRTYHRLVSLRALHLPT

SEQ ID NO: 285 - protein, 33 aa, Artificial Sequence
  pNOP257632
RRKSLGHPLLAMGPQTWALLTHPPQAPTWVAWS

SEQ ID NO: 286 - protein, 72 aa, Artificial Sequence
  pNOP69709
ACPPYDPSPISRLPSGAGFSHPDGAPSSSVFATPSAFPGSPKLPSFPVLSSCPTTVRSLPVESHREGSGGLR

SEQ ID NO: 287 - protein, 131 aa, Artificial Sequence
  pNOP16127 alt splice c
KAAVRHCRGPFFKVDSLWAICPPAAQWTPTQASASPRSWILGSAGASLARNPVSPTAPGRAQVAPRPPPPQPPPRRVRATDSPITSGVFSAGRRMRSWASCPPSHLCSMPTLIFLISSKTTQTGQAVANKS

SEQ ID NO: 288 - protein, 41 aa, Artificial Sequence
  pNOP189145
LLGPNLRPLRAAVLCPLAHCPPTLSPECLPVLSPSPAPSLH

SEQ ID NO: 289 - protein, 119 aa, Artificial Sequence
  pNOP21288
SRRRARCLALTRLVSSSSSSHPRCPPKCLRRTPLDWPLPIPWSPASPRHRPPIPPILVLRGPLRSPRCWAPHLVLGLASQGNSTLPHLAPPDTSPPHLTHSSNPAAPRWITWLCLRALG

SEQ ID NO: 290 - protein, 114 aa, Artificial Sequence
  pNOP23772
NRRAPPQSHPLSTAIPTMSPIWMCDSSRPHLLKNPPRPLPPWHLLLPVPLLSPWLNFPPNPWLSHPSPHLCHWPHPLNQPDPSPVPGPLKKVKIPVLLASRNGKECAGSGFGCC

SEQ ID NO: 291 - protein, 32 aa, Artificial Sequence
  pNOP269687
VRTPTDWLLKGFGAWRYQVFPHRNPQPHRPLN

SEQ ID NO: 292 - protein, 105 aa, Artificial Sequence
  pNOP29324
GQGLDLRAHPGSLPHQEPYLQDQSLALSIPHLHHPALKSQRDLHNYLPPAPSFPLRPSSLPPIQGPPNLRGQPWSRLLGGSHLLLPSLQIPCLARVWDLGIPQTT

SEQ ID NO: 293 - protein, 79 aa, Artificial Sequence
  pNOP58594
SKSLASFSGENGCTCSVWGALCSTPSDSCCLTRWLTFIVPLPSIPWATRPRASIGASAPTIVAAAIAVLLVRTTGGRSL

SEQ ID NO: 294 - protein, 76 aa, Artificial Sequence
  pNOP62730
GIPTQHQAGTSGRAMCPGSPVSEEGGQWGANRGTRNQQPPPAGRPSLRSWASALAEATPGKECATQHWAGVRGAAS

SEQ ID NO: 295 - protein, 165 aa, Artificial Sequence
  pNOP8118
YRATTSQTRTCPPVWAGSAWGWNHAYGGSASSTAPRSPGQKPTAAALKSSAAAAATGTPHAAAAAAESGSTPDPTLPGAWDPDLSPPGPPGLPTSTWGLPWTTDRPPPGARGRASTSGPTPAPCPTRSLIYRTSPWPCPSHTSTIQPSRAKETFTITFPQLPASH

SEQ ID NO: 296 - protein, 58 aa, Artificial Sequence
  pNOP106859
HPGLCLLKLFAHHPLPLASSPLTLILAHPHALSPVTHLPHCISHPDPSPLKLPLRLGL

SEQ ID NO: 297 - protein, 148 aa, Artificial Sequence
  pNOP11179
APCQGPKWAAPQFCPVPWDGCICGHPLSHAFHFPSGSRGAFPKAPCPSAWSPATPWDQQPFWARPHLGQASKHKLHSSHRELPPIGQPPGAQQRVHRGELWAVPTTPSVGSATTCTRRIPPLPVPWSLTAIRHHLSCRKARRPRDWNG

SEQ ID NO: 298 - protein, 41 aa, Artificial Sequence
  pNOP188940
KTWRPMTPTWMTCSMETSLTCWHILILSWTLGTRRISSMST

SEQ ID NO: 299 - protein, 34 aa, Artificial Sequence
  pNOP243509
GVSHAHSLCCCSQEPEWRDGGSGGAAEHEDPQLL

SEQ ID NO: 300 - protein, 107 aa, Artificial Sequence
  pNOP28077
PQGTSTHRAAPWGPAAGPQGRAMGCPHYALRRFCHHLHPTDPSPTCPMEPHSDQASPLLSKSEKTQGLEWVALWRQLNSQVPRTQACPALAKQSWRSNGSASDYESC

SEQ ID NO: 301 - protein, 24 aa, Artificial Sequence
  pNOP363905
GWVSSPHFAGGWGVPSSPARGASR

SEQ ID NO: 302 - protein, 96 aa, Artificial Sequence
  pNOP36658
GPYTCPPRRTWRVLLGSPLVCCMVGRRMGAGGPRTMWCGQGHLLRDLTALLPLHQARCLHPLPLTWMSTALPLPLRDCQRFLPIHENTAAAMPRAQ

SEQ ID NO: 303 - protein, 23 aa, Artificial Sequence
  pNOP390234
VEARPPLLGHRTRAALWGCPQAS

SEQ ID NO: 304 - protein, 17 aa, Artificial Sequence
  pNOP493996
GAATLPPVRGAAPVTPA

SEQ ID NO: 305 - protein, 77 aa, Artificial Sequence
  pNOP61039
GHQEPATTSCWQALAQKLGICSCRSYSGQRMCNSALGGGPRGCELRSTGTLTASWLGWSRNYRVPPATRRMQQQGSL

SEQ ID NO: 306 - protein, 62 aa, Artificial Sequence
  pNOP96015
VLSSSSSYRHSSCSGSCSRVRQYARPHPTRSLGPRPLPSRASWAANLNLGASLDHRQAPSRS

SEQ ID NO: 307 - protein, 60 aa, Artificial Sequence
  pNOP102126
TTVFIQHPTPRVLPCQLVWSWSTGPRRALSLAAPILWPWKLGSCPVRIPSWMTILMPTRP

SEQ ID NO: 308 - protein, 298 aa, Artificial Sequence
  pNOP1069
FKAFTGKAAAAAAATYAAGPETAAAAAAATAAAAPSRTGGNPAATAAGSWSTDKPSSGSQAPGPYASQQPPRPPGPAAVPSTTPGAPGHAGPCPGGCVAAAAPWSFGPPGPSQTGAYDPVPGAQFPPAGTAGSGPYGTQAGHSPAAAAATTAPTARVHGRAVPSSAESDVTQWAAQTERSAHGLFTAASAAAAAATATATSAAAAAAATTATATSAATASTAATAAAASTTAAATASTAATAATTATATTTAAVSTAAATAADGPFKPESNFTVSSATTAAASGTWPWHASKASSTLF

SEQ ID NO: 309 - protein, 58 aa, Artificial Sequence
  pNOP108932
VPRWREFPPVCQALVSQCLVQLVLPSSLSCGTMYRKDWDLGALRFLVRAHLRDPVFTL

SEQ ID NO: 310 - protein, 57 aa, Artificial Sequence
  pNOP110054
GEAQGGGGWTPPFSLPIHHCYPQGRARTCCQFPWPGAKARTEHDGQPGYPDGHRAIF

SEQ ID NO: 311 - protein, 56 aa, Artificial Sequence
  pNOP114830
PSAPCASELVPPAAAIACVAPMSTILLVPSVPSACSSRTRPCCVQCIRSRGPVSKS

SEQ ID NO: 312 - protein, 53 aa, Artificial Sequence
  pNOP127724
TRTASGLWNPWPRRQPYATAEALSSRWTPFGQSALQQPNGLLPRPLPVPVPGF

SEQ ID NO: 313 - protein, 50 aa, Artificial Sequence
  pNOP137298
CLQSPPDPSGISGRAPEPGLGPKAPGATPCPGFGTFSSKSPRHLSPWLLH

SEQ ID NO: 314 - protein, 50 aa, Artificial Sequence
  pNOP139704
PSPGCSVPPSWHSRVRALWDTGWSQPSSSSSNNSTNSKGPWQGCPIFSRV

SEQ ID NO: 315 - protein, 47 aa, Artificial Sequence
  pNOP154481
PLWRSTPNASRQQGRAHHVKNRKSHVHRWPPHHPLSSNPTSLTRSLI

SEQ ID NO: 316 - protein, 47 aa, Artificial Sequence
  pNOP155302
RSPTPMRCCSQRAPPGQALSQRRGKLRVLVGRKRVWKARAQTLALIG

SEQ ID NO: 317 - protein, 44 aa, Artificial Sequence
  pNOP172213
SHCKGQDGGFERHQESDGSGQHWGGTWYEQTASVSASPEALGGT

SEQ ID NO: 318 - protein, 43 aa, Artificial Sequence
  pNOP178870 alt splice d
TISAWHWWFHGATAEIPHTHEKGACCTGGGVEWGWAARRGDTC

SEQ ID NO: 319 - protein, 42 aa, Artificial Sequence
  pNOP179906
ALPQAPTPGARPSAFAGPLWTGPCLSPGAPLPHGTAHLSPLS

SEQ ID NO: 320 - protein, 42 aa, Artificial Sequence
  pNOP182619
LPANVLAGSALNAKCAKPAGNLGMTLRCWFVRRVTKDTILSA

SEQ ID NO: 321 - protein, 41 aa, Artificial Sequence
  pNOP187538
FGSRSSATPCGRRRKQLQQLQEQWGLQAAGVLSPAALPLSS

SEQ ID NO: 322 - protein, 124 aa, Artificial Sequence
  pNOP18835 alt splice c
KAAVRHCRGPFFKVDSLWAICPPAAQWTPTQASASPRSWILARNPVSPTAPGRAQVAPRPPPPQPPPRRVRATDSPITSGVFSAGRRMRSWASCPPSHLCSMPTLIFLISSKTTQTGQAVANKS

SEQ ID NO: 323 - protein, 40 aa, Artificial Sequence
  pNOP193752
CRTCVWYVAALAGGQRATSLPVRSALSAITLTVSTARSPR

SEQ ID NO: 324 - protein, 121 aa, Artificial Sequence
  pNOP20115
GLFSQFGWVPTAAFPGSCRCPTARFAPATDAHPATSSCPPATPGSIHGYGVQSRAYAKWAAWRAGRLGTPAELTASAITEAHGHHATFHVHEAAAIGNAAAAGKQLLPRYRPGQICCRRYH

SEQ ID NO: 325 - protein, 39 aa, Artificial Sequence
  pNOP201536
ELLCSAPSLTALRPFLPSACQSSVPVQLPVSTDTPASVC

SEQ ID NO: 326 - protein, 121 aa, Artificial Sequence
  pNOP20393
TCWLPCLHPLTIRLRMSGWRVMRIAILLTALCQLHPLRASWGRRPLVSLIWAQAGGSKRTGPSPLSSPSFLGPASQSSQIPNLMGPLAWRSLESCLSQLGKRAKEVRCQSCSQSLLLQPRT

SEQ ID NO: 327 - protein, 38 aa, Artificial Sequence
  pNOP209010
EPWGRGRQSFRAPALAPTFWGVPEGPRGEEGRAWGILS

SEQ ID NO: 328 - protein, 38 aa, Artificial Sequence
  pNOP209424
GGEGAAAQLPSPFPHQTGSQQQFPRKTPASWRSPWRTW

SEQ ID NO: 329 - protein, 38 aa, Artificial Sequence
  pNOP211152
LPHILPGPPTAHRPQGRLEVQVVCVLYAVWGCFPWLPL

SEQ ID NO: 330 - protein, 36 aa, Artificial Sequence
  pNOP224854
EEEATAARAQEEQTGGHVPCLLAGSLLWEGAAGPEP

SEQ ID NO: 331 - protein, 34 aa, Artificial Sequence
  pNOP245157
LLTLIALPVRRRRKKMMTPCRIPWFSSPTQTNLS

SEQ ID NO: 332 - protein, 33 aa, Artificial Sequence
  pNOP257396
RLPCAPGPRGAGPCDPYGGLPRMQADSRAGLTM

SEQ ID NO: 333 - protein, 32 aa, Artificial Sequence
  pNOP264714
LHTLWALCQPGDLPYLSCSLRRRGPTNPVPPL

SEQ ID NO: 334 - protein, 30 aa, Artificial Sequence
  pNOP284778
HHSAGRTAAHVPCGGPCVPRHRTAAASPDG

SEQ ID NO: 335 - protein, 30 aa, Artificial Sequence
  pNOP287872
PLCPLWQWLPSQWAEPAEGGLWKWGAAHWP

SEQ ID NO: 336 - protein, 29 aa, Artificial Sequence
  pNOP298931
NHPWRNCLLTLGSARRAGCAGPVGRAQQN

SEQ ID NO: 337 - protein, 29 aa, Artificial Sequence
  pNOP303477
VAPSWGQGPSLAMTDSPGHLHQPRLPLWM

SEQ ID NO: 338 - protein, 28 aa, Artificial Sequence
  pNOP310713
MDRWCLRHPNSASSRNLGKSHVPWEPSQ

SEQ ID NO: 339 - protein, 27 aa, Artificial Sequence
  pNOP318057
CHQIPFLLHSHPSSQLRPHRPCLLWGS

SEQ ID NO: 340 - protein, 27 aa, Artificial Sequence
  pNOP324899
PADTTLVAAPHPTPIGAAEDGEWRHPI

SEQ ID NO: 341 - protein, 26 aa, Artificial Sequence
  pNOP334374
GLTCFPTTGGLAHVPAAGGVTPVATT

SEQ ID NO: 342 - protein, 26 aa, Artificial Sequence
  pNOP336175
KGTEGYFRGEESRPAGCLAYTPSQSD

SEQ ID NO: 343 - protein, 25 aa, Artificial Sequence
  pNOP352206
MASPHLKSWGSTPRMLPLPGIVKGH

SEQ ID NO: 344 - protein, 23 aa, Artificial Sequence
  pNOP376012
ARQPLDGLRWHHALHPHNPHHGG

SEQ ID NO: 345 - protein, 22 aa, Artificial Sequence
  pNOP408074
VTRRHHPRRCPPPHPHRCSRRW

SEQ ID NO: 346 - protein, 21 aa, Artificial Sequence
  pNOP412059
ELLSLSPLSQSPGRSDYPLRC

SEQ ID NO: 347 - protein, 88 aa, Artificial Sequence
  pNOP44778
ALSPWALYSSFSSSSSCNSNSNFSSSSSSSYNSNSNFSSNSFNSSNSSSSFNNSSSNSFNSSNSSYNSNSNNNSSSFNSSSNSSRWAF

SEQ ID NO: 348 - protein, 19 aa, Artificial Sequence
  pNOP465144
TQPFLQRPLRGPLHIREGR

SEQ ID NO: 349 - protein, 18 aa, Artificial Sequence
  pNOP483870
RTLPAPFPLGTFSCQSPY

SEQ ID NO: 350 - protein, 18 aa, Artificial Sequence
  pNOP487229
VAQEDPPCWKSLSSRVGL

SEQ ID NO: 351 - protein, 17 aa, Artificial Sequence
  pNOP490058
APVGGPPKRGDATAAPT

SEQ ID NO: 352 - protein, 16 aa, Artificial Sequence
  pNOP513338
AVRPFLQLGWAGQALD

SEQ ID NO: 353 - protein, 15 aa, Artificial Sequence
  pNOP548811
LTIVRCWDSYQRRQS

SEQ ID NO: 354 - protein, 15 aa, Artificial Sequence
  pNOP558727
TGGPAAGGGARTLGP

SEQ ID NO: 355 - protein, 80 aa, Artificial Sequence
  pNOP56040
DRWQSSSNSSRVLEYRQTKLWVPSPRALCLPAATKASWSSSCPLNHPRGPRACWALPRWLCCSSSTLELWAPRALTDRCL

SEQ ID NO: 356 - protein, 13 aa, Artificial Sequence
  pNOP608986
QGTARHASLLFLS

SEQ ID NO: 357 - protein, 65 aa, Artificial Sequence
  pNOP85659
AWGTTSVPSARGAAVVPIWGAILVASADATRSPSSSTLTHHHSCGPTGPVSFGGVRVPLWCQRGQ

SEQ ID NO: 358 - protein, 57 aa, Artificial Sequence
  pNOP109806
EAPKLSISEHPILGPCPYSSNSNNCGSNNRQQQQPPCDLPCQLAFHQLLDLNLAAKP

SEQ ID NO: 359 - protein, 56 aa, Artificial Sequence
  pNOP116135
WGSQMRLSCTRWRLRKFQNLNAQPWNPVPPVLSLPQWGTFPAPPPALPQPWMTSLA

SEQ ID NO: 360 - protein, 55 aa, Artificial Sequence
  pNOP118804
PSRRAVGGRRMSGKWQSLWSSLAQPCDLTRYRETCVAAVSVMRRVTGPLMGLPVC

SEQ ID NO: 361 - protein, 55 aa, Artificial Sequence
  pNOP118816
PTGPTSPHSPAARGTGQPAPRCCPHHFHWQPHYPRRLVYLCGRVPEAAGGLGAWP

SEQ ID NO: 362 - protein, 53 aa, Artificial Sequence
  pNOP127343
SGPCKIIQGHNLPNQDLSSSLGRVCLGLESCLRWVSFEHSSKESWPKTHSCGT

SEQ ID NO: 363 - protein, 50 aa, Artificial Sequence
  pNOP137386
CSVAWLYPEEPTRHLEPPETGEPRPRATHSAQLYLQCLQSGCATALGPTS

SEQ ID NO: 364 - protein, 49 aa, Artificial Sequence
  pNOP142770
GPQKPREMEAQKGRNSPHRRKEMMVQILQMKNPVASRAKPIHQDLRMGA

SEQ ID NO: 365 - protein, 49 aa, Artificial Sequence
  pNOP143520
LCLLPALRGKACGACCTSRAGAHEGERARAPVLSLRRCVADRNWHGLAA

SEQ ID NO: 366 - protein, 49 aa, Artificial Sequence
  pNOP144316
PNRAGEATAAPATTRAADSAADPAQHPAAGEGNSCSSCRSSGASRQLGC

SEQ ID NO: 367 - protein, 49 aa, Artificial Sequence
  pNOP144483
PVRLTDRPYISAFPRSQGHWAARPPLLPPPFSLAAPLPPPACLPLRTGS

SEQ ID NO: 368 - protein, 47 aa, Artificial Sequence
  pNOP152835
GRSAQDPLPLWSLELSEMDELRSFEATRQGSPPTHNLFPERDEGEER

SEQ ID NO: 369 - protein, 46 aa, Artificial Sequence
  pNOP161094 alt splice b
SSGERFQQLTKPPTCKRPKITGQLTASTRCRSRLRARSTSRPRWAT

SEQ ID NO: 370 - protein, 45 aa, Artificial Sequence
  pNOP165656
QRIPYFLPKTTHGGTACSLLEVQGVPGVPGLWGGLSRTESQLGVV

SEQ ID NO: 371 - protein, 44 aa, Artificial Sequence
  pNOP169094
GKTQPLWMGLMLRVHSQSLDRPLAVWLVNLKAPLCSWTPRSWPL

SEQ ID NO: 372 - protein, 44 aa, Artificial Sequence
  pNOP172370 alt splice e
SQLLLPLRLWLLTLIALPVRRRRKKMMTPCRIPWFSSPTQTNLS

SEQ ID NO: 373 - protein, 44 aa, Artificial Sequence
  pNOP172794
TRRGKALTLWGLTTPACPTPAPASAQLSAAAATSEASRTTAAAS

SEQ ID NO: 374 - protein, 128 aa, Artificial Sequence
  pNOP17361
RSRLVYTASPGRLCVPSSALPKKLAVSSQKLMLRSSSWLQSSRARSRNNWIRSGNSRRSTLISWQNIGTSSSNNSSSSSNNSNSTQLCWLSALPRVPGCSPSSLVSCSLAMGCSHHRGLRVGKPEVFA

SEQ ID NO: 375 - protein, 43 aa, Artificial Sequence
  pNOP174645
EEGAAEEAAAFSTVAACPAAAATAAAAFPTVCTRPCPGHVFAT

SEQ ID NO: 376 - protein, 43 aa, Artificial Sequence
  pNOP175361
GVAVPYPAAPTDAAEGARGADWCTPQVPEGSVCQAAHCQKSWP

SEQ ID NO: 377 - protein, 42 aa, Artificial Sequence
  pNOP183568
PRGSRGDLAVICRTMWQLGVARSGVLVIPPSLVPTRPLLLRE

SEQ ID NO: 378 - protein, 42 aa, Artificial Sequence
  pNOP185368
TRVELYCLLSNNSSSKWHLALACQQSLFNTFLALEPWVQPSS

SEQ ID NO: 379 - protein, 41 aa, Artificial Sequence
  pNOP191904 alt splice f
STPLVPKGTVTLSHRWLPPSWRHPSALHQKLTALTLSLSPL

SEQ ID NO: 380 - protein, 40 aa, Artificial Sequence
  pNOP194798
GLICAPPAGSALCFLRGSAWVHDPEPSGPPTAHARAAHAK

SEQ ID NO: 381 - protein, 40 aa, Artificial Sequence
  pNOP198849
SRSNWQCSSSWQTASSQIQTWTNLLQKISLIPLQRPRWWL

SEQ ID NO: 382 - protein, 40 aa, Artificial Sequence
  pNOP198864
SSAATVNGGCMQAVRASSQRTMWSRQPMKALTVSPASPTW

SEQ ID NO: 383 - protein, 40 aa, Artificial Sequence
  pNOP199023
SYGGPCAAPDAGRLISSWGWPARGIPHYPTWHPQTPALHT

SEQ ID NO: 384 - protein, 40 aa, Artificial Sequence
  pNOP199159 alt splice d
TISAWHWWFHGATAEIPHTHEKGACCTGGGVEWGWAARRG

SEQ ID NO: 385 - protein, 38 aa, Artificial Sequence
  pNOP211037
LKGMRRRSNSGEGARRANWRTCSLLTCRKPSLGRSCWT

SEQ ID NO: 386 - protein, 38 aa, Artificial Sequence
  pNOP214330
TGFPQKNCPRWNPRTCSSSSRMFWALNENSIWVVEPLA

SEQ ID NO: 387 - protein, 38 aa, Artificial Sequence
  pNOP215253
WSPFLLSVRHSFSIPWFPKTPLLPSALLLPYHCPFPPR

SEQ ID NO: 388 - protein, 37 aa, Artificial Sequence
  pNOP215460
AAESRPDPLCWDTGQEQPCGVAPKQAEWPHPGARVLP

SEQ ID NO: 389 - protein, 37 aa, Artificial Sequence
  pNOP217529
GPAPSHPSRDPQTSGANLGAASWEGLTCCCPACRYLV

SEQ ID NO: 390 - protein, 37 aa, Artificial Sequence
  pNOP217538
GPFCSWGGPAKLWTRDPKSQGRWRLRKEGTPHIAERR

SEQ ID NO: 391 - protein, 37 aa, Artificial Sequence
  pNOP218359
ITARGGELSKLFIPLWAPPPYGAATHDQPHWLCPIRA

SEQ ID NO: 392 - protein, 37 aa, Artificial Sequence
  pNOP218743
KSTQWLSSTLAPSFGTRWPTGGRKSTKSRIEASTCSE

SEQ ID NO: 393 - protein, 37 aa, Artificial Sequence
  pNOP220563
QGSGTLGSPRQPSRNPEARAEQPGTWASGPGEWTGGA

SEQ ID NO: 394 - protein, 37 aa, Artificial Sequence
  pNOP223482
YSSGPTAATATFWWGWIPGWPFRGLLPWQPCSSKPRT

SEQ ID NO: 395 - protein, 35 aa, Artificial Sequence
  pNOP240334
WAAGIPGWAQGHFLAVGTQLRRPPLGPREDHQLTC

SEQ ID NO: 396 - protein, 34 aa, Artificial Sequence
  pNOP248474
SPLSLSLVSRHPMGSTAILGPAPPWASLKAQTTQ

SEQ ID NO: 397 - protein, 33 aa, Artificial Sequence
  pNOP251217
CQCQFSWLRAPPGLSRPGGGWLPVHGVGGLYGC

SEQ ID NO: 398 - protein, 33 aa, Artificial Sequence
  pNOP257143
RFPSSSPQEMERSALEAASAAADHPEGQWAAGG

SEQ ID NO: 399 - protein, 33 aa, Artificial Sequence
  pNOP258695 alt splice f
STPLAVPDQSLKSSHTTNAFSHPLSHLILTTTL

SEQ ID NO: 400 - protein, 33 aa, Artificial Sequence
  pNOP259446
VGSMEGRQAWYPSRAHSQCYHRSPWAPCHLPCA

SEQ ID NO: 401 - protein, 32 aa, Artificial Sequence
  pNOP261027
CHCPLSRGLRGHAHLLEPPHQQSSLLLSLFYW

SEQ ID NO: 402 - protein, 32 aa, Artificial Sequence
  pNOP261872
EGLLWGHGRTTSSPADPQPTEWPRRILPAGKV

SEQ ID NO: 403 - protein, 31 aa, Artificial Sequence
  pNOP270434
AAAQCTERTGTWGHSVSWSGPTSETPFLPCK

SEQ ID NO: 404 - protein, 31 aa, Artificial Sequence
  pNOP276046
MPSLGTQCHQSSPFPNGGPFLPRPQPCPSPG

SEQ ID NO: 405 - protein, 31 aa, Artificial Sequence
  pNOP277209
PVLLYQLWASLSRGLPGHCSDCPQTCWLAVP

SEQ ID NO: 406 - protein, 31 aa, Artificial Sequence
  pNOP277754
RARCSVRCMPRAAKGWARDLYATQGTRAPAM

SEQ ID NO: 407 - protein, 31 aa, Artificial Sequence
  pNOP279143
SKSSSRAWRTWSSLTPLPRPCGIASLSLWLP

SEQ ID NO: 408 - protein, 30 aa, Artificial Sequence
  pNOP285042
IEQQSSSNTPHQGSYPANWFGAGQPAPVEH

SEQ ID NO: 409 - protein, 29 aa, Artificial Sequence
  pNOP302234
SPHSLGTHNSCLSNPSPSLSPALCSCSHL

SEQ ID NO: 410 - protein, 27 aa, Artificial Sequence
  pNOP318220
CPPSHQLMPSSNAWLHPWLWCPIKGIC

SEQ ID NO: 411 - protein, 27 aa, Artificial Sequence
  pNOP318964
EAQAGYRAAEQDPETTGSGPETAEGAH

SEQ ID NO: 412 - protein, 27 aa, Artificial Sequence
  pNOP323435
LNHCPGWRAVKTIYSAMGATPLWSCHS

SEQ ID NO: 413 - protein, 27 aa, Artificial Sequence
  pNOP323658
LRQDFHRRTAQDGIQGPAAALQGCSGL

SEQ ID NO: 414 - protein, 27 aa, Artificial Sequence
  pNOP325001
PDHVTTAQAAPTARTAWPPRRGRIGGF

SEQ ID NO: 415 - protein, 27 aa, Artificial Sequence
  pNOP325387
PMTISLILRTISTRSPATVEPGIVGNG

SEQ ID NO: 416 - protein, 27 aa, Artificial Sequence
  pNOP325875
PWSPGSNPPPDGQGTKHRRPSRFFRGH

SEQ ID NO: 417 - protein, 26 aa, Artificial Sequence
  pNOP341158
RSLLSPPILASLPPLAVAAQSMGRAS

SEQ ID NO: 418 - protein, 26 aa, Artificial Sequence
  pNOP343442
TWTWTCGCTSTVPFGPRRCMRPRAGH

SEQ ID NO: 419 - protein, 26 aa, Artificial Sequence
  pNOP344075
WACPSAEPGPGPVGAPQLCPLVHGGV

SEQ ID NO: 420 - protein, 25 aa, Artificial Sequence
  pNOP356926
SQARLPRLVKPLQTNHEALEKGSSS

SEQ ID NO: 421 - protein, 24 aa, Artificial Sequence
  pNOP362881
FWESQASGDSSGLQWGSGAALCSL

SEQ ID NO: 422 - protein, 24 aa, Artificial Sequence
  pNOP363170
GGPLEVGRCPLALTTIPSCLPRIT

SEQ ID NO: 423 - protein, 24 aa, Artificial Sequence
  pNOP364735
IITFFSTGGVALVSTGRVTPISCT

SEQ ID NO: 424 - protein, 24 aa, Artificial Sequence
  pNOP370861
RMMKSLLTWVWVWMWPRVMMNLAP

SEQ ID NO: 425 - protein, 95 aa, Artificial Sequence
  pNOP37587
GISEHLHRRDQHPLQQAVCALQVISVPAAAHRMEEQRVPGSLPYPGPGALCSQGPRKAHNGYRVHWHHHSERGGQPAGENLRRAESRHLHVPNKQ

SEQ ID NO: 426 - protein, 23 aa, Artificial Sequence
  pNOP378675
GAALVPSPWGTILISLAWRASPV

SEQ ID NO: 427 - protein, 23 aa, Artificial Sequence
  pNOP378896
GFQDNSSSKLACSTQQVEEAMGS

SEQ ID NO: 428 - protein, 23 aa, Artificial Sequence
  pNOP386633
RHPQCPVTLRSQAPQVKGCLALT

SEQ ID NO: 429 - protein, 23 aa, Artificial Sequence
  pNOP388467
SMKLTSGSMRSGCSIPSSSYRCS

SEQ ID NO: 430 - protein, 22 aa, Artificial Sequence
  pNOP394670
EQRAAGVCNQSHRAGPGGPGLH

SEQ ID NO: 431 - protein, 22 aa, Artificial Sequence
  pNOP404863
RTGRATCTGGPHTTHSHQIRHR

SEQ ID NO: 432 - protein, 22 aa, Artificial Sequence
  pNOP405923
SPRWRRVDATLLLANSPLLPPR

SEQ ID NO: 433 - protein, 22 aa, Artificial Sequence
  pNOP406378 alt splice f
STPLAVPDQSLKSSHTTNGPIP

SEQ ID NO: 434 - protein, 21 aa, Artificial Sequence
  pNOP410165
AVDHLLRPHLCPTCWLSPLFP

SEQ ID NO: 435 - protein, 21 aa, Artificial Sequence
  pNOP413106
GEAKLPSPCSRPHLLGSPGRP

SEQ ID NO: 436 - protein, 21 aa, Artificial Sequence
  pNOP414691
HLTKRTKSSSSPAGESPKERS

SEQ ID NO: 437 - protein, 21 aa, Artificial Sequence
  pNOP421083
QRGQNHHHLQPANPQRRGANL

SEQ ID NO: 438 - protein, 21 aa, Artificial Sequence
  pNOP421373
RASGPGGIRSSPTETLSPTGP

SEQ ID NO: 439 - protein, 21 aa, Artificial Sequence
  pNOP425823
TWPPSPRFPVGGNFHPSARPW

SEQ ID NO: 440 - protein, 90 aa, Artificial Sequence
  pNOP43053
PLGVWHYLDSLVAPSLIQLWPNSSNSNILVGLDPWLALQGASSLATLLFEASDLIQGFYRKGSCSCSSNVCSWPRNCSSSSSSNSSSSTF

SEQ ID NO: 441 - protein, 20 aa, Artificial Sequence
  pNOP438522
PAALPGTLTIPVPLTVWPKS

SEQ ID NO: 442 - protein, 19 aa, Artificial Sequence
  pNOP458695
PAPHSRWRKPWAARQWIIF

SEQ ID NO: 443 - protein, 19 aa, Artificial Sequence
  pNOP466225
VSEGRGALWADGACRASHS

SEQ ID NO: 444 - protein, 87 aa, Artificial Sequence
  pNOP46646
PASYPCSLRTCWSMRRRSCRRSSSFQHSCSLPSSSSNSSSSIPYCLHQALPRPCLCHMRALLPVWLGPNSSFPWVLQVPDSQVCPSH

SEQ ID NO: 445 - protein, 18 aa, Artificial Sequence
  pNOP468251
APERSCGRRTGSGPARPC

SEQ ID NO: 446 - protein, 18 aa, Artificial Sequence
  pNOP473253
GSWWEGKGSGRQEPRHWP

SEQ ID NO: 447 - protein, 18 aa, Artificial Sequence
  pNOP481442
QKPRSQSRAAWYLGIWTR

SEQ ID NO: 448 - protein, 18 aa, Artificial Sequence
  pNOP487911
VTVGCPHPGDTHQPSTRS

SEQ ID NO: 449 - protein, 17 aa, Artificial Sequence
  pNOP490152
AREWGFDLAWWTCSIWG

SEQ ID NO: 450 - protein, 17 aa, Artificial Sequence
  pNOP490194
ARQDGELTGSQRVTPAH

SEQ ID NO: 451 - protein, 17 aa, Artificial Sequence
  pNOP494542 alt splice g
GIAPIPPACGVTPVSTA

SEQ ID NO: 452 - protein, 17 aa, Artificial Sequence
  pNOP494543 alt splice g
GIAPVPAAGGIAPLSAA

SEQ ID NO: 453 - protein, 17 aa, Artificial Sequence
  pNOP501743 alt splice h
NPHTLQTAPYPEQHQHV

SEQ ID NO: 454 - protein, 17 aa, Artificial Sequence
  pNOP502714
PLCNPRNQGPCNVKPNH

SEQ ID NO: 455 - protein, 17 aa, Artificial Sequence
  pNOP506673
RVTHVSTTGGISSVPTI

SEQ ID NO: 456 - protein, 17 aa, Artificial Sequence
  pNOP507548
SLPASSQPAHFCSGSDQ

SEQ ID NO: 457 - protein, 17 aa, Artificial Sequence
  pNOP508277
SSQQPYEAPYPEQHQHV

SEQ ID NO: 458 - protein, 16 aa, Artificial Sequence
  pNOP512482
AGSGRVYGAAWHSLAT

SEQ ID NO: 459 - protein, 16 aa, Artificial Sequence
  pNOP513379
AWPPQSSGPGSWEVAL

SEQ ID NO: 460 - protein, 16 aa, Artificial Sequence
  pNOP513605
CGAWQRGDRGKQKTQA

SEQ ID NO: 461 - protein, 16 aa, Artificial Sequence
  pNOP514247
CSGFTARAWTDPWQFG

SEQ ID NO: 462 - protein, 16 aa, Artificial Sequence
  pNOP517078
GALYTSGRAVSNRNYP

SEQ ID NO: 463 - protein, 16 aa, Artificial Sequence
  pNOP518512
GVGPAVHHLTCALCQH

SEQ ID NO: 464 - protein, 16 aa, Artificial Sequence
  pNOP522295
LAPVSSGVPWGEPRAQ

SEQ ID NO: 465 - protein, 16 aa, Artificial Sequence
  pNOP523824
LTLLRHPPGWPGVKDT

SEQ ID NO: 466 - protein, 83 aa, Artificial Sequence
  pNOP52423
SHGRISEQAAATTAAAAATTATALSCAGSQPFPESPAAHQAPWSAAPWPWAAATTGASGWASRRSSPDPWGYGTTWTAWWPLP

SEQ ID NO: 467 - protein, 16 aa, Artificial Sequence
  pNOP526117
PICSAPIDSSAPTSAP

SEQ ID NO: 468 - protein, 16 aa, Artificial Sequence
  pNOP530549
SAEPCGSWEWPGAECW

SEQ ID NO: 469 - protein, 16 aa, Artificial Sequence
  pNOP530881
SFPHLQAPQWGRLLPS

SEQ ID NO: 470 - protein, 15 aa, Artificial Sequence
  pNOP537026
ALLLSSGGSTLSGTR

SEQ ID NO: 471 - protein, 15 aa, Artificial Sequence
  pNOP548556
LRGAQSTRAAGATAL

SEQ ID NO: 472 - protein, 15 aa, Artificial Sequence
  pNOP550374 alt splice h
NPHTLQTRFHIHYLI

SEQ ID NO: 473 - protein, 81 aa, Artificial Sequence
  pNOP55230
QQAGWAGAETTGYPQQQGGCSSKEAFDTEAQAGTEGKRQVGELPKEAAEGGRGQGQRGLAETAETGAVPAAPNGACYHRQF

SEQ ID NO: 474 - protein, 14 aa, Artificial Sequence
  pNOP563434
ARAELFCCLPAGLH

SEQ ID NO: 475 - protein, 14 aa, Artificial Sequence
  pNOP566785
EPDQQADQGGRHSP

SEQ ID NO: 476 - protein, 14 aa, Artificial Sequence
  pNOP568806
GKQGSNLSPSWRPP

SEQ ID NO: 477 - protein, 14 aa, Artificial Sequence
  pNOP569843
GVWPGLRPLTPAAL

SEQ ID NO: 478 - protein, 14 aa, Artificial Sequence
  pNOP570795
HRSPSGYRRQATGW

SEQ ID NO: 479 - protein, 14 aa, Artificial Sequence
  pNOP573651
KSQSPSTFASKVCG

SEQ ID NO: 480 - protein, 14 aa, Artificial Sequence
  pNOP575068
LLWPRGRHSPSGWD

SEQ ID NO: 481 - protein, 14 aa, Artificial Sequence
  pNOP580906
RACSPGSGCGCGQG

SEQ ID NO: 482 - protein, 14 aa, Artificial Sequence
  pNOP580931
RAGGAPQGCCLCPG

SEQ ID NO: 483 - protein, 14 aa, Artificial Sequence
  pNOP581766
RIPWPRGQSRYTRT

SEQ ID NO: 484 - protein, 14 aa, Artificial Sequence
  pNOP584053
SFLPITRYPSLPVP

SEQ ID NO: 485 - protein, 14 aa, Artificial Sequence
  pNOP588394
VRPAQPTCGRGLCP

SEQ ID NO: 486 - protein, 14 aa, Artificial Sequence
  pNOP589969
YLLTCLQRAPWSRA

SEQ ID NO: 487 - protein, 13 aa, Artificial Sequence
  pNOP591792
ATRPLTSATGLIP

SEQ ID NO: 488 - protein, 13 aa, Artificial Sequence
  pNOP594808
EKRLTCCDSSLSI

SEQ ID NO: 489 - protein, 13 aa, Artificial Sequence
  pNOP594895
ELPLSQWPLNQER

SEQ ID NO: 490 - protein, 13 aa, Artificial Sequence
  pNOP595078
EPLHRGRCGAGSR

SEQ ID NO: 491 - protein, 13 aa, Artificial Sequence
  pNOP596763
GGCISGGGSLCSV

SEQ ID NO: 492 - protein, 13 aa, Artificial Sequence
  pNOP607374 alt splice a
PGSSPHQQGAEAG

SEQ ID NO: 493 - protein, 77 aa, Artificial Sequence
  pNOP60941
ENLEGPAGLTIGVLHGRQAYGGRRAQNYVVWTRPSSQGSHSAAPTAPGSVPPSLAAHLDVHGFTTSPARLPAVPSYP

SEQ ID NO: 494 - protein, 13 aa, Artificial Sequence
  pNOP614310
SLWRLLHLQSWCP

SEQ ID NO: 495 - protein, 12 aa, Artificial Sequence
  pNOP621656
ASAWSSWSCPVH

SEQ ID NO: 496 - protein, 12 aa, Artificial Sequence
  pNOP626830
GAVPREPRPGRH

SEQ ID NO: 497 - protein, 12 aa, Artificial Sequence
  pNOP636166
MQSVPSLQETWE

SEQ ID NO: 498 - protein, 12 aa, Artificial Sequence
  pNOP637952
PACRGRRGAELS

SEQ ID NO: 499 - protein, 12 aa, Artificial Sequence
  pNOP638098
PCLVDLQHLGMS

SEQ ID NO: 500 - protein, 12 aa, Artificial Sequence
  pNOP638632
PLFSPTLTPSVP

SEQ ID NO: 501 - protein, 12 aa, Artificial Sequence
  pNOP640173
QIFTPRAWRYPH

SEQ ID NO: 502 - protein, 12 aa, Artificial Sequence
  pNOP643882
RTGPAKVNCFFH

SEQ ID NO: 503 - protein, 12 aa, Artificial Sequence
  pNOP645741
SPHLLPIPLAWG

SEQ ID NO: 504 - protein, 12 aa, Artificial Sequence
  pNOP648045
TPRYPGPRHVRP

SEQ ID NO: 505 - protein, 11 aa, Artificial Sequence
  pNOP652166
AGHWGQEGYLQ

SEQ ID NO: 506 - protein, 11 aa, Artificial Sequence
  pNOP654960
CYVDRRPCQVH

SEQ ID NO: 507 - protein, 11 aa, Artificial Sequence
  pNOP660899
GWGREGIPSAQ

SEQ ID NO: 508 - protein, 11 aa, Artificial Sequence
  pNOP663294
ISPTQAPCPAP

SEQ ID NO: 509 - protein, 11 aa, Artificial Sequence
  pNOP671528
PIPQTPLPLAG

SEQ ID NO: 510 - protein, 11 aa, Artificial Sequence
  pNOP672236
PRTFWAPNSPC

SEQ ID NO: 511 - protein, 11 aa, Artificial Sequence
  pNOP675830
RLSPGRVESHH

SEQ ID NO: 512 - protein, 11 aa, Artificial Sequence
  pNOP679479
SQTTRESRGPT

SEQ ID NO: 513 - protein, 11 aa, Artificial Sequence
  pNOP679892
SSLMQCCLAIP

SEQ ID NO: 514 - protein, 11 aa, Artificial Sequence
  pNOP682972
VGMGSPTRVRR

SEQ ID NO: 515 - protein, 11 aa, Artificial Sequence
  pNOP684498
WLRAALGWHLV

SEQ ID NO: 516 - protein, 73 aa, Artificial Sequence
  pNOP68935
PTLPATSTSHAFLYGCEQPATGRRLPSFLSASTLSWVPALTAATATTVAATTGNSSNLHAICHVSSLSINSWT

SEQ ID NO: 517 - protein, 10 aa, Artificial Sequence
  pNOP704364
MWRLPCTEDC

SEQ ID NO: 518 - protein, 10 aa, Artificial Sequence
  pNOP706242
PAESSALGEG

SEQ ID NO: 519 - protein, 10 aa, Artificial Sequence
  pNOP708910
QKLAWPCCVT

SEQ ID NO: 520 - protein, 10 aa, Artificial Sequence
  pNOP709657
QSPLPAKGQR

SEQ ID NO: 521 - protein, 10 aa, Artificial Sequence
  pNOP713389
RWCGAHGVRN

SEQ ID NO: 522 - protein, 10 aa, Artificial Sequence
  pNOP715424 alt splice e
SQLLLPLRLW

SEQ ID NO: 523 - protein, 10 aa, Artificial Sequence
  pNOP718753
TWHLRKPGDQ

SEQ ID NO: 524 - protein, 68 aa, Artificial Sequence
  pNOP78569
EHLGGGGPSFPSSGLRPVGARGPGPLPCHPPHSSGQHPSLPRYQTLWGPWPGGPWKAACHNLGKGQRK

SEQ ID NO: 525 - protein, 67 aa, Artificial Sequence
  pNOP81414
IPTRSGLRTTLSVTAVTKPREVRLSAPLLSSIPRCVADFHPQSLAIPPLTSPMLCTLHAKGSQRVGT

SEQ ID NO: 526 - protein, 65 aa, Artificial Sequence
  pNOP85855
DPGRGTDECGGCPAPRTANQVLPVPANWCHQQLQSHALPQCLPFCLCHPCQVHVLQGQDHAVSNA

SEQ ID NO: 527 - protein, 61 aa, Artificial Sequence
  pNOP98767
TAPACLRHIRAPSQARPTPPTASSLCTPSHLSTGGCAPNGRTTCTWLAPVSRAWGSMQPRT

SEQ ID NO: 528 - protein, 22 aa, Artificial Sequence
  pNOP402895
QKMILTKQIKTKPTDTFLQILR

SEQ ID NO: 529 - protein, 44 aa, Artificial Sequence
  pNOP173513
YQSRVLPQTEQDAKKGQNVSLLGKYILHTRTRGNLRKSRKWKSM

SEQ ID NO: 530 - protein, 53 aa, Artificial Sequence
  pNOP127569
SWKGTNWCNDMCIFITSGQIFKGTRGPRFLWGSKDQRQKGSNYSQSEALCVLL

SEQ ID NO: 531 - protein, 43 aa, Artificial Sequence
  pNOP175050
GFWIQSIKTITRYTIFVLKDIMTPPNLIAELHNILLKTITHHS

SEQ ID NO: 532 - protein, 32 aa, Artificial Sequence
  pNOP268063
RYIPPIQDPHDGKTSSCTLSSLSRYLCVVISK

SEQ ID NO: 533 - protein, 32 aa, Artificial Sequence
  pNOP266820
QKQKEISRGWIRLRLDLYLSKHYCYGISCRKT

SEQ ID NO: 534 - protein, 21 aa, Artificial Sequence
  pNOP421008
QPSSKRSLAETKGDIKRMDST

SEQ ID NO: 535 - protein, 40 aa, Artificial Sequence
  pNOP197013
NYSNVQWRNLQSSVCGLPAKGEDIFLQFRTHTTGRQVHVL

SEQ ID NO: 536 - protein, 27 aa, Artificial Sequence
  pNOP325196
PIFIQTLLLWDFLQKDLKAYTGTILMM

SEQ ID NO: 537 - protein, 15 aa, Artificial Sequence
  pNOP546300
KMEVYVIKKSIAFAV

SEQ ID NO: 538 - protein, 21 aa, Artificial Sequence
  pNOP410561
CLKLFQCSVAELAILSLWSAS

SEQ ID NO: 539 - protein, 15 aa, Artificial Sequence
  pNOP547556
LFPVRGAMCIIIATC

SEQ ID NO: 540 - protein, 15 aa, Artificial Sequence
  pNOP554260
RIIWIIDQWHCCFTR

SEQ ID NO: 541 - protein, 49 aa, Artificial Sequence
  pNOP143081
HQMLVTMNLIIIDILTPLTLIQRMNLLMKISIHKLQKSEFFFIKRDKTP

SEQ ID NO: 542 - protein, 13 aa, Artificial Sequence
  pNOP606239
NLSNPFVKILTNG

SEQ ID NO: 543 - protein, 10 aa, Artificial Sequence
  pNOP699983
KPLQDIQSLC

SEQ ID NO: 544 - protein, 17 aa, Artificial Sequence
  pNOP494212
GEAVLHKNSRGAVKSRG

SEQ ID NO: 545 - protein, 20 aa, Artificial Sequence
  pNOP445691
VKMTIMLQQFTVKLERDELV

SEQ ID NO: 546 - protein, 14 aa, Artificial Sequence
  pNOP571289
IHSSYQDQRKPQKK

SEQ ID NO: 547 - protein, 11 aa, Artificial Sequence
  pNOP682176
TSGTVVSQDDV

SEQ ID NO: 548 - protein, 60 aa, Artificial Sequence
  pNOP102380
WSGGEKRRRRRPRRLQLQGGGLSRLSPFPGLGTPESWSLPFYCLQHGGGGGGTSRDPGRF

SEQ ID NO: 549 - protein, 112 aa, Artificial Sequence
  pNOP25104
TSRPPPPHPPWPGLRRPPAEAAVRRIIRLLPIPLPPLPGLWLLRRSRPSRCNHPAAAAAAITRLRSRAKRRQSEGHQLPPSPEPFPSCRRSPATSSFCHLSPPFSSATGSQT

SEQ ID NO: 550 - protein, 26 aa, Artificial Sequence
  pNOP341110
RSAYTNYKSLNFFLSRGIKHHENKLE

SEQ ID NO: 551 - protein, 22 aa, Artificial Sequence
  pNOP401700
PGAGGRSGGGGGRGGCSSREGV

SEQ ID NO: 552 - protein, 81 aa, Artificial Sequence
  pNOP55619
VACHHFQGWERRRVGLSPSTASNTAAAAAAHPGTRAGFKPPVRRRRTPRGPGSGGRRRRQPFGGLFVFSPFRCRRCQASGC

SEQ ID NO: 553 - protein, 77 aa, Artificial Sequence
  pNOP61010
GEAGPVAATIQQPPQQPLPGCGPEPSGGRARGISYRQVQSHFHPAEEAPPPAASAISLLLFLQPQAPRHDSHHQRDR

SEQ ID NO: 554 - protein, 13 aa, Artificial Sequence
  pNOP612548
RSRQIQRLAVQLL

SEQ ID NO: 555 - protein, 11 aa, Artificial Sequence
  pNOP672549
PTTARTYQTLL

SEQ ID NO: 556 - protein, 11 aa, Artificial Sequence
  pNOP673116
QGISSTYFNKK

SEQ ID NO: 557 - protein, 11 aa, Artificial Sequence
  pNOP676378
RQSQPILFSKF

SEQ ID NO: 558 - protein, 11 aa, Artificial Sequence
  pNOP685797
YVHIYYIGANF

SEQ ID NO: 559 - protein, 140 aa, Artificial Sequence
  pNOP13160
AVLLMGNCQCCSPTPSTSWLRSSKSPRRTSQFLCPSRLAAGLGVRLWQCPHTRSPHPPPAMRVQTRPLRSAVLSTRHCARLSAQPTRRGPPALSPPTSPRCFLERMTACCVLTAYATTVLSVIWVLSSAQACCTWLRMGC

SEQ ID NO: 560 - protein, 43 aa, Artificial Sequence
  pNOP179558
WSRTSPCGGAKGSASAGSTSSGSLTGGNALAPTRPSASEDCWP

SEQ ID NO: 561 - protein, 22 aa, Artificial Sequence
  pNOP403156
QRGWESGCGSAHTLAALTHPQQ

SEQ ID NO: 562 - protein, 60 aa, Artificial Sequence
  pNOP100723
LLTSSFFLTMQSPIISQILLNIKPLANSGICTFEQEMSLFRKEKQMTKMMMKMGKTIRAQ

SEQ ID NO: 563 - protein, 125 aa, Artificial Sequence
  pNOP18284
ASVWSCLSRNTLSYAQKTSEMRMFLSVNHGILPKPNLLRKLNCGPCPSAQSGLSLGMCLCLWFAWPLYLQMQIKVMMRRIQTTQRTVELKTILTWKRKKKMSLWKCPMVNQVATTLSSSITMTCG

SEQ ID NO: 564 - protein, 47 aa, Artificial Sequence
  pNOP152717
GLQHQVEVHMDNRWEFWGLQGSRHHLHIPAHIQLDPLSYSSQQHPCL

SEQ ID NO: 565 - protein, 51 aa, Artificial Sequence
  pNOP136779
WVAIRQAFHLCRAQLMALLAWAACSHFTLGGLHPTIFRQVCLASRASHHRV

SEQ ID NO: 566 - protein, 90 aa, Artificial Sequence
  pNOP42654
ETSASCNIQATAGYYHSSSISTYSCSTITSSDSRINNCRLTALISASTASTCPSHNTLITSTSNRASGLATYTNNSSNGSTSTLTSRCSS

SEQ ID NO: 567 - protein, 96 aa, Artificial Sequence
  pNOP36509
CLGFKPTVCQCTAAVLSCTVSSNHILSRTDMSNSLWCDITLFTDNSTNCTELCPAKSSVYPGATDFHSSSTRSGGTASRSSDYNSCTRAASALAAI

SEQ ID NO: 568 - protein, 91 aa, Artificial Sequence
  pNOP42302
TRASPPRSSSAIAVRASCCPYGSTSTASRSPTQRCRLARAAASTATEVTFGSSEMQGHTMGFWLTKLNYLCHLSMLTDSLFLPISHCQCIL

SEQ ID NO: 569 - protein, 31 aa, Artificial Sequence
  pNOP279502
SSLRITGDWTSSGRSTKIWKTTQMCRKTWSG

SEQ ID NO: 570 - protein, 105 aa, Artificial Sequence
  pNOP29645
RRRRGGVGRRGVRPGRVRPGGTGRRGGDGGRAAAARAALGELARALPGHLLQSQSARRAARMAQLRRRAAALPNAAAWHGPPHPQLPSVYSERAMPPGCPEPSQA

SEQ ID NO: 571 - protein, 104 aa, Artificial Sequence
  pNOP30388
RRRRGGVGRRGVRPGRVRPGGTGRRGGDGGRAAAARAALGELARALPGHLLQSQSARRAARMAQLRRRAAALPNAAAWHGPPHPQLPRSPLALQRCRDTRWASG

SEQ ID NO: 572 - protein, 50 aa, Artificial Sequence
  pNOP140916
TRASPPRSSSAIAVRASCCPYGSTSTASRSPTQRCRLARAAASTATECIL

SEQ ID NO: 573 - protein, 81 aa, Artificial Sequence
  pNOP54515
ELQETGHRQVALRRSGRPPKCAERPGAADTGAHCTSTDGRLKISVETYTVSSQLLMVLMSLDLDTGLVPSLVSKCLILRVK

SEQ ID NO: 574 - protein, 29 aa, Artificial Sequence
  pNOP301515
RTAYFCQYHTASVYSERAMPPGCPEPSQA

SEQ ID NO: 575 - protein, 10 aa, Artificial Sequence
  pNOP700435
KSDASRLSGA

SEQ ID NO: 576 - DNA, 23 bp, Artificial Sequence
  sequence comprising a linker amino acid encoding sequence
  CDS (1)..(21)
ctatacaggcgaatgagattatg

SEQ ID NO: 577 - protein, 7 aa, Artificial Sequence
  Synthetic Construct
LYRRMRL

SEQ ID NO: 578 - DNA, 23 bp, Artificial Sequence
  sequence comprising a linker amino acid encoding sequence
  CDS (2)..(22)
ctatacaggcgaatgagattatg

SEQ ID NO: 579 - protein, 4 aa, Artificial Sequence
  Synthetic Construct
YTGE

SEQ ID NO: 580 - DNA, 66 bp, Artificial Sequence
  p21.3 seq
  CDS (1)..(66)
gcagttgggctccgcgccgtggagcagcagcagctccgccactcgggcgctgcccatcatcatgac

SEQ ID NO: 581 - protein, 22 aa, Artificial Sequence
  Synthetic Construct
AVGLRAVEQQQLRHSGAAHHHD

SEQ ID NO: 582 - DNA, 63 bp, Artificial Sequence
  frameshift
  CDS (1)..(63)
cagttgggctccgcgccgtggagcagcagcagctccgccactcgggcgctgcccatcatcatg

SEQ ID NO: 583 - protein, 21 aa, Artificial Sequence
  Synthetic Construct
QLGSAPWSSSSSATRALPIIM